(19) United States
(12) Patent Application Publication
(10) Pub. No.: US 2025/0340837 A1
(43) Pub. Date: Nov. 6, 2025

(54) CELLS

(71) Applicant: University of Geneva, Geneve (CH)

(72) Inventors: Francis Rousset, Geneve (CH); Pascal Senn, Geneve (CH)

(73) Assignee: University of Geneva, Geneve (CH)

(21) Appl. No.: 18/854,234

(22) PCT Filed: Apr. 6, 2023
(86) PCT No.: PCT/EP2023/059243

§ 371 Date: Oct. 4, 2024

(30) Foreign Application Priority Data
Apr. 7, 2022 (EP) 22167241.3

(51) Int. Cl. C12N 5/0797; A61K 35/30

(52) U.S. Cl. CPC C12N 5/0623; A61K 35/30; C12N 2501/105; C12N 2501/11; C12N 2501/13; C12N 2501/15; C12N 2501/235; C12N 2501/415; C12N 2501/91

(57) ABSTRACT

The present invention relates to auditory neuroprogenitor (ANPG) cells. The ANPG cells may be capable of extended self-renewal, capable of differentiating into an auditory neuron, and/or maintain self-renewal and/or differentiation capacity after freeze-thawing. The present invention also relates to methods for producing ANPG cells and uses of ANPG cells.

### FIELD OF THE INVENTION

The present invention relates to auditory neuroprogenitor (ANPG) cells. The ANPG cells may be capable of extended self-renewal, capable of differentiating into an auditory neuron, and/or maintain self-renewal and/or differentiation capacity after freeze-thawing. The present invention also relates to methods for producing ANPG cells and uses of ANPG cells.

### BACKGROUND TO THE INVENTION

Nearly 460 million individuals are affected by sensorineural hearing loss (SNHL), one of the most common human sensory disorders. Because of the lack of regenerative capacity of the sensory epithelia and spiral ganglion neurons (SGNs), SNHL is permanent. Studies aiming at otoprotection and regeneration have typically focusing on the sensory epithelium as a primary target. In the recent past, the SGNs have been more intensively studied, and damage of auditory neurons has been reported in multiple common conditions including noise-induced hearing loss (Kujawa and Liberman, 2009), presbycusis (Moser and Starr, 2016), head trauma (Vartiainen et al., 1985), or endolymphatic hydrops (Bixenstine et al., 2008).

The auditory synapse is affected in early SNHL, with a decreased number of synaptic ribbons between inner hair cells (IHCs) and auditory neurons leading to diminished sound discrimination without detectable increase in hearing thresholds (Kujawa and Liberman, 2009). This so-called hidden hearing loss can lead to tinnitus and usually precedes the complete degeneration of auditory neurons (Xiong et al., 2019). In case of SNHL, the IHCs can be bypassed by cochlear implants, which directly stimulate the SGNs. As opposed to IHC loss, the loss of SGNs cannot be compensated in humans (Michelson et al., 1973).

Regeneration of the sensory epithelium occurs naturally in lower vertebrates through proliferation and transdifferentiation of the supporting cells (Corwin and Cotanche, 1988; Ryals and Rubel, 1988). In this process, the realignment of the auditory synapse between the newly formed IHC and auditory neurons is essential and relies on the high plasticity of auditory neurons. In contrast, the mature mammalian cochlea lacks the capacity for efficient regeneration (Warchol et al., 1993). Inner ear progenitor cells are present in mammals; however, their number rapidly decreases with age (Oshima et al., 2007; Moon et al., 2018).

It has been described that stem and progenitor cells can be isolated from the inner ear and cultured as neurospheres (Oshima et al., 2009). In vitro, self-renewal has been reported for neurospheres derived from neonatal mouse and human fetal spiral ganglion cells (SGCs; Senn et al., 2007; Roccio et al., 2018). Previous studies have identified the characteristics of differentiation, neurite outgrowth, and their electrophysiological activity (Li et al., 2016). In vitro, the newly formed mature auditory neurons respond to glutamate, firing action potentials and exhibiting similar features as auditory neuron explants (Martinez-Monedero et al., 2008; Chen et al., 2012). Auditory progenitor neurospheres are therefore a useful tool for explorative studies aimed at development of cellular therapies and improvement of the efficiency of cochlear implantation.

Previous studies have been able to use the neurospheres for at least five to seven passages, but they generally rapidly lose their self-renewal abilities and reach senescence, limiting their use (Oshima et al., 2007; Senn et al., 2007). The reason of this early sentence is still poorly understood. Therefore, overcoming this barrier could have major consequences for the development of future cell therapies. Previous studies have also attempted to phenotypically characterized phoenix auditory neurons by comparing transcriptional profiles of neurospheres and differentiated neurons (Rousset et al., 2020).

Currently, there is a lack of auditory neuroprogenitor (ANPG) cells capable of extended self-renewal, capable of differentiating into an auditory neuron, and/or maintain self-renewal and/or differentiation capacity after freeze-thawing. Neither is there any characterisation of such ANPG cells, in particular regarding properties that may confer extended self-renewal, differentiation capacity, and maintenance of self-renewal and/or differentiation capacity after freeze-thawing. It is therefore desirable to identify and characterise such ANPG cells, which may have utility producing such ANPG and ANPG-like cells, treating and/or preventing various pathological conditions, as well as in research setting such as for screening therapeutics.

### SUMMARY OF THE INVENTION

The present invention relates to auditory neuroprogenitor (ANPG) cells. Advantageously, the ANPG cells may be capable of extended self-renewal, capable of differentiating into an auditory neuron, and/or maintain self-renewal and/or differentiation capacity after freeze-thawing. The present invention also relates to methods for producing ANPG cells and uses of ANPG cells.

The present inventors have phenotypically and functionally characterised novel ANPG cells and shown that such cells exhibit robust self-renewal properties (up to at least 40 passages, with greater than 10¹²-fold (i.e. in the order of trillion-fold) theoretical amplification, and with no self-renewal restrictions observed) and are superior to cells known in the prior art. At any passage, the ANPG cells of the present invention could be easily frozen, thawed, and reliably differentiated into mature auditory neurons and glial cells, expressing their phenotypic markers and exhibiting similar functional properties as native auditory neurons and human sphere-derived auditory neurons.

This new model overcomes the limits of the previously described neurospheres, including that previously described neurospheres reach the limits of their intrinsic ability to propagate and become senescent very rapidly. Thus, the newly identified and characterised cells allow high-throughput screening assays, and represent a unique tool to understand auditory neuroprogenitor self-renewal and regeneration in mammals. The present invention also allows the robust and efficient production of the ANPG cells of the invention, or cells having similar properties to ANPG cells of the invention. A further advantage of the present invention is the great reduction in animal numbers needed to produce auditory neurons and their precursors due to the ability to greatly expand the ANPG cells of the invention whilst maintaining the phenotypic and functional properties of the ANPG cells. This has major implications in 3R efforts to replace, reduce and refine the use of animals in research and therapeutic settings.

Further advantages of the cells of the invention include that, in contrast to prior art cells, extrinsic factors such as genetic transformation or conditional reprogramming of progenitors may not be required to bypass the natural self-renewal barrier of these cells. Further, genomic instability, which is a relatively frequent phenomenon following cell immortalization, is no longer an issue for the cells of the present invention. Further, the characterisation of the cells of the invention provides evidence of why auditory neuroprogenitors of the invention exhibit such high self-renewal capacity compared to previously described models, suggesting that the intrinsic properties of the cells confer the advantageous features seen.

Therefore, the present invention provides the following.
[1] An auditory neuroprogenitor (ANPG) cell, wherein the ANPG cell:
   (a) is capable of self-renewal for at least 10 passages, at least 20 passages, at least 30 passages or at least 40 passages;
   (b) is capable of differentiating into an auditory neuron and/or glial cell; and/or
   (c) maintains self-renewal and/or differentiation capacity after freeze-thawing; further wherein the ANPG cell comprises:
   (d) an overexpression of one or more genes selected from Myc, Sox2, Lgr5, Wnt7a, Wnt7b, Bmi1, Rtkn2 and a gene set out in Table 1; and/or
   (e) an underexpression of one or more genes selected from Lgr6, Frb, Nkd2, Tgfbr2, Tgfbr3, Hmox1, Cyba, Sox10, Plp1, Lpr5, dkk3, Nrf2 and/or a gene set out in Table 2.
[2] The ANPG cell according to [1] wherein the ANPG cell:
   (a) is a human or mouse cell; and/or
   (b) is isolated from a spiral ganglion, optionally wherein the spiral ganglion is from an A.B6 Tyr⁺-Cyba^{nmf333}/J (A/J) mouse; and/or
   (c) is any progenitor cell that is capable of differentiating into one or more cells of the auditory system, such as a cell selected from an auditory progenitor such as a human fetal auditory progenitor, an auditory neuron, a sensory epithelial cell, a spiral ganglion neuron (SGN), a sensory epithelium of the cochlea and/or a hair cell (HC).
[3] A stem cell or progenitor cell capable of differentiating into an ANPG cell according to [1] or [2], optionally wherein the stem cell or progenitor cell is a human or mouse cell.
[4] An ANPG cell deposited under ATCC accession number PTA-127156.
[5] The stem cell, progenitor cell or ANPG cell according to any one of [1]-[4], wherein the stem cell, progenitor cell or ANPG:
   (a) is genetically transformed; or
   (b) is not genetically transformed.
[6] A neurosphere comprising an ANPG cell according to any one of [1]-[5], optionally wherein the ANPG cell or neurosphere exhibits dose-dependent Ca²⁺ mobilisation in response to Glutamate, ATP, Ionomycin and/or Thapsigargin.
[7] A method of obtaining, maintaining and/or expanding an ANPG cell or neurosphere according to any one of [1]-[6], comprising:
   (a) differentiating the stem cell or progenitor cell of [3] into an ANPG cell or neurosphere in a first culture medium; and/or
   (b) isolating an ANPG cell from a spiral ganglion, optionally wherein the spiral ganglion is from an A.B6 Tyr⁺-Cyba^{nmf333}/J (A/J) mouse, and culturing the isolated ANPG cell in a first culture medium;
   optionally wherein the first culture medium contains
      i. a WNT agonist such as CHIR99021, and a TGFβ antagonist such as LDN193189 and/or SB431542; or
      ii. a WNT agonist such as CHIR99021, and at least one modulator selected from Isoxazole9 (ISX-9), Halofuginone, Sulfasalazine, AUDA, FPS-ZM1, CGP 57380, PD 169316, TA-02, Sorafenib Tosylate, Deguelin, Bosutinib (SKI-606), Ponatinib (AP24534), Axitinib, Sunitinib Malate, Imatinib (STI571), Dorsomorphin 2HCl, Dorsomorphin, L-Quebrachitol, A-83-01, TP0427736 HCl, SB431542, SB525334, SB505124, Galunisertib, GW788388, Pirfenidone, DMH1, LDN-212854, ML347, RepSox, K02288, Vactosertib, SD-208, LDN-214117, SIS3 HCl, LY 3200882, ITD-1, BIBF-0775, LDN-193189 2HCl, LY2109761, LY364947, L-685458, Nirogacestat, Avagacestat, Semagacestat, DAPT (GSI-IX), MK-0752, Dibenzazepine, Crenigacestat, LY411575, MDL-28170 and any combination thereof; or
      iii. at least one modulator selected from Isoxazole9 (ISX-9), Halofuginone, Sulfasalazine, AUDA, FPS-ZM1, CGP 57380, PD 169316, TA-02, Sorafenib Tosylate, Deguelin, Bosutinib (SKI-606), Ponatinib (AP24534), Axitinib, Sunitinib Malate, Imatinib (STI571), Dorsomorphin 2HCl, Dorsomorphin, L-Quebrachitol, A-83-01, TP0427736 HCl, SB431542, SB525334, SB505124, Galunisertib, GW788388, Pirfenidone, DMH1, LDN-212854, ML347, RepSox, K02288, Vactosertib, SD-208, LDN-214117, SIS3 HCl, LY 3200882, ITD-1, BIBF-0775, LDN-193189 2HCl, LY2109761, LY364947, L-685458, Nirogacestat, Avagacestat, Semagacestat, DAPT (GSI-IX), MK-0752, Dibenzazepine, Crenigacestat, LY411575, MDL-28170 and any combination thereof.
[8] The method according to [7], wherein the first culture medium contains at least one growth factor, optionally wherein:
   (a) the at least one growth factor is a mitogenic growth factor; and/or
   (b) the at least one growth factor is selected from one or more of FGF, EGF, IGF and Heparan Sulfate.
[9] The method according to [7] or [8], further comprising freezing the ANPG cell or neurosphere, and optionally thawing the frozen ANPG cell or neurosphere.
[10] A method of obtaining an auditory neuron and/or glial cell, comprising differentiating a culture of an ANPG cell or neurosphere according to any one of [1]-[9].
[11] The method according to [10], wherein the ANPG cell or neurosphere is cultured in a second culture medium, further wherein:
   (a) the concentration of a growth factor in the second culture medium is less than the concentration of the same growth factor in the first culture medium according to [7] or [8]; and/or
   (b) the second culture medium comprises leukemia inhibitory factor (LIF) and/or one or more neurotrophinin, optionally wherein the neurotrophinin is selected from one or more of nerve growth factor (NGF), brain-derived neurotrophic factor (BDNF), neurotrophin-3 (NT-3) and neurotrophin-4 (NT-4).
[12] An ANPG cell, neurosphere auditory neuron or glial cell obtainable by the method according to any one of [7]-[11], optionally wherein the ANPG cell, neurosphere or auditory neuron exhibits dose-dependent Ca²⁺ mobilisation in response to Glutamate, ATP, Ionomycin and/or Thapsigargin.
[13] The stem cell, progenitor cell, ANPG cell, neurosphere, auditory neuron or glial cell according to any one of [1]-[12], for use in treating or preventing hearing loss, preferably wherein the hearing loss is sensorineural hearing loss, more preferably wherein sensory epithelia, sensory epithelium of the cochlea, olfactory epithelium and/or spiral ganglion neurons are generated and/or regenerated.
[14] A non-human organism:
   (a) comprising a stem cell, progenitor cell, ANPG cell, neurosphere, auditory neuron or glial cell according to any one of [1]-[13]; and/or
   (b) treated with a stem cell, progenitor cell, ANPG cell, neurosphere, auditory neuron or glial cell according to any one of [1]-[13],
   optionally wherein the non-human organism is a murine organism.
[15] A method for assessing the safety, efficacy and/or toxicity of a test substance, comprising treating the stem cell, progenitor cell, ANPG cell, neurosphere, auditory neuron or glial cell according to any one of [1]-[13] or the non-human organism according to [14] with the test substance or a control, and comparing:
   (a) cell morphology, cell viability, cell proliferation, cell differentiation, and/or cell death of the stem cell, progenitor cell, ANPG cell, neurosphere, auditory neuron or glial cell treated with the test substance and treated with the control; or
   (b) morphology, behaviour, growth, locomotion, organ function, blood composition, urine composition, hearing, and/or lifespan of the non-human organism treated with the test substance and treated with the control.

### DESCRIPTION OF THE FIGURES

FIG. 1 High intrinsic propagation potential of phoenix auditory neuroprogenitors. (A) Auditory neuroprogenitors were isolated from the mouse pup spiral ganglion and cultured as single cell suspension. Upon growth factor addition (FGF 10 ng/mL, EGF 20 ng/mL, IGF 50 ng/mL, Heparan Sulfate 50 ng/mL), auditory neuroprogenitors form neurospheres that can be propagated. (B) Bright field microscopy pictures of spheres obtained from A.B6 Tyr⁺-Cyba^{nmf333}/J (A/J) mouse pups after 3 days in culture following the indicated passage. Scale bar: 500 μm.

FIG. 2 The high proliferation rate of phoenix neuroprogenitors was confirmed with systematic counting of cells up to passage 10. #1, #2, #3 stands for independent cultures from 3 different litters. G (dotted line) represents the average generation time for the culture and is equal to 5,697 days.

FIG. 3 Propagation and differentiation of phoenix auditory neuroprogenitors following freezing/thawing cycles. (A) Upon thawing, progenitors rapidly form highly propagating auditory neurospheres which can be easily differentiated into auditory neurons like cells with appropriate coating and growth factor removal. (B) Bright field microscopy pictures of phoenix neuroprogenitors as cultured at the indicated passage in ultra-low attachment flasks following freezing and thawing cycles in three different laboratories. Scale bar: 500 μm. (C) Bright field pictures and (D) BIII-tubulin staining of differentiated auditory neurons on Matrigel. Scale bar 100 μm. At any passage in each of the 3 institutions, phoenix neurospheres were differentiated into auditory neuronal cells exhibiting a majority of bipolar neurons. UNIGE: University of Geneva; UT: University of Tübingen; MUI: University of Innsbruck.

FIG. 4 Phoenix auditory neurons exhibit glutamatergic and purinergic Ca²⁺ transients. (A-C) Kinetics of Ca²⁺ transients induced by (A) Glutamate (higher trace for the higher concentration), (B) ATP (higher trace for the higher concentration), (C) Kainate in phoenix auditory neurons after 7 days of differentiation. Phoenix auditory neurons were loaded with Ca²⁺ sensitive ratiometric probe (FLUO-8) and stimulated with increasing concentrations of glutamate (0.02-50 M), ATP (0.005-10 μM) or kainate (0.02-50 μM). (D) Dose response curves were extracted from the max-min amplitude values, allowing the determination of an EC50 value (in order of highest to lowest effect: Ionomycin, ATP, Glutamate, Thapsigargin and Kainate (no effect for Kainate). (E). Ionomycin and Thapsigargin were used as positive control. Data represent the average+/−SEM of 3 independent experiments.

FIG. 5 Transcriptomic comparison of low and high stemness auditory neuroprogenitors (ANPGs) organoids. ANPGs were isolated from the mouse pup spiral ganglion and cultured as single cell suspension. Upon growth factor addition (FGF, EGF, IGF, heparan sulfate), auditory neuroprogenitor form organoids in suspension (see bright-field microscopy pictures). Following DNA staining, at passage 2 (P2), about 10% of the presenescent C57Bl/6 ANPG cells are still engaged in cell cycle. At the same passage, A/J ANPG exhibit relatively similar proportion of cycling cells (about 15%); however, whereas C57Bl/6 reach senescence (around passage 3), A/J ANPG increase their proliferation rate along passages to about 35%. This suggests the existence of a high sternness subpopulation, which is enriched with passages, in A/J derived organoids. Transcriptomic comparison was performed between low sternness ANPG organoids, obtained from C57Bl/6 (left, at passage 2) and A/J (middle, at passage 2) and high stemness phoenix ANPG obtained from A/J ANPG at passage 5 (right).

FIG. 6 Relative distance between low and high stemness ANPG organoids samples. (A) tree recapitulating distance between samples. Low propagating ANPG: S01 to S04 represent C57Bl/6 passage 2 quadruplicate, S05 to S08 represent A/J passage 2 quadruplicate. High propagating ANPGs: S10 to S12 represent A/J passage 5 triplicate. (B) Multidimensional scanning plot based on the fold changes between all the pairs of samples. (C) Heatmap showing relative gene expression level in low stemness ANPG from C57Bl/6 passage 2 (lane 1) and A/J passage 2 (lane 2) and high stemness A/J passage 5 ANPG (lane 3). The data shows relatively similar transcriptomic signature between C57BL/6 and A/J ANPG at passage 2, whereas the pattern of gene expression in A/J (phoenix) ANPG is more distant.

FIG. 7 Bulk RNAseq comparison of low (passage 2 C57Bl/6 and A/J) and high (passage 5 A/J) stemness ANPG. (A) The differentially expressed (DE) genes p-values are corrected for multiple testing error with a 5% FDR (false discovery rate) following quasi-likelihood statistical test. The correction used is Benjamini-Hochberg (BH). The Table gives the differentially expressed genes statistics (FDR5%) and the number of genes with a fold change >2. (B-D). Mean difference plots (MDplots) of expression data showing significantly differentially expressed genes with a FDR of 5%, highlighted in large dots for down and up differentially expressed genes. The horizontal lines between 0 and 5 and 0 and −5 represents the fold change 2 threshold. (B) C57Bl/6 P2 vs. A/J P2, (C) C57Bl/6 P2 vs. A/J P5 and (D) A/J P2 vs. A/J P5. (E) Venn diagrams representation of the differentially expressed genes with an FDR <5% in C57Bl/6 vs. A/J at passage 2, C57Bl/6 vs. A/J at passage 5 and A/J at passage 2 vs. A/J at passage 5. Relatively similar gene expression profile could be observed between passage 2 A/J and C57Bl/6, however major changes were observed in passage 5 A/J.

FIG. 8 Gene ontologies enriched in high stemness ANPGs organoids from A/J passage 5. (A) Main relevant gene ontologies showing a significantly enrichment in phoenix ANPGs vs. C57Bl/6 and A/J ANPGs. B) Gene ontology (GO) network. Each node represents a GO term, edges are drawn when there are shared genes between two GO terms. (C-G) Heatmaps showing relative expression level of genes belonging to the cell cycle (C), cell growth (D), telomeres extension (E), Ribosomes (F) and oxidative phosphorylation (G), in low stemness ANPG from C57Bl/6 passage 2 (lane 1) and A/J passage 2 (lane 2) and high stemness A/J passage 5 ANPG (lane 3). All genes displayed are significantly differentially expressed (FDR<5%) between low stemness (C57Bl/6 and A/J ANPGs) and phoenix ANPGs.

FIG. 9 Gene ontologies enriched in low stemness ANPG organoids from C57Bl/6 and A/J passage 2. (A) Main relevant gene ontologies showing a significantly enrichment in C57Bl/6 and A/J ANPGs vs. phoenix ANPGs. (B) Gene ontology network. Each node represents a GO term, edges are drawn when there are shared genes between two GO terms. (C-H) Heatmaps showing relative expression level of genes related to the peripheral nervous system development (C), gliogenesis (D), NADPH oxidase (E), TGF-b pathway (F), WNT pathway and TYROBP pathway (H) in low stemness ANPG from C57Bl/6 passage 2 (lane 1) and A/J passage 2 (lane 2) and high stemness A/J passage 5 ANPG (lane 3). All genes displayed are significantly differentially expressed (FDR<5%) between low stemness (C57Bl/6 and A/J ANPGs) and phoenix ANPGs.

FIG. 10 Molecular targets for stemness induction in low propagating ANPGs. Bar graphs showing the expression level of a non exhaustive number of candidate genes, differentially expressed in passage 5 A/J vs. passage 2 A/J and C57Bl/6 derived organoids and thus possibly involved in A/J ANPG stemness, (CPM=count per million reads). This suggests that inducing expression (rescue) of candidate genes from panel (A) or alternatively knocking down the candidate genes from panel (B) or a combination of the two represents an approach to induce stemness in low propagating ANPGs. In each graphs, left bar is Bl6, middle bar is AJ p2, and right bar is AJ p5.

FIG. 11 Gene ontology network of low propagation ANPGs and phoenix ANPGs and derived neurons. Networks built from differentially regulated gene ontologies between low stemness (C57Bl/6 and A/J p2), high stemness (A/J p5) and derived auditory neurons. Each node represents a GO term, edges are drawn when there are shared genes between two GO terms.

FIG. 12 Workflow for the organotypic spiral ganglion explant culture and the in vitro phoenix sphere derived neuron assays. The upper row shows spiral ganglion explant (SGE) cultures from 7-day old NMRI mouse pups in 96 well plates, cultured in presence of NT-3 and BDNF alone or in combination for 96 h. After fixation, specimens were stained with DAPI and for β-III-tubulin (TuJI antibody) to address the effect of neurotrophins on neurite outgrowth and neuron numbers, quantified with a modified Sholl analysis routine. For each explant, the neurite length index (NLI) was computed, and associated intersection counts served as approximation for neuron numbers. The lower rows show phoenix auditory neuroprogenitor sphere dissociation and induction of differentiation. To assess the outgrowth morphology, cells were seeded at 7,000 cells/well with presence of NT-3 or BDNF for 72 h. Differentiated cells were stained for β-III-tubulin, GFAP and DAPI for morphological analysis. Parameters such as the total neurite outgrowth, the number of neurons, the number of glial cells and the mean neurite length were assessed using a custom-made Imaris procedure. For a functional evaluation by means of a calcium response assay to glutamate, 30,000 cells were seeded per well and differentiated with supplementation of BDNF, NT-3 or combinations thereof. Via a fluorescent calcium binding dye, the cellular response to glutamate was quantified.

FIG. 13 Effect of BDNF and NT-3 alone or in combination on organotypic spiral ganglion explant culture morphology. Spiral ganglion explants (SGE) of freshly dissected P7 old mouse pups were seeded in poly-D-lysin and laminin coated 96 well plates and incubated without (n=94) or with BDNF, NT-3 or combinations of these for 96 h. Neurite outgrowth (A-C) and intersections (D-F) were quantified with representative explant micrographs underneath (G) for the given treatments. Numbers indicate the amount of neurotrophin added in ng/ml. (A-C) With BDNF, NT-3 or combinations thereof stimulated neurite outgrowth was significantly enhanced compared to spontaneous neurite outgrowth in P7 SGEs. In controls, a substantial outgrowth with a mean NLI of 20828 (±5647/4442; n=94), was registered. The back transformed mean log values rose significantly with rising BDNF (A) or NT-3 (B) content. A further increase could be observed when combining 25 ng/mL BDNF and 50 ng/mL NT-3 (C). (D-F) Bar graphs show the intersection quantification, representing the approximated number of neurons with outgrowth. Neuron numbers rose significantly in a dose-dependent manner upon addition of BDNF (D), NT-3 (E) or combinations thereof. (A-F) Bars represent geometric means in μm; error bars indicate ±upper and lower 95% confidence intervals for the mean. The shaded area in the bottom section of each graph represents control levels (i.e. levels for samples that were not treated with BDNF and/or NT-3). One-way ANOVA, followed by Tukey-Kramer post-hoc test. Dunn non-parametric comparison with control, *p<0.05, **p<0.01, ***p<0.001.

FIG. 14 Impact of BDNF and NT-3 supplementation on morphology of phoenix auditory neurons. Phoenix neuroprogenitors were differentiated for 72 h in presence of various concentrations of BDNF or NT-3 in 96-well plates. The effect of BDNF and NT-3 supplementation on the number of neurons (exclusively beta-III-tubulin positive cells, (A), the number of glial cells (exclusively GFAP positive cells, B), the total neurite outgrowth (C) and the mean neurite length per neuron exhibiting outgrowth (D), were assessed using a custom-made Imaris procedure. The number of neurons as well as the number of glial cells and the total outgrowth increased in general in a dose depended manner, peaking at 100 ng/mL BDNF or NT-3 respectively (A-C). On the contrary, the mean neurite length within a well slightly decreased with higher neurotrophin concentrations (D). (A-D) The untreated control is represented by the bar in the left of each of the four graphs, BDNF treated specimens in the bars in the middle of each of the four graphs, and NT-3 treated by the bars on the right of each of the four graphs. The dashed horizontal grey line marks the median of the control group. The numbers on the x-axis denote neurotrophin concentrations in ng/ml. The horizontal lines in the boxes denote the median value and the dots the mean value. Whiskers extend from minimum to maximum. Stars indicate significant differences from control (One-way ANOVA, followed by Dunnett's post-hoc test, *p<0.05, **<0.01, *p<0.001). Exemplary images of differentiated cells after 72 h in culture and immunohistochemical staining without treatment (E) and supplementation of 100 ng/mL BDNF (F) and 100 ng/mL NT-3 (G). (E-G) Scale bar: 100 μm.

FIG. 15 Impact of BDNF and NT-3 alone or in combination on phoenix auditory neurons' glutamatergic function. Phoenix neuroprogenitors were plated on poly-ornithine and laminin coated 96 well plates and differentiated for 7 days with or without BDNF and NT-3 alone or in combination. (A) To assess effect of neurotrophins, the auditory neurons were stimulated twice with glutamate: first with 10 μM (EC90) and 10 min later with 100 μM, eliciting a maximal glutamatergic response. Traces show the Ca2+ mobilization kinetics with 50 ng/mL NT-3+25 ng/mL BDNF (top light curve) or without neurotrophins (bottom dark curve) upon glutamate stimulation as recorded with Ca2+ sensitive ratiometric probe (FLUO-8). Arrows show the max-min amplitude of the response following the first (10 μM) or the second (100 μM) glutamate stimulation. (B-C) Heatmap showing the effect of BDNF and NT-3 alone or in combination on Ca2+ release (min-max amplitude) induced by 10 μM glutamate (B) and 100 μM glutamate (C). The values are expressed relatively to the Ca2+ mobilization elicited in auditory neurons differentiated in absence of neurotrophins (the dark trace in A is represented in the lower left square in B and C). Data represent the average+/−SEM of 3 independent experiments. *p<0.05; **p<0.01 with two ways ANOVA followed by Turkey's multiple comparison test versus control.

FIG. 16 Schematic comparison of different preparation and culture methods for auditory neurons and the yield of experimental units per mouse. The two most widely used methods for assays on auditory neurons are organotypic spiral ganglion explant preparations and spiral ganglion single cell dissociations. Both methods yield only a limited number of experimental units per sacrificed mouse. Roughly 10-14 SGE can be extracted from two inner ears while single cell preparations can yield, depending on seeding densities and well sizes, approximately 5-30 experimental units. On the contrary, dissociated cell preparations gathered from the A/J mouse can be cultured as spheroids, expanded and passaged, as well as stored and reused in a cell bank without the need of mutagenesis. Therefore, almost indefinite experimental units can be generated.

FIG. 17 High throughput evaluation of auditory neurogenesis in an all in one assay. Stable phoenix cell lines expressing mcherry fluorescent marker gene upon neural specific synapsin promoter can be established through lentiviral transduction. Micrograph images show representative examples of phoenix differentiated for 6 days without (control) or with neurotrophins (BDNF and NT-3). Features including total mcherry positive cell number or mcherry intensity/cell or/well can be easily extrapolated using cytation device (biotek). Density: 7500 cells/well. In each of the four graphs at the bottom, the left bar is the “control” group and the right bar is the “NT-3+BDNF” group.

FIG. 18 3D auditory neuron organoids generation from phoenix ANPGs neurospheres. (A) Phoenix ANPGs neurospheres, cultured in classical growth medium (DMEM:F12+N2+B27+ Growth Factors (FGF 10 ng/mL, EGF 20 ng/mL, IGF 50 ng/mL, Heparan Sulfate 50 ng/mL) (GF)) were transferred in differentiation medium (in absence of GF, but with leukemia inhibitory factor (LIF) 10 ng/ml, NT3 50 ng/ml, BDNF 10 ng/ml) for up to 21 days, At day 7, 14 and 21, organoids were harvested for quantitative assessment of neurogenesis gene expression (qPCR). (B-G) Bar graphs showing relative mRNA expression of (B) Tubb3, (C) Prph, (D) Trkb, (E) Gfap, (F) and S100b genes at the indicated time points. Undifferentiated neurospheres in proliferation media (growth) were used as control. Robust induction of neural marker genes (Tubb3, Prph and TrkB) as well as glial markers (Gfap and S100b) was observed after 7 days of differentiation in the 3D setup with gene expression lasting up to day 21. Results represent the average of 4 independently generated phoenix cell lines.

FIG. 19 Neurosphere growth induction in low propagating ANPGs from C57Bl/6 mouse. (A) C57Bl/6 ANPG, exhibiting a limited self-renewal capacity (typically to 2-3 passages) were treated with WNT agonist (CHIR99021; 3 μM) or TGFb antagonist (dual SMAD inhibitors; LDN193189 0.5 μM, SB431542 10 μM) aiming at replicating phoenix ANPG pattern of gene expression and subsequent stemness phenotype. The WNT and SMAD pathways were the two main differentially expressed pathways between low (C57Bl/6) and high stemness (A/J, phoenix) ANPG. (B) Picture showing representative triplicate of ANPG spheres cultured for 1 month in a 96 well plate. Phoenix cells are used as positive control. C57Bl/6: ANPGs were cultured in DMEM:F12+N2 and B27 supplements without growth factors. +GF: DMEM:F12+N2 and B27+FGF 10 ng/mL+EGF 20 ng/mL+IGF 50 ng/mL+Heparan Sulfate 50 ng/mL. GF+DS:DMEM:F12+N2 and B27+IGF+EFG+HS+FGF+LDN193189 0.5 μM+SB431542 10 μM. GF+CHIR: DMEM:F12+N2 and B27+IGF+EFG+HS+FGF+CHIR99021 3 μM. +GF+CHIR+DS: DMEM:F12+N2 and B27+IGF+EFG+HS+FGF+LDN193189 0.5 μM+SB431542 10 μM+CHIR99021 3 μM. Phoenix+GF: phoenix ANPG cultured in standard conditions (DMEM:F12+N2 and B27+IGF+EFG+HS+FGF). (C) Graph showing the average sphere area at different time points (up to 38 days). By both enhancing WNT pathway and repressing the dual smads, growth of low stemness C57Bl/6 ANPG (grey empty circles) was enhanced dramatically compared to previous “state of the art” conditions (solid squares), to a level approaching phoenix ANPGs (black lozenges). (D) Following DNA staining, flow cytometry was performed to determine the percentage of cells engaged in the cell cycle (phase S/G2M; proliferating). Whereas C57Bl/6 ANPGs cultured only in presence of growth factors (GF) were virtually senescent, ANPGs treated with WNT agonist (CHIR) and dual smad inhibitors (DS) exhibited significant (>20%) fraction of cycling ANPGs. (E) After reprogramming, ANPGs were differentiated on Matrigel coating following removal of mitogenic factors. BDNF (10 ng/mL), NT-3 (50 ng/mL) and LIF (10 ng/mL) were added during the 7 days of differentiation. Cells were fixed for immunostaining with BIII-tubulin (green) and S-100 (red), respectively staining neuron and glial cells. Representative pictures of differentiated C57Bl/6 (low stemness) and phoenix ANPGs, respectively used as negative and positive controls for stemness induction and of differentiated stemness-induced ANPGs. Stemness induction was induced by CHIR99021; 3 μM(CHIR); dual SMAD inhibitors LDN193189 0.5 μM and SB431542 10 μM (DS) and both treatments together (CHIR+DS). In every cases, treated ANPGs were able to differentiate into neurons (expressing BIII-tubulin) and glial cells (expressing S-100).

FIG. 20 Induction of self-propagation in low sternness ANPGs from C57Bl/6 mouse. (A) C57Bl/6 ANPG, exhibiting a limited self-renewal capacity (typically to 2-3 passages) were treated with WNT agonist (CHIR99021; 3 μM) or TGFb antagonist (dual SMAD inhibitors; LDN193189 0.5 μM, SB431542 10 μM) aiming at replicating phoenix ANPG pattern of gene expression and subsequent stemness phenotype. The WNT and SMAD pathways were the two main differentially expressed pathways between low (C57Bl/6) and high stemness (A/J, phoenix) ANPG. (B) Representative pictures of ANPG spheres 4 days following the first passage cultured in “standard” conditions (GF) or (C, left) in conditions to induce stemness (GF+LDN193189 0.5 μM+SB431542 10 μM+CHIR99021 3 μM). (C, right) Representative pictures of stemness induced ANPG spheres, cultured in presence of GF+LDN193189 0.5 μM+SB431542 10 μM+CHIR99021 3 μM, 4 days following the 10th passage. (D) At every passage, following dissociation with Accutase, ANPGs were counted with a fast read counting chamber. Graph showing the number of cells/passage up to passage 13. Whereas C57Bl/6 ANPGs cultured in standard conditions were not able to expand (see bottom line representing +GF; and middle line representing +GF+CHIR), stemness induced ANPGs could be expanded beyond 10 passages reaching about 2 million cells at passage 13 (see top line representing +GF+CHIR+DS). (E-F) Immunostaining for the neural otic progenitor marker Sox2 (red) and proliferation marker K176 (green) in C57Bl/6 ANPG neurospheres cultured in previously described classical conditions (DMEM:F12+N2 and B27 supplements +IGF+EFG+HS+FGF) (E) or reprogrammed with WNT agonist (CHIR99021; 3 μM) and TGFb antagonist (dual SMAD inhibitors; LDN193189 0.5 M, SB431542 10 μM). DAPI counter staining of the nuclei was also performed (blue). Reprogrammed ANPGs exhibit strong KI67 staining demonstrating important proliferation.

FIG. 21 Phenotypical characterization of stemness induced Auditory Neurons (ANs). (A) C57Bl/6 ANPG, were treated with WNT agonist (CHIR99021; 3 μM) or TGFb antagonist (dual SMAD inhibitors; LDN193189 0.5 μM, SB431542 10 μM) aiming at replicating phoenix ANPG stemness phenotype. After 38 days of induction, stemness-induced ANPGs were differentiated on Matrigel coating following removal of mitogenic factors. BDNF (10 ng/mL), NT-3 (50 ng/mL) and LIF (10 ng/mL) were added during the 7 days of differentiation. (B-E) Immunostainings in differentiated ANs were compared to stemness-induced ANPGs neurospheres at the same passage. (B-B′) B-III tubulin (TUJ, green) and Sox2 (red) stainings were respectively performed in (B) ANPGs and (B′) ANs. (C-C′) B-III tubulin (TUJ, green) and Nestin (Nes, red) stainings were respectively performed in (C) ANPGs and (C′) ANs. D-D′) B-III tubulin (TUJ, green) and S100 (red) stainings were respectively performed in (D) ANPGs and (D′) ANs. E-E′) B-III tubulin (TUJ) and KI67 stainings were respectively performed in (E) ANPGs and (E′) ANs. TUJ staining appears in green in ANPGs and red in ANs and KI67 appears in red in ANPGs and in green in ANs. The results demonstrate strong induction of neuronal markers (TUJ and S100) upon differentiation, whereas the expression of stem cells/neuroprogenitors markers Nestin and the proliferation marker K167 strongly decreases. Note that the expression of Sox2 also remains in some differentiating cells, suggesting that a proportion of the stemness-induced ANs are not fully mature. (F-G) Excitatory potential of stemness-induced ANs was tested using live Ca²⁺ imaging. Stemness-induced ANPGs were differentiated for 7 days in absence of growth factors. The resulting ANs cells were loaded with the Ca2+ sensitive ratiometric probe FLUO-8 and treated with glutamate 100 uM. (C) Representative picture of siANs before glutamate treatment shows no fluorescence. (D) Representative picture of siANs 1 second following glutamate addition. Upon glutamate addition, siANs exhibit robust Ca²⁺ response represented in purple. (H) Gluatamate-induced Ca²⁺ response in Phoenix and Regenix (stemness-induced ANPGs), following glutamate increments (0-100 M, ½ serial dilutions). Both phoenix and stemness-induced ANs exhibited robust Ca²+ response with EC50 within the M range. Top curve corresponds to “Regenix” and bottom curve corresponds to “Phoenix”.

FIG. 22 Neither freeze/thaw cycles nor removal of reprogramming factors affect proliferation of stemness-induced C57Bl/6 ANPGs. Following DNA staining, flow cytometry was performed to determine the percentage of cells engaged in the cell cycle (phase S/G2M; proliferating) in reprogrammed C57Bl/6 ANPGs at passage 20 (A). At passage 20 after 2 weeks removal of WNT agonist (CHIR99021; 3 μM) or TGFb antagonist (dual SMAD inhibitors; LDN1931890, 5 M, SB431542 10 μM) treatment (B). At passage 20 following one freeze and thaw cycle (C). And at passage 20 following one freeze and thaw cycle and after 2 weeks removal of WNT agonist (CHIR99021; 3 μM) or TGFb antagonist (dual SMAD inhibitors; LDN193189 0.5 μM, SB431542 10 μM) treatment (D). In any condition, a significant amount of progenitor cells are proliferating, demonstrating that stemness-induced ANPGs, like phoenix cells, can survive to freeze/thaw cycles and that reprogramming is long lasting, even following reprogramming factors withdrawal.

FIG. 23 Stemness reprogramming in C57Bl6 ANPGs. (A) Main relevant gene ontologies significantly enriched in low stemness (C57Bl/6 and A/J passage2) ANPGs vs. phoenix ANPGs. These pathways, including kinases (top bar and bottom four bars) and TGFB/Smad (second and third bards from top) are suggested to be intrinsic repressors of stemness. Therefore inhibition of these pathways has relevance to reprogram stemness. (B) Pharmacological modulators were selected to refine ANPG stemness reprogramming: CPD 8-10, 16 and 17 are “other” pathways; CPD 4-7 and 13-15 and 18-20 are kinases; CPD 11, 12 and 35-58 relate to TGFb/smad; and CPD 21-30 relate to gamma secretase. (C) C57Bl/6 ANPGs were plated at 10,000 cells/well of a 96 well plate and treated with the WNT agonist (CHIR99021; 3 μM) and the above mentioned compounds (B) for 30 days. Graph representing the average growth (% ANPGs cultured in presence of GF+CHIR) after 30 days. (D) Macroscopic overview after 30 days of progenitor sphere triplicates as cultured in 96 well plate in presence of some of the “hits” compounds or their combination. In addition to the CHIR+DS condition, “hits” for stemness reprogramming included, but were not limited to, 12, 48, 12+25 and 48+25. TGFB/Smad inhibitor families were particularly promising “hits”. Combination with compounds of the gamma secretase pathway may enhance ANPG growth. Data represent the average±SEM of three independent experiments.

FIG. 24 ANPG stemness reprogramming does not alter their ability to differentiate into auditory neuron and glial cells. (A-I) Immunostaining for the neural otic progenitor marker Sox2 (red) and Nestin (Nes; green) in stemness-induced C57Bl6/J ANPG neurospheres. (A′-I′) Immunostaining for the differentiated neuron (BIII tubulin, green) and glial cells (S100) 7 days following removal of the growth and reprogramming factors. DAPI counterstaining of the nuclei was also performed in both progenitors and differentiated cells (blue). ANPGs treated with most of the tested hits exhibit unaltered neural stem cells markers expression (Sox2 and Nestin) (A-I), whereas they express markers of neurons and glial cells upon differentiation (A′-I′). Data are representative of 3 independent experiments.

FIG. 25 Stemness-Induced (SI) ANPGs exhibits comparable transcriptomic profile as phoenix cells. The transcriptomic comparison of SI-ANPGs to low stemness ANPGs (C57Bl6 and A/J passage 2) and phoenix ANPGs was performed with the statistical analysis R/Bioconductor package edgeR 1.34.1. with a multiple testing Benjamini and Hochberg correction FDR 5% and a fold change threshold of 2, exact test. (A) The Venn diagram represents the overlap of differentially expressed genes between SI-ANPGs vs. C57Bl6 and SI-ANPGs vs. Phoenix. Whereas more than 4700 genes are differentially expressed between SI-ANPGs and C57Bl6, only about 500 are different in phoenix and thus, SI-ANPGs, originating from C57Bl6 background are transcriptionally much closer from phoenix following reprogramming. (B) Multidimensional Scale Plot (MDS) The MDS plot gives an indication of the similarity, based on the fold changes between all the pairs of samples. It shows segregation between low stemness ANPGs, namely C57Bl6 and A/J passage2 (black and red dots respectively (2 red dots in the bottom left quadrant—red dots have a lower log FC dim 1 compared to the 3 black dots; 2 red dots in the top left quadrant—red dots have a higher log FC dim 1 compared to the 1 black dot)) and high stemness ANPGs, namely phoenix and SI-ANPGs (green and blue dots respectively (3 blue dots in the bottom right quadrant—blue dots have a higher log FC dim 1 compared to the 1 green dot); 1 blue dot in the top right quadrant—blue dot has a higher log FC dim 1 than compared to the 2 green dots)).

FIG. 26 Bar graphs showing main relevant gene ontologies enriched in C57Bl/6 (A) vs. SI-ANPGs (B) and enriched in C57Bl/6 (C) vs. phoenix (D). Relatively comparable ontology profiles can be observed between phoenix and SI-ANPGs when compared to the presenescent C57Bl6ANPGs.

FIG. 27 Human fetal auditory neuroprogenitors stemness induction. (A) Human fetal ANPGs were isolated 10 weeks post abortion and were plated at 10,000 cells/well of a 96 well plate. They were treated with WNT agonist (CHIR99021; 3 μM) and/or TGFβ Smad antagonist (dual SMAD inhibitors; LDN193189 0.5 μM, SB431542 10 μM) aiming at replicating phoenix ANPG pattern of gene expression and subsequent stemness phenotype. (B) Growth of ANPG neurospheres was followed twice a week over 32 days and a macroscopic overview of some conditions of progenitors sphere triplicates is given at day 32. (C) The bar graph shows the average growth/day upon 32 days. Phoenix cells are used as positive control. Human ANPGs: ANPGs were cultured in DMEM:F12+N2 and B27 supplements without growth factors. +GF: DMEM:F12+N2 and B27+IGF+EFG+HS+FGF. +DS: DMEM:F12+N2 and B27+IGF+EFG+HS+FGF+LDN193189 0.5 μM+SB431542 10 μM. +CHIR: DMEM:F12+N2 and B27+IGF+EFG+HS+FGF+CHIR99021 3 μM. +CHIR+DS: DMEM:F12+N2 and B27+IGF+EFG+HS+FGF+LDN193189 0.5 M+SB431542 10 μM+CHIR99021 3 μM. Phoenix+GF: phoenix ANPG cultured in standard conditions (DMEM:F12+N2 and B27+IGF+EFG+HS+FGF). Data are representative from three independent experiments and show the average±SEM of three technical replicates. (D) Immunostaining for the neural otic progenitor marker Sox2 (red) and Nestin (Nes; green) in stemness-induced human fetal ANPG neurospheres (+CHIR+DS). DAPI counterstaining of the nuclei was also performed (blue). (E) Immunostaining for the differentiated neuron (BIII tubulin, green) and glial cells (S100) after 7 days removal of the growth and reprogramming factors. Human fetal stemness-induced ANPGs express markers of neural stem cells and upon differentiation markers of mature neurons and glial cells. Data are representative from 3 independent experiments. (F-H) Bright field microscopic pictures from human fetal ANPGs at passage 5, cultured in presence of GF (F), GF+CHIR (G) and GF+CHIR+DS. (H) CHIR+DS treatment allows significant expansion of the progenitors whereas ANPGs treated with GF cannot be propagated significantly.

FIG. 28 Stemness reprogramming and regeneration in C57Bl6/J hair cell progenitors. C57Bl/6 otic progenitors were plated at 10,000 cells/well of a 96 well plate and treated with WNT agonist (CHIR99021; 3 μM) and the above mentioned compounds for 30 days. (A) Graph representing the average growth (% otic progenitors cultured in presence of GF+CHIR) after 30 days. (B) Macroscopic overview after 30 days of the progenitor sphere triplicates as cultred in 96 well plate in presence of some of the “hits” compounds or their combination. In addition to the CHIR+DS condition, “hits” including 12, 51 and 12+6 are particularly good candidates for stemness reprogramming and auditory hair cells regeneration. Note that the best “hits” tended to belong to the TGFB/Smad inhibitors family. Combination with compounds of the gamma secretase pathway or kinases inhibitors may enhance hair cell progenitor growth. Data represent the average±SEM of three independent experiments.

FIG. 29 Hair cell progenitors stemness reprogramming do not alter their ability to differentiate into myosin 7a expressing auditory hair cells. (A) Immunostaining for the otic progenitor marker Sox2 (red) and Nestin (Nes; green) in stemness-induced human fetal ANPG neurospheres (+CHIR+DS). (B-I) Immunostaining for the differentiated hair cells expressing Myosin 7a (red) after 7 days removal of the growth and reprogramming factors. DAPI counterstaining of the nuclei was performed in both progenitors and differentiated cells (blue). ANPGs treated with all of the tested hits exhibit marker of auditory hair cells upon differentiation. Data are representative from 3 independent experiments.

FIG. 30 Gene-engineered stemness-induced ANPGs as high throughput platform to assess neurogenesis. Stemness-Induced ANPGs were transduced with reporter genes allowing both neurogenesis (mCherry) and gliogenesis (GFP) to be followed in living cells. Stemness-Induced ANPGs are differentiated in a 96 well plate±neurogenic compounds. Readouts include both fluorescence and functional assays on a single experimental plate.

FIG. 31 Development of an “all-in-one” high throughput assay to assess auditory neurogenesis. (A) Stemness-induced ANPGs transduced with Synapsin-mCherry and GFAP-GFP reporter genes are differentiated in a 96 well plate±NT-3 (50 ng/mL) and BDNF (10 ng/mL). (B) Bright field and (C and D) fluorescence microscopy on Stemness-induced neurospheres transduced by reporter genes. In undifferentiated ANPGs, virtually no or very few reporter gene expression can be visualized. However, upon differentiation (E), mcherry is expressed in neural cell (synapsin; red) and GFP (GFAP; green) can be visualized in glial cells. (F) Graph showing the impact of the cell number (7500-32,000 phoenix ANPGs) plated in a well of a 96 well plate on the glutamatergic function of stemness-induced differentiated with or without BDNF/NT-3 combination for 7 days. (G) Readouts also include numbering of the mCherry (G) and GFP (H) expressing cells. Upon neurotrophin treatment, significant differences are observed regarding the glutamatergic function and neurogenesis (F and G respectively), whereas virtually no impact is observed on the gliogenesis (H).

FIG. 32 “All-in-one” phoenix platform highlights combination of BDNF and NT-3 as a potent promoter of auditory neurogenesis. Phoenix ANPGs were differentiated in a 96 well plate±10 compounds with potent neurogenic activity for 7 days. The neurogenic potential of the compounds was assessed following mCherry fluorescence (expressed upon the SYN promoter). (A-C) Fluorescence microscopy pictures of phoenix cells differentiated in presence of GF (control) (A) GF+LIF (B) and GF+LIF+BDNF+NT3 (C). Glutamatergic function followed on living phoenix auditory neurons (E and F). Bar graph represent (D) the number of mcherry expressing cells/well and (E) the maximal amplitude of the glutamatergic response of phoenix ANs upon stimulation with 100 uM glutamate. (F) Ca2+ mobilization in response to glutamate increments (0.01-100 uM) allowing EC50 calculation. Data are summarized in (G). LIF: Leukemia inhibiting factor; 10 ng/mL. BDNF: Brain derived neurotrofic factor; 50 ng/mL. NT-3: Neurotrophin-3; 50 ng/mL. CNTF: Ciliary neurotrophic factor; 50 ng/mL. NGF: Nerve growth factor; 50 ng/mL. RA: Retinoic acid; 10 uM. Creatine; 5 mM. GDNF: Glial derived neurotrophic factor; 50 ng/mL. ROCKi: Rho kinase inhibitor; 10 uM. TGFB: Transforming growth factor B; 50 ng/mL. **; p<0.01. ***; p<0.001. ****; p<0.0001 (one way ANOVA+Dunnett's multiple comparison test). This experiments demonstrates that the phoenix platform can be further used for screening, including for high-throughput screening.

### DETAILED DESCRIPTION OF THE INVENTION

As used in this specification and the appended claims, the singular forms “a”, “an”, and “the” include plural references unless the content clearly dictates otherwise. Thus, for example, reference to “an ANPG cell” includes “ANPG cells”.

### Cells and Tissues

Provided is an auditory neuroprogenitor (ANPG) cell, a neurosphere comprising the ANPG cells, an auditory neuron and a glial cell. An ANPG cell is any progenitor cell that is capable of differentiating into one or more cells of the auditory system, such as a cell selected from an auditory progenitor such as a human fetal auditory progenitor, an auditory neuron, a sensory epithelial cell, a spiral ganglion neuron (SGN), a sensory epithelium of the cochlea and/or a hair cell (HC). HCs are also known as auditory hair cells. A neurosphere is any 3D cell cluster, such as an organoid, comprising neural cells, and preferably is made up of at least 10%, at least 20%, at least 30%, at least 40%, at least 50%, at least 60%, at least 70%, at least 80%, at least 90%, at least 95%, or at least 99% ANPG cells. An auditory neuron is any neuron of the auditory system, such as a vestibulocochlear nerve, a cranial nerve, the vestibular nerve. The auditory neuron may have structural (e.g. markers of, and/or morphology of) characteristics of a naturally occurring auditory neuron, and/or functional characteristics of a naturally occurring auditory neuron. A glial cell is a non-neuronal cell in the central or peripheral nervous system that does not produce electrical impulses. The glial cell may have structural (e.g. markers of, and/or morphology of) characteristics of a naturally occurring glial cell, and/or functional characteristics of a naturally occurring glial cell.

The ANPG cell may be capable of self-renewal. The ANPG cell may be capable of self-renewal for at least 1 passage, at least 2 passages, at least 5 passages, at least 10 passages, at least 15 passages, at least 20 passages, at least 25 passages, at least 30 passages, at least 40 passages, at least 50 passages, at least 60 passages, at least 70 passages, at least 80 passages, at least 90 passages, at least 100 passages, or over an indefinite number of passages. The ANPG cell may be capable of self-renewal for 1-2 passages, 1-5 passages, 1-10 passages, 1-15 passages, 1-20 passages, 1-25 passages, 1-30 passages, 1-40 passages, 1-50 passages, 1-60 passages, 1-70 passages, 1-80 passages, 1-90 passages, 1-100 passages, 2-5 passages, 2-10 passages, 2-15 passages, 2-20 passages, 2-25 passages, 2-30 passages, 2-40 passages, 2-50 passages, 2-60 passages, 2-70 passages, 2-80 passages, 2-90 passages, 2-100 passages, 5-10 passages, 5-15 passages, 5-20 passages, 5-25 passages, 5-30 passages, 5-40 passages, 5-50 passages, 5-60 passages, 5-70 passages, 5-80 passages, 5-90 passages, 5-100 passages, 10-15 passages, 10-20 passages, 10-25 passages, 10-30 passages, 10-40 passages, 10-50 passages, 10-60 passages, 10-70 passages, 10-80 passages, 10-90 passages, 10-100 passages, 15-20 passages, 15-25 passages, 15-30 passages, 15-40 passages, 15-50 passages, 15-60 passages, 15-70 passages, 15-80 passages, 15-90 passages, 15-100 passages, 20-25 passages, 20-30 passages, 20-40 passages, 20-50 passages, 20-60 passages, 20-70 passages, 20-80 passages, 20-90 passages, 20-100 passages, 25-30 passages, 25-40 passages, 25-50 passages, 25-60 passages, 25-70 passages, 25-80 passages, 25-90 passages, 25-100 passages, 30-40 passages, 30-50 passages, 30-60 passages, 30-70 passages, 30-80 passages, 30-90 passages, 30-100 passages, 40-50 passages, 40-60 passages, 40-70 passages, 40-80 passages, 40-90 passages, 40-100 passages, 50-60 passages, 50-70 passages, 50-80 passages, 50-90 passages, 50-100 passages, 60-70 passages, 60-80 passages, 60-90 passages, 60-100 passages, 70-80 passages, 70-90 passages, 70-100 passages, 80-90 passages, 80-100 passages, or 90-100 passages. Cells may be maintained in culture with proliferation media in ultra-low attachment plates. Passaging may be carried out as necessary, for example twice a week, for example using enzymatic digestion with Accutase™ followed by mechanical dissociation as previously described (Oshima et al., 2009).

The ANPG cell may substantially maintain its self-renewal and/or differentiation capacity after freeze-thawing, for example after at least 1, at least 2, at least 3, at least 4, at least 5, at least 6, at least 7, at least 8, at least 9 or at least 10 freeze-thaw cycle. A single freeze-thaw cycle involves: (i) freezing the ANPG cell using standard methodologies known in the art (e.g. freezing the ANPG cell in an appropriate buffer to at least −4° C., at least −20° C., at least −70° C. or at least −195° C.); (ii) optionally storing the frozen cell, for example for at least 1 day, at least 1 week, at least 1 month, at least 1 year, at least 5 years, at least 10 years or at least 20 years); and (iii) thawing the frozen ANPG cell using standard methodologies known in the art, for example thawing the cell to about 25° C., about 32° C. or about 37° C. Before and/or after a freeze-thaw cycle, cells may be maintained in culture with proliferation media in ultra-low attachment plates. Passaging may be carried out as necessary, for example twice a week, for example using enzymatic digestion with Accutase™ followed by mechanical dissociation as previously described (Oshima et al., 2009).

The ANPG cell may be differentiated from a stem cell or a progenitor cell. Accordingly, also provided is a stem cell or a progenitor cell capable of differentiating into an ANPG cell. Differentiation may be achieved via direct conversion of the stem and/or progenitor cell into the ANPG cell. Alternatively, differentiation may be achieved by conversion of the stem and/or progenitor cell via one or more intermediate cell types. At least 1%, at least 10%, at least 25%, at least 50%, at least 75%, at least 80%, at least 90%, at least 95% or at least 99% of the stem cell and/or progenitor cell may be converted into ANPG cells. Differentiation may be performed following passaging by seeding 200,000 or 32,000 cells to be differentiated such as neuroprogenitors cells, respectively, in a 6- or 96-well plate coated with Matrigel (1:100 dilution, hESC qualified, Corning, Sigma-Aldrich). Differentiation may be carried out after withdrawal of mitogenic growth factors in presence of differentiation medium (DMEM/F12, N2, B27, penicillin streptomycin, LIF 10 ng/ml, NT3 25 ng/ml, BDNF 10 ng/ml) for 1-7 days. The differentiation medium may be changed twice a week.

The stem cell may be any undifferentiated or partially differentiated cell that can differentiate into various types of cells and/or are generally able to proliferate to produce more of the same stem cell in a process termed self-renewal. The stem cell may be totipotent, which means that the stem cell can differentiate into embryonic and extraembryonic cell types. Such cells can construct a complete, viable organism. The stem cell may be totipotent-like, meaning that it may be able to differentiate into embryonic and some extraembryonic cell types. The stem cell may be pluripotent, which means that the stem cell can differentiate into all intra-embryonic cells derived from any of the three germ layers (i.e. cells derived from endoderm, ectoderm and mesoderm). The stem cell may be multipotent, meaning that it can differentiate into a number of cell types, typically cell types of a closely related family of cells, such as blood cells. The stem cell may be oligopotent, meaning that it can differentiate into only a few cell types, and typically fewer cell types than multipotent stem cells. The stem cell may be unipotent, meaning that it can differentiate into only one cell type.

As used herein, “stem cell” is also intended to encompass a progenitor cell, which is a cell that tends to differentiate into a specific type of cell. The differentiation capacity of a progenitor cells tends to be more limited than the differentiation capacity of a stem cell. A progenitor cell may or may not be capable of self-renewal. The stem cell may be able to differentiate into at least one type of cell, at least two types of cells, at least 5 types of cells, at least 10 types of cells, at least 20 types of cells, at least 50 types of cells or at least 100 types of cells. A population of stem cells (i.e. a plurality of stem cells) may be used and may comprise only one of the types of stem cells described herein (i.e. a homogenous population of stem cells), or two or more of the types of stem cells described herein (i.e. a heterogeneous population of stem cells).

The stem cell may express one or more markers of pluripotency. For example, the stem cell may express one or more markers including Oct4, Nanog, SSEA-1, SSEA-4, SOX2, TRA-1-60, STELLA and AP. The stem cell may have tri-germ layer differentiation capacity, meaning that the stem cell is able to differentiate into the cells of the ectodermal, endodermal and mesodermal lineages. The stem cell may be cultured and/or maintained as a colony of stem cells. Methods for culturing and/or maintaining suitable stem cells will be apparent to the skilled person.

The stem cell may be obtained from an isolated cell or tissue. The isolated cell or tissue may be any suitable cell or tissue such as a stem cell as described herein, a progenitor cell, and/or a somatic cell, or a tissue containing such cells. Examples of somatic cells include blood cells (such as monocytes, macrophages, neutrophils, basophils, eosinophils, erythroblasts, megakaryocytes, dendritic cells, T-cells, B-cells and NK-cells), neurons, neuroglial cells, skeletal muscle cells, cardiac muscle cells, smooth muscle cells, chondrocytes, osteoblasts, osteoclasts, osteocytes, fibroblasts, keratinocytes, melanocytes, endothelial cells, epithelial cells, including for example urinary or cheek cells, and adipocytes. A single isolated cell or a population of isolated cells may be used. A population of isolated cells may be made up of a single type of cell set out herein (i.e. a homogenous starting cell population), or two or more of the types of cells described herein (i.e. a heterogeneous starting cell population).

The stem cell may be an induced pluripotent stem cell. An induced pluripotent stem cell is a cell induced to have pluripotency by reprogramming a somatic cell by a known method. Specifically, a cell induced to have pluripotency may be obtained by reprogramming somatic cells such as fibroblast, skin cell, peripheral blood mononuclear cell and the like by the introduction of any combinations of a plurality of reprogramming factors selected from genes such as Oct3/4, Sox2, Klf4, Myc (c-Myc, N-Myc, L-Myc), Glis1, Nanog, Sall4, lin28, Esrrb and the like. Examples of preferable combination of reprogramming factors can include: Oct3/4, Sox2, Klf4, and Myc (c-Myc or L-Myc); and Oct3/4, Sox2, Klf4, Lin28 and L-Myc. Other suitable combinations of reprogramming factors would be known the skilled person. It is also possible to obtain and use established induced pluripotent stem cell lines. While the somatic cell used for obtaining induced pluripotent stem cell is not particularly limited, fibroblast, blood-lineage cell (e.g., peripheral blood mononuclear cell or T cell, cord blood-derived cell) and the like can be used. As the fibroblast, those derived from dermis and the like can be used.

When an induced pluripotent stem cell is produced by reprogramming by the expression of several kinds of reprogramming factors, the means for gene expression is not particularly limited. Gene transfer or direct injection of protein, which are methods well known to the skilled person, can be used. Specific examples of the aforementioned gene transfer methods include an infection method using a virus vector (e.g., retrovirus vector, lentivirus vector, Sendaivirus vector, adenovirus vector, adeno-associated virus vector), a calcium phosphate method, lipofection method, RetroNectin method, electroporation method, each using a plasmid vector (e.g., plasmid vector, episomal vector) or RNA vector, and the like.

Any of the cells or tissues set out herein (for example: the ANPG cell; stem cell, pluripotent cell or progenitor cell capable of differentiating into an ANPG cell; neurosphere and/or organoid comprising an ANPG cell; auditory neuron; glial cell; sensory epithelial cell; SGN; sensory epithelium of the cochlea; and/or a HC) may be a mammalian cell or tissue, such as rodent, bovine, caprine, equine, ovine, porcine, leporine, canine, feline, non-human primate and human cell or tissue. Preferably, the cell or tissue is a human cell, or a mouse cell or tissue, such as a cell or tissue isolated from the A.B6 Tyr⁺-Cyba^{nmf333}/J (A/J) mouse. In one preferred embodiment, the ANPG cell may be isolated from a spiral ganglion of any one of the aforementioned mammals. By “isolated cell” or “isolated tissue” it is to be understood that the population of cells or tissue have been previously removed from the organism. The isolated cell or tissue may be cultured, stored and/or manipulated ex vivo and/or in vitro using standard techniques known in the art, prior to being used in the invention. Alternatively, the isolated cell or tissue may be used directly in the method of the invention.

By way of example, the ANPG cell may be a human or mouse cell. The ANPG cell may be isolated from the spiral ganglion of a human, or a mouse such as an A/J mouse. In one specific embodiment, the ANPG cell may be an ANPG cell deposited on 19 Nov. 2021 by Pascal SENN and Francis ROUSSET, University of Geneva, 1 Michel Servet, Geneva, Switzerland 1211, with the American Type Culture Collection (ATCC) located at 10801 University Boulevard, Manassas, Virginia, 20110-2209 USA under the terms of the Budapest Treaty and accorded the ATCC accession number PTA-127156. Alternatively, the ANPG cell may be an ANPG cell derived from the cell deposited accorded the ATCC accession number PTA-127156. Here, “derived from” indicates that the parental ANPG cell deposited accorded the ATCC accession number PTA-127156 is modified or adapted in a such a way to substantially retain the characteristics of the ANPG cell deposited accorded the ATCC accession number PTA-127156. Such modifications or adaptions may occur spontaneously during cell culture or may be engineered, for example by genetic engineering techniques known to one of skill in the art. One of skill in the art would readily be able to determine whether or not a characteristics present in the parental ANPG cell is substantially retained in the derived ANPG cell, for example by comparing the self-renewal capacity, differentiation capacity, and/or gene expression profile of the parental and derived ANPG cell using appropriate methods known in the art.

Any of the cells set out herein (for example: the ANPG cell; stem cell, pluripotent cell or progenitor cell capable of differentiating into an ANPG cell; ANPG cells comprised in a neurosphere and/or comprised in an organoid; auditory neuron; glial cell; sensory epithelial cell; SGN; sensory epithelium of the cochlea; and/or a HC) may be free of artificially introduced and/or heterologous nucleic acid. Alternatively or in addition, any of the cells set out herein may be free of artificially introduced and/or heterologous protein. A heterologous nucleic acid is a nucleic acid that is not naturally present in the cell. A heterologous protein is a protein that is not naturally present and/or expressed in the cell. As such, in one embodiment the cells set out herein are not genetically transformed.

Alternatively, any of the cells set out herein (for example: the ANPG cell; stem cell, pluripotent cell or progenitor cell capable of differentiating into an ANPG cell; ANPG cells comprised in a neurosphere and/or comprised in an organoid; auditory neuron; glial cell; sensory epithelial cell; SGN; sensory epithelium of the cochlea; and/or a HC) may: overexpress of one or more genes selected from Myc, Sox2, Lgr5, Wnt7a, Wnt7b, Bmi1, Rtkn2; and/or underexpress one or more genes selected from Lgr6, Frb, Nkd2, Tgfbr2, Tgfbr3, Hmox1, Cyba, Sox10, Plp1, Lpr5, dkk3 and Nrf2. Alternatively, any of the cells set out herein may: overexpress of one or more genes selected from Table 1; and/or underexpress one or more genes selected from Table 2. Overexpression may be achieved by any suitable means known in the art. For example, the cells may be stably or transiently genetically transformed with the gene to be overexpressed, and/or with a construct that results in functional activation of the gene to be overexpressed. Similarly, underexpression may be achieved by any suitable means known in the art. For example, the cells may be stably or transiently genetically transformed with a construct that results in functional inactivation of the gene to be underexpressed.

Any of the cells or tissues set out herein (for example: the ANPG cell; stem cell, pluripotent cell or progenitor cell capable of differentiating into an ANPG cell; neurosphere and/or organoid comprising an ANPG cell; auditory neuron; glial cell; sensory epithelial cell; SGN; sensory epithelium of the cochlea; and/or a HC) may have substantially the same or superior functional properties compared to the same cell or tissue from an alternative source. For example, an ANPG cell isolated from a human may have the same or superior functional properties as an ANPG isolated from an A/J mouse. The ANPG cell, neurosphere comprising ANPG cells or auditory neuron of the present invention may exhibit Ca²⁺ mobilisation in response to Glutamate, ATP, Ionomycin and/or Thapsigargin. The ANPG cell, neurosphere comprising ANPG cells or auditory neuron of the present invention may exhibit a dose dependent Ca²⁺ mobilisation in response to Glutamate, ATP, Ionomycin and/or Thapsigargin. Ca²⁺ mobilization may be measured in response to several physiological stimuli, such as glutamate, ATP, kainate, Ionomycin and/or Thapsigargin, for example in cells obtained after day 7 of the differentiation protocol set out herein.

### Overexpressed and Underexpressed Genes

Any of the cells or tissues of the invention (for example: the ANPG cell; stem cell, pluripotent cell or progenitor cell capable of differentiating into an ANPG cell; ANPG cells comprised in a neurosphere and/or comprised in an organoid; auditory neuron; glial cell; sensory epithelial cell; SGN; sensory epithelium of the cochlea; and/or a HC) may overexpress and/or underexpress one or more genes. In one particular embodiment, the ANPG cell or ANPG cells comprised in a neurosphere may overexpress and/or underexpress said one or more genes. Overexpression and/or underexpression of said one or more genes may confer enhanced self-renewal capacity and/or enhanced differentiation capacity as defined herein, compared to the same cell or tissue not overexpressing and/or underexpressing the same genes. Alternatively, or in addition, overexpression and/or underexpression of said one or more genes may allow maintenance of self-renewal and/or differentiation capacity after freeze-thawing. Before and/or after a freeze-thaw cycle, cells may be maintained in culture with proliferation media in ultra-low attachment plates. Passaging may be carried out as necessary, for example twice a week, for example using enzymatic digestion with Accutase™ followed by mechanical dissociation as previously described (Oshima et al., 2009).

Whether or not a particular gene is overexpressed or underexpressed, including the level of expression or the absence of expression, may be measured by measuring pre-mRNA, mRNA, cDNA, and/or protein levels of the gene that is overexpressed or underexpressed. Any suitable method known to the skilled person may be used. For example, nucleic acid levels, such as the levels of mRNA or cDNA, may be measured by qRT-PCR and/or RNAseq. Protein levels may be measured by immunohistochemistry, immunocytochemistry, western blot, fluorescence-activated cell sorting (FACS), and/or mass spectrometry. The expression level of the gene in the cell or tissue may be compared to the expression level of the same gene in a reference cell or tissue. The reference cell or tissue may be the same or similar cell type isolated from the same species or a different specifies. Alternatively or in addition, the reference cell or tissue may be at an earlier or later passage. Alternatively or in addition, the reference cell or tissue may be the cell or tissue prior to overexpression of a gene (for example by transformation with the overexpressed gene and/or with a construct that results in functional activation of the gene to be overexpressed). Alternatively or in addition, the reference cell or tissue may be the cell or tissue prior to underexpression of a gene (for example by transformation with a construct that results in functional inactivation of the gene to be underexpression). As such, overexpression and underexpression may be determined. A gene is overexpressed when its expression level is higher in the cell or tissue of the invention compared to a reference cell or tissue. A gene is underexpressed when its expression level is lower in the cell or tissue of the invention compared to a reference cell of tissue. Gene expression may be analysed for a single cell or a population of cells. Where a population of cells is analysed, gene expression may represent the mean expression of that gene in the population of cells. The population of cells may be substantially homogenous.

A gene may be overexpressed by about 1.25 times, about 1.5 times, about 2 times, about 2.5 times, about 3 times, about 5 times, about 10 times, about 25 times, about 50 times, about 100 times, about 200 times, about 500 times, about 1000 times, about 1500 times, about 2000 times or about 5000 times. These values may be combined to obtain a range such as: 1.25-1.5 times, 1.25-2 times, 1.25-2.5 times, 1.25-3 times, 1.25-5 times, 1.25-10 times, 1.25-25 times, 1.25-50 times, 1.25-100 times, 1.25-200 times, 1.25-500 times, 1.25-1000 times, 1.25-1500 times, 1.25-2000 times, 1.25-5000 times, 1.5-2 times, 1.5-2.5 times, 1.5-3 times, 1.5-5 times, 1.5-10 times, 1.5-25 times, 1.5-50 times, 1.5-100 times, 1.5-200 times, 1.5-500 times, 1.5-1000 times, 1.5-1500 times, 1.5-2000 times, 1.5-5000 times, 2-2.5 times, 2-3 times, 2-5 times, 2-10 times, 2-25 times, 2-50 times, 2-100 times, 2-200 times, 2-500 times, 2-1000 times, 2-1500 times, 2-2000 times, 2-5000 times, 2.5-3 times, 2.5-5 times, 2.5-10 times, 2.5-25 times, 2.5-50 times, 2.5-100 times, 2.5-200 times, 2.5-500 times, 2.5-1000 times, 2.5-1500 times, 2.5-2000 times, 2.5-5000 times, 3-5 times, 3-10 times, 3-25 times, 3-50 times, 3-100 times, 3-200 times, 3-500 times, 3-1000 times, 3-1500 times, 3-2000 times, 3-5000 times, 5-10 times, 5-25 times, 5-50 times, 5-100 times, 5-200 times, 5-500 times, 5-1000 times, 5-1500 times, 5-2000 times, 5-5000 times, 10-25 times, 10-50 times, 10-100 times, 10-200 times, 10-500 times, 10-1000 times, 10-1500 times, 10-2000 times, 10-5000 times, 25-50 times, 25-100 times, 25-200 times, 25-500 times, 25-1000 times, 25-1500 times, 25-2000 times, 25-5000 times, 50-100 times, 50-200 times, 50-500 times, 50-1000 times, 50-1500 times, 50-2000 times, 50-5000 times, 100-200 times, 100-500 times, 100-1000 times, 100-1500 times, 100-2000 times, 100-5000 times, 200-500 times, 200-1000 times, 200-1500 times, 200-2000 times, 200-5000 times, 500-1000 times, 500-1500 times, 500-2000 times, 500-5000 times, 1000-1500 times, 1000-2000 times, 1000-5000 times, 1500-2000 times, 1500-5000 times, or 2000-5000 times overexpression. Where multiple genes are overexpressed, each gene may be overexpressed by the substantially the same amount or a different amount.

A gene may be underexpressed by about 1.25 times, about 1.5 times, about 2 times, about 2.5 times, about 3 times, about 5 times, about 10 times, about 25 times, about 50 times, about 100 times, about 200 times, about 500 times, about 1000 times, about 1500 times, about 2000 times or about 5000 times. These values may be combined to obtain a range such as: 1.25-1.5 times, 1.25-2 times, 1.25-2.5 times, 1.25-3 times, 1.25-5 times, 1.25-10 times, 1.25-25 times, 1.25-50 times, 1.25-100 times, 1.25-200 times, 1.25-500 times, 1.25-1000 times, 1.25-1500 times, 1.25-2000 times, 1.25-5000 times, 1.5-2 times, 1.5-2.5 times, 1.5-3 times, 1.5-5 times, 1.5-10 times, 1.5-25 times, 1.5-50 times, 1.5-100 times, 1.5-200 times, 1.5-500 times, 1.5-1000 times, 1.5-1500 times, 1.5-2000 times, 1.5-5000 times, 2-2.5 times, 2-3 times, 2-5 times, 2-10 times, 2-25 times, 2-50 times, 2-100 times, 2-200 times, 2-500 times, 2-1000 times, 2-1500 times, 2-2000 times, 2-5000 times, 2.5-3 times, 2.5-5 times, 2.5-10 times, 2.5-25 times, 2.5-50 times, 2.5-100 times, 2.5-200 times, 2.5-500 times, 2.5-1000 times, 2.5-1500 times, 2.5-2000 times, 2.5-5000 times, 3-5 times, 3-10 times, 3-25 times, 3-50 times, 3-100 times, 3-200 times, 3-500 times, 3-1000 times, 3-1500 times, 3-2000 times, 3-5000 times, 5-10 times, 5-25 times, 5-50 times, 5-100 times, 5-200 times, 5-500 times, 5-1000 times, 5-1500 times, 5-2000 times, 5-5000 times, 10-25 times, 10-50 times, 10-100 times, 10-200 times, 10-500 times, 10-1000 times, 10-1500 times, 10-2000 times, 10-5000 times, 25-50 times, 25-100 times, 25-200 times, 25-500 times, 25-1000 times, 25-1500 times, 25-2000 times, 25-5000 times, 50-100 times, 50-200 times, 50-500 times, 50-1000 times, 50-1500 times, 50-2000 times, 50-5000 times, 100-200 times, 100-500 times, 100-1000 times, 100-1500 times, 100-2000 times, 100-5000 times, 200-500 times, 200-1000 times, 200-1500 times, 200-2000 times, 200-5000 times, 500-1000 times, 500-1500 times, 500-2000 times, 500-5000 times, 1000-1500 times, 1000-2000 times, 1000-5000 times, 1500-2000 times, 1500-5000 times, or 2000-5000 times underexpression. Where multiple genes are underexpressed, each gene may be underexpressed by the substantially the same amount or a different amount.

Any of the cells or tissues of the invention (for example: the ANPG cell; stem cell, pluripotent cell or progenitor cell capable of differentiating into an ANPG cell; ANPG cells comprised in a neurosphere and/or comprised in an organoid; auditory neuron; glial cell; sensory epithelial cell; SGN; sensory epithelium of the cochlea; and/or a HC) may comprise an overexpression of one or more genes selected from Myc, Sox2, Lgr5, Wnt7a, Wnt7b, Bmi1, and Rtkn2. For example, and for reference, any of the cells or tissues of the invention may overexpress:

{Myc} {Sox2} {Lgr5} {Wnt7a} {Wnt7b} {Bmi1} {Rtkn2} {Myc,Sox2} {Myc,Lgr5} {Myc,Wnt7a} {Myc,Wnt7b} {Myc,Bmi1} {Myc,Rtkn2} {Sox2,Lgr5} {Sox2,Wnt7a} {Sox2,Wnt7b} {Sox2,Bmi1} {Sox2,Rtkn2} {Lgr5,Wnt7a} {Lgr5,Wnt7b} {Lgr5,Bmi1} {Lgr5,Rtkn2} {Wnt7a,Wnt7b} {Wnt7a,Bmi1} {Wnt7a,Rtkn2} {Wnt7b,Bmi1} {Wnt7b,Rtkn2} {Bmi1,Rtkn2} {Myc,Sox2,Lgr5} {Myc,Sox2,Wnt7a} {Myc,Sox2,Wnt7b} {Myc,Sox2,Bmi1} {Myc,Sox2,Rtkn2} {Myc,Lgr5,Wnt7a} {Myc,Lgr5,Wnt7b} {Myc,Lgr5,Bmi1} {Myc,Lgr5,Rtkn2} {Myc,Wnt7a,Wnt7b} {Myc,Wnt7a,Bmi1} {Myc,Wnt7a,Rtkn2} {Myc,Wnt7b,Bmi1} {Myc,Wnt7b,Rtkn2} {Myc,Bmi1,Rtkn2} {Sox2,Lgr5,Wnt7a} {Sox2,Lgr5,Wnt7b} {Sox2,Lgr5,Bmi1} {Sox2,Lgr5,Rtkn2} {Sox2,Wnt7a,Wnt7b} {Sox2,Wnt7a,Bmi1} {Sox2,Wnt7a,Rtkn2} {Sox2,Wnt7b,Bmi1} {Sox2,Wnt7b,Rtkn2} {Sox2,Bmi1,Rtkn2} {Lgr5,Wnt7a,Wnt7b} {Lgr5,Wnt7a,Bmi1} {Lgr5,Wnt7a,Rtkn2} {Lgr5,Wnt7b,Bmi1} {Lgr5,Wnt7b,Rtkn2} {Lgr5,Bmi1,Rtkn2} {Wnt7a,Wnt7b,Bmi1} {Wnt7a,Wnt7b,Rtkn2} {Wnt7a,Bmi1,Rtkn2} {Wnt7b,Bmi1,Rtkn2} {Myc,Sox2,Lgr5,Wnt7a} {Myc,Sox2,Lgr5,Wnt7b} {Myc,Sox2,Lgr5,Bmi1} {Myc,Sox2,Lgr5,Rtkn2} {Myc,Sox2,Wnt7a,Wnt7b} {Myc,Sox2,Wnt7a,Bmi1} {Myc,Sox2,Wnt7a,Rtkn2} {Myc,Sox2,Wnt7b,Bmi1} {Myc,Sox2,Wnt7b,Rtkn2} {Myc,Sox2,Bmi1,Rtkn2} {Myc,Lgr5,Wnt7a,Wnt7b} {Myc,Lgr5,Wnt7a,Bmi1} {Myc,Lgr5,Wnt7a,Rtkn2} {Myc,Lgr5,Wnt7b,Bmi1} {Myc,Lgr5,Wnt7b,Rtkn2} {Myc,Lgr5,Bmi1,Rtkn2} {Myc,Wnt7a,Wnt7b,Bmi1} {Myc,Wnt7a,Wnt7b,Rtkn2} {Myc,Wnt7a,Bmi1,Rtkn2} {Myc,Wnt7b,Bmi1,Rtkn2} {Sox2,Lgr5,Wnt7a,Wnt7b} {Sox2,Lgr5,Wnt7a,Bmi1} {Sox2,Lgr5,Wnt7a,Rtkn2} {Sox2,Lgr5,Wnt7b,Bmi1} {Sox2,Lgr5,Wnt7b,Rtkn2} {Sox2,Lgr5,Bmi1,Rtkn2} {Sox2,Wnt7a,Wnt7b,Bmi1} {Sox2,Wnt7a,Wnt7b,Rtkn2} {Sox2,Wnt7a,Bmi1,Rtkn2} {Sox2,Wnt7b,Bmi1,Rtkn2} {Lgr5,Wnt7a,Wnt7b,Bmi1} {Lgr5,Wnt7a,Wnt7b,Rtkn2} {Lgr5,Wnt7a,Bmi1,Rtkn2} {Lgr5,Wnt7b,Bmi1,Rtkn2} {Wnt7a,Wnt7b,Bmi1,Rtkn2} {Myc,Sox2,Lgr5,Wnt7a,Wnt7b} {Myc,Sox2,Lgr5,Wnt7a,Bmi1} {Myc,Sox2,Lgr5,Wnt7a,Rtkn2} {Myc,Sox2,Lgr5,Wnt7b,Bmi1} {Myc,Sox2,Lgr5,Wnt7b,Rtkn2} {Myc,Sox2,Lgr5,Bmi1,Rtkn2} {Myc,Sox2,Wnt7a,Wnt7b,Bmi1} {Myc,Sox2,Wnt7a,Wnt7b,Rtkn2} {Myc,Sox2,Wnt7a,Bmi1,Rtkn2} {Myc,Sox2,Wnt7b,Bmi1,Rtkn2} {Myc,Lgr5,Wnt7a,Wnt7b,Bmi1} {Myc,Lgr5,Wnt7a,Wnt7b,Rtkn2} {Myc,Lgr5,Wnt7a,Bmi1,Rtkn2} {Myc,Lgr5,Wnt7b,Bmi1,Rtkn2} {Myc,Wnt7a,Wnt7b,Bmi1,Rtkn2} {Sox2,Lgr5,Wnt7a,Wnt7b,Bmi1} {Sox2,Lgr5,Wnt7a,Wnt7b,Rtkn2} {Sox2,Lgr5,Wnt7a,Bmi1,Rtkn2} {Sox2,Lgr5,Wnt7b,Bmi1,Rtkn2} {Sox2,Wnt7a,Wnt7b,Bmi1,Rtkn2} {Lgr5,Wnt7a,Wnt7b,Bmi1,Rtkn2} {Myc,Sox2,Lgr5,Wnt7a,Wnt7b,Bmi1} {Myc,Sox2,Lgr5,Wnt7a,Wnt7b,Rtkn2} {Myc,Sox2,Lgr5,Wnt7a,Bmi1,Rtkn2} {Myc,Sox2,Lgr5,Wnt7b,Bmi1,Rtkn2} {Myc,Sox2,Wnt7a,Wnt7b,Bmi1,Rtkn2} {Myc,Lgr5,Wnt7a,Wnt7b,Bmi1,Rtkn2} {Sox2,Lgr5,Wnt7a,Wnt7b,Bmi1,Rtkn2} or {Myc,Sox2,Lgr5,Wnt7a,Wnt7b,Bmi1,Rtkn2}.

Alternatively or in addition, any of the cells or tissues of the invention (for example: the ANPG cell; stem cell, pluripotent cell or progenitor cell capable of differentiating into an ANPG cell; ANPG cells comprised in a neurosphere and/or comprised in an organoid; auditory neuron; glial cell; sensory epithelial cell; SGN; sensory epithelium of the cochlea; and/or a HC) may overexpress one or more gene involved in the cell cycle, cell growth, ribosomes, telomere extension or oxphos, as set out in Table 1. As such, the overexpressed gene may be one or more gene set out in Table 1. By way of example, the overexpressed gene may be {Cdc25c}, {Mrpl32}, {Ccne2 and Pcna}, or {Bmi1, Rtkn2, Rpl15 and Atp5g2}.

TABLE 1
Genes that may be overexpressed, grouped by ontogeny

| | | | | Telomere | | | |
|---|---|---|---|---|---|---|---|
| Cell Cycle | | | | Extension | Ribosomes | | Oxphos |
| Cdc25c | Atad5 | Cenpe | Ccng2 | Pole2 | Mrpl32 | Rps26 | Atp5g2 |
| Ccne2 | Dtl | Plk1 | Ezh2 | Prim1 | Rpl22l1 | Rpl22 | Ndufs4 |
| Trip13 | Donson | Clspn | Cenpf | Rpa3 | Rsl24d1 | Rps27a | Ndufc2 |
| E2f7 | Birc5 | Pole | Mcm5 | Rfc4 | Mrpl15 | Rps23 | Atp5e |
| Ticrr | Rad51 | Cdc25b | Ube2s | Prim2 | Mrpl3 | Rpl41 | Ndufv2 |
| Spdl1 | Brca2 | Timeless | Foxm1 | Rfc5 | Mrpl18 | Rpl14 | Atp5o |
| Fbxo5 | Dbf4 | Cdt1 | Lmnb1 | Pole | Mrpl30 | Rpl5 | |
| Chek1 | Lsm11 | Ccnf | Lig1 | Pola1 | Mrpl28 | Rpl18a | |
| Cks2 | Cdc45 | Rbl1 | Mcm3 | Pola2 | Mrpl12 | Rpl32 | |
| Cdca5 | Mastl | Rpa2 | Cdkn1b | Pole4 | Rpl35 | Rpl17 | |
| Ttk | Anapc15 | Iqgap3 | Mcm2 | Dkc1 | Rpl27 | Rpl11 | |
| Cdc6 | Aurkb | Pola1 | Apex1 | Rfc3 | Rpl10a | Rps24 | |
| Stil | Cit | Cdkn2c | Mcm6 | Pold1 | Rpl36 | Rps5 | |
| Kif14 | Ccnb1 | Bub1b | Nasp | Pold2 | Rpl27a | Rps15 | |
| Skp2 | Miip | Tipin | Mcm4 | Rfc2 | Rpl36a | Rpl9 | |
| Chek2 | Ube2c | Tacc3 | Tfdp1 | Pcna | Rps16 | Rpl7a | |
| Ndc80 | Kntc1 | Cdk1 | Topbp1 | Cell Growth | Rpl15 | Rps9 | |
| Fam83d | Brca1 | Rcc1 | Mcm7 | Bmi1 | Rps13 | Rpl3 | |
| Cdc7 | Mtbp | Ccna2 | Pcna | Rtkn2 | Rpl23a | Rpl8 | |
| Blm | Rnaseh2b | Cdkn2d | Phb2 | Myc | Rps25 | Rpsa | |
| Bub1 | Cks1b | Usp37 | Egfr | Mki67 | Rpl31 | Rpl7 | |
| | Mad2l1 | | | Pcna | | | |

Alternatively or in addition, any of the cells or tissues of the invention (for example: the ANPG cell; stem cell, pluripotent cell or progenitor cell capable of differentiating into an ANPG cell; ANPG cells comprised in a neurosphere and/or comprised in an organoid; auditory neuron; glial cell; sensory epithelial cell; SGN; sensory epithelium of the cochlea; and/or a HC) may comprise an underexpression of one or more genes selected from Lgr6, Frb, Nkd2, Tgfbr2, Tgfbr3, Hmox1, Cyba, Sox10, Plp1, Lpr5, dkk3 and Nrf2. For example, and for reference, any of the cells or tissues of the invention may underexpress: {Lgr6} {Frb} {Nkd2} {Tgfbr2} {Tgfbr3} {Hmox1} {Cyba} {Sox10} {Plp1} {Lpr5} {dkk3} {Nrf2} {Lgr6,Frb} {Lgr6,Nkd2} {Lgr6,Tgfbr2} {Lgr6,Tgfbr3} {Lgr6,Hmox1} {Lgr6,Cyba} {Lgr6,Sox10} {Lgr6,Plp1} {Lgr6,Lpr5} {Lgr6,dkk3} {Lgr6,Nrf2} {Frb,Nkd2} {Frb,Tgfbr2} {Frb,Tgfbr3} {Frb,Hmox1} {Frb,Cyba} {Frb,Sox10} {Frb,Plp1} {Frb,Lpr5} {Frb,dkk3} {Frb,Nrf2} {Nkd2,Tgfbr2} {Nkd2,Tgfbr3} {Nkd2,Hmox1} {Nkd2,Cyba} {Nkd2,Sox10} {Nkd2,Plp1} {Nkd2,Lpr5} {Nkd2,dkk3} {Nkd2,Nrf2} {Tgfbr2,Tgfbr3} {Tgfbr2,Hmox1} {Tgfbr2,Cyba} {Tgfbr2,Sox10} {Tgfbr2,Plp1} {Tgfbr2,Lpr5} {Tgfbr2,dkk3} {Tgfbr2,Nrf2} {Tgfbr3,Hmox1} {Tgfbr3,Cyba} {Tgfbr3,Sox10} {Tgfbr3,Plp1} {Tgfbr3,Lpr5} {Tgfbr3,dkk3} {Tgfbr3,Nrf2} {Hmox1,Cyba} {Hmox1,Sox10} {Hmox1,Plp1} {Hmox1,Lpr5} {Hmox1,dkk3} {Hmox1,Nrf2} {Cyba,Sox10} {Cyba,Plp1} {Cyba,Lpr5} {Cyba,dkk3} {Cyba,Nrf2} {Sox10,Plp1} {Sox10,Lpr5} {Sox10,dkk3} {Sox10,Nrf2} {Plp1,Lpr5} {Plp1,dkk3} {Plp1,Nrf2} {Lpr5,dkk3} {Lpr5,Nrf2} {dkk3,Nrf2} {Lgr6,Frb,Nkd2} {Lgr6,Frb,Tgfbr2} {Lgr6,Frb,Tgfbr3} {Lgr6,Frb,Hmox1} {Lgr6,Frb,Cyba} {Lgr6,Frb,Sox10} {Lgr6,Frb,Plp1} {Lgr6,Frb,Lpr5} {Lgr6,Frb,dkk3} {Lgr6,Frb,Nrf2} {Lgr6,Nkd2,Tgfbr2} {Lgr6,Nkd2,Tgfbr3} {Lgr6,Nkd2,Hmox1} {Lgr6,Nkd2,Cyba} {Lgr6,Nkd2,Sox10} {Lgr6,Nkd2,Plp1} {Lgr6,Nkd2,Lpr5} {Lgr6,Nkd2,dkk3} {Lgr6,Nkd2,Nrf2} {Lgr6,Tgfbr2,Tgfbr3} {Lgr6,Tgfbr2,Hmox1} {Lgr6,Tgfbr2,Cyba} {Lgr6,Tgfbr2,Sox10} {Lgr6,Tgfbr2,Plp1} {Lgr6,Tgfbr2,Lpr5} {Lgr6,Tgfbr2,dkk3} {Lgr6,Tgfbr2,Nrf2} {Lgr6,Tgfbr3,Hmox1} {Lgr6,Tgfbr3,Cyba} {Lgr6,Tgfbr3,Sox10} {Lgr6,Tgfbr3,Plp1} {Lgr6,Tgfbr3,Lpr5} {Lgr6,Tgfbr3,dkk3} {Lgr6,Tgfbr3,Nrf2} {Lgr6,Hmox1,Cyba} {Lgr6,Hmox1,Sox10} {Lgr6,Hmox1,Plp1} {Lgr6,Hmox1,Lpr5} {Lgr6,Hmox1,dkk3} {Lgr6,Hmox1,Nrf2} {Lgr6,Cyba,Sox10} {Lgr6,Cyba,Plp1} {Lgr6,Cyba,Lpr5} {Lgr6,Cyba,dkk3} {Lgr6,Cyba,Nrf2} {Lgr6,Sox10,Plp1} {Lgr6,Sox10,Lpr5} {Lgr6,Sox10,dkk3} {Lgr6,Sox10,Nrf2} {Lgr6,Plp1,Lpr5} {Lgr6,Plp1,dkk3} {Lgr6,Plp1,Nrf2} {Lgr6,Lpr5,dkk3} {Lgr6,Lpr5,Nrf2} {Lgr6,dkk3,Nrf2} {Frb,Nkd2,Tgfbr2} {Frb,Nkd2,Tgfbr3} {Frb,Nkd2,Hmox1} {Frb,Nkd2,Cyba} {Frb,Nkd2,Sox10} {Frb,Nkd2,Plp1} {Frb,Nkd2,Lpr5} {Frb,Nkd2,dkk3} {Frb,Nkd2,Nrf2} {Frb,Tgfbr2,Tgfbr3} {Frb,Tgfbr2,Hmox1} {Frb,Tgfbr2,Cyba} {Frb,Tgfbr2,Sox10} {Frb,Tgfbr2,Plp1} {Frb,Tgfbr2,Lpr5} {Frb,Tgfbr2,dkk3} {Frb,Tgfbr2,Nrf2} {Frb,Tgfbr3,Hmox1} {Frb,Tgfbr3,Cyba} {Frb,Tgfbr3,Sox10} {Frb,Tgfbr3,Plp1} {Frb,Tgfbr3,Lpr5} {Frb,Tgfbr3,dkk3} {Frb,Tgfbr3,Nrf2} {Frb,Hmox1,Cyba} {Frb,Hmox1,Sox10} {Frb,Hmox1,Plp1} {Frb,Hmox1,Lpr5} {Frb,Hmox1,dkk3} {Frb,Hmox1,Nrf2} {Frb,Cyba,Sox10} {Frb,Cyba,Plp1} {Frb,Cyba,Lpr5} {Frb,Cyba,dkk3} {Frb,Cyba,Nrf2} {Frb,Sox10,Plp1} {Frb,Sox10,Lpr5} {Frb,Sox10,dkk3} {Frb,Sox10,Nrf2} {Frb,Plp1,Lpr5} {Frb,Plp1,dkk3} {Frb,Plp1,Nrf2} {Frb,Lpr5,dkk3} {Frb,Lpr5,Nrf2} {Frb,dkk3,Nrf2} {Nkd2,Tgfbr2,Tgfbr3} {Nkd2,Tgfbr2,Hmox1} {Nkd2,Tgfbr2,Cyba} {Nkd2,Tgfbr2,Sox10} {Nkd2,Tgfbr2,Plp1} {Nkd2,Tgfbr2,Lpr5} {Nkd2,Tgfbr2,dkk3} {Nkd2,Tgfbr2,Nrf2} {Nkd2,Tgfbr3,Hmox1} {Nkd2,Tgfbr3,Cyba} {Nkd2,Tgfbr3,Sox10} {Nkd2,Tgfbr3,Plp1} {Nkd2,Tgfbr3,Lpr5} {Nkd2,Tgfbr3,dkk3} {Nkd2,Tgfbr3,Nrf2} {Nkd2,Hmox1,Cyba} {Nkd2,Hmox1,Sox10} {Nkd2,Hmox1,Plp1} {Nkd2,Hmox1,Lpr5} {Nkd2,Hmox1,dkk3} {Nkd2,Hmox1,Nrf2} {Nkd2,Cyba,Sox10} {Nkd2,Cyba,Plp1} {Nkd2,Cyba,Lpr5} {Nkd2,Cyba,dkk3} {Nkd2,Cyba,Nrf2} {Nkd2,Sox10,Plp1} {Nkd2,Sox10,Lpr5} {Nkd2,Sox10,dkk3} {Nkd2,Sox10,Nrf2} {Nkd2,Plp1,Lpr5} {Nkd2,Plp1,dkk3} {Nkd2,Plp1,Nrf2} {Nkd2,Lpr5,dkk3} {Nkd2,Lpr5,Nrf2} {Nkd2,dkk3,Nrf2} {Tgfbr2,Tgfbr3,Hmox1} {Tgfbr2,Tgfbr3,Cyba} {Tgfbr2,Tgfbr3,Sox10} {Tgfbr2,Tgfbr3,Plp1} {Tgfbr2,Tgfbr3,Lpr5} {Tgfbr2,Tgfbr3,dkk3} {Tgfbr2,Tgfbr3,Nrf2} {Tgfbr2,Hmox1,Cyba} {Tgfbr2,Hmox1,Sox10} {Tgfbr2,Hmox1,Plp1} {Tgfbr2,Hmox1,Lpr5} {Tgfbr2,Hmox1,dkk3} {Tgfbr2,Hmox1,Nrf2} {Tgfbr2,Cyba,Sox10} {Tgfbr2,Cyba,Plp1} {Tgfbr2,Cyba,Lpr5} {Tgfbr2,Cyba,dkk3} {Tgfbr2,Cyba,Nrf2} {Tgfbr2,Sox10,Plp1} {Tgfbr2,Sox10,Lpr5} {Tgfbr2,Sox10,dkk3} {Tgfbr2,Sox10,Nrf2} {Tgfbr2,Plp1,Lpr5} {Tgfbr2,Plp1,dkk3} {Tgfbr2,Plp1,Nrf2} {Tgfbr2,Lpr5,dkk3} {Tgfbr2,Lpr5,Nrf2} {Tgfbr2,dkk3,Nrf2} {Tgfbr3,Hmox1,Cyba} {Tgfbr3,Hmox1,Sox10} {Tgfbr3,Hmox1,Plp1} {Tgfbr3,Hmox1,Lpr5} {Tgfbr3,Hmox1,dkk3} {Tgfbr3,Hmox1,Nrf2} {Tgfbr3,Cyba,Sox10} {Tgfbr3,Cyba,Plp1} {Tgfbr3,Cyba,Lpr5} {Tgfbr3,Cyba,dkk3} {Tgfbr3,Cyba,Nrf2} {Tgfbr3,Sox10,Plp1} {Tgfbr3,Sox10,Lpr5} {Tgfbr3,Sox10,dkk3} {Tgfbr3,Sox10,Nrf2} {Tgfbr3,Plp1,Lpr5} {Tgfbr3,Plp1,dkk3} {Tgfbr3,Plp1,Nrf2} {Tgfbr3,Lpr5,dkk3} {Tgfbr3,Lpr5,Nrf2} {Tgfbr3,dkk3,Nrf2} {Hmox1,Cyba,Sox10} {Hmox1,Cyba,Plp1} {Hmox1,Cyba,Lpr5} {Hmox1,Cyba,dkk3} {Hmox1,Cyba,Nrf2} {Hmox1,Sox10,Plp1} {Hmox1,Sox10,Lpr5} {Hmox1,Sox10,dkk3} {Hmox1,Sox10,Nrf2} {Hmox1,Plp1,Lpr5} {Hmox1,Plp1,dkk3} {Hmox1,Plp1,Nrf2} {Hmox1,Lpr5,dkk3} {Hmox1,Lpr5,Nrf2} {Hmox1,dkk3,Nrf2} {Cyba,Sox10,Plp1} {Cyba,Sox10,Lpr5} {Cyba,Sox10,dkk3} {Cyba,Sox10,Nrf2} {Cyba,Plp1,Lpr5} {Cyba,Plp1,dkk3} {Cyba,Plp1,Nrf2} {Cyba,Lpr5,dkk3} {Cyba,Lpr5,Nrf2} {Cyba,dkk3,Nrf2} {Sox10,Plp1,Lpr5} {Sox10,Plp1,dkk3} {Sox10,Plp1,Nrf2} {Sox10,Lpr5,dkk3} {Sox10,Lpr5,Nrf2} {Sox10,dkk3,Nrf2} {Plp1,Lpr5,dkk3} {Plp1,Lpr5,Nrf2} {Plp1,dkk3,Nrf2} {Lpr5,dkk3,Nrf2} {Lgr6,Frb,Nkd2,Tgfbr2} {Lgr6,Frb,Nkd2,Tgfbr3} {Lgr6,Frb,Nkd2,Hmox1} {Lgr6,Frb,Nkd2,Cyba} {Lgr6,Frb,Nkd2,Sox10} {Lgr6,Frb,Nkd2,Plp1} {Lgr6,Frb,Nkd2,Lpr5} {Lgr6,Frb,Nkd2,dkk3} {Lgr6,Frb,Nkd2,Nrf2} {Lgr6,Frb,Tgfbr2,Tgfbr3} {Lgr6,Frb,Tgfbr2,Hmox1} {Lgr6,Frb,Tgfbr2,Cyba} {Lgr6,Frb,Tgfbr2,Sox10} {Lgr6,Frb,Tgfbr2,Plp1} {Lgr6,Frb,Tgfbr2,Lpr5} {Lgr6,Frb,Tgfbr2,dkk3} {Lgr6,Frb,Tgfbr2,Nrf2} {Lgr6,Frb,Tgfbr3,Hmox1} {Lgr6,Frb,Tgfbr3,Cyba} {Lgr6,Frb,Tgfbr3,Sox10} {Lgr6,Frb,Tgfbr3,Plp1} {Lgr6,Frb,Tgfbr3,Lpr5} {Lgr6,Frb,Tgfbr3,dkk3} {Lgr6,Frb,Tgfbr3,Nrf2} {Lgr6,Frb,Hmox1,Cyba} {Lgr6,Frb,Hmox1,Sox10} {Lgr6,Frb,Hmox1,Plp1} {Lgr6,Frb,Hmox1,Lpr5} {Lgr6,Frb,Hmox1,dkk3} {Lgr6,Frb,Hmox1,Nrf2} {Lgr6,Frb,Cyba,Sox10} {Lgr6,Frb,Cyba,Plp1} {Lgr6,Frb,Cyba,Lpr5} {Lgr6,Frb,Cyba,dkk3} {Lgr6,Frb,Cyba,Nrf2} {Lgr6,Frb,Sox10,Plp1} {Lgr6,Frb,Sox10,Lpr5} {Lgr6,Frb,Sox10,dkk3} {Lgr6,Frb,Sox10,Nrf2} {Lgr6,Frb,Plp1,Lpr5} {Lgr6,Frb,Plp1,dkk3} {Lgr6,Frb,Plp1,Nrf2} {Lgr6,Frb,Lpr5,dkk3} {Lgr6,Frb,Lpr5,Nrf2} {Lgr6,Frb,dkk3,Nrf2} {Lgr6,Nkd2,Tgfbr2,Tgfbr3} {Lgr6,Nkd2,Tgfbr2,Hmox1} {Lgr6,Nkd2,Tgfbr2,Cyba} {Lgr6,Nkd2,Tgfbr2,Sox10} {Lgr6,Nkd2,Tgfbr2,Plp1} {Lgr6,Nkd2,Tgfbr2,Lpr5} {Lgr6,Nkd2,Tgfbr2,dkk3} {Lgr6,Nkd2,Tgfbr2,Nrf2} {Lgr6,Nkd2,Tgfbr3,Hmox1} {Lgr6,Nkd2,Tgfbr3,Cyba} {Lgr6,Nkd2,Tgfbr3,Sox10} {Lgr6,Nkd2,Tgfbr3,Plp1} {Lgr6,Nkd2,Tgfbr3,Lpr5} {Lgr6,Nkd2,Tgfbr3,dkk3} {Lgr6,Nkd2,Tgfbr3,Nrf2} {Lgr6,Nkd2,Hmox1,Cyba} {Lgr6,Nkd2,Hmox1,Sox10} {Lgr6,Nkd2,Hmox1,Plp1} {Lgr6,Nkd2,Hmox1,Lpr5} {Lgr6,Nkd2,Hmox1,dkk3} {Lgr6,Nkd2,Hmox1,Nrf2} {Lgr6,Nkd2,Cyba,Sox10} {Lgr6,Nkd2,Cyba,Plp1} {Lgr6,Nkd2,Cyba,Lpr5} {Lgr6,Nkd2,Cyba,dkk3} {Lgr6,Nkd2,Cyba,Nrf2} {Lgr6,Nkd2,Sox10,Plp1} {Lgr6,Nkd2,Sox10,Lpr5} {Lgr6,Nkd2,Sox10,dkk3} {Lgr6,Nkd2,Sox10,Nrf2} {Lgr6,Nkd2,Plp1,Lpr5} {Lgr6,Nkd2,Plp1,dkk3} {Lgr6,Nkd2,Plp1,Nrf2} {Lgr6,Nkd2,Lpr5,dkk3} {Lgr6,Nkd2,Lpr5,Nrf2} {Lgr6,Nkd2,dkk3,Nrf2} {Lgr6,Tgfbr2,Tgfbr3,Hmox1} {Lgr6,Tgfbr2,Tgfbr3,Cyba} {Lgr6,Tgfbr2,Tgfbr3,Sox10} {Lgr6,Tgfbr2,Tgfbr3,Plp1} {Lgr6,Tgfbr2,Tgfbr3,Lpr5} {Lgr6,Tgfbr2,Tgfbr3,dkk3} {Lgr6,Tgfbr2,Tgfbr3,Nrf2} {Lgr6,Tgfbr2,Hmox1,Cyba} {Lgr6,Tgfbr2,Hmox1,Sox10} {Lgr6,Tgfbr2,Hmox1,Plp1} {Lgr6,Tgfbr2,Hmox1,Lpr5} {Lgr6,Tgfbr2,Hmox1,dkk3} {Lgr6,Tgfbr2,Hmox1,Nrf2} {Lgr6,Tgfbr2,Cyba,Sox10} {Lgr6,Tgfbr2,Cyba,Plp1} {Lgr6,Tgfbr2,Cyba,Lpr5} {Lgr6,Tgfbr2,Cyba,dkk3} {Lgr6,Tgfbr2,Cyba,Nrf2} {Lgr6,Tgfbr2,Sox10,Plp1} {Lgr6,Tgfbr2,Sox10,Lpr5} {Lgr6,Tgfbr2,Sox10,dkk3} {Lgr6,Tgfbr2,Sox10,Nrf2} {Lgr6,Tgfbr2,Plp1,Lpr5} {Lgr6,Tgfbr2,Plp1,dkk3} {Lgr6,Tgfbr2,Plp1,Nrf2} {Lgr6,Tgfbr2,Lpr5,dkk3} {Lgr6,Tgfbr2,Lpr5,Nrf2} {Lgr6,Tgfbr2,dkk3,Nrf2} {Lgr6,Tgfbr3,Hmox1,Cyba} {Lgr6,Tgfbr3,Hmox1,Sox10} {Lgr6,Tgfbr3,Hmox1,Plp1} {Lgr6,Tgfbr3,Hmox1,Lpr5} {Lgr6,Tgfbr3,Hmox1,dkk3} {Lgr6,Tgfbr3,Hmox1,Nrf2} {Lgr6,Tgfbr3,Cyba,Sox10} {Lgr6,Tgfbr3,Cyba,Plp1} {Lgr6,Tgfbr3,Cyba,Lpr5} {Lgr6,Tgfbr3,Cyba,dkk3} {Lgr6,Tgfbr3,Cyba,Nrf2} {Lgr6,Tgfbr3,Sox10,Plp1} {Lgr6,Tgfbr3,Sox10,Lpr5} {Lgr6,Tgfbr3,Sox10,dkk3} {Lgr6,Tgfbr3,Sox10,Nrf2} {Lgr6,Tgfbr3,Plp1,Lpr5} {Lgr6,Tgfbr3,Plp1,dkk3} {Lgr6,Tgfbr3,Plp1,Nrf2} {Lgr6,Tgfbr3,Lpr5,dkk3} {Lgr6,Tgfbr3,Lpr5,Nrf2} {Lgr6,Tgfbr3,dkk3,Nrf2} {Lgr6,Hmox1,Cyba,Sox10} {Lgr6,Hmox1,Cyba,Plp1} {Lgr6,Hmox1,Cyba,Lpr5} {Lgr6,Hmox1,Cyba,dkk3} {Lgr6,Hmox1,Cyba,Nrf2} {Lgr6,Hmox1,Sox10,Plp1} {Lgr6,Hmox1,Sox10,Lpr5} {Lgr6,Hmox1,Sox10,dkk3} {Lgr6,Hmox1,Sox10,Nrf2} {Lgr6,Hmox1,Plp1,Lpr5} {Lgr6,Hmox1,Plp1,dkk3} {Lgr6,Hmox1,Plp1,Nrf2} {Lgr6,Hmox1,Lpr5,dkk3} {Lgr6,Hmox1,Lpr5,Nrf2} {Lgr6,Hmox1,dkk3,Nrf2} {Lgr6,Cyba,Sox10,Plp1} {Lgr6,Cyba,Sox10,Lpr5} {Lgr6,Cyba,Sox10,dkk3} {Lgr6,Cyba,Sox10,Nrf2} {Lgr6,Cyba,Plp1,Lpr5} {Lgr6,Cyba,Plp1,dkk3} {Lgr6,Cyba,Plp1,Nrf2} {Lgr6,Cyba,Lpr5,dkk3} {Lgr6,Cyba,Lpr5,Nrf2} {Lgr6,Cyba,dkk3,Nrf2} {Lgr6,Sox10,Plp1,Lpr5} {Lgr6,Sox10,Plp1,dkk3} {Lgr6,Sox10,Plp1,Nrf2} {Lgr6,Sox10,Lpr5,dkk3} {Lgr6,Sox10,Lpr5,Nrf2} {Lgr6,Sox10,dkk3,Nrf2} {Lgr6,Plp1,Lpr5,dkk3} {Lgr6,Plp1,Lpr5,Nrf2} {Lgr6,Plp1,dkk3,Nrf2} {Lgr6,Lpr5,dkk3,Nrf2} {Frb,Nkd2,Tgfbr2,Tgfbr3} {Frb,Nkd2,Tgfbr2,Hmox1} {Frb,Nkd2,Tgfbr2,Cyba} {Frb,Nkd2,Tgfbr2,Sox10} {Frb,Nkd2,Tgfbr2,Plp1} {Frb,Nkd2,Tgfbr2,Lpr5} {Frb,Nkd2,Tgfbr2,dkk3} {Frb,Nkd2,Tgfbr2,Nrf2} {Frb,Nkd2,Tgfbr3,Hmox1} {Frb,Nkd2,Tgfbr3,Cyba} {Frb,Nkd2,Tgfbr3,Sox10} {Frb,Nkd2,Tgfbr3,Plp1} {Frb,Nkd2,Tgfbr3,Lpr5} {Frb,Nkd2,Tgfbr3,dkk3} {Frb,Nkd2,Tgfbr3,Nrf2} {Frb,Nkd2,Hmox1,Cyba} {Frb,Nkd2,Hmox1,Sox10} {Frb,Nkd2,Hmox1,Plp1} {Frb,Nkd2,Hmox1,Lpr5} {Frb,Nkd2,Hmox1,dkk3} {Frb,Nkd2,Hmox1,Nrf2} {Frb,Nkd2,Cyba,Sox10} {Frb,Nkd2,Cyba,Plp1} {Frb,Nkd2,Cyba,Lpr5} {Frb,Nkd2,Cyba,dkk3} {Frb,Nkd2,Cyba,Nrf2} {Frb,Nkd2,Sox10,Plp1} {Frb,Nkd2,Sox10,Lpr5} {Frb,Nkd2,Sox10,dkk3} {Frb,Nkd2,Sox10,Nrf2} {Frb,Nkd2,Plp1,Lpr5} {Frb,Nkd2,Plp1,dkk3} {Frb,Nkd2,Plp1,Nrf2} {Frb,Nkd2,Lpr5,dkk3} {Frb,Nkd2,Lpr5,Nrf2} {Frb,Nkd2,dkk3,Nrf2} {Frb,Tgfbr2,Tgfbr3,Hmox1} {Frb,Tgfbr2,Tgfbr3,Cyba} {Frb,Tgfbr2,Tgfbr3,Sox10} {Frb,Tgfbr2,Tgfbr3,Plp1} {Frb,Tgfbr2,Tgfbr3,Lpr5} {Frb,Tgfbr2,Tgfbr3,dkk3} {Frb,Tgfbr2,Tgfbr3,Nrf2} {Frb,Tgfbr2,Hmox1,Cyba} {Frb,Tgfbr2,Hmox1,Sox10} {Frb,Tgfbr2,Hmox1,Plp1} {Frb,Tgfbr2,Hmox1,Lpr5} {Frb,Tgfbr2,Hmox1,dkk3} {Frb,Tgfbr2,Hmox1,Nrf2} {Frb,Tgfbr2,Cyba,Sox10} {Frb,Tgfbr2,Cyba,Plp1} {Frb,Tgfbr2,Cyba,Lpr5} {Frb,Tgfbr2,Cyba,dkk3} {Frb,Tgfbr2,Cyba,Nrf2} {Frb,Tgfbr2,Sox10,Plp1} {Frb,Tgfbr2,Sox10,Lpr5} {Frb,Tgfbr2,Sox10,dkk3} {Frb,Tgfbr2,Sox10,Nrf2} {Frb,Tgfbr2,Plp1,Lpr5} {Frb,Tgfbr2,Plp1,dkk3} {Frb,Tgfbr2,Plp1,Nrf2} {Frb,Tgfbr2,Lpr5,dkk3} {Frb,Tgfbr2,Lpr5,Nrf2} {Frb,Tgfbr2,dkk3,Nrf2} {Frb,Tgfbr3,Hmox1,Cyba} {Frb,Tgfbr3,Hmox1,Sox10} {Frb,Tgfbr3,Hmox1,Plp1} {Frb,Tgfbr3,Hmox1,Lpr5} {Frb,Tgfbr3,Hmox1,dkk3} {Frb,Tgfbr3,Hmox1,Nrf2} {Frb,Tgfbr3,Cyba,Sox10} {Frb,Tgfbr3,Cyba,Plp1} {Frb,Tgfbr3,Cyba,Lpr5} {Frb,Tgfbr3,Cyba,dkk3} {Frb,Tgfbr3,Cyba,Nrf2} {Frb,Tgfbr3,Sox10,Plp1} {Frb,Tgfbr3,Sox10,Lpr5} {Frb,Tgfbr3,Sox10,dkk3} {Frb,Tgfbr3,Sox10,Nrf2} {Frb,Tgfbr3,Plp1,Lpr5} {Frb,Tgfbr3,Plp1,dkk3} {Frb,Tgfbr3,Plp1,Nrf2} {Frb,Tgfbr3,Lpr5,dkk3} {Frb,Tgfbr3,Lpr5,Nrf2} {Frb,Tgfbr3,dkk3,Nrf2} {Frb,Hmox1,Cyba,Sox10} {Frb,Hmox1,Cyba,Plp1} {Frb,Hmox1,Cyba,Lpr5} {Frb,Hmox1,Cyba,dkk3} {Frb,Hmox1,Cyba,Nrf2} {Frb,Hmox1,Sox10,Plp1} {Frb,Hmox1,Sox10,Lpr5} {Frb,Hmox1,Sox10,dkk3} {Frb,Hmox1,Sox10,Nrf2} {Frb,Hmox1,Plp1,Lpr5} {Frb,Hmox1,Plp1,dkk3} {Frb,Hmox1,Plp1,Nrf2} {Frb,Hmox1,Lpr5,dkk3} {Frb,Hmox1,Lpr5,Nrf2} {Frb,Hmox1,dkk3,Nrf2} {Frb,Cyba,Sox10,Plp1} {Frb,Cyba,Sox10,Lpr5} {Frb,Cyba,Sox10,dkk3} {Frb,Cyba,Sox10,Nrf2} {Frb,Cyba,Plp1,Lpr5} {Frb,Cyba,Plp1,dkk3} {Frb,Cyba,Plp1,Nrf2} {Frb,Cyba,Lpr5,dkk3} {Frb,Cyba,Lpr5,Nrf2} {Frb,Cyba,dkk3,Nrf2} {Frb,Sox10,Plp1,Lpr5} {Frb,Sox10,Plp1,dkk3} {Frb,Sox10,Plp1,Nrf2} {Frb,Sox10,Lpr5,dkk3} {Frb,Sox10,Lpr5,Nrf2} {Frb,Sox10,dkk3,Nrf2} {Frb,Plp1,Lpr5,dkk3} {Frb,Plp1,Lpr5,Nrf2} {Frb,Plp1,dkk3,Nrf2} {Frb,Lpr5,dkk3,Nrf2} {Nkd2,Tgfbr2,Tgfbr3,Hmox1} {Nkd2,Tgfbr2,Tgfbr3,Cyba} {Nkd2,Tgfbr2,Tgfbr3,Sox10} {Nkd2,Tgfbr2,Tgfbr3,Plp1} {Nkd2,Tgfbr2,Tgfbr3,Lpr5} {Nkd2,Tgfbr2,Tgfbr3,dkk3} {Nkd2,Tgfbr2,Tgfbr3,Nrf2} {Nkd2,Tgfbr2,Hmox1,Cyba} {Nkd2,Tgfbr2,Hmox1,Sox10} {Nkd2,Tgfbr2,Hmox1,Plp1} {Nkd2,Tgfbr2,Hmox1,Lpr5} {Nkd2,Tgfbr2,Hmox1,dkk3} {Nkd2,Tgfbr2,Hmox1,Nrf2} {Nkd2,Tgfbr2,Cyba,Sox10} {Nkd2,Tgfbr2,Cyba,Plp1} {Nkd2,Tgfbr2,Cyba,Lpr5} {Nkd2,Tgfbr2,Cyba,dkk3} {Nkd2,Tgfbr2,Cyba,Nrf2} {Nkd2,Tgfbr2,Sox10,Plp1} {Nkd2,Tgfbr2,Sox10,Lpr5} {Nkd2,Tgfbr2,Sox10,dkk3} {Nkd2,Tgfbr2,Sox10,Nrf2} {Nkd2,Tgfbr2,Plp1,Lpr5} {Nkd2,Tgfbr2,Plp1,dkk3} {Nkd2,Tgfbr2,Plp1,Nrf2} {Nkd2,Tgfbr2,Lpr5,dkk3} {Nkd2,Tgfbr2,Lpr5,Nrf2} {Nkd2,Tgfbr2,dkk3,Nrf2} {Nkd2,Tgfbr3,Hmox1,Cyba} {Nkd2,Tgfbr3,Hmox1,Sox10} {Nkd2,Tgfbr3,Hmox1,Plp1} {Nkd2,Tgfbr3,Hmox1,Lpr5} {Nkd2,Tgfbr3,Hmox1,dkk3} {Nkd2,Tgfbr3,Hmox1,Nrf2} {Nkd2,Tgfbr3,Cyba,Sox10} {Nkd2,Tgfbr3,Cyba,Plp1} {Nkd2,Tgfbr3,Cyba,Lpr5} {Nkd2,Tgfbr3,Cyba,dkk3} {Nkd2,Tgfbr3,Cyba,Nrf2} {Nkd2,Tgfbr3,Sox10,Plp1} {Nkd2,Tgfbr3,Sox10,Lpr5} {Nkd2,Tgfbr3,Sox10,dkk3} {Nkd2,Tgfbr3,Sox10,Nrf2} {Nkd2,Tgfbr3,Plp1,Lpr5} {Nkd2,Tgfbr3,Plp1,dkk3} {Nkd2,Tgfbr3,Plp1,Nrf2} {Nkd2,Tgfbr3,Lpr5,dkk3} {Nkd2,Tgfbr3,Lpr5,Nrf2} {Nkd2,Tgfbr3,dkk3,Nrf2} {Nkd2,Hmox1,Cyba,Sox10} {Nkd2,Hmox1,Cyba,Plp1} {Nkd2,Hmox1,Cyba,Lpr5} {Nkd2,Hmox1,Cyba,dkk3} {Nkd2,Hmox1,Cyba,Nrf2} {Nkd2,Hmox1,Sox10,Plp1} {Nkd2,Hmox1,Sox10,Lpr5} {Nkd2,Hmox1,Sox10,dkk3} {Nkd2,Hmox1,Sox10,Nrf2} {Nkd2,Hmox1,Plp1,Lpr5} {Nkd2,Hmox1,Plp1,dkk3} {Nkd2,Hmox1,Plp1,Nrf2} {Nkd2,Hmox1,Lpr5,dkk3} {Nkd2,Hmox1,Lpr5,Nrf2} {Nkd2,Hmox1,dkk3,Nrf2} {Nkd2,Cyba,Sox10,Plp1} {Nkd2,Cyba,Sox10,Lpr5} {Nkd2,Cyba,Sox10,dkk3} {Nkd2,Cyba,Sox10,Nrf2} {Nkd2,Cyba,Plp1,Lpr5} {Nkd2,Cyba,Plp1,dkk3} {Nkd2,Cyba,Plp1,Nrf2} {Nkd2,Cyba,Lpr5,dkk3} {Nkd2,Cyba,Lpr5,Nrf2} {Nkd2,Cyba,dkk3,Nrf2} {Nkd2,Sox10,Plp1,Lpr5} {Nkd2,Sox10,Plp1,dkk3} {Nkd2,Sox10,Plp1,Nrf2} {Nkd2,Sox10,Lpr5,dkk3} {Nkd2,Sox10,Lpr5,Nrf2} {Nkd2,Sox10,dkk3,Nrf2} {Nkd2,Plp1,Lpr5,dkk3} {Nkd2,Plp1,Lpr5,Nrf2} {Nkd2,Plp1,dkk3,Nrf2} {Nkd2,Lpr5,dkk3,Nrf2} {Tgfbr2,Tgfbr3,Hmox1,Cyba} {Tgfbr2,Tgfbr3,Hmox1,Sox10} {Tgfbr2,Tgfbr3,Hmox1,Plp1} {Tgfbr2,Tgfbr3,Hmox1,Lpr5} {Tgfbr2,Tgfbr3,Hmox1,dkk3} {Tgfbr2,Tgfbr3,Hmox1,Nrf2} {Tgfbr2,Tgfbr3,Cyba,Sox10} {Tgfbr2,Tgfbr3,Cyba,Plp1} {Tgfbr2,Tgfbr3,Cyba,Lpr5} {Tgfbr2,Tgfbr3,Cyba,dkk3} {Tgfbr2,Tgfbr3,Cyba,Nrf2} {Tgfbr2,Tgfbr3,Sox10,Plp1} {Tgfbr2,Tgfbr3,Sox10,Lpr5} {Tgfbr2,Tgfbr3,Sox10,dkk3} {Tgfbr2,Tgfbr3,Sox10,Nrf2} {Tgfbr2,Tgfbr3,Plp1,Lpr5} {Tgfbr2,Tgfbr3,Plp1,dkk3} {Tgfbr2,Tgfbr3,Plp1,Nrf2} {Tgfbr2,Tgfbr3,Lpr5,dkk3} {Tgfbr2,Tgfbr3,Lpr5,Nrf2} {Tgfbr2,Tgfbr3,dkk3,Nrf2} {Tgfbr2,Hmox1,Cyba,Sox10} {Tgfbr2,Hmox1,Cyba,Plp1} {Tgfbr2,Hmox1,Cyba,Lpr5} {Tgfbr2,Hmox1,Cyba,dkk3} {Tgfbr2,Hmox1,Cyba,Nrf2} {Tgfbr2,Hmox1,Sox10,Plp1} {Tgfbr2,Hmox1,Sox10,Lpr5} {Tgfbr2,Hmox1,Sox10,dkk3} {Tgfbr2,Hmox1,Sox10,Nrf2} {Tgfbr2,Hmox1,Plp1,Lpr5} {Tgfbr2,Hmox1,Plp1,dkk3} {Tgfbr2,Hmox1,Plp1,Nrf2} {Tgfbr2,Hmox1,Lpr5,dkk3} {Tgfbr2,Hmox1,Lpr5,Nrf2} {Tgfbr2,Hmox1,dkk3,Nrf2} {Tgfbr2,Cyba,Sox10,Plp1} {Tgfbr2,Cyba,Sox10,Lpr5} {Tgfbr2,Cyba,Sox10,dkk3} {Tgfbr2,Cyba,Sox10,Nrf2} {Tgfbr2,Cyba,Plp1,Lpr5} {Tgfbr2,Cyba,Plp1,dkk3} {Tgfbr2,Cyba,Plp1,Nrf2} {Tgfbr2,Cyba,Lpr5,dkk3} {Tgfbr2,Cyba,Lpr5,Nrf2} {Tgfbr2,Cyba,dkk3,Nrf2} {Tgfbr2,Sox10,Plp1,Lpr5} {Tgfbr2,Sox10,Plp1,dkk3} {Tgfbr2,Sox10,Plp1,Nrf2} {Tgfbr2,Sox10,Lpr5,dkk3} {Tgfbr2,Sox10,Lpr5,Nrf2} {Tgfbr2,Sox10,dkk3,Nrf2} {Tgfbr2,Plp1,Lpr5,dkk3} {Tgfbr2,Plp1,Lpr5,Nrf2} {Tgfbr2,Plp1,dkk3,Nrf2} {Tgfbr2,Lpr5,dkk3,Nrf2} {Tgfbr3,Hmox1,Cyba,Sox10} {Tgfbr3,Hmox1,Cyba,Plp1} {Tgfbr3,Hmox1,Cyba,Lpr5} {Tgfbr3,Hmox1,Cyba,dkk3} {Tgfbr3,Hmox1,Cyba,Nrf2} {Tgfbr3,Hmox1,Sox10,Plp1} {Tgfbr3,Hmox1,Sox10,Lpr5} {Tgfbr3,Hmox1,Sox10,dkk3} {Tgfbr3,Hmox1,Sox10,Nrf2} {Tgfbr3,Hmox1,Plp1,Lpr5} {Tgfbr3,Hmox1,Plp1,dkk3} {Tgfbr3,Hmox1,Plp1,Nrf2} {Tgfbr3,Hmox1,Lpr5,dkk3} {Tgfbr3,Hmox1,Lpr5,Nrf2} {Tgfbr3,Hmox1,dkk3,Nrf2} {Tgfbr3,Cyba,Sox10,Plp1} {Tgfbr3,Cyba,Sox10,Lpr5} {Tgfbr3,Cyba,Sox10,dkk3} {Tgfbr3,Cyba,Sox10,Nrf2} {Tgfbr3,Cyba,Plp1,Lpr5} {Tgfbr3,Cyba,Plp1,dkk3} {Tgfbr3,Cyba,Plp1,Nrf2} {Tgfbr3,Cyba,Lpr5,dkk3} {Tgfbr3,Cyba,Lpr5,Nrf2} {Tgfbr3,Cyba,dkk3,Nrf2} {Tgfbr3,Sox10,Plp1,Lpr5} {Tgfbr3,Sox10,Plp1,dkk3} {Tgfbr3,Sox10,Plp1,Nrf2} {Tgfbr3,Sox10,Lpr5,dkk3} {Tgfbr3,Sox10,Lpr5,Nrf2} {Tgfbr3,Sox10,dkk3,Nrf2} {Tgfbr3,Plp1,Lpr5,dkk3} {Tgfbr3,Plp1,Lpr5,Nrf2} {Tgfbr3,Plp1,dkk3,Nrf2} {Tgfbr3,Lpr5,dkk3,Nrf2} {Hmox1,Cyba,Sox10,Plp1} {Hmox1,Cyba,Sox10,Lpr5} {Hmox1,Cyba,Sox10,dkk3} {Hmox1,Cyba,Sox10,Nrf2} {Hmox1,Cyba,Plp1,Lpr5} {Hmox1,Cyba,Plp1,dkk3} {Hmox1,Cyba,Plp1,Nrf2} {Hmox1,Cyba,Lpr5,dkk3} {Hmox1,Cyba,Lpr5,Nrf2} {Hmox1,Cyba,dkk3,Nrf2} {Hmox1,Sox10,Plp1,Lpr5} {Hmox1,Sox10,Plp1,dkk3} {Hmox1,Sox10,Plp1,Nrf2} {Hmox1,Sox10,Lpr5,dkk3} {Hmox1,Sox10,Lpr5,Nrf2} {Hmox1,Sox10,dkk3,Nrf2} {Hmox1,Plp1,Lpr5,dkk3} {Hmox1,Plp1,Lpr5,Nrf2} {Hmox1,Plp1,dkk3,Nrf2} {Hmox1,Lpr5,dkk3,Nrf2} {Cyba,Sox10,Plp1,Lpr5} {Cyba,Sox10,Plp1,dkk3} {Cyba,Sox10,Plp1,Nrf2} {Cyba,Sox10,Lpr5,dkk3} {Cyba,Sox10,Lpr5,Nrf2} {Cyba,Sox10,dkk3,Nrf2} {Cyba,Plp1,Lpr5,dkk3} {Cyba,Plp1,Lpr5,Nrf2} {Cyba,Plp1,dkk3,Nrf2} {Cyba,Lpr5,dkk3,Nrf2} {Sox10,Plp1,Lpr5,dkk3} {Sox10,Plp1,Lpr5,Nrf2} {Sox10,Plp1,dkk3,Nrf2} {Sox10,Lpr5,dkk3,Nrf2} {Plp1,Lpr5,dkk3,Nrf2} {Lgr6,Frb,Nkd2,Tgfbr2,Tgfbr3} {Lgr6,Frb,Nkd2,Tgfbr2,Hmox1} {Lgr6,Frb,Nkd2,Tgfbr2,Cyba} {Lgr6,Frb,Nkd2,Tgfbr2,Sox10} {Lgr6,Frb,Nkd2,Tgfbr2,Plp1} {Lgr6,Frb,Nkd2,Tgfbr2,Lpr5} {Lgr6,Frb,Nkd2,Tgfbr2,dkk3} {Lgr6,Frb,Nkd2,Tgfbr2,Nrf2} {Lgr6,Frb,Nkd2,Tgfbr3,Hmox1} {Lgr6,Frb,Nkd2,Tgfbr3,Cyba} {Lgr6,Frb,Nkd2,Tgfbr3,Sox10} {Lgr6,Frb,Nkd2,Tgfbr3,Plp1} {Lgr6,Frb,Nkd2,Tgfbr3,Lpr5} {Lgr6,Frb,Nkd2,Tgfbr3,dkk3} {Lgr6,Frb,Nkd2,Tgfbr3,Nrf2} {Lgr6,Frb,Nkd2,Hmox1,Cyba} {Lgr6,Frb,Nkd2,Hmox1,Sox10} {Lgr6,Frb,Nkd2,Hmox1,Plp1} {Lgr6,Frb,Nkd2,Hmox1,Lpr5} {Lgr6,Frb,Nkd2,Hmox1,dkk3} {Lgr6,Frb,Nkd2,Hmox1,Nrf2} {Lgr6,Frb,Nkd2,Cyba,Sox10} {Lgr6,Frb,Nkd2,Cyba,Plp1} {Lgr6,Frb,Nkd2,Cyba,Lpr5} {Lgr6,Frb,Nkd2,Cyba,dkk3} {Lgr6,Frb,Nkd2,Cyba,Nrf2} {Lgr6,Frb,Nkd2,Sox10,Plp1} {Lgr6,Frb,Nkd2,Sox10,Lpr5} {Lgr6,Frb,Nkd2,Sox10,dkk3} {Lgr6,Frb,Nkd2,Sox10,Nrf2} {Lgr6,Frb,Nkd2,Plp1,Lpr5} {Lgr6,Frb,Nkd2,Plp1,dkk3} {Lgr6,Frb,Nkd2,Plp1,Nrf2} {Lgr6,Frb,Nkd2,Lpr5,dkk3} {Lgr6,Frb,Nkd2,Lpr5,Nrf2} {Lgr6,Frb,Nkd2,dkk3,Nrf2} {Lgr6,Frb,Tgfbr2,Tgfbr3,Hmox1} {Lgr6,Frb,Tgfbr2,Tgfbr3,Cyba} {Lgr6,Frb,Tgfbr2,Tgfbr3,Sox10} {Lgr6,Frb,Tgfbr2,Tgfbr3,Plp1} {Lgr6,Frb,Tgfbr2,Tgfbr3,Lpr5} {Lgr6,Frb,Tgfbr2,Tgfbr3,dkk3} {Lgr6,Frb,Tgfbr2,Tgfbr3,Nrf2} {Lgr6,Frb,Tgfbr2,Hmox1,Cyba} {Lgr6,Frb,Tgfbr2,Hmox1,Sox10} {Lgr6,Frb,Tgfbr2,Hmox1,Plp1} {Lgr6,Frb,Tgfbr2,Hmox1,Lpr5} {Lgr6,Frb,Tgfbr2,Hmox1,dkk3} {Lgr6,Frb,Tgfbr2,Hmox1,Nrf2} {Lgr6,Frb,Tgfbr2,Cyba,Sox10} {Lgr6,Frb,Tgfbr2,Cyba,Plp1} {Lgr6,Frb,Tgfbr2,Cyba,Lpr5} {Lgr6,Frb,Tgfbr2,Cyba,dkk3} {Lgr6,Frb,Tgfbr2,Cyba,Nrf2} {Lgr6,Frb,Tgfbr2,Sox10,Plp1} {Lgr6,Frb,Tgfbr2,Sox10,Lpr5} {Lgr6,Frb,Tgfbr2,Sox10,dkk3} {Lgr6,Frb,Tgfbr2,Sox10,Nrf2} {Lgr6,Frb,Tgfbr2,Plp1,Lpr5} {Lgr6,Frb,Tgfbr2,Plp1,dkk3} {Lgr6,Frb,Tgfbr2,Plp1,Nrf2} {Lgr6,Frb,Tgfbr2,Lpr5,dkk3} {Lgr6,Frb,Tgfbr2,Lpr5,Nrf2} {Lgr6,Frb,Tgfbr2,dkk3,Nrf2} {Lgr6,Frb,Tgfbr3,Hmox1,Cyba} {Lgr6,Frb,Tgfbr3,Hmox1,Sox10} {Lgr6,Frb,Tgfbr3,Hmox1,Plp1} {Lgr6,Frb,Tgfbr3,Hmox1,Lpr5} {Lgr6,Frb,Tgfbr3,Hmox1,dkk3} {Lgr6,Frb,Tgfbr3,Hmox1,Nrf2} {Lgr6,Frb,Tgfbr3,Cyba,Sox10} {Lgr6,Frb,Tgfbr3,Cyba,Plp1} {Lgr6,Frb,Tgfbr3,Cyba,Lpr5} {Lgr6,Frb,Tgfbr3,Cyba,dkk3} {Lgr6,Frb,Tgfbr3,Cyba,Nrf2} {Lgr6,Frb,Tgfbr3,Sox10,Plp1} {Lgr6,Frb,Tgfbr3,Sox10,Lpr5} {Lgr6,Frb,Tgfbr3,Sox10,dkk3} {Lgr6,Frb,Tgfbr3,Sox10,Nrf2} {Lgr6,Frb,Tgfbr3,Plp1,Lpr5} {Lgr6,Frb,Tgfbr3,Plp1,dkk3} {Lgr6,Frb,Tgfbr3,Plp1,Nrf2} {Lgr6,Frb,Tgfbr3,Lpr5,dkk3} {Lgr6,Frb,Tgfbr3,Lpr5,Nrf2} {Lgr6,Frb,Tgfbr3,dkk3,Nrf2} {Lgr6,Frb,Hmox1,Cyba,Sox10} {Lgr6,Frb,Hmox1,Cyba,Plp1} {Lgr6,Frb,Hmox1,Cyba,Lpr5} {Lgr6,Frb,Hmox1,Cyba,dkk3} {Lgr6,Frb,Hmox1,Cyba,Nrf2} {Lgr6,Frb,Hmox1,Sox10,Plp1} {Lgr6,Frb,Hmox1,Sox10,Lpr5} {Lgr6,Frb,Hmox1,Sox10,dkk3} {Lgr6,Frb,Hmox1,Sox10,Nrf2} {Lgr6,Frb,Hmox1,Plp1,Lpr5} {Lgr6,Frb,Hmox1,Plp1,dkk3} {Lgr6,Frb,Hmox1,Plp1,Nrf2} {Lgr6,Frb,Hmox1,Lpr5,dkk3} {Lgr6,Frb,Hmox1,Lpr5,Nrf2} {Lgr6,Frb,Hmox1,dkk3,Nrf2} {Lgr6,Frb,Cyba,Sox10,Plp1} {Lgr6,Frb,Cyba,Sox10,Lpr5} {Lgr6,Frb,Cyba,Sox10,dkk3} {Lgr6,Frb,Cyba,Sox10,Nrf2} {Lgr6,Frb,Cyba,Plp1,Lpr5} {Lgr6,Frb,Cyba,Plp1,dkk3} {Lgr6,Frb,Cyba,Plp1,Nrf2} {Lgr6,Frb,Cyba,Lpr5,dkk3} {Lgr6,Frb,Cyba,Lpr5,Nrf2} {Lgr6,Frb,Cyba,dkk3,Nrf2} {Lgr6,Frb,Sox10,Plp1,Lpr5} {Lgr6,Frb,Sox10,Plp1,dkk3} {Lgr6,Frb,Sox10,Plp1,Nrf2} {Lgr6,Frb,Sox10,Lpr5,dkk3} {Lgr6,Frb,Sox10,Lpr5,Nrf2} {Lgr6,Frb,Sox10,dkk3,Nrf2} {Lgr6,Frb,Plp1,Lpr5,dkk3} {Lgr6,Frb,Plp1,Lpr5,Nrf2} {Lgr6,Frb,Plp1,dkk3,Nrf2} {Lgr6,Frb,Lpr5,dkk3,Nrf2} {Lgr6,Nkd2,Tgfbr2,Tgfbr3,Hmox1} {Lgr6,Nkd2,Tgfbr2,Tgfbr3,Cyba} {Lgr6,Nkd2,Tgfbr2,Tgfbr3,Sox10} {Lgr6,Nkd2,Tgfbr2,Tgfbr3,Plp1} {Lgr6,Nkd2,Tgfbr2,Tgfbr3,Lpr5} {Lgr6,Nkd2,Tgfbr2,Tgfbr3,dkk3} {Lgr6,Nkd2,Tgfbr2,Tgfbr3,Nrf2} {Lgr6,Nkd2,Tgfbr2,Hmox1,Cyba} {Lgr6,Nkd2,Tgfbr2,Hmox1,Sox10} {Lgr6,Nkd2,Tgfbr2,Hmox1,Plp1} {Lgr6,Nkd2,Tgfbr2,Hmox1,Lpr5} {Lgr6,Nkd2,Tgfbr2,Hmox1,dkk3} {Lgr6,Nkd2,Tgfbr2,Hmox1,Nrf2} {Lgr6,Nkd2,Tgfbr2,Cyba,Sox10} {Lgr6,Nkd2,Tgfbr2,Cyba,Plp1} {Lgr6,Nkd2,Tgfbr2,Cyba,Lpr5} {Lgr6,Nkd2,Tgfbr2,Cyba,dkk3} {Lgr6,Nkd2,Tgfbr2,Cyba,Nrf2} {Lgr6,Nkd2,Tgfbr2,Sox10,Plp1} {Lgr6,Nkd2,Tgfbr2,Sox10,Lpr5} {Lgr6,Nkd2,Tgfbr2,Sox10,dkk3} {Lgr6,Nkd2,Tgfbr2,Sox10,Nrf2} {Lgr6,Nkd2,Tgfbr2,Plp1,Lpr5} {Lgr6,Nkd2,Tgfbr2,Plp1,dkk3} {Lgr6,Nkd2,Tgfbr2,Plp1,Nrf2} {Lgr6,Nkd2,Tgfbr2,Lpr5,dkk3} {Lgr6,Nkd2,Tgfbr2,Lpr5,Nrf2} {Lgr6,Nkd2,Tgfbr2,dkk3,Nrf2} {Lgr6,Nkd2,Tgfbr3,Hmox1,Cyba} {Lgr6,Nkd2,Tgfbr3,Hmox1,Sox10} {Lgr6,Nkd2,Tgfbr3,Hmox1,Plp1} {Lgr6,Nkd2,Tgfbr3,Hmox1,Lpr5} {Lgr6,Nkd2,Tgfbr3,Hmox1,dkk3} {Lgr6,Nkd2,Tgfbr3,Hmox1,Nrf2} {Lgr6,Nkd2,Tgfbr3,Cyba,Sox10} {Lgr6,Nkd2,Tgfbr3,Cyba,Plp1} {Lgr6,Nkd2,Tgfbr3,Cyba,Lpr5} {Lgr6,Nkd2,Tgfbr3,Cyba,dkk3} {Lgr6,Nkd2,Tgfbr3,Cyba,Nrf2} {Lgr6,Nkd2,Tgfbr3,Sox10,Plp1} {Lgr6,Nkd2,Tgfbr3,Sox10,Lpr5} {Lgr6,Nkd2,Tgfbr3,Sox10,dkk3} {Lgr6,Nkd2,Tgfbr3,Sox10,Nrf2} {Lgr6,Nkd2,Tgfbr3,Plp1,Lpr5} {Lgr6,Nkd2,Tgfbr3,Plp1,dkk3} {Lgr6,Nkd2,Tgfbr3,Plp1,Nrf2} {Lgr6,Nkd2,Tgfbr3,Lpr5,dkk3} {Lgr6,Nkd2,Tgfbr3,Lpr5,Nrf2} {Lgr6,Nkd2,Tgfbr3,dkk3,Nrf2} {Lgr6,Nkd2,Hmox1,Cyba,Sox10} {Lgr6,Nkd2,Hmox1,Cyba,Plp1} {Lgr6,Nkd2,Hmox1,Cyba,Lpr5} {Lgr6,Nkd2,Hmox1,Cyba,dkk3} {Lgr6,Nkd2,Hmox1,Cyba,Nrf2} {Lgr6,Nkd2,Hmox1,Sox10,Plp1} {Lgr6,Nkd2,Hmox1,Sox10,Lpr5} {Lgr6,Nkd2,Hmox1,Sox10,dkk3} {Lgr6,Nkd2,Hmox1,Sox10,Nrf2} {Lgr6,Nkd2,Hmox1,Plp1,Lpr5} {Lgr6,Nkd2,Hmox1,Plp1,dkk3} {Lgr6,Nkd2,Hmox1,Plp1,Nrf2} {Lgr6,Nkd2,Hmox1,Lpr5,dkk3} {Lgr6,Nkd2,Hmox1,Lpr5,Nrf2} {Lgr6,Nkd2,Hmox1,dkk3,Nrf2} {Lgr6,Nkd2,Cyba,Sox10,Plp1} {Lgr6,Nkd2,Cyba,Sox10,Lpr5} {Lgr6,Nkd2,Cyba,Sox10,dkk3} {Lgr6,Nkd2,Cyba,Sox10,Nrf2} {Lgr6,Nkd2,Cyba,Plp1,Lpr5} {Lgr6,Nkd2,Cyba,Plp1,dkk3} {Lgr6,Nkd2,Cyba,Plp1,Nrf2} {Lgr6,Nkd2,Cyba,Lpr5,dkk3} {Lgr6,Nkd2,Cyba,Lpr5,Nrf2} {Lgr6,Nkd2,Cyba,dkk3,Nrf2} {Lgr6,Nkd2,Sox10,Plp1,Lpr5} {Lgr6,Nkd2,Sox10,Plp1,dkk3} {Lgr6,Nkd2,Sox10,Plp1,Nrf2} {Lgr6,Nkd2,Sox10,Lpr5,dkk3} {Lgr6,Nkd2,Sox10,Lpr5,Nrf2} {Lgr6,Nkd2,Sox10,dkk3,Nrf2} {Lgr6,Nkd2,Plp1,Lpr5,dkk3} {Lgr6,Nkd2,Plp1,Lpr5,Nrf2} {Lgr6,Nkd2,Plp1,dkk3,Nrf2} {Lgr6,Nkd2,Lpr5,dkk3,Nrf2} {Lgr6,Tgfbr2,Tgfbr3,Hmox1,Cyba} {Lgr6,Tgfbr2,Tgfbr3,Hmox1,Sox10} {Lgr6,Tgfbr2,Tgfbr3,Hmox1,Plp1} {Lgr6,Tgfbr2,Tgfbr3,Hmox1,Lpr5} {Lgr6,Tgfbr2,Tgfbr3,Hmox1,dkk3} {Lgr6,Tgfbr2,Tgfbr3,Hmox1,Nrf2} {Lgr6,Tgfbr2,Tgfbr3,Cyba,Sox10} {Lgr6,Tgfbr2,Tgfbr3,Cyba,Plp1} {Lgr6,Tgfbr2,Tgfbr3,Cyba,Lpr5} {Lgr6,Tgfbr2,Tgfbr3,Cyba,dkk3} {Lgr6,Tgfbr2,Tgfbr3,Cyba,Nrf2} {Lgr6,Tgfbr2,Tgfbr3,Sox10,Plp1} {Lgr6,Tgfbr2,Tgfbr3,Sox10,Lpr5} {Lgr6,Tgfbr2,Tgfbr3,Sox10,dkk3} {Lgr6,Tgfbr2,Tgfbr3,Sox10,Nrf2} {Lgr6,Tgfbr2,Tgfbr3,Plp1,Lpr5} {Lgr6,Tgfbr2,Tgfbr3,Plp1,dkk3} {Lgr6,Tgfbr2,Tgfbr3,Plp1,Nrf2} {Lgr6,Tgfbr2,Tgfbr3,Lpr5,dkk3} {Lgr6,Tgfbr2,Tgfbr3,Lpr5,Nrf2} {Lgr6,Tgfbr2,Tgfbr3,dkk3,Nrf2} {Lgr6,Tgfbr2,Hmox1,Cyba,Sox10} {Lgr6,Tgfbr2,Hmox1,Cyba,Plp1} {Lgr6,Tgfbr2,Hmox1,Cyba,Lpr5} {Lgr6,Tgfbr2,Hmox1,Cyba,dkk3} {Lgr6,Tgfbr2,Hmox1,Cyba,Nrf2} {Lgr6,Tgfbr2,Hmox1,Sox10,Plp1} {Lgr6,Tgfbr2,Hmox1,Sox10,Lpr5} {Lgr6,Tgfbr2,Hmox1,Sox10,dkk3} {Lgr6,Tgfbr2,Hmox1,Sox10,Nrf2} {Lgr6,Tgfbr2,Hmox1,Plp1,Lpr5} {Lgr6,Tgfbr2,Hmox1,Plp1,dkk3} {Lgr6,Tgfbr2,Hmox1,Plp1,Nrf2} {Lgr6,Tgfbr2,Hmox1,Lpr5,dkk3} {Lgr6,Tgfbr2,Hmox1,Lpr5,Nrf2} {Lgr6,Tgfbr2,Hmox1,dkk3,Nrf2} {Lgr6,Tgfbr2,Cyba,Sox10,Plp1} {Lgr6,Tgfbr2,Cyba,Sox10,Lpr5} {Lgr6,Tgfbr2,Cyba,Sox10,dkk3} {Lgr6,Tgfbr2,Cyba,Sox10,Nrf2} {Lgr6,Tgfbr2,Cyba,Plp1,Lpr5} {Lgr6,Tgfbr2,Cyba,Plp1,dkk3} {Lgr6,Tgfbr2,Cyba,Plp1,Nrf2} {Lgr6,Tgfbr2,Cyba,Lpr5,dkk3} {Lgr6,Tgfbr2,Cyba,Lpr5,Nrf2} {Lgr6,Tgfbr2,Cyba,dkk3,Nrf2} {Lgr6,Tgfbr2,Sox10,Plp1,Lpr5} {Lgr6,Tgfbr2,Sox10,Plp1,dkk3} {Lgr6,Tgfbr2,Sox10,Plp1,Nrf2} {Lgr6,Tgfbr2,Sox10,Lpr5,dkk3} {Lgr6,Tgfbr2,Sox10,Lpr5,Nrf2} {Lgr6,Tgfbr2,Sox10,dkk3,Nrf2} {Lgr6,Tgfbr2,Plp1,Lpr5,dkk3} {Lgr6,Tgfbr2,Plp1,Lpr5,Nrf2} {Lgr6,Tgfbr2,Plp1,dkk3,Nrf2} {Lgr6,Tgfbr2,Lpr5,dkk3,Nrf2} {Lgr6,Tgfbr3,Hmox1,Cyba,Sox10} {Lgr6,Tgfbr3,Hmox1,Cyba,Plp1} {Lgr6,Tgfbr3,Hmox1,Cyba,Lpr5} {Lgr6,Tgfbr3,Hmox1,Cyba,dkk3} {Lgr6,Tgfbr3,Hmox1,Cyba,Nrf2} {Lgr6,Tgfbr3,Hmox1,Sox10,Plp1} {Lgr6,Tgfbr3,Hmox1,Sox10,Lpr5} {Lgr6,Tgfbr3,Hmox1,Sox10,dkk3} {Lgr6,Tgfbr3,Hmox1,Sox10,Nrf2} {Lgr6,Tgfbr3,Hmox1,Plp1,Lpr5} {Lgr6,Tgfbr3,Hmox1,Plp1,dkk3} {Lgr6,Tgfbr3,Hmox1,Plp1,Nrf2} {Lgr6,Tgfbr3,Hmox1,Lpr5,dkk3} {Lgr6,Tgfbr3,Hmox1,Lpr5,Nrf2} {Lgr6,Tgfbr3,Hmox1,dkk3,Nrf2} {Lgr6,Tgfbr3,Cyba,Sox10,Plp1} {Lgr6,Tgfbr3,Cyba,Sox10,Lpr5} {Lgr6,Tgfbr3,Cyba,Sox10,dkk3} {Lgr6,Tgfbr3,Cyba,Sox10,Nrf2} {Lgr6,Tgfbr3,Cyba,Plp1,Lpr5} {Lgr6,Tgfbr3,Cyba,Plp1,dkk3} {Lgr6,Tgfbr3,Cyba,Plp1,Nrf2} {Lgr6,Tgfbr3,Cyba,Lpr5,dkk3} {Lgr6,Tgfbr3,Cyba,Lpr5,Nrf2} {Lgr6,Tgfbr3,Cyba,dkk3,Nrf2} {Lgr6,Tgfbr3,Sox10,Plp1,Lpr5} {Lgr6,Tgfbr3,Sox10,Plp1,dkk3} {Lgr6,Tgfbr3,Sox10,Plp1,Nrf2} {Lgr6,Tgfbr3,Sox10,Lpr5,dkk3} {Lgr6,Tgfbr3,Sox10,Lpr5,Nrf2} {Lgr6,Tgfbr3,Sox10,dkk3,Nrf2} {Lgr6,Tgfbr3,Plp1,Lpr5,dkk3} {Lgr6,Tgfbr3,Plp1,Lpr5,Nrf2} {Lgr6,Tgfbr3,Plp1,dkk3,Nrf2} {Lgr6,Tgfbr3,Lpr5,dkk3,Nrf2} {Lgr6,Hmox1,Cyba,Sox10,Plp1} {Lgr6,Hmox1,Cyba,Sox10,Lpr5} {Lgr6,Hmox1,Cyba,Sox10,dkk3} {Lgr6,Hmox1,Cyba,Sox10,Nrf2} {Lgr6,Hmox1,Cyba,Plp1,Lpr5} {Lgr6,Hmox1,Cyba,Plp1,dkk3} {Lgr6,Hmox1,Cyba,Plp1,Nrf2} {Lgr6,Hmox1,Cyba,Lpr5,dkk3} {Lgr6,Hmox1,Cyba,Lpr5,Nrf2} {Lgr6,Hmox1,Cyba,dkk3,Nrf2} {Lgr6,Hmox1,Sox10,Plp1,Lpr5} {Lgr6,Hmox1,Sox10,Plp1,dkk3} {Lgr6,Hmox1,Sox10,Plp1,Nrf2} {Lgr6,Hmox1,Sox10,Lpr5,dkk3} {Lgr6,Hmox1,Sox10,Lpr5,Nrf2} {Lgr6,Hmox1,Sox10,dkk3,Nrf2} {Lgr6,Hmox1,Plp1,Lpr5,dkk3} {Lgr6,Hmox1,Plp1,Lpr5,Nrf2} {Lgr6,Hmox1,Plp1,dkk3,Nrf2} {Lgr6,Hmox1,Lpr5,dkk3,Nrf2} {Lgr6,Cyba,Sox10,Plp1,Lpr5} {Lgr6,Cyba,Sox10,Plp1,dkk3} {Lgr6,Cyba,Sox10,Plp1,Nrf2} {Lgr6,Cyba,Sox10,Lpr5,dkk3} {Lgr6,Cyba,Sox10,Lpr5,Nrf2} {Lgr6,Cyba,Sox10,dkk3,Nrf2} {Lgr6,Cyba,Plp1,Lpr5,dkk3} {Lgr6,Cyba,Plp1,Lpr5,Nrf2} {Lgr6,Cyba,Plp1,dkk3,Nrf2} {Lgr6,Cyba,Lpr5,dkk3,Nrf2} {Lgr6,Sox10,Plp1,Lpr5,dkk3} {Lgr6,Sox10,Plp1,Lpr5,Nrf2} {Lgr6,Sox10,Plp1,dkk3,Nrf2} {Lgr6,Sox10,Lpr5,dkk3,Nrf2} {Lgr6,Plp1,Lpr5,dkk3,Nrf2} {Frb,Nkd2,Tgfbr2,Tgfbr3,Hmox1} {Frb,Nkd2,Tgfbr2,Tgfbr3,Cyba} {Frb,Nkd2,Tgfbr2,Tgfbr3,Sox10} {Frb,Nkd2,Tgfbr2,Tgfbr3,Plp1} {Frb,Nkd2,Tgfbr2,Tgfbr3,Lpr5} {Frb,Nkd2,Tgfbr2,Tgfbr3,dkk3} {Frb,Nkd2,Tgfbr2,Tgfbr3,Nrf2} {Frb,Nkd2,Tgfbr2,Hmox1,Cyba} {Frb,Nkd2,Tgfbr2,Hmox1,Sox10} {Frb,Nkd2,Tgfbr2,Hmox1,Plp1} {Frb,Nkd2,Tgfbr2,Hmox1,Lpr5} {Frb,Nkd2,Tgfbr2,Hmox1,dkk3} {Frb,Nkd2,Tgfbr2,Hmox1,Nrf2} {Frb,Nkd2,Tgfbr2,Cyba,Sox10} {Frb,Nkd2,Tgfbr2,Cyba,Plp1} {Frb,Nkd2,Tgfbr2,Cyba,Lpr5} {Frb,Nkd2,Tgfbr2,Cyba,dkk3} {Frb,Nkd2,Tgfbr2,Cyba,Nrf2} {Frb,Nkd2,Tgfbr2,Sox10,Plp1} {Frb,Nkd2,Tgfbr2,Sox10,Lpr5} {Frb,Nkd2,Tgfbr2,Sox10,dkk3} {Frb,Nkd2,Tgfbr2,Sox10,Nrf2} {Frb,Nkd2,Tgfbr2,Plp1,Lpr5} {Frb,Nkd2,Tgfbr2,Plp1,dkk3} {Frb,Nkd2,Tgfbr2,Plp1,Nrf2} {Frb,Nkd2,Tgfbr2,Lpr5,dkk3} {Frb,Nkd2,Tgfbr2,Lpr5,Nrf2} {Frb,Nkd2,Tgfbr2,dkk3,Nrf2} {Frb,Nkd2,Tgfbr3,Hmox1,Cyba} {Frb,Nkd2,Tgfbr3,Hmox1,Sox10} {Frb,Nkd2,Tgfbr3,Hmox1,Plp1} {Frb,Nkd2,Tgfbr3,Hmox1,Lpr5} {Frb,Nkd2,Tgfbr3,Hmox1,dkk3} {Frb,Nkd2,Tgfbr3,Hmox1,Nrf2} {Frb,Nkd2,Tgfbr3,Cyba,Sox10} {Frb,Nkd2,Tgfbr3,Cyba,Plp1} {Frb,Nkd2,Tgfbr3,Cyba,Lpr5} {Frb,Nkd2,Tgfbr3,Cyba,dkk3} {Frb,Nkd2,Tgfbr3,Cyba,Nrf2} {Frb,Nkd2,Tgfbr3,Sox10,Plp1} {Frb,Nkd2,Tgfbr3,Sox10,Lpr5} {Frb,Nkd2,Tgfbr3,Sox10,dkk3} {Frb,Nkd2,Tgfbr3,Sox10,Nrf2} {Frb,Nkd2,Tgfbr3,Plp1,Lpr5} {Frb,Nkd2,Tgfbr3,Plp1,dkk3} {Frb,Nkd2,Tgfbr3,Plp1,Nrf2} {Frb,Nkd2,Tgfbr3,Lpr5,dkk3} {Frb,Nkd2,Tgfbr3,Lpr5,Nrf2} {Frb,Nkd2,Tgfbr3,dkk3,Nrf2} {Frb,Nkd2,Hmox1,Cyba,Sox10} {Frb,Nkd2,Hmox1,Cyba,Plp1} {Frb,Nkd2,Hmox1,Cyba,Lpr5} {Frb,Nkd2,Hmox1,Cyba,dkk3} {Frb,Nkd2,Hmox1,Cyba,Nrf2} {Frb,Nkd2,Hmox1,Sox10,Plp1} {Frb,Nkd2,Hmox1,Sox10,Lpr5} {Frb,Nkd2,Hmox1,Sox10,dkk3} {Frb,Nkd2,Hmox1,Sox10,Nrf2} {Frb,Nkd2,Hmox1,Plp1,Lpr5} {Frb,Nkd2,Hmox1,Plp1,dkk3} {Frb,Nkd2,Hmox1,Plp1,Nrf2} {Frb,Nkd2,Hmox1,Lpr5,dkk3} {Frb,Nkd2,Hmox1,Lpr5,Nrf2} {Frb,Nkd2,Hmox1,dkk3,Nrf2} {Frb,Nkd2,Cyba,Sox10,Plp1} {Frb,Nkd2,Cyba,Sox10,Lpr5} {Frb,Nkd2,Cyba,Sox10,dkk3} {Frb,Nkd2,Cyba,Sox10,Nrf2} {Frb,Nkd2,Cyba,Plp1,Lpr5} {Frb,Nkd2,Cyba,Plp1,dkk3} {Frb,Nkd2,Cyba,Plp1,Nrf2} {Frb,Nkd2,Cyba,Lpr5,dkk3} {Frb,Nkd2,Cyba,Lpr5,Nrf2} {Frb,Nkd2,Cyba,dkk3,Nrf2} {Frb,Nkd2,Sox10,Plp1,Lpr5} {Frb,Nkd2,Sox10,Plp1,dkk3} {Frb,Nkd2,Sox10,Plp1,Nrf2} {Frb,Nkd2,Sox10,Lpr5,dkk3} {Frb,Nkd2,Sox10,Lpr5,Nrf2} {Frb,Nkd2,Sox10,dkk3,Nrf2} {Frb,Nkd2,Plp1,Lpr5,dkk3} {Frb,Nkd2,Plp1,Lpr5,Nrf2} {Frb,Nkd2,Plp1,dkk3,Nrf2} {Frb,Nkd2,Lpr5,dkk3,Nrf2} {Frb,Tgfbr2,Tgfbr3,Hmox1,Cyba} {Frb,Tgfbr2,Tgfbr3,Hmox1,Sox10} {Frb,Tgfbr2,Tgfbr3,Hmox1,Plp1} {Frb,Tgfbr2,Tgfbr3,Hmox1,Lpr5} {Frb,Tgfbr2,Tgfbr3,Hmox1,dkk3} {Frb,Tgfbr2,Tgfbr3,Hmox1,Nrf2} {Frb,Tgfbr2,Tgfbr3,Cyba,Sox10} {Frb,Tgfbr2,Tgfbr3,Cyba,Plp1} {Frb,Tgfbr2,Tgfbr3,Cyba,Lpr5} {Frb,Tgfbr2,Tgfbr3,Cyba,dkk3} {Frb,Tgfbr2,Tgfbr3,Cyba,Nrf2} {Frb,Tgfbr2,Tgfbr3,Sox10,Plp1} {Frb,Tgfbr2,Tgfbr3,Sox10,Lpr5} {Frb,Tgfbr2,Tgfbr3,Sox10,dkk3} {Frb,Tgfbr2,Tgfbr3,Sox10,Nrf2} {Frb,Tgfbr2,Tgfbr3,Plp1,Lpr5} {Frb,Tgfbr2,Tgfbr3,Plp1,dkk3} {Frb,Tgfbr2,Tgfbr3,Plp1,Nrf2} {Frb,Tgfbr2,Tgfbr3,Lpr5,dkk3} {Frb,Tgfbr2,Tgfbr3,Lpr5,Nrf2} {Frb,Tgfbr2,Tgfbr3,dkk3,Nrf2} {Frb,Tgfbr2,Hmox1,Cyba,Sox10} {Frb,Tgfbr2,Hmox1,Cyba,Plp1} {Frb,Tgfbr2,Hmox1,Cyba,Lpr5} {Frb,Tgfbr2,Hmox1,Cyba,dkk3} {Frb,Tgfbr2,Hmox1,Cyba,Nrf2} {Frb,Tgfbr2,Hmox1,Sox10,Plp1} {Frb,Tgfbr2,Hmox1,Sox10,Lpr5} {Frb,Tgfbr2,Hmox1,Sox10,dkk3} {Frb,Tgfbr2,Hmox1,Sox10,Nrf2} {Frb,Tgfbr2,Hmox1,Plp1,Lpr5} {Frb,Tgfbr2,Hmox1,Plp1,dkk3} {Frb,Tgfbr2,Hmox1,Plp1,Nrf2} {Frb,Tgfbr2,Hmox1,Lpr5,dkk3} {Frb,Tgfbr2,Hmox1,Lpr5,Nrf2} {Frb,Tgfbr2,Hmox1,dkk3,Nrf2} {Frb,Tgfbr2,Cyba,Sox10,Plp1} {Frb,Tgfbr2,Cyba,Sox10,Lpr5} {Frb,Tgfbr2,Cyba,Sox10,dkk3} {Frb,Tgfbr2,Cyba,Sox10,Nrf2} {Frb,Tgfbr2,Cyba,Plp1,Lpr5} {Frb,Tgfbr2,Cyba,Plp1,dkk3} {Frb,Tgfbr2,Cyba,Plp1,Nrf2} {Frb,Tgfbr2,Cyba,Lpr5,dkk3} {Frb,Tgfbr2,Cyba,Lpr5,Nrf2} {Frb,Tgfbr2,Cyba,dkk3,Nrf2} {Frb,Tgfbr2,Sox10,Plp1,Lpr5} {Frb,Tgfbr2,Sox10,Plp1,dkk3} {Frb,Tgfbr2,Sox10,Plp1,Nrf2} {Frb,Tgfbr2,Sox10,Lpr5,dkk3} {Frb,Tgfbr2,Sox10,Lpr5,Nrf2} {Frb,Tgfbr2,Sox10,dkk3,Nrf2} {Frb,Tgfbr2,Plp1,Lpr5,dkk3} {Frb,Tgfbr2,Plp1,Lpr5,Nrf2} {Frb,Tgfbr2,Plp1,dkk3,Nrf2} {Frb,Tgfbr2,Lpr5,dkk3,Nrf2} {Frb,Tgfbr3,Hmox1,Cyba,Sox10} {Frb,Tgfbr3,Hmox1,Cyba,Plp1} {Frb,Tgfbr3,Hmox1,Cyba,Lpr5} {Frb,Tgfbr3,Hmox1,Cyba,dkk3} {Frb,Tgfbr3,Hmox1,Cyba,Nrf2} {Frb,Tgfbr3,Hmox1,Sox10,Plp1} {Frb,Tgfbr3,Hmox1,Sox10,Lpr5} {Frb,Tgfbr3,Hmox1,Sox10,dkk3} {Frb,Tgfbr3,Hmox1,Sox10,Nrf2} {Frb,Tgfbr3,Hmox1,Plp1,Lpr5} {Frb,Tgfbr3,Hmox1,Plp1,dkk3} {Frb,Tgfbr3,Hmox1,Plp1,Nrf2} {Frb,Tgfbr3,Hmox1,Lpr5,dkk3} {Frb,Tgfbr3,Hmox1,Lpr5,Nrf2} {Frb,Tgfbr3,Hmox1,dkk3,Nrf2} {Frb,Tgfbr3,Cyba,Sox10,Plp1} {Frb,Tgfbr3,Cyba,Sox10,Lpr5} {Frb,Tgfbr3,Cyba,Sox10,dkk3} {Frb,Tgfbr3,Cyba,Sox10,Nrf2} {Frb,Tgfbr3,Cyba,Plp1,Lpr5} {Frb,Tgfbr3,Cyba,Plp1,dkk3} {Frb,Tgfbr3,Cyba,Plp1,Nrf2} {Frb,Tgfbr3,Cyba,Lpr5,dkk3} {Frb,Tgfbr3,Cyba,Lpr5,Nrf2} {Frb,Tgfbr3,Cyba,dkk3,Nrf2} {Frb,Tgfbr3,Sox10,Plp1,Lpr5} {Frb,Tgfbr3,Sox10,Plp1,dkk3} {Frb,Tgfbr3,Sox10,Plp1,Nrf2} {Frb,Tgfbr3,Sox10,Lpr5,dkk3} {Frb,Tgfbr3,Sox10,Lpr5,Nrf2} {Frb,Tgfbr3,Sox10,dkk3,Nrf2} {Frb,Tgfbr3,Plp1,Lpr5,dkk3} {Frb,Tgfbr3,Plp1,Lpr5,Nrf2} {Frb,Tgfbr3,Plp1,dkk3,Nrf2} {Frb,Tgfbr3,Lpr5,dkk3,Nrf2} {Frb,Hmox1,Cyba,Sox10,Plp1} {Frb,Hmox1,Cyba,Sox10,Lpr5} {Frb,Hmox1,Cyba,Sox10,dkk3} {Frb,Hmox1,Cyba,Sox10,Nrf2} {Frb,Hmox1,Cyba,Plp1,Lpr5} {Frb,Hmox1,Cyba,Plp1,dkk3} {Frb,Hmox1,Cyba,Plp1,Nrf2} {Frb,Hmox1,Cyba,Lpr5,dkk3} {Frb,Hmox1,Cyba,Lpr5,Nrf2} {Frb,Hmox1,Cyba,dkk3,Nrf2} {Frb,Hmox1,Sox10,Plp1,Lpr5} {Frb,Hmox1,Sox10,Plp1,dkk3} {Frb,Hmox1,Sox10,Plp1,Nrf2} {Frb,Hmox1,Sox10,Lpr5,dkk3} {Frb,Hmox1,Sox10,Lpr5,Nrf2} {Frb,Hmox1,Sox10,dkk3,Nrf2} {Frb,Hmox1,Plp1,Lpr5,dkk3} {Frb,Hmox1,Plp1,Lpr5,Nrf2} {Frb,Hmox1,Plp1,dkk3,Nrf2} {Frb,Hmox1,Lpr5,dkk3,Nrf2} {Frb,Cyba,Sox10,Plp1,Lpr5} {Frb,Cyba,Sox10,Plp1,dkk3} {Frb,Cyba,Sox10,Plp1,Nrf2} {Frb,Cyba,Sox10,Lpr5,dkk3} {Frb,Cyba,Sox10,Lpr5,Nrf2} {Frb,Cyba,Sox10,dkk3,Nrf2} {Frb,Cyba,Plp1,Lpr5,dkk3} {Frb,Cyba,Plp1,Lpr5,Nrf2} {Frb,Cyba,Plp1,dkk3,Nrf2} {Frb,Cyba,Lpr5,dkk3,Nrf2} {Frb,Sox10,Plp1,Lpr5,dkk3} {Frb,Sox10,Plp1,Lpr5,Nrf2} {Frb,Sox10,Plp1,dkk3,Nrf2} {Frb,Sox10,Lpr5,dkk3,Nrf2} {Frb,Plp1,Lpr5,dkk3,Nrf2} {Nkd2,Tgfbr2,Tgfbr3,Hmox1,Cyba} {Nkd2,Tgfbr2,Tgfbr3,Hmox1,Sox10} {Nkd2,Tgfbr2,Tgfbr3,Hmox1,Plp1} {Nkd2,Tgfbr2,Tgfbr3,Hmox1,Lpr5} {Nkd2,Tgfbr2,Tgfbr3,Hmox1,dkk3} {Nkd2,Tgfbr2,Tgfbr3,Hmox1,Nrf2} {Nkd2,Tgfbr2,Tgfbr3,Cyba,Sox10} {Nkd2,Tgfbr2,Tgfbr3,Cyba,Plp1} {Nkd2,Tgfbr2,Tgfbr3,Cyba,Lpr5} {Nkd2,Tgfbr2,Tgfbr3,Cyba,dkk3} {Nkd2,Tgfbr2,Tgfbr3,Cyba,Nrf2} {Nkd2,Tgfbr2,Tgfbr3,Sox10,Plp1} {Nkd2,Tgfbr2,Tgfbr3,Sox10,Lpr5} {Nkd2,Tgfbr2,Tgfbr3,Sox10,dkk3} {Nkd2,Tgfbr2,Tgfbr3,Sox10,Nrf2} {Nkd2,Tgfbr2,Tgfbr3,Plp1,Lpr5} {Nkd2,Tgfbr2,Tgfbr3,Plp1,dkk3} {Nkd2,Tgfbr2,Tgfbr3,Plp1,Nrf2} {Nkd2,Tgfbr2,Tgfbr3,Lpr5,dkk3} {Nkd2,Tgfbr2,Tgfbr3,Lpr5,Nrf2} {Nkd2,Tgfbr2,Tgfbr3,dkk3,Nrf2} {Nkd2,Tgfbr2,Hmox1,Cyba,Sox10} {Nkd2,Tgfbr2,Hmox1,Cyba,Plp1} {Nkd2,Tgfbr2,Hmox1,Cyba,Lpr5} {Nkd2,Tgfbr2,Hmox1,Cyba,dkk3} {Nkd2,Tgfbr2,Hmox1,Cyba,Nrf2} {Nkd2,Tgfbr2,Hmox1,Sox10,Plp1} {Nkd2,Tgfbr2,Hmox1,Sox10,Lpr5} {Nkd2,Tgfbr2,Hmox1,Sox10,dkk3} {Nkd2,Tgfbr2,Hmox1,Sox10,Nrf2} {Nkd2,Tgfbr2,Hmox1,Plp1,Lpr5} {Nkd2,Tgfbr2,Hmox1,Plp1,dkk3} {Nkd2,Tgfbr2,Hmox1,Plp1,Nrf2} {Nkd2,Tgfbr2,Hmox1,Lpr5,dkk3} {Nkd2,Tgfbr2,Hmox1,Lpr5,Nrf2} {Nkd2,Tgfbr2,Hmox1,dkk3,Nrf2} {Nkd2,Tgfbr2,Cyba,Sox10,Plp1} {Nkd2,Tgfbr2,Cyba,Sox10,Lpr5} {Nkd2,Tgfbr2,Cyba,Sox10,dkk3} {Nkd2,Tgfbr2,Cyba,Sox10,Nrf2} {Nkd2,Tgfbr2,Cyba,Plp1,Lpr5} {Nkd2,Tgfbr2,Cyba,Plp1,dkk3} {Nkd2,Tgfbr2,Cyba,Plp1,Nrf2} {Nkd2,Tgfbr2,Cyba,Lpr5,dkk3} {Nkd2,Tgfbr2,Cyba,Lpr5,Nrf2} {Nkd2,Tgfbr2,Cyba,dkk3,Nrf2} {Nkd2,Tgfbr2,Sox10,Plp1,Lpr5} {Nkd2,Tgfbr2,Sox10,Plp1,dkk3} {Nkd2,Tgfbr2,Sox10,Plp1,Nrf2} {Nkd2,Tgfbr2,Sox10,Lpr5,dkk3} {Nkd2,Tgfbr2,Sox10,Lpr5,Nrf2} {Nkd2,Tgfbr2,Sox10,dkk3,Nrf2} {Nkd2,Tgfbr2,Plp1,Lpr5,dkk3} {Nkd2,Tgfbr2,Plp1,Lpr5,Nrf2} {Nkd2,Tgfbr2,Plp1,dkk3,Nrf2} {Nkd2,Tgfbr2,Lpr5,dkk3,Nrf2} {Nkd2,Tgfbr3,Hmox1,Cyba,Sox10} {Nkd2,Tgfbr3,Hmox1,Cyba,Plp1} {Nkd2,Tgfbr3,Hmox1,Cyba,Lpr5} {Nkd2,Tgfbr3,Hmox1,Cyba,dkk3} {Nkd2,Tgfbr3,Hmox1,Cyba,Nrf2} {Nkd2,Tgfbr3,Hmox1,Sox10,Plp1} {Nkd2,Tgfbr3,Hmox1,Sox10,Lpr5} {Nkd2,Tgfbr3,Hmox1,Sox10,dkk3} {Nkd2,Tgfbr3,Hmox1,Sox10,Nrf2} {Nkd2,Tgfbr3,Hmox1,Plp1,Lpr5} {Nkd2,Tgfbr3,Hmox1,Plp1,dkk3} {Nkd2,Tgfbr3,Hmox1,Plp1,Nrf2} {Nkd2,Tgfbr3,Hmox1,Lpr5,dkk3} {Nkd2,Tgfbr3,Hmox1,Lpr5,Nrf2} {Nkd2,Tgfbr3,Hmox1,dkk3,Nrf2} {Nkd2,Tgfbr3,Cyba,Sox10,Plp1} {Nkd2,Tgfbr3,Cyba,Sox10,Lpr5} {Nkd2,Tgfbr3,Cyba,Sox10,dkk3} {Nkd2,Tgfbr3,Cyba,Sox10,Nrf2} {Nkd2,Tgfbr3,Cyba,Plp1,Lpr5} {Nkd2,Tgfbr3,Cyba,Plp1,dkk3} {Nkd2,Tgfbr3,Cyba,Plp1,Nrf2} {Nkd2,Tgfbr3,Cyba,Lpr5,dkk3} {Nkd2,Tgfbr3,Cyba,Lpr5,Nrf2} {Nkd2,Tgfbr3,Cyba,dkk3,Nrf2} {Nkd2,Tgfbr3,Sox10,Plp1,Lpr5} {Nkd2,Tgfbr3,Sox10,Plp1,dkk3} {Nkd2,Tgfbr3,Sox10,Plp1,Nrf2} {Nkd2,Tgfbr3,Sox10,Lpr5,dkk3} {Nkd2,Tgfbr3,Sox10,Lpr5,Nrf2} {Nkd2,Tgfbr3,Sox10,dkk3,Nrf2} {Nkd2,Tgfbr3,Plp1,Lpr5,dkk3} {Nkd2,Tgfbr3,Plp1,Lpr5,Nrf2} {Nkd2,Tgfbr3,Plp1,dkk3,Nrf2} {Nkd2,Tgfbr3,Lpr5,dkk3,Nrf2} {Nkd2,Hmox1,Cyba,Sox10,Plp1} {Nkd2,Hmox1,Cyba,Sox10,Lpr5} {Nkd2,Hmox1,Cyba,Sox10,dkk3} {Nkd2,Hmox1,Cyba,Sox10,Nrf2} {Nkd2,Hmox1,Cyba,Plp1,Lpr5} {Nkd2,Hmox1,Cyba,Plp1,dkk3} {Nkd2,Hmox1,Cyba,Plp1,Nrf2} {Nkd2,Hmox1,Cyba,Lpr5,dkk3} {Nkd2,Hmox1,Cyba,Lpr5,Nrf2} {Nkd2,Hmox1,Cyba,dkk3,Nrf2} {Nkd2,Hmox1,Sox10,Plp1,Lpr5} {Nkd2,Hmox1,Sox10,Plp1,dkk3} {Nkd2,Hmox1,Sox10,Plp1,Nrf2} {Nkd2,Hmox1,Sox10,Lpr5,dkk3} {Nkd2,Hmox1,Sox10,Lpr5,Nrf2} {Nkd2,Hmox1,Sox10,dkk3,Nrf2} {Nkd2,Hmox1,Plp1,Lpr5,dkk3} {Nkd2,Hmox1,Plp1,Lpr5,Nrf2} {Nkd2,Hmox1,Plp1,dkk3,Nrf2} {Nkd2,Hmox1,Lpr5,dkk3,Nrf2} {Nkd2,Cyba,Sox10,Plp1,Lpr5} {Nkd2,Cyba,Sox10,Plp1,dkk3} {Nkd2,Cyba,Sox10,Plp1,Nrf2} {Nkd2,Cyba,Sox10,Lpr5,dkk3} {Nkd2,Cyba,Sox10,Lpr5,Nrf2} {Nkd2,Cyba,Sox10,dkk3,Nrf2} {Nkd2,Cyba,Plp1,Lpr5,dkk3} {Nkd2,Cyba,Plp1,Lpr5,Nrf2} {Nkd2,Cyba,Plp1,dkk3,Nrf2} {Nkd2,Cyba,Lpr5,dkk3,Nrf2} {Nkd2,Sox10,Plp1,Lpr5,dkk3} {Nkd2,Sox10,Plp1,Lpr5,Nrf2} {Nkd2,Sox10,Plp1,dkk3,Nrf2} {Nkd2,Sox10,Lpr5,dkk3,Nrf2} {Nkd2,Plp1,Lpr5,dkk3,Nrf2} {Tgfbr2,Tgfbr3,Hmox1,Cyba,Sox10} {Tgfbr2,Tgfbr3,Hmox1,Cyba,Plp1} {Tgfbr2,Tgfbr3,Hmox1,Cyba,Lpr5} {Tgfbr2,Tgfbr3,Hmox1,Cyba,dkk3} {Tgfbr2,Tgfbr3,Hmox1,Cyba,Nrf2} {Tgfbr2,Tgfbr3,Hmox1,Sox10,Plp1} {Tgfbr2,Tgfbr3,Hmox1,Sox10,Lpr5} {Tgfbr2,Tgfbr3,Hmox1,Sox10,dkk3} {Tgfbr2,Tgfbr3,Hmox1,Sox10,Nrf2} {Tgfbr2,Tgfbr3,Hmox1,Plp1,Lpr5} {Tgfbr2,Tgfbr3,Hmox1,Plp1,dkk3} {Tgfbr2,Tgfbr3,Hmox1,Plp1,Nrf2} {Tgfbr2,Tgfbr3,Hmox1,Lpr5,dkk3} {Tgfbr2,Tgfbr3,Hmox1,Lpr5,Nrf2} {Tgfbr2,Tgfbr3,Hmox1,dkk3,Nrf2} {Tgfbr2,Tgfbr3,Cyba,Sox10,Plp1} {Tgfbr2,Tgfbr3,Cyba,Sox10,Lpr5} {Tgfbr2,Tgfbr3,Cyba,Sox10,dkk3} {Tgfbr2,Tgfbr3,Cyba,Sox10,Nrf2} {Tgfbr2,Tgfbr3,Cyba,Plp1,Lpr5} {Tgfbr2,Tgfbr3,Cyba,Plp1,dkk3} {Tgfbr2,Tgfbr3,Cyba,Plp1,Nrf2} {Tgfbr2,Tgfbr3,Cyba,Lpr5,dkk3} {Tgfbr2,Tgfbr3,Cyba,Lpr5,Nrf2} {Tgfbr2,Tgfbr3,Cyba,dkk3,Nrf2} {Tgfbr2,Tgfbr3,Sox10,Plp1,Lpr5} {Tgfbr2,Tgfbr3,Sox10,Plp1,dkk3} {Tgfbr2,Tgfbr3,Sox10,Plp1,Nrf2} {Tgfbr2,Tgfbr3,Sox10,Lpr5,dkk3} {Tgfbr2,Tgfbr3,Sox10,Lpr5,Nrf2} {Tgfbr2,Tgfbr3,Sox10,dkk3,Nrf2} {Tgfbr2,Tgfbr3,Plp1,Lpr5,dkk3} {Tgfbr2,Tgfbr3,Plp1,Lpr5,Nrf2} {Tgfbr2,Tgfbr3,Plp1,dkk3,Nrf2} {Tgfbr2,Tgfbr3,Lpr5,dkk3,Nrf2} {Tgfbr2,Hmox1,Cyba,Sox10,Plp1} {Tgfbr2,Hmox1,Cyba,Sox10,Lpr5} {Tgfbr2,Hmox1,Cyba,Sox10,dkk3} {Tgfbr2,Hmox1,Cyba,Sox10,Nrf2} {Tgfbr2,Hmox1,Cyba,Plp1,Lpr5} {Tgfbr2,Hmox1,Cyba,Plp1,dkk3} {Tgfbr2,Hmox1,Cyba,Plp1,Nrf2} {Tgfbr2,Hmox1,Cyba,Lpr5,dkk3} {Tgfbr2,Hmox1,Cyba,Lpr5,Nrf2} {Tgfbr2,Hmox1,Cyba,dkk3,Nrf2} {Tgfbr2,Hmox1,Sox10,Plp1,Lpr5} {Tgfbr2,Hmox1,Sox10,Plp1,dkk3} {Tgfbr2,Hmox1,Sox10,Plp1,Nrf2} {Tgfbr2,Hmox1,Sox10,Lpr5,dkk3} {Tgfbr2,Hmox1,Sox10,Lpr5,Nrf2} {Tgfbr2,Hmox1,Sox10,dkk3,Nrf2} {Tgfbr2,Hmox1,Plp1,Lpr5,dkk3} {Tgfbr2,Hmox1,Plp1,Lpr5,Nrf2} {Tgfbr2,Hmox1,Plp1,dkk3,Nrf2} {Tgfbr2,Hmox1,Lpr5,dkk3,Nrf2} {Tgfbr2,Cyba,Sox10,Plp1,Lpr5} {Tgfbr2,Cyba,Sox10,Plp1,dkk3} {Tgfbr2,Cyba,Sox10,Plp1,Nrf2} {Tgfbr2,Cyba,Sox10,Lpr5,dkk3} {Tgfbr2,Cyba,Sox10,Lpr5,Nrf2} {Tgfbr2,Cyba,Sox10,dkk3,Nrf2} {Tgfbr2,Cyba,Plp1,Lpr5,dkk3} {Tgfbr2,Cyba,Plp1,Lpr5,Nrf2} {Tgfbr2,Cyba,Plp1,dkk3,Nrf2} {Tgfbr2,Cyba,Lpr5,dkk3,Nrf2} {Tgfbr2,Sox10,Plp1,Lpr5,dkk3} {Tgfbr2,Sox10,Plp1,Lpr5,Nrf2} {Tgfbr2,Sox10,Plp1,dkk3,Nrf2} {Tgfbr2,Sox10,Lpr5,dkk3,Nrf2} {Tgfbr2,Plp1,Lpr5,dkk3,Nrf2} {Tgfbr3,Hmox1,Cyba,Sox10,Plp1} {Tgfbr3,Hmox1,Cyba,Sox10,Lpr5} {Tgfbr3,Hmox1,Cyba,Sox10,dkk3} {Tgfbr3,Hmox1,Cyba,Sox10,Nrf2} {Tgfbr3,Hmox1,Cyba,Plp1,Lpr5} {Tgfbr3,Hmox1,Cyba,Plp1,dkk3} {Tgfbr3,Hmox1,Cyba,Plp1,Nrf2} {Tgfbr3,Hmox1,Cyba,Lpr5,dkk3} {Tgfbr3,Hmox1,Cyba,Lpr5,Nrf2} {Tgfbr3,Hmox1,Cyba,dkk3,Nrf2} {Tgfbr3,Hmox1,Sox10,Plp1,Lpr5} {Tgfbr3,Hmox1,Sox10,Plp1,dkk3} {Tgfbr3,Hmox1,Sox10,Plp1,Nrf2} {Tgfbr3,Hmox1,Sox10,Lpr5,dkk3} {Tgfbr3,Hmox1,Sox10,Lpr5,Nrf2} {Tgfbr3,Hmox1,Sox10,dkk3,Nrf2} {Tgfbr3,Hmox1,Plp1,Lpr5,dkk3} {Tgfbr3,Hmox1,Plp1,Lpr5,Nrf2} {Tgfbr3,Hmox1,Plp1,dkk3,Nrf2} {Tgfbr3,Hmox1,Lpr5,dkk3,Nrf2} {Tgfbr3,Cyba,Sox10,Plp1,Lpr5} {Tgfbr3,Cyba,Sox10,Plp1,dkk3} {Tgfbr3,Cyba,Sox10,Plp1,Nrf2} {Tgfbr3,Cyba,Sox10,Lpr5,dkk3} {Tgfbr3,Cyba,Sox10,Lpr5,Nrf2} {Tgfbr3,Cyba,Sox10,dkk3,Nrf2} {Tgfbr3,Cyba,Plp1,Lpr5,dkk3} {Tgfbr3,Cyba,Plp1,Lpr5,Nrf2} {Tgfbr3,Cyba,Plp1,dkk3,Nrf2} {Tgfbr3,Cyba,Lpr5,dkk3,Nrf2} {Tgfbr3,Sox10,Plp1,Lpr5,dkk3} {Tgfbr3,Sox10,Plp1,Lpr5,Nrf2} {Tgfbr3,Sox10,Plp1,dkk3,Nrf2} {Tgfbr3,Sox10,Lpr5,dkk3,Nrf2} {Tgfbr3,Plp1,Lpr5,dkk3,Nrf2} {Hmox1,Cyba,Sox10,Plp1,Lpr5} {Hmox1,Cyba,Sox10,Plp1,dkk3} {Hmox1,Cyba,Sox10,Plp1,Nrf2} {Hmox1,Cyba,Sox10,Lpr5,dkk3} {Hmox1,Cyba,Sox10,Lpr5,Nrf2} {Hmox1,Cyba,Sox10,dkk3,Nrf2} {Hmox1,Cyba,Plp1,Lpr5,dkk3} {Hmox1,Cyba,Plp1,Lpr5,Nrf2} {Hmox1,Cyba,Plp1,dkk3,Nrf2} {Hmox1,Cyba,Lpr5,dkk3,Nrf2} {Hmox1,Sox10,Plp1,Lpr5,dkk3} {Hmox1,Sox10,Plp1,Lpr5,Nrf2} {Hmox1,Sox10,Plp1,dkk3,Nrf2} {Hmox1,Sox10,Lpr5,dkk3,Nrf2} {Hmox1,Plp1,Lpr5,dkk3,Nrf2} {Cyba,Sox10,Plp1,Lpr5,dkk3} {Cyba,Sox10,Plp1,Lpr5,Nrf2} {Cyba,Sox10,Plp1,dkk3,Nrf2} {Cyba,Sox10,Lpr5,dkk3,Nrf2} {Cyba,Plp1,Lpr5,dkk3,Nrf2} {Sox10,Plp1,Lpr5,dkk3,Nrf2} {Lgr6,Frb,Nkd2,Tgfbr2,Tgfbr3,Hmox1} {Lgr6,Frb,Nkd2,Tgfbr2,Tgfbr3,Cyba} {Lgr6,Frb,Nkd2,Tgfbr2,Tgfbr3,Sox10} {Lgr6,Frb,Nkd2,Tgfbr2,Tgfbr3,Plp1} {Lgr6,Frb,Nkd2,Tgfbr2,Tgfbr3,Lpr5} {Lgr6,Frb,Nkd2,Tgfbr2,Tgfbr3,dkk3} {Lgr6,Frb,Nkd2,Tgfbr2,Tgfbr3,Nrf2} {Lgr6,Frb,Nkd2,Tgfbr2,Hmox1,Cyba} {Lgr6,Frb,Nkd2,Tgfbr2,Hmox1,Sox10} {Lgr6,Frb,Nkd2,Tgfbr2,Hmox1,Plp1} {Lgr6,Frb,Nkd2,Tgfbr2,Hmox1,Lpr5} {Lgr6,Frb,Nkd2,Tgfbr2,Hmox1,dkk3} {Lgr6,Frb,Nkd2,Tgfbr2,Hmox1,Nrf2} {Lgr6,Frb,Nkd2,Tgfbr2,Cyba,Sox10} {Lgr6,Frb,Nkd2,Tgfbr2,Cyba,Plp1} {Lgr6,Frb,Nkd2,Tgfbr2,Cyba,Lpr5} {Lgr6,Frb,Nkd2,Tgfbr2,Cyba,dkk3} {Lgr6,Frb,Nkd2,Tgfbr2,Cyba,Nrf2} {Lgr6,Frb,Nkd2,Tgfbr2,Sox10,Plp1} {Lgr6,Frb,Nkd2,Tgfbr2,Sox10,Lpr5} {Lgr6,Frb,Nkd2,Tgfbr2,Sox10,dkk3} {Lgr6,Frb,Nkd2,Tgfbr2,Sox10,Nrf2} {Lgr6,Frb,Nkd2,Tgfbr2,Plp1,Lpr5} {Lgr6,Frb,Nkd2,Tgfbr2,Plp1,dkk3} {Lgr6,Frb,Nkd2,Tgfbr2,Plp1,Nrf2} {Lgr6,Frb,Nkd2,Tgfbr2,Lpr5,dkk3} {Lgr6,Frb,Nkd2,Tgfbr2,Lpr5,Nrf2} {Lgr6,Frb,Nkd2,Tgfbr2,dkk3,Nrf2} {Lgr6,Frb,Nkd2,Tgfbr3,Hmox1,Cyba} {Lgr6,Frb,Nkd2,Tgfbr3,Hmox1,Sox10} {Lgr6,Frb,Nkd2,Tgfbr3,Hmox1,Plp1} {Lgr6,Frb,Nkd2,Tgfbr3,Hmox1,Lpr5} {Lgr6,Frb,Nkd2,Tgfbr3,Hmox1,dkk3} {Lgr6,Frb,Nkd2,Tgfbr3,Hmox1,Nrf2} {Lgr6,Frb,Nkd2,Tgfbr3,Cyba,Sox10} {Lgr6,Frb,Nkd2,Tgfbr3,Cyba,Plp1} {Lgr6,Frb,Nkd2,Tgfbr3,Cyba,Lpr5} {Lgr6,Frb,Nkd2,Tgfbr3,Cyba,dkk3} {Lgr6,Frb,Nkd2,Tgfbr3,Cyba,Nrf2} {Lgr6,Frb,Nkd2,Tgfbr3,Sox10,Plp1} {Lgr6,Frb,Nkd2,Tgfbr3,Sox10,Lpr5} {Lgr6,Frb,Nkd2,Tgfbr3,Sox10,dkk3} {Lgr6,Frb,Nkd2,Tgfbr3,Sox10,Nrf2} {Lgr6,Frb,Nkd2,Tgfbr3,Plp1,Lpr5} {Lgr6,Frb,Nkd2,Tgfbr3,Plp1,dkk3} {Lgr6,Frb,Nkd2,Tgfbr3,Plp1,Nrf2} {Lgr6,Frb,Nkd2,Tgfbr3,Lpr5,dkk3} {Lgr6,Frb,Nkd2,Tgfbr3,Lpr5,Nrf2} {Lgr6,Frb,Nkd2,Tgfbr3,dkk3,Nrf2} {Lgr6,Frb,Nkd2,Hmox1,Cyba,Sox10} {Lgr6,Frb,Nkd2,Hmox1,Cyba,Plp1} {Lgr6,Frb,Nkd2,Hmox1,Cyba,Lpr5} {Lgr6,Frb,Nkd2,Hmox1,Cyba,dkk3} {Lgr6,Frb,Nkd2,Hmox1,Cyba,Nrf2} {Lgr6,Frb,Nkd2,Hmox1,Sox10,Plp1} {Lgr6,Frb,Nkd2,Hmox1,Sox10,Lpr5} {Lgr6,Frb,Nkd2,Hmox1,Sox10,dkk3} {Lgr6,Frb,Nkd2,Hmox1,Sox10,Nrf2} {Lgr6,Frb,Nkd2,Hmox1,Plp1,Lpr5} {Lgr6,Frb,Nkd2,Hmox1,Plp1,dkk3} {Lgr6,Frb,Nkd2,Hmox1,Plp1,Nrf2} {Lgr6,Frb,Nkd2,Hmox1,Lpr5,dkk3} {Lgr6,Frb,Nkd2,Hmox1,Lpr5,Nrf2} {Lgr6,Frb,Nkd2,Hmox1,dkk3,Nrf2} {Lgr6,Frb,Nkd2,Cyba,Sox10,Plp1} {Lgr6,Frb,Nkd2,Cyba,Sox10,Lpr5} {Lgr6,Frb,Nkd2,Cyba,Sox10,dkk3} {Lgr6,Frb,Nkd2,Cyba,Sox10,Nrf2} {Lgr6,Frb,Nkd2,Cyba,Plp1,Lpr5} {Lgr6,Frb,Nkd2,Cyba,Plp1,dkk3} {Lgr6,Frb,Nkd2,Cyba,Plp1,Nrf2} {Lgr6,Frb,Nkd2,Cyba,Lpr5,dkk3} {Lgr6,Frb,Nkd2,Cyba,Lpr5,Nrf2} {Lgr6,Frb,Nkd2,Cyba,dkk3,Nrf2} {Lgr6,Frb,Nkd2,Sox10,Plp1,Lpr5} {Lgr6,Frb,Nkd2,Sox10,Plp1,dkk3} {Lgr6,Frb,Nkd2,Sox10,Plp1,Nrf2} {Lgr6,Frb,Nkd2,Sox10,Lpr5,dkk3} {Lgr6,Frb,Nkd2,Sox10,Lpr5,Nrf2} {Lgr6,Frb,Nkd2,Sox10,dkk3,Nrf2} {Lgr6,Frb,Nkd2,Plp1,Lpr5,dkk3} {Lgr6,Frb,Nkd2,Plp1,Lpr5,Nrf2} {Lgr6,Frb,Nkd2,Plp1,dkk3,Nrf2} {Lgr6,Frb,Nkd2,Lpr5,dkk3,Nrf2} {Lgr6,Frb,Tgfbr2,Tgfbr3,Hmox1,Cyba} {Lgr6,Frb,Tgfbr2,Tgfbr3,Hmox1,Sox10} {Lgr6,Frb,Tgfbr2,Tgfbr3,Hmox1,Plp1} {Lgr6,Frb,Tgfbr2,Tgfbr3,Hmox1,Lpr5} {Lgr6,Frb,Tgfbr2,Tgfbr3,Hmox1,dkk3} {Lgr6,Frb,Tgfbr2,Tgfbr3,Hmox1,Nrf2} {Lgr6,Frb,Tgfbr2,Tgfbr3,Cyba,Sox10} {Lgr6,Frb,Tgfbr2,Tgfbr3,Cyba,Plp1} {Lgr6,Frb,Tgfbr2,Tgfbr3,Cyba,Lpr5} {Lgr6,Frb,Tgfbr2,Tgfbr3,Cyba,dkk3} {Lgr6,Frb,Tgfbr2,Tgfbr3,Cyba,Nrf2} {Lgr6,Frb,Tgfbr2,Tgfbr3,Sox10,Plp1} {Lgr6,Frb,Tgfbr2,Tgfbr3,Sox10,Lpr5} {Lgr6,Frb,Tgfbr2,Tgfbr3,Sox10,dkk3} {Lgr6,Frb,Tgfbr2,Tgfbr3,Sox10,Nrf2} {Lgr6,Frb,Tgfbr2,Tgfbr3,Plp1,Lpr5} {Lgr6,Frb,Tgfbr2,Tgfbr3,Plp1,dkk3} {Lgr6,Frb,Tgfbr2,Tgfbr3,Plp1,Nrf2} {Lgr6,Frb,Tgfbr2,Tgfbr3,Lpr5,dkk3} {Lgr6,Frb,Tgfbr2,Tgfbr3,Lpr5,Nrf2} {Lgr6,Frb,Tgfbr2,Tgfbr3,dkk3,Nrf2} {Lgr6,Frb,Tgfbr2,Hmox1,Cyba,Sox10} {Lgr6,Frb,Tgfbr2,Hmox1,Cyba,Plp1} {Lgr6,Frb,Tgfbr2,Hmox1,Cyba,Lpr5} {Lgr6,Frb,Tgfbr2,Hmox1,Cyba,dkk3} {Lgr6,Frb,Tgfbr2,Hmox1,Cyba,Nrf2} {Lgr6,Frb,Tgfbr2,Hmox1,Sox10,Plp1} {Lgr6,Frb,Tgfbr2,Hmox1,Sox10,Lpr5} {Lgr6,Frb,Tgfbr2,Hmox1,Sox10,dkk3} {Lgr6,Frb,Tgfbr2,Hmox1,Sox10,Nrf2} {Lgr6,Frb,Tgfbr2,Hmox1,Plp1,Lpr5} {Lgr6,Frb,Tgfbr2,Hmox1,Plp1,dkk3} {Lgr6,Frb,Tgfbr2,Hmox1,Plp1,Nrf2} {Lgr6,Frb,Tgfbr2,Hmox1,Lpr5,dkk3} {Lgr6,Frb,Tgfbr2,Hmox1,Lpr5,Nrf2} {Lgr6,Frb,Tgfbr2,Hmox1,dkk3,Nrf2} {Lgr6,Frb,Tgfbr2,Cyba,Sox10,Plp1} {Lgr6,Frb,Tgfbr2,Cyba,Sox10,Lpr5} {Lgr6,Frb,Tgfbr2,Cyba,Sox10,dkk3} {Lgr6,Frb,Tgfbr2,Cyba,Sox10,Nrf2} {Lgr6,Frb,Tgfbr2,Cyba,Plp1,Lpr5} {Lgr6,Frb,Tgfbr2,Cyba,Plp1,dkk3} {Lgr6,Frb,Tgfbr2,Cyba,Plp1,Nrf2} {Lgr6,Frb,Tgfbr2,Cyba,Lpr5,dkk3} {Lgr6,Frb,Tgfbr2,Cyba,Lpr5,Nrf2} {Lgr6,Frb,Tgfbr2,Cyba,dkk3,Nrf2} {Lgr6,Frb,Tgfbr2,Sox10,Plp1,Lpr5} {Lgr6,Frb,Tgfbr2,Sox10,Plp1,dkk3} {Lgr6,Frb,Tgfbr2,Sox10,Plp1,Nrf2} {Lgr6,Frb,Tgfbr2,Sox10,Lpr5,dkk3} {Lgr6,Frb,Tgfbr2,Sox10,Lpr5,Nrf2} {Lgr6,Frb,Tgfbr2,Sox10,dkk3,Nrf2} {Lgr6,Frb,Tgfbr2,Plp1,Lpr5,dkk3} {Lgr6,Frb,Tgfbr2,Plp1,Lpr5,Nrf2} {Lgr6,Frb,Tgfbr2,Plp1,dkk3,Nrf2} {Lgr6,Frb,Tgfbr2,Lpr5,dkk3,Nrf2} {Lgr6,Frb,Tgfbr3,Hmox1,Cyba,Sox10} {Lgr6,Frb,Tgfbr3,Hmox1,Cyba,Plp1} {Lgr6,Frb,Tgfbr3,Hmox1,Cyba,Lpr5} {Lgr6,Frb,Tgfbr3,Hmox1,Cyba,dkk3} {Lgr6,Frb,Tgfbr3,Hmox1,Cyba,Nrf2} {Lgr6,Frb,Tgfbr3,Hmox1,Sox10,Plp1} {Lgr6,Frb,Tgfbr3,Hmox1,Sox10,Lpr5} {Lgr6,Frb,Tgfbr3,Hmox1,Sox10,dkk3} {Lgr6,Frb,Tgfbr3,Hmox1,Sox10,Nrf2} {Lgr6,Frb,Tgfbr3,Hmox1,Plp1,Lpr5} {Lgr6,Frb,Tgfbr3,Hmox1,Plp1,dkk3} {Lgr6,Frb,Tgfbr3,Hmox1,Plp1,Nrf2} {Lgr6,Frb,Tgfbr3,Hmox1,Lpr5,dkk3} {Lgr6,Frb,Tgfbr3,Hmox1,Lpr5,Nrf2} {Lgr6,Frb,Tgfbr3,Hmox1,dkk3,Nrf2} {Lgr6,Frb,Tgfbr3,Cyba,Sox10,Plp1} {Lgr6,Frb,Tgfbr3,Cyba,Sox10,Lpr5} {Lgr6,Frb,Tgfbr3,Cyba,Sox10,dkk3} {Lgr6,Frb,Tgfbr3,Cyba,Sox10,Nrf2} {Lgr6,Frb,Tgfbr3,Cyba,Plp1,Lpr5} {Lgr6,Frb,Tgfbr3,Cyba,Plp1,dkk3} {Lgr6,Frb,Tgfbr3,Cyba,Plp1,Nrf2} {Lgr6,Frb,Tgfbr3,Cyba,Lpr5,dkk3} {Lgr6,Frb,Tgfbr3,Cyba,Lpr5,Nrf2} {Lgr6,Frb,Tgfbr3,Cyba,dkk3,Nrf2} {Lgr6,Frb,Tgfbr3,Sox10,Plp1,Lpr5} {Lgr6,Frb,Tgfbr3,Sox10,Plp1,dkk3} {Lgr6,Frb,Tgfbr3,Sox10,Plp1,Nrf2} {Lgr6,Frb,Tgfbr3,Sox10,Lpr5,dkk3} {Lgr6,Frb,Tgfbr3,Sox10,Lpr5,Nrf2} {Lgr6,Frb,Tgfbr3,Sox10,dkk3,Nrf2} {Lgr6,Frb,Tgfbr3,Plp1,Lpr5,dkk3} {Lgr6,Frb,Tgfbr3,Plp1,Lpr5,Nrf2} {Lgr6,Frb,Tgfbr3,Plp1,dkk3,Nrf2} {Lgr6,Frb,Tgfbr3,Lpr5,dkk3,Nrf2} {Lgr6,Frb,Hmox1,Cyba,Sox10,Plp1} {Lgr6,Frb,Hmox1,Cyba,Sox10,Lpr5} {Lgr6,Frb,Hmox1,Cyba,Sox10,dkk3} {Lgr6,Frb,Hmox1,Cyba,Sox10,Nrf2} {Lgr6,Frb,Hmox1,Cyba,Plp1,Lpr5} {Lgr6,Frb,Hmox1,Cyba,Plp1,dkk3} {Lgr6,Frb,Hmox1,Cyba,Plp1,Nrf2} {Lgr6,Frb,Hmox1,Cyba,Lpr5,dkk3} {Lgr6,Frb,Hmox1,Cyba,Lpr5,Nrf2} {Lgr6,Frb,Hmox1,Cyba,dkk3,Nrf2} {Lgr6,Frb,Hmox1,Sox10,Plp1,Lpr5} {Lgr6,Frb,Hmox1,Sox10,Plp1,dkk3} {Lgr6,Frb,Hmox1,Sox10,Plp1,Nrf2} {Lgr6,Frb,Hmox1,Sox10,Lpr5,dkk3} {Lgr6,Frb,Hmox1,Sox10,Lpr5,Nrf2} {Lgr6,Frb,Hmox1,Sox10,dkk3,Nrf2} {Lgr6,Frb,Hmox1,Plp1,Lpr5,dkk3} {Lgr6,Frb,Hmox1,Plp1,Lpr5,Nrf2} {Lgr6,Frb,Hmox1,Plp1,dkk3,Nrf2} {Lgr6,Frb,Hmox1,Lpr5,dkk3,Nrf2} {Lgr6,Frb,Cyba,Sox10,Plp1,Lpr5} {Lgr6,Frb,Cyba,Sox10,Plp1,dkk3} {Lgr6,Frb,Cyba,Sox10,Plp1,Nrf2} {Lgr6,Frb,Cyba,Sox10,Lpr5,dkk3} {Lgr6,Frb,Cyba,Sox10,Lpr5,Nrf2} {Lgr6,Frb,Cyba,Sox10,dkk3,Nrf2} {Lgr6,Frb,Cyba,Plp1,Lpr5,dkk3} {Lgr6,Frb,Cyba,Plp1,Lpr5,Nrf2} {Lgr6,Frb,Cyba,Plp1,dkk3,Nrf2} {Lgr6,Frb,Cyba,Lpr5,dkk3,Nrf2} {Lgr6,Frb,Sox10,Plp1,Lpr5,dkk3} {Lgr6,Frb,Sox10,Plp1,Lpr5,Nrf2} {Lgr6,Frb,Sox10,Plp1,dkk3,Nrf2} {Lgr6,Frb,Sox10,Lpr5,dkk3,Nrf2} {Lgr6,Frb,Plp1,Lpr5,dkk3,Nrf2} {Lgr6,Nkd2,Tgfbr2,Tgfbr3,Hmox1,Cyba} {Lgr6,Nkd2,Tgfbr2,Tgfbr3,Hmox1,Sox10} {Lgr6,Nkd2,Tgfbr2,Tgfbr3,Hmox1,Plp1} {Lgr6,Nkd2,Tgfbr2,Tgfbr3,Hmox1,Lpr5} {Lgr6,Nkd2,Tgfbr2,Tgfbr3,Hmox1,dkk3} {Lgr6,Nkd2,Tgfbr2,Tgfbr3,Hmox1,Nrf2} {Lgr6,Nkd2,Tgfbr2,Tgfbr3,Cyba,Sox10} {Lgr6,Nkd2,Tgfbr2,Tgfbr3,Cyba,Plp1} {Lgr6,Nkd2,Tgfbr2,Tgfbr3,Cyba,Lpr5} {Lgr6,Nkd2,Tgfbr2,Tgfbr3,Cyba,dkk3} {Lgr6,Nkd2,Tgfbr2,Tgfbr3,Cyba,Nrf2} {Lgr6,Nkd2,Tgfbr2,Tgfbr3,Sox10,Plp1} {Lgr6,Nkd2,Tgfbr2,Tgfbr3,Sox10,Lpr5} {Lgr6,Nkd2,Tgfbr2,Tgfbr3,Sox10,dkk3} {Lgr6,Nkd2,Tgfbr2,Tgfbr3,Sox10,Nrf2} {Lgr6,Nkd2,Tgfbr2,Tgfbr3,Plp1,Lpr5} {Lgr6,Nkd2,Tgfbr2,Tgfbr3,Plp1,dkk3} {Lgr6,Nkd2,Tgfbr2,Tgfbr3,Plp1,Nrf2} {Lgr6,Nkd2,Tgfbr2,Tgfbr3,Lpr5,dkk3} {Lgr6,Nkd2,Tgfbr2,Tgfbr3,Lpr5,Nrf2} {Lgr6,Nkd2,Tgfbr2,Tgfbr3,dkk3,Nrf2} {Lgr6,Nkd2,Tgfbr2,Hmox1,Cyba,Sox10} {Lgr6,Nkd2,Tgfbr2,Hmox1,Cyba,Plp1} {Lgr6,Nkd2,Tgfbr2,Hmox1,Cyba,Lpr5} {Lgr6,Nkd2,Tgfbr2,Hmox1,Cyba,dkk3} {Lgr6,Nkd2,Tgfbr2,Hmox1,Cyba,Nrf2} {Lgr6,Nkd2,Tgfbr2,Hmox1,Sox10,Plp1} {Lgr6,Nkd2,Tgfbr2,Hmox1,Sox10,Lpr5} {Lgr6,Nkd2,Tgfbr2,Hmox1,Sox10,dkk3} {Lgr6,Nkd2,Tgfbr2,Hmox1,Sox10,Nrf2} {Lgr6,Nkd2,Tgfbr2,Hmox1,Plp1,Lpr5} {Lgr6,Nkd2,Tgfbr2,Hmox1,Plp1,dkk3} {Lgr6,Nkd2,Tgfbr2,Hmox1,Plp1,Nrf2} {Lgr6,Nkd2,Tgfbr2,Hmox1,Lpr5,dkk3} {Lgr6,Nkd2,Tgfbr2,Hmox1,Lpr5,Nrf2} {Lgr6,Nkd2,Tgfbr2,Hmox1,dkk3,Nrf2} {Lgr6,Nkd2,Tgfbr2,Cyba,Sox10,Plp1} {Lgr6,Nkd2,Tgfbr2,Cyba,Sox10,Lpr5} {Lgr6,Nkd2,Tgfbr2,Cyba,Sox10,dkk3} {Lgr6,Nkd2,Tgfbr2,Cyba,Sox10,Nrf2} {Lgr6,Nkd2,Tgfbr2,Cyba,Plp1,Lpr5} {Lgr6,Nkd2,Tgfbr2,Cyba,Plp1,dkk3} {Lgr6,Nkd2,Tgfbr2,Cyba,Plp1,Nrf2} {Lgr6,Nkd2,Tgfbr2,Cyba,Lpr5,dkk3} {Lgr6,Nkd2,Tgfbr2,Cyba,Lpr5,Nrf2} {Lgr6,Nkd2,Tgfbr2,Cyba,dkk3,Nrf2} {Lgr6,Nkd2,Tgfbr2,Sox10,Plp1,Lpr5} {Lgr6,Nkd2,Tgfbr2,Sox10,Plp1,dkk3} {Lgr6,Nkd2,Tgfbr2,Sox10,Plp1,Nrf2} {Lgr6,Nkd2,Tgfbr2,Sox10,Lpr5,dkk3} {Lgr6,Nkd2,Tgfbr2,Sox10,Lpr5,Nrf2} {Lgr6,Nkd2,Tgfbr2,Sox10,dkk3,Nrf2} {Lgr6,Nkd2,Tgfbr2,Plp1,Lpr5,dkk3} {Lgr6,Nkd2,Tgfbr2,Plp1,Lpr5,Nrf2} {Lgr6,Nkd2,Tgfbr2,Plp1,dkk3,Nrf2} {Lgr6,Nkd2,Tgfbr2,Lpr5,dkk3,Nrf2} {Lgr6,Nkd2,Tgfbr3,Hmox1,Cyba,Sox10} {Lgr6,Nkd2,Tgfbr3,Hmox1,Cyba,Plp1} {Lgr6,Nkd2,Tgfbr3,Hmox1,Cyba,Lpr5} {Lgr6,Nkd2,Tgfbr3,Hmox1,Cyba,dkk3} {Lgr6,Nkd2,Tgfbr3,Hmox1,Cyba,Nrf2} {Lgr6,Nkd2,Tgfbr3,Hmox1,Sox10,Plp1} {Lgr6,Nkd2,Tgfbr3,Hmox1,Sox10,Lpr5} {Lgr6,Nkd2,Tgfbr3,Hmox1,Sox10,dkk3} {Lgr6,Nkd2,Tgfbr3,Hmox1,Sox10,Nrf2} {Lgr6,Nkd2,Tgfbr3,Hmox1,Plp1,Lpr5} {Lgr6,Nkd2,Tgfbr3,Hmox1,Plp1,dkk3} {Lgr6,Nkd2,Tgfbr3,Hmox1,Plp1,Nrf2} {Lgr6,Nkd2,Tgfbr3,Hmox1,Lpr5,dkk3} {Lgr6,Nkd2,Tgfbr3,Hmox1,Lpr5,Nrf2} {Lgr6,Nkd2,Tgfbr3,Hmox1,dkk3,Nrf2} {Lgr6,Nkd2,Tgfbr3,Cyba,Sox10,Plp1} {Lgr6,Nkd2,Tgfbr3,Cyba,Sox10,Lpr5} {Lgr6,Nkd2,Tgfbr3,Cyba,Sox10,dkk3} {Lgr6,Nkd2,Tgfbr3,Cyba,Sox10,Nrf2} {Lgr6,Nkd2,Tgfbr3,Cyba,Plp1,Lpr5} {Lgr6,Nkd2,Tgfbr3,Cyba,Plp1,dkk3} {Lgr6,Nkd2,Tgfbr3,Cyba,Plp1,Nrf2} {Lgr6,Nkd2,Tgfbr3,Cyba,Lpr5,dkk3} {Lgr6,Nkd2,Tgfbr3,Cyba,Lpr5,Nrf2} {Lgr6,Nkd2,Tgfbr3,Cyba,dkk3,Nrf2} {Lgr6,Nkd2,Tgfbr3,Sox10,Plp1,Lpr5} {Lgr6,Nkd2,Tgfbr3,Sox10,Plp1,dkk3} {Lgr6,Nkd2,Tgfbr3,Sox10,Plp1,Nrf2} {Lgr6,Nkd2,Tgfbr3,Sox10,Lpr5,dkk3} {Lgr6,Nkd2,Tgfbr3,Sox10,Lpr5,Nrf2} {Lgr6,Nkd2,Tgfbr3,Sox10,dkk3,Nrf2} {Lgr6,Nkd2,Tgfbr3,Plp1,Lpr5,dkk3} {Lgr6,Nkd2,Tgfbr3,Plp1,Lpr5,Nrf2} {Lgr6,Nkd2,Tgfbr3,Plp1,dkk3,Nrf2} {Lgr6,Nkd2,Tgfbr3,Lpr5,dkk3,Nrf2} {Lgr6,Nkd2,Hmox1,Cyba,Sox10,Plp1} {Lgr6,Nkd2,Hmox1,Cyba,Sox10,Lpr5} {Lgr6,Nkd2,Hmox1,Cyba,Sox10,dkk3} {Lgr6,Nkd2,Hmox1,Cyba,Sox10,Nrf2} {Lgr6,Nkd2,Hmox1,Cyba,Plp1,Lpr5} {Lgr6,Nkd2,Hmox1,Cyba,Plp1,dkk3} {Lgr6,Nkd2,Hmox1,Cyba,Plp1,Nrf2} {Lgr6,Nkd2,Hmox1,Cyba,Lpr5,dkk3} {Lgr6,Nkd2,Hmox1,Cyba,Lpr5,Nrf2} {Lgr6,Nkd2,Hmox1,Cyba,dkk3,Nrf2} {Lgr6,Nkd2,Hmox1,Sox10,Plp1,Lpr5} {Lgr6,Nkd2,Hmox1,Sox10,Plp1,dkk3} {Lgr6,Nkd2,Hmox1,Sox10,Plp1,Nrf2} {Lgr6,Nkd2,Hmox1,Sox10,Lpr5,dkk3} {Lgr6,Nkd2,Hmox1,Sox10,Lpr5,Nrf2} {Lgr6,Nkd2,Hmox1,Sox10,dkk3,Nrf2} {Lgr6,Nkd2,Hmox1,Plp1,Lpr5,dkk3} {Lgr6,Nkd2,Hmox1,Plp1,Lpr5,Nrf2} {Lgr6,Nkd2,Hmox1,Plp1,dkk3,Nrf2} {Lgr6,Nkd2,Hmox1,Lpr5,dkk3,Nrf2} {Lgr6,Nkd2,Cyba,Sox10,Plp1,Lpr5} {Lgr6,Nkd2,Cyba,Sox10,Plp1,dkk3} {Lgr6,Nkd2,Cyba,Sox10,Plp1,Nrf2} {Lgr6,Nkd2,Cyba,Sox10,Lpr5,dkk3} {Lgr6,Nkd2,Cyba,Sox10,Lpr5,Nrf2} {Lgr6,Nkd2,Cyba,Sox10,dkk3,Nrf2} {Lgr6,Nkd2,Cyba,Plp1,Lpr5,dkk3} {Lgr6,Nkd2,Cyba,Plp1,Lpr5,Nrf2} {Lgr6,Nkd2,Cyba,Plp1,dkk3,Nrf2} {Lgr6,Nkd2,Cyba,Lpr5,dkk3,Nrf2} {Lgr6,Nkd2,Sox10,Plp1,Lpr5,dkk3} {Lgr6,Nkd2,Sox10,Plp1,Lpr5,Nrf2} {Lgr6,Nkd2,Sox10,Plp1,dkk3,Nrf2} {Lgr6,Nkd2,Sox10,Lpr5,dkk3,Nrf2} {Lgr6,Nkd2,Plp1,Lpr5,dkk3,Nrf2} {Lgr6,Tgfbr2,Tgfbr3,Hmox1,Cyba,Sox10} {Lgr6,Tgfbr2,Tgfbr3,Hmox1,Cyba,Plp1} {Lgr6,Tgfbr2,Tgfbr3,Hmox1,Cyba,Lpr5} {Lgr6,Tgfbr2,Tgfbr3,Hmox1,Cyba,dkk3} {Lgr6,Tgfbr2,Tgfbr3,Hmox1,Cyba,Nrf2} {Lgr6,Tgfbr2,Tgfbr3,Hmox1,Sox10,Plp1} {Lgr6,Tgfbr2,Tgfbr3,Hmox1,Sox10,Lpr5} {Lgr6,Tgfbr2,Tgfbr3,Hmox1,Sox10,dkk3} {Lgr6,Tgfbr2,Tgfbr3,Hmox1,Sox10,Nrf2} {Lgr6,Tgfbr2,Tgfbr3,Hmox1,Plp1,Lpr5} {Lgr6,Tgfbr2,Tgfbr3,Hmox1,Plp1,dkk3} {Lgr6,Tgfbr2,Tgfbr3,Hmox1,Plp1,Nrf2} {Lgr6,Tgfbr2,Tgfbr3,Hmox1,Lpr5,dkk3} {Lgr6,Tgfbr2,Tgfbr3,Hmox1,Lpr5,Nrf2} {Lgr6,Tgfbr2,Tgfbr3,Hmox1,dkk3,Nrf2} {Lgr6,Tgfbr2,Tgfbr3,Cyba,Sox10,Plp1} {Lgr6,Tgfbr2,Tgfbr3,Cyba,Sox10,Lpr5} {Lgr6,Tgfbr2,Tgfbr3,Cyba,Sox10,dkk3} {Lgr6,Tgfbr2,Tgfbr3,Cyba,Sox10,Nrf2} {Lgr6,Tgfbr2,Tgfbr3,Cyba,Plp1,Lpr5} {Lgr6,Tgfbr2,Tgfbr3,Cyba,Plp1,dkk3} {Lgr6,Tgfbr2,Tgfbr3,Cyba,Plp1,Nrf2} {Lgr6,Tgfbr2,Tgfbr3,Cyba,Lpr5,dkk3} {Lgr6,Tgfbr2,Tgfbr3,Cyba,Lpr5,Nrf2} {Lgr6,Tgfbr2,Tgfbr3,Cyba,dkk3,Nrf2} {Lgr6,Tgfbr2,Tgfbr3,Sox10,Plp1,Lpr5} {Lgr6,Tgfbr2,Tgfbr3,Sox10,Plp1,dkk3} {Lgr6,Tgfbr2,Tgfbr3,Sox10,Plp1,Nrf2} {Lgr6,Tgfbr2,Tgfbr3,Sox10,Lpr5,dkk3} {Lgr6,Tgfbr2,Tgfbr3,Sox10,Lpr5,Nrf2} {Lgr6,Tgfbr2,Tgfbr3,Sox10,dkk3,Nrf2} {Lgr6,Tgfbr2,Tgfbr3,Plp1,Lpr5,dkk3} {Lgr6,Tgfbr2,Tgfbr3,Plp1,Lpr5,Nrf2} {Lgr6,Tgfbr2,Tgfbr3,Plp1,dkk3,Nrf2} {Lgr6,Tgfbr2,Tgfbr3,Lpr5,dkk3,Nrf2} {Lgr6,Tgfbr2,Hmox1,Cyba,Sox10,Plp1} {Lgr6,Tgfbr2,Hmox1,Cyba,Sox10,Lpr5} {Lgr6,Tgfbr2,Hmox1,Cyba,Sox10,dkk3} {Lgr6,Tgfbr2,Hmox1,Cyba,Sox10,Nrf2} {Lgr6,Tgfbr2,Hmox1,Cyba,Plp1,Lpr5} {Lgr6,Tgfbr2,Hmox1,Cyba,Plp1,dkk3} {Lgr6,Tgfbr2,Hmox1,Cyba,Plp1,Nrf2} {Lgr6,Tgfbr2,Hmox1,Cyba,Lpr5,dkk3} {Lgr6,Tgfbr2,Hmox1,Cyba,Lpr5,Nrf2} {Lgr6,Tgfbr2,Hmox1,Cyba,dkk3,Nrf2} {Lgr6,Tgfbr2,Hmox1,Sox10,Plp1,Lpr5} {Lgr6,Tgfbr2,Hmox1,Sox10,Plp1,dkk3} {Lgr6,Tgfbr2,Hmox1,Sox10,Plp1,Nrf2} {Lgr6,Tgfbr2,Hmox1,Sox10,Lpr5,dkk3} {Lgr6,Tgfbr2,Hmox1,Sox10,Lpr5,Nrf2} {Lgr6,Tgfbr2,Hmox1,Sox10,dkk3,Nrf2} {Lgr6,Tgfbr2,Hmox1,Plp1,Lpr5,dkk3} {Lgr6,Tgfbr2,Hmox1,Plp1,Lpr5,Nrf2} {Lgr6,Tgfbr2,Hmox1,Plp1,dkk3,Nrf2} {Lgr6,Tgfbr2,Hmox1,Lpr5,dkk3,Nrf2} {Lgr6,Tgfbr2,Cyba,Sox10,Plp1,Lpr5} {Lgr6,Tgfbr2,Cyba,Sox10,Plp1,dkk3} {Lgr6,Tgfbr2,Cyba,Sox10,Plp1,Nrf2} {Lgr6,Tgfbr2,Cyba,Sox10,Lpr5,dkk3} {Lgr6,Tgfbr2,Cyba,Sox10,Lpr5,Nrf2} {Lgr6,Tgfbr2,Cyba,Sox10,dkk3,Nrf2} {Lgr6,Tgfbr2,Cyba,Plp1,Lpr5,dkk3} {Lgr6,Tgfbr2,Cyba,Plp1,Lpr5,Nrf2} {Lgr6,Tgfbr2,Cyba,Plp1,dkk3,Nrf2} {Lgr6,Tgfbr2,Cyba,Lpr5,dkk3,Nrf2} {Lgr6,Tgfbr2,Sox10,Plp1,Lpr5,dkk3} {Lgr6,Tgfbr2,Sox10,Plp1,Lpr5,Nrf2} {Lgr6,Tgfbr2,Sox10,Plp1,dkk3,Nrf2} {Lgr6,Tgfbr2,Sox10,Lpr5,dkk3,Nrf2} {Lgr6,Tgfbr2,Plp1,Lpr5,dkk3,Nrf2} {Lgr6,Tgfbr3,Hmox1,Cyba,Sox10,Plp1} {Lgr6,Tgfbr3,Hmox1,Cyba,Sox10,Lpr5} {Lgr6,Tgfbr3,Hmox1,Cyba,Sox10,dkk3} {Lgr6,Tgfbr3,Hmox1,Cyba,Sox10,Nrf2} {Lgr6,Tgfbr3,Hmox1,Cyba,Plp1,Lpr5} {Lgr6,Tgfbr3,Hmox1,Cyba,Plp1,dkk3} {Lgr6,Tgfbr3,Hmox1,Cyba,Plp1,Nrf2} {Lgr6,Tgfbr3,Hmox1,Cyba,Lpr5,dkk3} {Lgr6,Tgfbr3,Hmox1,Cyba,Lpr5,Nrf2} {Lgr6,Tgfbr3,Hmox1,Cyba,dkk3,Nrf2} {Lgr6,Tgfbr3,Hmox1,Sox10,Plp1,Lpr5} {Lgr6,Tgfbr3,Hmox1,Sox10,Plp1,dkk3} {Lgr6,Tgfbr3,Hmox1,Sox10,Plp1,Nrf2} {Lgr6,Tgfbr3,Hmox1,Sox10,Lpr5,dkk3} {Lgr6,Tgfbr3,Hmox1,Sox10,Lpr5,Nrf2} {Lgr6,Tgfbr3,Hmox1,Sox10,dkk3,Nrf2} {Lgr6,Tgfbr3,Hmox1,Plp1,Lpr5,dkk3} {Lgr6,Tgfbr3,Hmox1,Plp1,Lpr5,Nrf2} {Lgr6,Tgfbr3,Hmox1,Plp1,dkk3,Nrf2} {Lgr6,Tgfbr3,Hmox1,Lpr5,dkk3,Nrf2} {Lgr6,Tgfbr3,Cyba,Sox10,Plp1,Lpr5} {Lgr6,Tgfbr3,Cyba,Sox10,Plp1,dkk3} {Lgr6,Tgfbr3,Cyba,Sox10,Plp1,Nrf2} {Lgr6,Tgfbr3,Cyba,Sox10,Lpr5,dkk3} {Lgr6,Tgfbr3,Cyba,Sox10,Lpr5,Nrf2} {Lgr6,Tgfbr3,Cyba,Sox10,dkk3,Nrf2} {Lgr6,Tgfbr3,Cyba,Plp1,Lpr5,dkk3} {Lgr6,Tgfbr3,Cyba,Plp1,Lpr5,Nrf2} {Lgr6,Tgfbr3,Cyba,Plp1,dkk3,Nrf2} {Lgr6,Tgfbr3,Cyba,Lpr5,dkk3,Nrf2} {Lgr6,Tgfbr3,Sox10,Plp1,Lpr5,dkk3} {Lgr6,Tgfbr3,Sox10,Plp1,Lpr5,Nrf2} {Lgr6,Tgfbr3,Sox10,Plp1,dkk3,Nrf2} {Lgr6,Tgfbr3,Sox10,Lpr5,dkk3,Nrf2} {Lgr6,Tgfbr3,Plp1,Lpr5,dkk3,Nrf2} {Lgr6,Hmox1,Cyba,Sox10,Plp1,Lpr5} {Lgr6,Hmox1,Cyba,Sox10,Plp1,dkk3} {Lgr6,Hmox1,Cyba,Sox10,Plp1,Nrf2} {Lgr6,Hmox1,Cyba,Sox10,Lpr5,dkk3} {Lgr6,Hmox1,Cyba,Sox10,Lpr5,Nrf2} {Lgr6,Hmox1,Cyba,Sox10,dkk3,Nrf2} {Lgr6,Hmox1,Cyba,Plp1,Lpr5,dkk3} {Lgr6,Hmox1,Cyba,Plp1,Lpr5,Nrf2} {Lgr6,Hmox1,Cyba,Plp1,dkk3,Nrf2} {Lgr6,Hmox1,Cyba,Lpr5,dkk3,Nrf2} {Lgr6,Hmox1,Sox10,Plp1,Lpr5,dkk3} {Lgr6,Hmox1,Sox10,Plp1,Lpr5,Nrf2} {Lgr6,Hmox1,Sox10,Plp1,dkk3,Nrf2} {Lgr6,Hmox1,Sox10,Lpr5,dkk3,Nrf2} {Lgr6,Hmox1,Plp1,Lpr5,dkk3,Nrf2} {Lgr6,Cyba,Sox10,Plp1,Lpr5,dkk3} {Lgr6,Cyba,Sox10,Plp1,Lpr5,Nrf2} {Lgr6,Cyba,Sox10,Plp1,dkk3,Nrf2} {Lgr6,Cyba,Sox10,Lpr5,dkk3,Nrf2} {Lgr6,Cyba,Plp1,Lpr5,dkk3,Nrf2} {Lgr6,Sox10,Plp1,Lpr5,dkk3,Nrf2} {Frb,Nkd2,Tgfbr2,Tgfbr3,Hmox1,Cyba} {Frb,Nkd2,Tgfbr2,Tgfbr3,Hmox1,Sox10} {Frb,Nkd2,Tgfbr2,Tgfbr3,Hmox1,Plp1} {Frb,Nkd2,Tgfbr2,Tgfbr3,Hmox1,Lpr5} {Frb,Nkd2,Tgfbr2,Tgfbr3,Hmox1,dkk3} {Frb,Nkd2,Tgfbr2,Tgfbr3,Hmox1,Nrf2} {Frb,Nkd2,Tgfbr2,Tgfbr3,Cyba,Sox10} {Frb,Nkd2,Tgfbr2,Tgfbr3,Cyba,Plp1} {Frb,Nkd2,Tgfbr2,Tgfbr3,Cyba,Lpr5} {Frb,Nkd2,Tgfbr2,Tgfbr3,Cyba,dkk3} {Frb,Nkd2,Tgfbr2,Tgfbr3,Cyba,Nrf2} {Frb,Nkd2,Tgfbr2,Tgfbr3,Sox10,Plp1} {Frb,Nkd2,Tgfbr2,Tgfbr3,Sox10,Lpr5} {Frb,Nkd2,Tgfbr2,Tgfbr3,Sox10,dkk3} {Frb,Nkd2,Tgfbr2,Tgfbr3,Sox10,Nrf2} {Frb,Nkd2,Tgfbr2,Tgfbr3,Plp1,Lpr5} {Frb,Nkd2,Tgfbr2,Tgfbr3,Plp1,dkk3} {Frb,Nkd2,Tgfbr2,Tgfbr3,Plp1,Nrf2} {Frb,Nkd2,Tgfbr2,Tgfbr3,Lpr5,dkk3} {Frb,Nkd2,Tgfbr2,Tgfbr3,Lpr5,Nrf2} {Frb,Nkd2,Tgfbr2,Tgfbr3,dkk3,Nrf2} {Frb,Nkd2,Tgfbr2,Hmox1,Cyba,Sox10} {Frb,Nkd2,Tgfbr2,Hmox1,Cyba,Plp1} {Frb,Nkd2,Tgfbr2,Hmox1,Cyba,Lpr5} {Frb,Nkd2,Tgfbr2,Hmox1,Cyba,dkk3} {Frb,Nkd2,Tgfbr2,Hmox1,Cyba,Nrf2} {Frb,Nkd2,Tgfbr2,Hmox1,Sox10,Plp1} {Frb,Nkd2,Tgfbr2,Hmox1,Sox10,Lpr5} {Frb,Nkd2,Tgfbr2,Hmox1,Sox10,dkk3} {Frb,Nkd2,Tgfbr2,Hmox1,Sox10,Nrf2} {Frb,Nkd2,Tgfbr2,Hmox1,Plp1,Lpr5} {Frb,Nkd2,Tgfbr2,Hmox1,Plp1,dkk3} {Frb,Nkd2,Tgfbr2,Hmox1,Plp1,Nrf2} {Frb,Nkd2,Tgfbr2,Hmox1,Lpr5,dkk3} {Frb,Nkd2,Tgfbr2,Hmox1,Lpr5,Nrf2} {Frb,Nkd2,Tgfbr2,Hmox1,dkk3,Nrf2} {Frb,Nkd2,Tgfbr2,Cyba,Sox10,Plp1} {Frb,Nkd2,Tgfbr2,Cyba,Sox10,Lpr5} {Frb,Nkd2,Tgfbr2,Cyba,Sox10,dkk3} {Frb,Nkd2,Tgfbr2,Cyba,Sox10,Nrf2} {Frb,Nkd2,Tgfbr2,Cyba,Plp1,Lpr5} {Frb,Nkd2,Tgfbr2,Cyba,Plp1,dkk3} {Frb,Nkd2,Tgfbr2,Cyba,Plp1,Nrf2} {Frb,Nkd2,Tgfbr2,Cyba,Lpr5,dkk3} {Frb,Nkd2,Tgfbr2,Cyba,Lpr5,Nrf2} {Frb,Nkd2,Tgfbr2,Cyba,dkk3,Nrf2} {Frb,Nkd2,Tgfbr2,Sox10,Plp1,Lpr5} {Frb,Nkd2,Tgfbr2,Sox10,Plp1,dkk3} {Frb,Nkd2,Tgfbr2,Sox10,Plp1,Nrf2} {Frb,Nkd2,Tgfbr2,Sox10,Lpr5,dkk3} {Frb,Nkd2,Tgfbr2,Sox10,Lpr5,Nrf2} {Frb,Nkd2,Tgfbr2,Sox10,dkk3,Nrf2} {Frb,Nkd2,Tgfbr2,Plp1,Lpr5,dkk3} {Frb,Nkd2,Tgfbr2,Plp1,Lpr5,Nrf2} {Frb,Nkd2,Tgfbr2,Plp1,dkk3,Nrf2} {Frb,Nkd2,Tgfbr2,Lpr5,dkk3,Nrf2} {Frb,Nkd2,Tgfbr3,Hmox1,Cyba,Sox10} {Frb,Nkd2,Tgfbr3,Hmox1,Cyba,Plp1} {Frb,Nkd2,Tgfbr3,Hmox1,Cyba,Lpr5} {Frb,Nkd2,Tgfbr3,Hmox1,Cyba,dkk3} {Frb,Nkd2,Tgfbr3,Hmox1,Cyba,Nrf2} {Frb,Nkd2,Tgfbr3,Hmox1,Sox10,Plp1} {Frb,Nkd2,Tgfbr3,Hmox1,Sox10,Lpr5} {Frb,Nkd2,Tgfbr3,Hmox1,Sox10,dkk3} {Frb,Nkd2,Tgfbr3,Hmox1,Sox10,Nrf2} {Frb,Nkd2,Tgfbr3,Hmox1,Plp1,Lpr5} {Frb,Nkd2,Tgfbr3,Hmox1,Plp1,dkk3} {Frb,Nkd2,Tgfbr3,Hmox1,Plp1,Nrf2} {Frb,Nkd2,Tgfbr3,Hmox1,Lpr5,dkk3} {Frb,Nkd2,Tgfbr3,Hmox1,Lpr5,Nrf2} {Frb,Nkd2,Tgfbr3,Hmox1,dkk3,Nrf2} {Frb,Nkd2,Tgfbr3,Cyba,Sox10,Plp1} {Frb,Nkd2,Tgfbr3,Cyba,Sox10,Lpr5} {Frb,Nkd2,Tgfbr3,Cyba,Sox10,dkk3} {Frb,Nkd2,Tgfbr3,Cyba,Sox10,Nrf2} {Frb,Nkd2,Tgfbr3,Cyba,Plp1,Lpr5} {Frb,Nkd2,Tgfbr3,Cyba,Plp1,dkk3} {Frb,Nkd2,Tgfbr3,Cyba,Plp1,Nrf2} {Frb,Nkd2,Tgfbr3,Cyba,Lpr5,dkk3} {Frb,Nkd2,Tgfbr3,Cyba,Lpr5,Nrf2} {Frb,Nkd2,Tgfbr3,Cyba,dkk3,Nrf2} {Frb,Nkd2,Tgfbr3,Sox10,Plp1,Lpr5} {Frb,Nkd2,Tgfbr3,Sox10,Plp1,dkk3} {Frb,Nkd2,Tgfbr3,Sox10,Plp1,Nrf2} {Frb,Nkd2,Tgfbr3,Sox10,Lpr5,dkk3} {Frb,Nkd2,Tgfbr3,Sox10,Lpr5,Nrf2} {Frb,Nkd2,Tgfbr3,Sox10,dkk3,Nrf2} {Frb,Nkd2,Tgfbr3,Plp1,Lpr5,dkk3} {Frb,Nkd2,Tgfbr3,Plp1,Lpr5,Nrf2} {Frb,Nkd2,Tgfbr3,Plp1,dkk3,Nrf2} {Frb,Nkd2,Tgfbr3,Lpr5,dkk3,Nrf2} {Frb,Nkd2,Hmox1,Cyba,Sox10,Plp1} {Frb,Nkd2,Hmox1,Cyba,Sox10,Lpr5} {Frb,Nkd2,Hmox1,Cyba,Sox10,dkk3} {Frb,Nkd2,Hmox1,Cyba,Sox10,Nrf2} {Frb,Nkd2,Hmox1,Cyba,Plp1,Lpr5} {Frb,Nkd2,Hmox1,Cyba,Plp1,dkk3} {Frb,Nkd2,Hmox1,Cyba,Plp1,Nrf2} {Frb,Nkd2,Hmox1,Cyba,Lpr5,dkk3} {Frb,Nkd2,Hmox1,Cyba,Lpr5,Nrf2} {Frb,Nkd2,Hmox1,Cyba,dkk3,Nrf2} {Frb,Nkd2,Hmox1,Sox10,Plp1,Lpr5} {Frb,Nkd2,Hmox1,Sox10,Plp1,dkk3} {Frb,Nkd2,Hmox1,Sox10,Plp1,Nrf2} {Frb,Nkd2,Hmox1,Sox10,Lpr5,dkk3} {Frb,Nkd2,Hmox1,Sox10,Lpr5,Nrf2} {Frb,Nkd2,Hmox1,Sox10,dkk3,Nrf2} {Frb,Nkd2,Hmox1,Plp1,Lpr5,dkk3} {Frb,Nkd2,Hmox1,Plp1,Lpr5,Nrf2} {Frb,Nkd2,Hmox1,Plp1,dkk3,Nrf2} {Frb,Nkd2,Hmox1,Lpr5,dkk3,Nrf2} {Frb,Nkd2,Cyba,Sox10,Plp1,Lpr5} {Frb,Nkd2,Cyba,Sox10,Plp1,dkk3} {Frb,Nkd2,Cyba,Sox10,Plp1,Nrf2} {Frb,Nkd2,Cyba,Sox10,Lpr5,dkk3} {Frb,Nkd2,Cyba,Sox10,Lpr5,Nrf2} {Frb,Nkd2,Cyba,Sox10,dkk3,Nrf2} {Frb,Nkd2,Cyba,Plp1,Lpr5,dkk3} {Frb,Nkd2,Cyba,Plp1,Lpr5,Nrf2} {Frb,Nkd2,Cyba,Plp1,dkk3,Nrf2} {Frb,Nkd2,Cyba,Lpr5,dkk3,Nrf2} {Frb,Nkd2,Sox10,Plp1,Lpr5,dkk3} {Frb,Nkd2,Sox10,Plp1,Lpr5,Nrf2} {Frb,Nkd2,Sox10,Plp1,dkk3,Nrf2} {Frb,Nkd2,Sox10,Lpr5,dkk3,Nrf2} {Frb,Nkd2,Plp1,Lpr5,dkk3,Nrf2} {Frb,Tgfbr2,Tgfbr3,Hmox1,Cyba,Sox10} {Frb,Tgfbr2,Tgfbr3,Hmox1,Cyba,Plp1} {Frb,Tgfbr2,Tgfbr3,Hmox1,Cyba,Lpr5} {Frb,Tgfbr2,Tgfbr3,Hmox1,Cyba,dkk3} {Frb,Tgfbr2,Tgfbr3,Hmox1,Cyba,Nrf2} {Frb,Tgfbr2,Tgfbr3,Hmox1,Sox10,Plp1} {Frb,Tgfbr2,Tgfbr3,Hmox1,Sox10,Lpr5} {Frb,Tgfbr2,Tgfbr3,Hmox1,Sox10,dkk3} {Frb,Tgfbr2,Tgfbr3,Hmox1,Sox10,Nrf2} {Frb,Tgfbr2,Tgfbr3,Hmox1,Plp1,Lpr5} {Frb,Tgfbr2,Tgfbr3,Hmox1,Plp1,dkk3} {Frb,Tgfbr2,Tgfbr3,Hmox1,Plp1,Nrf2} {Frb,Tgfbr2,Tgfbr3,Hmox1,Lpr5,dkk3} {Frb,Tgfbr2,Tgfbr3,Hmox1,Lpr5,Nrf2} {Frb,Tgfbr2,Tgfbr3,Hmox1,dkk3,Nrf2} {Frb,Tgfbr2,Tgfbr3,Cyba,Sox10,Plp1} {Frb,Tgfbr2,Tgfbr3,Cyba,Sox10,Lpr5} {Frb,Tgfbr2,Tgfbr3,Cyba,Sox10,dkk3} {Frb,Tgfbr2,Tgfbr3,Cyba,Sox10,Nrf2} {Frb,Tgfbr2,Tgfbr3,Cyba,Plp1,Lpr5} {Frb,Tgfbr2,Tgfbr3,Cyba,Plp1,dkk3} {Frb,Tgfbr2,Tgfbr3,Cyba,Plp1,Nrf2} {Frb,Tgfbr2,Tgfbr3,Cyba,Lpr5,dkk3} {Frb,Tgfbr2,Tgfbr3,Cyba,Lpr5,Nrf2} {Frb,Tgfbr2,Tgfbr3,Cyba,dkk3,Nrf2} {Frb,Tgfbr2,Tgfbr3,Sox10,Plp1,Lpr5} {Frb,Tgfbr2,Tgfbr3,Sox10,Plp1,dkk3} {Frb,Tgfbr2,Tgfbr3,Sox10,Plp1,Nrf2} {Frb,Tgfbr2,Tgfbr3,Sox10,Lpr5,dkk3} {Frb,Tgfbr2,Tgfbr3,Sox10,Lpr5,Nrf2} {Frb,Tgfbr2,Tgfbr3,Sox10,dkk3,Nrf2} {Frb,Tgfbr2,Tgfbr3,Plp1,Lpr5,dkk3} {Frb,Tgfbr2,Tgfbr3,Plp1,Lpr5,Nrf2} {Frb,Tgfbr2,Tgfbr3,Plp1,dkk3,Nrf2} {Frb,Tgfbr2,Tgfbr3,Lpr5,dkk3,Nrf2} {Frb,Tgfbr2,Hmox1,Cyba,Sox10,Plp1} {Frb,Tgfbr2,Hmox1,Cyba,Sox10,Lpr5} {Frb,Tgfbr2,Hmox1,Cyba,Sox10,dkk3} {Frb,Tgfbr2,Hmox1,Cyba,Sox10,Nrf2} {Frb,Tgfbr2,Hmox1,Cyba,Plp1,Lpr5} {Frb,Tgfbr2,Hmox1,Cyba,Plp1,dkk3} {Frb,Tgfbr2,Hmox1,Cyba,Plp1,Nrf2} {Frb,Tgfbr2,Hmox1,Cyba,Lpr5,dkk3} {Frb,Tgfbr2,Hmox1,Cyba,Lpr5,Nrf2} {Frb,Tgfbr2,Hmox1,Cyba,dkk3,Nrf2} {Frb,Tgfbr2,Hmox1,Sox10,Plp1,Lpr5} {Frb,Tgfbr2,Hmox1,Sox10,Plp1,dkk3} {Frb,Tgfbr2,Hmox1,Sox10,Plp1,Nrf2} {Frb,Tgfbr2,Hmox1,Sox10,Lpr5,dkk3} {Frb,Tgfbr2,Hmox1,Sox10,Lpr5,Nrf2} {Frb,Tgfbr2,Hmox1,Sox10,dkk3,Nrf2} {Frb,Tgfbr2,Hmox1,Plp1,Lpr5,dkk3} {Frb,Tgfbr2,Hmox1,Plp1,Lpr5,Nrf2} {Frb,Tgfbr2,Hmox1,Plp1,dkk3,Nrf2} {Frb,Tgfbr2,Hmox1,Lpr5,dkk3,Nrf2} {Frb,Tgfbr2,Cyba,Sox10,Plp1,Lpr5} {Frb,Tgfbr2,Cyba,Sox10,Plp1,dkk3} {Frb,Tgfbr2,Cyba,Sox10,Plp1,Nrf2} {Frb,Tgfbr2,Cyba,Sox10,Lpr5,dkk3} {Frb,Tgfbr2,Cyba,Sox10,Lpr5,Nrf2} {Frb,Tgfbr2,Cyba,Sox10,dkk3,Nrf2} {Frb,Tgfbr2,Cyba,Plp1,Lpr5,dkk3} {Frb,Tgfbr2,Cyba,Plp1,Lpr5,Nrf2} {Frb,Tgfbr2,Cyba,Plp1,dkk3,Nrf2} {Frb,Tgfbr2,Cyba,Lpr5,dkk3,Nrf2} {Frb,Tgfbr2,Sox10,Plp1,Lpr5,dkk3} {Frb,Tgfbr2,Sox10,Plp1,Lpr5,Nrf2} {Frb,Tgfbr2,Sox10,Plp1,dkk3,Nrf2} {Frb,Tgfbr2,Sox10,Lpr5,dkk3,Nrf2} {Frb,Tgfbr2,Plp1,Lpr5,dkk3,Nrf2} {Frb,Tgfbr3,Hmox1,Cyba,Sox10,Plp1} {Frb,Tgfbr3,Hmox1,Cyba,Sox10,Lpr5} {Frb,Tgfbr3,Hmox1,Cyba,Sox10,dkk3} {Frb,Tgfbr3,Hmox1,Cyba,Sox10,Nrf2} {Frb,Tgfbr3,Hmox1,Cyba,Plp1,Lpr5} {Frb,Tgfbr3,Hmox1,Cyba,Plp1,dkk3} {Frb,Tgfbr3,Hmox1,Cyba,Plp1,Nrf2} {Frb,Tgfbr3,Hmox1,Cyba,Lpr5,dkk3} {Frb,Tgfbr3,Hmox1,Cyba,Lpr5,Nrf2} {Frb,Tgfbr3,Hmox1,Cyba,dkk3,Nrf2} {Frb,Tgfbr3,Hmox1,Sox10,Plp1,Lpr5} {Frb,Tgfbr3,Hmox1,Sox10,Plp1,dkk3} {Frb,Tgfbr3,Hmox1,Sox10,Plp1,Nrf2} {Frb,Tgfbr3,Hmox1,Sox10,Lpr5,dkk3} {Frb,Tgfbr3,Hmox1,Sox10,Lpr5,Nrf2} {Frb,Tgfbr3,Hmox1,Sox10,dkk3,Nrf2} {Frb,Tgfbr3,Hmox1,Plp1,Lpr5,dkk3} {Frb,Tgfbr3,Hmox1,Plp1,Lpr5,Nrf2} {Frb,Tgfbr3,Hmox1,Plp1,dkk3,Nrf2} {Frb,Tgfbr3,Hmox1,Lpr5,dkk3,Nrf2} {Frb,Tgfbr3,Cyba,Sox10,Plp1,Lpr5} {Frb,Tgfbr3,Cyba,Sox10,Plp1,dkk3} {Frb,Tgfbr3,Cyba,Sox10,Plp1,Nrf2} {Frb,Tgfbr3,Cyba,Sox10,Lpr5,dkk3} {Frb,Tgfbr3,Cyba,Sox10,Lpr5,Nrf2} {Frb,Tgfbr3,Cyba,Sox10,dkk3,Nrf2} {Frb,Tgfbr3,Cyba,Plp1,Lpr5,dkk3} {Frb,Tgfbr3,Cyba,Plp1,Lpr5,Nrf2} {Frb,Tgfbr3,Cyba,Plp1,dkk3,Nrf2} {Frb,Tgfbr3,Cyba,Lpr5,dkk3,Nrf2} {Frb,Tgfbr3,Sox10,Plp1,Lpr5,dkk3} {Frb,Tgfbr3,Sox10,Plp1,Lpr5,Nrf2} {Frb,Tgfbr3,Sox10,Plp1,dkk3,Nrf2} {Frb,Tgfbr3,Sox10,Lpr5,dkk3,Nrf2} {Frb,Tgfbr3,Plp1,Lpr5,dkk3,Nrf2} {Frb,Hmox1,Cyba,Sox10,Plp1,Lpr5} {Frb,Hmox1,Cyba,Sox10,Plp1,dkk3} {Frb,Hmox1,Cyba,Sox10,Plp1,Nrf2} {Frb,Hmox1,Cyba,Sox10,Lpr5,dkk3} {Frb,Hmox1,Cyba,Sox10,Lpr5,Nrf2} {Frb,Hmox1,Cyba,Sox10,dkk3,Nrf2} {Frb,Hmox1,Cyba,Plp1,Lpr5,dkk3} {Frb,Hmox1,Cyba,Plp1,Lpr5,Nrf2} {Frb,Hmox1,Cyba,Plp1,dkk3,Nrf2} {Frb,Hmox1,Cyba,Lpr5,dkk3,Nrf2} {Frb,Hmox1,Sox10,Plp1,Lpr5,dkk3} {Frb,Hmox1,Sox10,Plp1,Lpr5,Nrf2} {Frb,Hmox1,Sox10,Plp1,dkk3,Nrf2} {Frb,Hmox1,Sox10,Lpr5,dkk3,Nrf2} {Frb,Hmox1,Plp1,Lpr5,dkk3,Nrf2} {Frb,Cyba,Sox10,Plp1,Lpr5,dkk3} {Frb,Cyba,Sox10,Plp1,Lpr5,Nrf2} {Frb,Cyba,Sox10,Plp1,dkk3,Nrf2} {Frb,Cyba,Sox10,Lpr5,dkk3,Nrf2} {Frb,Cyba,Plp1,Lpr5,dkk3,Nrf2} {Frb,Sox10,Plp1,Lpr5,dkk3,Nrf2} {Nkd2,Tgfbr2,Tgfbr3,Hmox1,Cyba,Sox10} {Nkd2,Tgfbr2,Tgfbr3,Hmox1,Cyba,Plp1} {Nkd2,Tgfbr2,Tgfbr3,Hmox1,Cyba,Lpr5} {Nkd2,Tgfbr2,Tgfbr3,Hmox1,Cyba,dkk3} {Nkd2,Tgfbr2,Tgfbr3,Hmox1,Cyba,Nrf2} {Nkd2,Tgfbr2,Tgfbr3,Hmox1,Sox10,Plp1} {Nkd2,Tgfbr2,Tgfbr3,Hmox1,Sox10,Lpr5} {Nkd2,Tgfbr2,Tgfbr3,Hmox1,Sox10,dkk3} {Nkd2,Tgfbr2,Tgfbr3,Hmox1,Sox10,Nrf2} {Nkd2,Tgfbr2,Tgfbr3,Hmox1,Plp1,Lpr5} {Nkd2,Tgfbr2,Tgfbr3,Hmox1,Plp1,dkk3} {Nkd2,Tgfbr2,Tgfbr3,Hmox1,Plp1,Nrf2} {Nkd2,Tgfbr2,Tgfbr3,Hmox1,Lpr5,dkk3} {Nkd2,Tgfbr2,Tgfbr3,Hmox1,Lpr5,Nrf2} {Nkd2,Tgfbr2,Tgfbr3,Hmox1,dkk3,Nrf2} {Nkd2,Tgfbr2,Tgfbr3,Cyba,Sox10,Plp1} {Nkd2,Tgfbr2,Tgfbr3,Cyba,Sox10,Lpr5} {Nkd2,Tgfbr2,Tgfbr3,Cyba,Sox10,dkk3} {Nkd2,Tgfbr2,Tgfbr3,Cyba,Sox10,Nrf2} {Nkd2,Tgfbr2,Tgfbr3,Cyba,Plp1,Lpr5} {Nkd2,Tgfbr2,Tgfbr3,Cyba,Plp1,dkk3} {Nkd2,Tgfbr2,Tgfbr3,Cyba,Plp1,Nrf2} {Nkd2,Tgfbr2,Tgfbr3,Cyba,Lpr5,dkk3} {Nkd2,Tgfbr2,Tgfbr3,Cyba,Lpr5,Nrf2} {Nkd2,Tgfbr2,Tgfbr3,Cyba,dkk3,Nrf2} {Nkd2,Tgfbr2,Tgfbr3,Sox10,Plp1,Lpr5} {Nkd2,Tgfbr2,Tgfbr3,Sox10,Plp1,dkk3} {Nkd2,Tgfbr2,Tgfbr3,Sox10,Plp1,Nrf2} {Nkd2,Tgfbr2,Tgfbr3,Sox10,Lpr5,dkk3} {Nkd2,Tgfbr2,Tgfbr3,Sox10,Lpr5,Nrf2} {Nkd2,Tgfbr2,Tgfbr3,Sox10,dkk3,Nrf2} {Nkd2,Tgfbr2,Tgfbr3,Plp1,Lpr5,dkk3} {Nkd2,Tgfbr2,Tgfbr3,Plp1,Lpr5,Nrf2} {Nkd2,Tgfbr2,Tgfbr3,Plp1,dkk3,Nrf2} {Nkd2,Tgfbr2,Tgfbr3,Lpr5,dkk3,Nrf2} {Nkd2,Tgfbr2,Hmox1,Cyba,Sox10,Plp1} {Nkd2,Tgfbr2,Hmox1,Cyba,Sox10,Lpr5} {Nkd2,Tgfbr2,Hmox1,Cyba,Sox10,dkk3} {Nkd2,Tgfbr2,Hmox1,Cyba,Sox10,Nrf2} {Nkd2,Tgfbr2,Hmox1,Cyba,Plp1,Lpr5} {Nkd2,Tgfbr2,Hmox1,Cyba,Plp1,dkk3} {Nkd2,Tgfbr2,Hmox1,Cyba,Plp1,Nrf2} {Nkd2,Tgfbr2,Hmox1,Cyba,Lpr5,dkk3} {Nkd2,Tgfbr2,Hmox1,Cyba,Lpr5,Nrf2} {Nkd2,Tgfbr2,Hmox1,Cyba,dkk3,Nrf2} {Nkd2,Tgfbr2,Hmox1,Sox10,Plp1,Lpr5} {Nkd2,Tgfbr2,Hmox1,Sox10,Plp1,dkk3} {Nkd2,Tgfbr2,Hmox1,Sox10,Plp1,Nrf2} {Nkd2,Tgfbr2,Hmox1,Sox10,Lpr5,dkk3} {Nkd2,Tgfbr2,Hmox1,Sox10,Lpr5,Nrf2} {Nkd2,Tgfbr2,Hmox1,Sox10,dkk3,Nrf2} {Nkd2,Tgfbr2,Hmox1,Plp1,Lpr5,dkk3} {Nkd2,Tgfbr2,Hmox1,Plp1,Lpr5,Nrf2} {Nkd2,Tgfbr2,Hmox1,Plp1,dkk3,Nrf2} {Nkd2,Tgfbr2,Hmox1,Lpr5,dkk3,Nrf2} {Nkd2,Tgfbr2,Cyba,Sox10,Plp1,Lpr5} {Nkd2,Tgfbr2,Cyba,Sox10,Plp1,dkk3} {Nkd2,Tgfbr2,Cyba,Sox10,Plp1,Nrf2} {Nkd2,Tgfbr2,Cyba,Sox10,Lpr5,dkk3} {Nkd2,Tgfbr2,Cyba,Sox10,Lpr5,Nrf2} {Nkd2,Tgfbr2,Cyba,Sox10,dkk3,Nrf2} {Nkd2,Tgfbr2,Cyba,Plp1,Lpr5,dkk3} {Nkd2,Tgfbr2,Cyba,Plp1,Lpr5,Nrf2} {Nkd2,Tgfbr2,Cyba,Plp1,dkk3,Nrf2} {Nkd2,Tgfbr2,Cyba,Lpr5,dkk3,Nrf2} {Nkd2,Tgfbr2,Sox10,Plp1,Lpr5,dkk3} {Nkd2,Tgfbr2,Sox10,Plp1,Lpr5,Nrf2} {Nkd2,Tgfbr2,Sox10,Plp1,dkk3,Nrf2} {Nkd2,Tgfbr2,Sox10,Lpr5,dkk3,Nrf2} {Nkd2,Tgfbr2,Plp1,Lpr5,dkk3,Nrf2} {Nkd2,Tgfbr3,Hmox1,Cyba,Sox10,Plp1} {Nkd2,Tgfbr3,Hmox1,Cyba,Sox10,Lpr5} {Nkd2,Tgfbr3,Hmox1,Cyba,Sox10,dkk3} {Nkd2,Tgfbr3,Hmox1,Cyba,Sox10,Nrf2} {Nkd2,Tgfbr3,Hmox1,Cyba,Plp1,Lpr5} {Nkd2,Tgfbr3,Hmox1,Cyba,Plp1,dkk3} {Nkd2,Tgfbr3,Hmox1,Cyba,Plp1,Nrf2} {Nkd2,Tgfbr3,Hmox1,Cyba,Lpr5,dkk3} {Nkd2,Tgfbr3,Hmox1,Cyba,Lpr5,Nrf2} {Nkd2,Tgfbr3,Hmox1,Cyba,dkk3,Nrf2} {Nkd2,Tgfbr3,Hmox1,Sox10,Plp1,Lpr5} {Nkd2,Tgfbr3,Hmox1,Sox10,Plp1,dkk3} {Nkd2,Tgfbr3,Hmox1,Sox10,Plp1,Nrf2} {Nkd2,Tgfbr3,Hmox1,Sox10,Lpr5,dkk3} {Nkd2,Tgfbr3,Hmox1,Sox10,Lpr5,Nrf2} {Nkd2,Tgfbr3,Hmox1,Sox10,dkk3,Nrf2} {Nkd2,Tgfbr3,Hmox1,Plp1,Lpr5,dkk3} {Nkd2,Tgfbr3,Hmox1,Plp1,Lpr5,Nrf2} {Nkd2,Tgfbr3,Hmox1,Plp1,dkk3,Nrf2} {Nkd2,Tgfbr3,Hmox1,Lpr5,dkk3,Nrf2} {Nkd2,Tgfbr3,Cyba,Sox10,Plp1,Lpr5} {Nkd2,Tgfbr3,Cyba,Sox10,Plp1,dkk3} {Nkd2,Tgfbr3,Cyba,Sox10,Plp1,Nrf2} {Nkd2,Tgfbr3,Cyba,Sox10,Lpr5,dkk3} {Nkd2,Tgfbr3,Cyba,Sox10,Lpr5,Nrf2} {Nkd2,Tgfbr3,Cyba,Sox10,dkk3,Nrf2} {Nkd2,Tgfbr3,Cyba,Plp1,Lpr5,dkk3} {Nkd2,Tgfbr3,Cyba,Plp1,Lpr5,Nrf2} {Nkd2,Tgfbr3,Cyba,Plp1,dkk3,Nrf2} {Nkd2,Tgfbr3,Cyba,Lpr5,dkk3,Nrf2} {Nkd2,Tgfbr3,Sox10,Plp1,Lpr5,dkk3} {Nkd2,Tgfbr3,Sox10,Plp1,Lpr5,Nrf2} {Nkd2,Tgfbr3,Sox10,Plp1,dkk3,Nrf2} {Nkd2,Tgfbr3,Sox10,Lpr5,dkk3,Nrf2} {Nkd2,Tgfbr3,Plp1,Lpr5,dkk3,Nrf2} {Nkd2,Hmox1,Cyba,Sox10,Plp1,Lpr5} {Nkd2,Hmox1,Cyba,Sox10,Plp1,dkk3} {Nkd2,Hmox1,Cyba,Sox10,Plp1,Nrf2} {Nkd2,Hmox1,Cyba,Sox10,Lpr5,dkk3} {Nkd2,Hmox1,Cyba,Sox10,Lpr5,Nrf2} {Nkd2,Hmox1,Cyba,Sox10,dkk3,Nrf2} {Nkd2,Hmox1,Cyba,Plp1,Lpr5,dkk3} {Nkd2,Hmox1,Cyba,Plp1,Lpr5,Nrf2} {Nkd2,Hmox1,Cyba,Plp1,dkk3,Nrf2} {Nkd2,Hmox1,Cyba,Lpr5,dkk3,Nrf2} {Nkd2,Hmox1,Sox10,Plp1,Lpr5,dkk3} {Nkd2,Hmox1,Sox10,Plp1,Lpr5,Nrf2} {Nkd2,Hmox1,Sox10,Plp1,dkk3,Nrf2} {Nkd2,Hmox1,Sox10,Lpr5,dkk3,Nrf2} {Nkd2,Hmox1,Plp1,Lpr5,dkk3,Nrf2} {Nkd2,Cyba,Sox10,Plp1,Lpr5,dkk3} {Nkd2,Cyba,Sox10,Plp1,Lpr5,Nrf2} {Nkd2,Cyba,Sox10,Plp1,dkk3,Nrf2} {Nkd2,Cyba,Sox10,Lpr5,dkk3,Nrf2} {Nkd2,Cyba,Plp1,Lpr5,dkk3,Nrf2} {Nkd2,Sox10,Plp1,Lpr5,dkk3,Nrf2} {Tgfbr2,Tgfbr3,Hmox1,Cyba,Sox10,Plp1} {Tgfbr2,Tgfbr3,Hmox1,Cyba,Sox10,Lpr5} {Tgfbr2,Tgfbr3,Hmox1,Cyba,Sox10,dkk3} {Tgfbr2,Tgfbr3,Hmox1,Cyba,Sox10,Nrf2} {Tgfbr2,Tgfbr3,Hmox1,Cyba,Plp1,Lpr5} {Tgfbr2,Tgfbr3,Hmox1,Cyba,Plp1,dkk3} {Tgfbr2,Tgfbr3,Hmox1,Cyba,Plp1,Nrf2} {Tgfbr2,Tgfbr3,Hmox1,Cyba,Lpr5,dkk3} {Tgfbr2,Tgfbr3,Hmox1,Cyba,Lpr5,Nrf2} {Tgfbr2,Tgfbr3,Hmox1,Cyba,dkk3,Nrf2} {Tgfbr2,Tgfbr3,Hmox1,Sox10,Plp1,Lpr5} {Tgfbr2,Tgfbr3,Hmox1,Sox10,Plp1,dkk3} {Tgfbr2,Tgfbr3,Hmox1,Sox10,Plp1,Nrf2} {Tgfbr2,Tgfbr3,Hmox1,Sox10,Lpr5,dkk3} {Tgfbr2,Tgfbr3,Hmox1,Sox10,Lpr5,Nrf2} {Tgfbr2,Tgfbr3,Hmox1,Sox10,dkk3,Nrf2} {Tgfbr2,Tgfbr3,Hmox1,Plp1,Lpr5,dkk3} {Tgfbr2,Tgfbr3,Hmox1,Plp1,Lpr5,Nrf2} {Tgfbr2,Tgfbr3,Hmox1,Plp1,dkk3,Nrf2} {Tgfbr2,Tgfbr3,Hmox1,Lpr5,dkk3,Nrf2} {Tgfbr2,Tgfbr3,Cyba,Sox10,Plp1,Lpr5} {Tgfbr2,Tgfbr3,Cyba,Sox10,Plp1,dkk3} {Tgfbr2,Tgfbr3,Cyba,Sox10,Plp1,Nrf2} {Tgfbr2,Tgfbr3,Cyba,Sox10,Lpr5,dkk3} {Tgfbr2,Tgfbr3,Cyba,Sox10,Lpr5,Nrf2} {Tgfbr2,Tgfbr3,Cyba,Sox10,dkk3,Nrf2} {Tgfbr2,Tgfbr3,Cyba,Plp1,Lpr5,dkk3} {Tgfbr2,Tgfbr3,Cyba,Plp1,Lpr5,Nrf2} {Tgfbr2,Tgfbr3,Cyba,Plp1,dkk3,Nrf2} {Tgfbr2,Tgfbr3,Cyba,Lpr5,dkk3,Nrf2} {Tgfbr2,Tgfbr3,Sox10,Plp1,Lpr5,dkk3} {Tgfbr2,Tgfbr3,Sox10,Plp1,Lpr5,Nrf2} {Tgfbr2,Tgfbr3,Sox10,Plp1,dkk3,Nrf2} {Tgfbr2,Tgfbr3,Sox10,Lpr5,dkk3,Nrf2} {Tgfbr2,Tgfbr3,Plp1,Lpr5,dkk3,Nrf2} {Tgfbr2,Hmox1,Cyba,Sox10,Plp1,Lpr5} {Tgfbr2,Hmox1,Cyba,Sox10,Plp1,dkk3} {Tgfbr2,Hmox1,Cyba,Sox10,Plp1,Nrf2} {Tgfbr2,Hmox1,Cyba,Sox10,Lpr5,dkk3} {Tgfbr2,Hmox1,Cyba,Sox10,Lpr5,Nrf2} {Tgfbr2,Hmox1,Cyba,Sox10,dkk3,Nrf2} {Tgfbr2,Hmox1,Cyba,Plp1,Lpr5,dkk3} {Tgfbr2,Hmox1,Cyba,Plp1,Lpr5,Nrf2} {Tgfbr2,Hmox1,Cyba,Plp1,dkk3,Nrf2} {Tgfbr2,Hmox1,Cyba,Lpr5,dkk3,Nrf2} {Tgfbr2,Hmox1,Sox10,Plp1,Lpr5,dkk3} {Tgfbr2,Hmox1,Sox10,Plp1,Lpr5,Nrf2} {Tgfbr2,Hmox1,Sox10,Plp1,dkk3,Nrf2} {Tgfbr2,Hmox1,Sox10,Lpr5,dkk3,Nrf2} {Tgfbr2,Hmox1,Plp1,Lpr5,dkk3,Nrf2} {Tgfbr2,Cyba,Sox10,Plp1,Lpr5,dkk3} {Tgfbr2,Cyba,Sox10,Plp1,Lpr5,Nrf2} {Tgfbr2,Cyba,Sox10,Plp1,dkk3,Nrf2} {Tgfbr2,Cyba,Sox10,Lpr5,dkk3,Nrf2} {Tgfbr2,Cyba,Plp1,Lpr5,dkk3,Nrf2} {Tgfbr2,Sox10,Plp1,Lpr5,dkk3,Nrf2} {Tgfbr3,Hmox1,Cyba,Sox10,Plp1,Lpr5} {Tgfbr3,Hmox1,Cyba,Sox10,Plp1,dkk3} {Tgfbr3,Hmox1,Cyba,Sox10,Plp1,Nrf2} {Tgfbr3,Hmox1,Cyba,Sox10,Lpr5,dkk3} {Tgfbr3,Hmox1,Cyba,Sox10,Lpr5,Nrf2} {Tgfbr3,Hmox1,Cyba,Sox10,dkk3,Nrf2} {Tgfbr3,Hmox1,Cyba,Plp1,Lpr5,dkk3} {Tgfbr3,Hmox1,Cyba,Plp1,Lpr5,Nrf2} {Tgfbr3,Hmox1,Cyba,Plp1,dkk3,Nrf2} {Tgfbr3,Hmox1,Cyba,Lpr5,dkk3,Nrf2} {Tgfbr3,Hmox1,Sox10,Plp1,Lpr5,dkk3} {Tgfbr3,Hmox1,Sox10,Plp1,Lpr5,Nrf2} {Tgfbr3,Hmox1,Sox10,Plp1,dkk3,Nrf2} {Tgfbr3,Hmox1,Sox10,Lpr5,dkk3,Nrf2} {Tgfbr3,Hmox1,Plp1,Lpr5,dkk3,Nrf2} {Tgfbr3,Cyba,Sox10,Plp1,Lpr5,dkk3} {Tgfbr3,Cyba,Sox10,Plp1,Lpr5,Nrf2} {Tgfbr3,Cyba,Sox10,Plp1,dkk3,Nrf2} {Tgfbr3,Cyba,Sox10,Lpr5,dkk3,Nrf2} {Tgfbr3,Cyba,Plp1,Lpr5,dkk3,Nrf2} {Tgfbr3,Sox10,Plp1,Lpr5,dkk3,Nrf2} {Hmox1,Cyba,Sox10,Plp1,Lpr5,dkk3} {Hmox1,Cyba,Sox10,Plp1,Lpr5,Nrf2} {Hmox1,Cyba,Sox10,Plp1,dkk3,Nrf2} {Hmox1,Cyba,Sox10,Lpr5,dkk3,Nrf2} {Hmox1,Cyba,Plp1,Lpr5,dkk3,Nrf2} {Hmox1,Sox10,Plp1,Lpr5,dkk3,Nrf2} {Cyba,Sox10,Plp1,Lpr5,dkk3,Nrf2} {Lgr6,Frb,Nkd2,Tgfbr2,Tgfbr3,Hmox1,Cyba} {Lgr6,Frb,Nkd2,Tgfbr2,Tgfbr3,Hmox1,Sox10} {Lgr6,Frb,Nkd2,Tgfbr2,Tgfbr3,Hmox1,Plp1} {Lgr6,Frb,Nkd2,Tgfbr2,Tgfbr3,Hmox1,Lpr5} {Lgr6,Frb,Nkd2,Tgfbr2,Tgfbr3,Hmox1,dkk3} {Lgr6,Frb,Nkd2,Tgfbr2,Tgfbr3,Hmox1,Nrf2} {Lgr6,Frb,Nkd2,Tgfbr2,Tgfbr3,Cyba,Sox10} {Lgr6,Frb,Nkd2,Tgfbr2,Tgfbr3,Cyba,Plp1} {Lgr6,Frb,Nkd2,Tgfbr2,Tgfbr3,Cyba,Lpr5} {Lgr6,Frb,Nkd2,Tgfbr2,Tgfbr3,Cyba,dkk3} {Lgr6,Frb,Nkd2,Tgfbr2,Tgfbr3,Cyba,Nrf2} {Lgr6,Frb,Nkd2,Tgfbr2,Tgfbr3,Sox10,Plp1} {Lgr6,Frb,Nkd2,Tgfbr2,Tgfbr3,Sox10,Lpr5} {Lgr6,Frb,Nkd2,Tgfbr2,Tgfbr3,Sox10,dkk3} {Lgr6,Frb,Nkd2,Tgfbr2,Tgfbr3,Sox10,Nrf2} {Lgr6,Frb,Nkd2,Tgfbr2,Tgfbr3,Plp1,Lpr5} {Lgr6,Frb,Nkd2,Tgfbr2,Tgfbr3,Plp1,dkk3} {Lgr6,Frb,Nkd2,Tgfbr2,Tgfbr3,Plp1,Nrf2} {Lgr6,Frb,Nkd2,Tgfbr2,Tgfbr3,Lpr5,dkk3} {Lgr6,Frb,Nkd2,Tgfbr2,Tgfbr3,Lpr5,Nrf2} {Lgr6,Frb,Nkd2,Tgfbr2,Tgfbr3,dkk3,Nrf2} {Lgr6,Frb,Nkd2,Tgfbr2,Hmox1,Cyba,Sox10} {Lgr6,Frb,Nkd2,Tgfbr2,Hmox1,Cyba,Plp1} {Lgr6,Frb,Nkd2,Tgfbr2,Hmox1,Cyba,Lpr5} {Lgr6,Frb,Nkd2,Tgfbr2,Hmox1,Cyba,dkk3} {Lgr6,Frb,Nkd2,Tgfbr2,Hmox1,Cyba,Nrf2} {Lgr6,Frb,Nkd2,Tgfbr2,Hmox1,Sox10,Plp1} {Lgr6,Frb,Nkd2,Tgfbr2,Hmox1,Sox10,Lpr5} {Lgr6,Frb,Nkd2,Tgfbr2,Hmox1,Sox10,dkk3} {Lgr6,Frb,Nkd2,Tgfbr2,Hmox1,Sox10,Nrf2} {Lgr6,Frb,Nkd2,Tgfbr2,Hmox1,Plp1,Lpr5} {Lgr6,Frb,Nkd2,Tgfbr2,Hmox1,Plp1,dkk3} {Lgr6,Frb,Nkd2,Tgfbr2,Hmox1,Plp1,Nrf2} {Lgr6,Frb,Nkd2,Tgfbr2,Hmox1,Lpr5,dkk3} {Lgr6,Frb,Nkd2,Tgfbr2,Hmox1,Lpr5,Nrf2} {Lgr6,Frb,Nkd2,Tgfbr2,Hmox1,dkk3,Nrf2} {Lgr6,Frb,Nkd2,Tgfbr2,Cyba,Sox10,Plp1} {Lgr6,Frb,Nkd2,Tgfbr2,Cyba,Sox10,Lpr5} {Lgr6,Frb,Nkd2,Tgfbr2,Cyba,Sox10,dkk3} {Lgr6,Frb,Nkd2,Tgfbr2,Cyba,Sox10,Nrf2} {Lgr6,Frb,Nkd2,Tgfbr2,Cyba,Plp1,Lpr5} {Lgr6,Frb,Nkd2,Tgfbr2,Cyba,Plp1,dkk3} {Lgr6,Frb,Nkd2,Tgfbr2,Cyba,Plp1,Nrf2} {Lgr6,Frb,Nkd2,Tgfbr2,Cyba,Lpr5,dkk3} {Lgr6,Frb,Nkd2,Tgfbr2,Cyba,Lpr5,Nrf2} {Lgr6,Frb,Nkd2,Tgfbr2,Cyba,dkk3,Nrf2} {Lgr6,Frb,Nkd2,Tgfbr2,Sox10,Plp1,Lpr5} {Lgr6,Frb,Nkd2,Tgfbr2,Sox10,Plp1,dkk3} {Lgr6,Frb,Nkd2,Tgfbr2,Sox10,Plp1,Nrf2} {Lgr6,Frb,Nkd2,Tgfbr2,Sox10,Lpr5,dkk3} {Lgr6,Frb,Nkd2,Tgfbr2,Sox10,Lpr5,Nrf2} {Lgr6,Frb,Nkd2,Tgfbr2,Sox10,dkk3,Nrf2} {Lgr6,Frb,Nkd2,Tgfbr2,Plp1,Lpr5,dkk3} {Lgr6,Frb,Nkd2,Tgfbr2,Plp1,Lpr5,Nrf2} {Lgr6,Frb,Nkd2,Tgfbr2,Plp1,dkk3,Nrf2} {Lgr6,Frb,Nkd2,Tgfbr2,Lpr5,dkk3,Nrf2} {Lgr6,Frb,Nkd2,Tgfbr3,Hmox1,Cyba,Sox10} {Lgr6,Frb,Nkd2,Tgfbr3,Hmox1,Cyba,Plp1} {Lgr6,Frb,Nkd2,Tgfbr3,Hmox1,Cyba,Lpr5} {Lgr6,Frb,Nkd2,Tgfbr3,Hmox1,Cyba,dkk3} {Lgr6,Frb,Nkd2,Tgfbr3,Hmox1,Cyba,Nrf2} {Lgr6,Frb,Nkd2,Tgfbr3,Hmox1,Sox10,Plp1} {Lgr6,Frb,Nkd2,Tgfbr3,Hmox1,Sox10,Lpr5} {Lgr6,Frb,Nkd2,Tgfbr3,Hmox1,Sox10,dkk3} {Lgr6,Frb,Nkd2,Tgfbr3,Hmox1,Sox10,Nrf2} {Lgr6,Frb,Nkd2,Tgfbr3,Hmox1,Plp1,Lpr5} {Lgr6,Frb,Nkd2,Tgfbr3,Hmox1,Plp1,dkk3} {Lgr6,Frb,Nkd2,Tgfbr3,Hmox1,Plp1,Nrf2} {Lgr6,Frb,Nkd2,Tgfbr3,Hmox1,Lpr5,dkk3} {Lgr6,Frb,Nkd2,Tgfbr3,Hmox1,Lpr5,Nrf2} {Lgr6,Frb,Nkd2,Tgfbr3,Hmox1,dkk3,Nrf2} {Lgr6,Frb,Nkd2,Tgfbr3,Cyba,Sox10,Plp1} {Lgr6,Frb,Nkd2,Tgfbr3,Cyba,Sox10,Lpr5} {Lgr6,Frb,Nkd2,Tgfbr3,Cyba,Sox10,dkk3} {Lgr6,Frb,Nkd2,Tgfbr3,Cyba,Sox10,Nrf2} {Lgr6,Frb,Nkd2,Tgfbr3,Cyba,Plp1,Lpr5} {Lgr6,Frb,Nkd2,Tgfbr3,Cyba,Plp1,dkk3} {Lgr6,Frb,Nkd2,Tgfbr3,Cyba,Plp1,Nrf2} {Lgr6,Frb,Nkd2,Tgfbr3,Cyba,Lpr5,dkk3} {Lgr6,Frb,Nkd2,Tgfbr3,Cyba,Lpr5,Nrf2} {Lgr6,Frb,Nkd2,Tgfbr3,Cyba,dkk3,Nrf2} {Lgr6,Frb,Nkd2,Tgfbr3,Sox10,Plp1,Lpr5} {Lgr6,Frb,Nkd2,Tgfbr3,Sox10,Plp1,dkk3} {Lgr6,Frb,Nkd2,Tgfbr3,Sox10,Plp1,Nrf2} {Lgr6,Frb,Nkd2,Tgfbr3,Sox10,Lpr5,dkk3} {Lgr6,Frb,Nkd2,Tgfbr3,Sox10,Lpr5,Nrf2} {Lgr6,Frb,Nkd2,Tgfbr3,Sox10,dkk3,Nrf2} {Lgr6,Frb,Nkd2,Tgfbr3,Plp1,Lpr5,dkk3} {Lgr6,Frb,Nkd2,Tgfbr3,Plp1,Lpr5,Nrf2} {Lgr6,Frb,Nkd2,Tgfbr3,Plp1,dkk3,Nrf2} {Lgr6,Frb,Nkd2,Tgfbr3,Lpr5,dkk3,Nrf2} {Lgr6,Frb,Nkd2,Hmox1,Cyba,Sox10,Plp1} {Lgr6,Frb,Nkd2,Hmox1,Cyba,Sox10,Lpr5} {Lgr6,Frb,Nkd2,Hmox1,Cyba,Sox10,dkk3} {Lgr6,Frb,Nkd2,Hmox1,Cyba,Sox10,Nrf2} {Lgr6,Frb,Nkd2,Hmox1,Cyba,Plp1,Lpr5} {Lgr6,Frb,Nkd2,Hmox1,Cyba,Plp1,dkk3} {Lgr6,Frb,Nkd2,Hmox1,Cyba,Plp1,Nrf2} {Lgr6,Frb,Nkd2,Hmox1,Cyba,Lpr5,dkk3} {Lgr6,Frb,Nkd2,Hmox1,Cyba,Lpr5,Nrf2} {Lgr6,Frb,Nkd2,Hmox1,Cyba,dkk3,Nrf2} {Lgr6,Frb,Nkd2,Hmox1,Sox10,Plp1,Lpr5} {Lgr6,Frb,Nkd2,Hmox1,Sox10,Plp1,dkk3} {Lgr6,Frb,Nkd2,Hmox1,Sox10,Plp1,Nrf2} {Lgr6,Frb,Nkd2,Hmox1,Sox10,Lpr5,dkk3} {Lgr6,Frb,Nkd2,Hmox1,Sox10,Lpr5,Nrf2} {Lgr6,Frb,Nkd2,Hmox1,Sox10,dkk3,Nrf2} {Lgr6,Frb,Nkd2,Hmox1,Plp1,Lpr5,dkk3} {Lgr6,Frb,Nkd2,Hmox1,Plp1,Lpr5,Nrf2} {Lgr6,Frb,Nkd2,Hmox1,Plp1,dkk3,Nrf2} {Lgr6,Frb,Nkd2,Hmox1,Lpr5,dkk3,Nrf2} {Lgr6,Frb,Nkd2,Cyba,Sox10,Plp1,Lpr5} {Lgr6,Frb,Nkd2,Cyba,Sox10,Plp1,dkk3} {Lgr6,Frb,Nkd2,Cyba,Sox10,Plp1,Nrf2} {Lgr6,Frb,Nkd2,Cyba,Sox10,Lpr5,dkk3} {Lgr6,Frb,Nkd2,Cyba,Sox10,Lpr5,Nrf2} {Lgr6,Frb,Nkd2,Cyba,Sox10,dkk3,Nrf2} {Lgr6,Frb,Nkd2,Cyba,Plp1,Lpr5,dkk3} {Lgr6,Frb,Nkd2,Cyba,Plp1,Lpr5,Nrf2} {Lgr6,Frb,Nkd2,Cyba,Plp1,dkk3,Nrf2} {Lgr6,Frb,Nkd2,Cyba,Lpr5,dkk3,Nrf2} {Lgr6,Frb,Nkd2,Sox10,Plp1,Lpr5,dkk3} {Lgr6,Frb,Nkd2,Sox10,Plp1,Lpr5,Nrf2} {Lgr6,Frb,Nkd2,Sox10,Plp1,dkk3,Nrf2} {Lgr6,Frb,Nkd2,Sox10,Lpr5,dkk3,Nrf2} {Lgr6,Frb,Nkd2,Plp1,Lpr5,dkk3,Nrf2} {Lgr6,Frb,Tgfbr2,Tgfbr3,Hmox1,Cyba,Sox10} {Lgr6,Frb,Tgfbr2,Tgfbr3,Hmox1,Cyba,Plp1} {Lgr6,Frb,Tgfbr2,Tgfbr3,Hmox1,Cyba,Lpr5} {Lgr6,Frb,Tgfbr2,Tgfbr3,Hmox1,Cyba,dkk3} {Lgr6,Frb,Tgfbr2,Tgfbr3,Hmox1,Cyba,Nrf2} {Lgr6,Frb,Tgfbr2,Tgfbr3,Hmox1,Sox10,Plp1} {Lgr6,Frb,Tgfbr2,Tgfbr3,Hmox1,Sox10,Lpr5} {Lgr6,Frb,Tgfbr2,Tgfbr3,Hmox1,Sox10,dkk3} {Lgr6,Frb,Tgfbr2,Tgfbr3,Hmox1,Sox10,Nrf2} {Lgr6,Frb,Tgfbr2,Tgfbr3,Hmox1,Plp1,Lpr5} {Lgr6,Frb,Tgfbr2,Tgfbr3,Hmox1,Plp1,dkk3} {Lgr6,Frb,Tgfbr2,Tgfbr3,Hmox1,Plp1,Nrf2} {Lgr6,Frb,Tgfbr2,Tgfbr3,Hmox1,Lpr5,dkk3} {Lgr6,Frb,Tgfbr2,Tgfbr3,Hmox1,Lpr5,Nrf2} {Lgr6,Frb,Tgfbr2,Tgfbr3,Hmox1,dkk3,Nrf2} {Lgr6,Frb,Tgfbr2,Tgfbr3,Cyba,Sox10,Plp1} {Lgr6,Frb,Tgfbr2,Tgfbr3,Cyba,Sox10,Lpr5} {Lgr6,Frb,Tgfbr2,Tgfbr3,Cyba,Sox10,dkk3} {Lgr6,Frb,Tgfbr2,Tgfbr3,Cyba,Sox10,Nrf2} {Lgr6,Frb,Tgfbr2,Tgfbr3,Cyba,Plp1,Lpr5} {Lgr6,Frb,Tgfbr2,Tgfbr3,Cyba,Plp1,dkk3} {Lgr6,Frb,Tgfbr2,Tgfbr3,Cyba,Plp1,Nrf2} {Lgr6,Frb,Tgfbr2,Tgfbr3,Cyba,Lpr5,dkk3} {Lgr6,Frb,Tgfbr2,Tgfbr3,Cyba,Lpr5,Nrf2} {Lgr6,Frb,Tgfbr2,Tgfbr3,Cyba,dkk3,Nrf2} {Lgr6,Frb,Tgfbr2,Tgfbr3,Sox10,Plp1,Lpr5} {Lgr6,Frb,Tgfbr2,Tgfbr3,Sox10,Plp1,dkk3} {Lgr6,Frb,Tgfbr2,Tgfbr3,Sox10,Plp1,Nrf2} {Lgr6,Frb,Tgfbr2,Tgfbr3,Sox10,Lpr5,dkk3} {Lgr6,Frb,Tgfbr2,Tgfbr3,Sox10,Lpr5,Nrf2} {Lgr6,Frb,Tgfbr2,Tgfbr3,Sox10,dkk3,Nrf2} {Lgr6,Frb,Tgfbr2,Tgfbr3,Plp1,Lpr5,dkk3} {Lgr6,Frb,Tgfbr2,Tgfbr3,Plp1,Lpr5,Nrf2} {Lgr6,Frb,Tgfbr2,Tgfbr3,Plp1,dkk3,Nrf2} {Lgr6,Frb,Tgfbr2,Tgfbr3,Lpr5,dkk3,Nrf2} {Lgr6,Frb,Tgfbr2,Hmox1,Cyba,Sox10,Plp1} {Lgr6,Frb,Tgfbr2,Hmox1,Cyba,Sox10,Lpr5} {Lgr6,Frb,Tgfbr2,Hmox1,Cyba,Sox10,dkk3} {Lgr6,Frb,Tgfbr2,Hmox1,Cyba,Sox10,Nrf2} {Lgr6,Frb,Tgfbr2,Hmox1,Cyba,Plp1,Lpr5} {Lgr6,Frb,Tgfbr2,Hmox1,Cyba,Plp1,dkk3} {Lgr6,Frb,Tgfbr2,Hmox1,Cyba,Plp1,Nrf2} {Lgr6,Frb,Tgfbr2,Hmox1,Cyba,Lpr5,dkk3} {Lgr6,Frb,Tgfbr2,Hmox1,Cyba,Lpr5,Nrf2} {Lgr6,Frb,Tgfbr2,Hmox1,Cyba,dkk3,Nrf2} {Lgr6,Frb,Tgfbr2,Hmox1,Sox10,Plp1,Lpr5} {Lgr6,Frb,Tgfbr2,Hmox1,Sox10,Plp1,dkk3} {Lgr6,Frb,Tgfbr2,Hmox1,Sox10,Plp1,Nrf2} {Lgr6,Frb,Tgfbr2,Hmox1,Sox10,Lpr5,dkk3} {Lgr6,Frb,Tgfbr2,Hmox1,Sox10,Lpr5,Nrf2} {Lgr6,Frb,Tgfbr2,Hmox1,Sox10,dkk3,Nrf2} {Lgr6,Frb,Tgfbr2,Hmox1,Plp1,Lpr5,dkk3} {Lgr6,Frb,Tgfbr2,Hmox1,Plp1,Lpr5,Nrf2} {Lgr6,Frb,Tgfbr2,Hmox1,Plp1,dkk3,Nrf2} {Lgr6,Frb,Tgfbr2,Hmox1,Lpr5,dkk3,Nrf2} {Lgr6,Frb,Tgfbr2,Cyba,Sox10,Plp1,Lpr5} {Lgr6,Frb,Tgfbr2,Cyba,Sox10,Plp1,dkk3} {Lgr6,Frb,Tgfbr2,Cyba,Sox10,Plp1,Nrf2} {Lgr6,Frb,Tgfbr2,Cyba,Sox10,Lpr5,dkk3} {Lgr6,Frb,Tgfbr2,Cyba,Sox10,Lpr5,Nrf2} {Lgr6,Frb,Tgfbr2,Cyba,Sox10,dkk3,Nrf2} {Lgr6,Frb,Tgfbr2,Cyba,Plp1,Lpr5,dkk3} {Lgr6,Frb,Tgfbr2,Cyba,Plp1,Lpr5,Nrf2} {Lgr6,Frb,Tgfbr2,Cyba,Plp1,dkk3,Nrf2} {Lgr6,Frb,Tgfbr2,Cyba,Lpr5,dkk3,Nrf2} {Lgr6,Frb,Tgfbr2,Sox10,Plp1,Lpr5,dkk3} {Lgr6,Frb,Tgfbr2,Sox10,Plp1,Lpr5,Nrf2} {Lgr6,Frb,Tgfbr2,Sox10,Plp1,dkk3,Nrf2} {Lgr6,Frb,Tgfbr2,Sox10,Lpr5,dkk3,Nrf2} {Lgr6,Frb,Tgfbr2,Plp1,Lpr5,dkk3,Nrf2} {Lgr6,Frb,Tgfbr3,Hmox1,Cyba,Sox10,Plp1} {Lgr6,Frb,Tgfbr3,Hmox1,Cyba,Sox10,Lpr5} {Lgr6,Frb,Tgfbr3,Hmox1,Cyba,Sox10,dkk3} {Lgr6,Frb,Tgfbr3,Hmox1,Cyba,Sox10,Nrf2} {Lgr6,Frb,Tgfbr3,Hmox1,Cyba,Plp1,Lpr5} {Lgr6,Frb,Tgfbr3,Hmox1,Cyba,Plp1,dkk3} {Lgr6,Frb,Tgfbr3,Hmox1,Cyba,Plp1,Nrf2} {Lgr6,Frb,Tgfbr3,Hmox1,Cyba,Lpr5,dkk3} {Lgr6,Frb,Tgfbr3,Hmox1,Cyba,Lpr5,Nrf2} {Lgr6,Frb,Tgfbr3,Hmox1,Cyba,dkk3,Nrf2} {Lgr6,Frb,Tgfbr3,Hmox1,Sox10,Plp1,Lpr5} {Lgr6,Frb,Tgfbr3,Hmox1,Sox10,Plp1,dkk3} {Lgr6,Frb,Tgfbr3,Hmox1,Sox10,Plp1,Nrf2} {Lgr6,Frb,Tgfbr3,Hmox1,Sox10,Lpr5,dkk3} {Lgr6,Frb,Tgfbr3,Hmox1,Sox10,Lpr5,Nrf2} {Lgr6,Frb,Tgfbr3,Hmox1,Sox10,dkk3,Nrf2} {Lgr6,Frb,Tgfbr3,Hmox1,Plp1,Lpr5,dkk3} {Lgr6,Frb,Tgfbr3,Hmox1,Plp1,Lpr5,Nrf2} {Lgr6,Frb,Tgfbr3,Hmox1,Plp1,dkk3,Nrf2} {Lgr6,Frb,Tgfbr3,Hmox1,Lpr5,dkk3,Nrf2} {Lgr6,Frb,Tgfbr3,Cyba,Sox10,Plp1,Lpr5} {Lgr6,Frb,Tgfbr3,Cyba,Sox10,Plp1,dkk3} {Lgr6,Frb,Tgfbr3,Cyba,Sox10,Plp1,Nrf2} {Lgr6,Frb,Tgfbr3,Cyba,Sox10,Lpr5,dkk3} {Lgr6,Frb,Tgfbr3,Cyba,Sox10,Lpr5,Nrf2} {Lgr6,Frb,Tgfbr3,Cyba,Sox10,dkk3,Nrf2} {Lgr6,Frb,Tgfbr3,Cyba,Plp1,Lpr5,dkk3} {Lgr6,Frb,Tgfbr3,Cyba,Plp1,Lpr5,Nrf2} {Lgr6,Frb,Tgfbr3,Cyba,Plp1,dkk3,Nrf2} {Lgr6,Frb,Tgfbr3,Cyba,Lpr5,dkk3,Nrf2} {Lgr6,Frb,Tgfbr3,Sox10,Plp1,Lpr5,dkk3} {Lgr6,Frb,Tgfbr3,Sox10,Plp1,Lpr5,Nrf2} {Lgr6,Frb,Tgfbr3,Sox10,Plp1,dkk3,Nrf2} {Lgr6,Frb,Tgfbr3,Sox10,Lpr5,dkk3,Nrf2} {Lgr6,Frb,Tgfbr3,Plp1,Lpr5,dkk3,Nrf2} {Lgr6,Frb,Hmox1,Cyba,Sox10,Plp1,Lpr5} {Lgr6,Frb,Hmox1,Cyba,Sox10,Plp1,dkk3} {Lgr6,Frb,Hmox1,Cyba,Sox10,Plp1,Nrf2} {Lgr6,Frb,Hmox1,Cyba,Sox10,Lpr5,dkk3} {Lgr6,Frb,Hmox1,Cyba,Sox10,Lpr5,Nrf2} {Lgr6,Frb,Hmox1,Cyba,Sox10,dkk3,Nrf2} {Lgr6,Frb,Hmox1,Cyba,Plp1,Lpr5,dkk3} {Lgr6,Frb,Hmox1,Cyba,Plp1,Lpr5,Nrf2} {Lgr6,Frb,Hmox1,Cyba,Plp1,dkk3,Nrf2} {Lgr6,Frb,Hmox1,Cyba,Lpr5,dkk3,Nrf2} {Lgr6,Frb,Hmox1,Sox10,Plp1,Lpr5,dkk3} {Lgr6,Frb,Hmox1,Sox10,Plp1,Lpr5,Nrf2} {Lgr6,Frb,Hmox1,Sox10,Plp1,dkk3,Nrf2} {Lgr6,Frb,Hmox1,Sox10,Lpr5,dkk3,Nrf2} {Lgr6,Frb,Hmox1,Plp1,Lpr5,dkk3,Nrf2} {Lgr6,Frb,Cyba,Sox10,Plp1,Lpr5,dkk3} {Lgr6,Frb,Cyba,Sox10,Plp1,Lpr5,Nrf2} {Lgr6,Frb,Cyba,Sox10,Plp1,dkk3,Nrf2} {Lgr6,Frb,Cyba,Sox10,Lpr5,dkk3,Nrf2} {Lgr6,Frb,Cyba,Plp1,Lpr5,dkk3,Nrf2} {Lgr6,Frb,Sox10,Plp1,Lpr5,dkk3,Nrf2} {Lgr6,Nkd2,Tgfbr2,Tgfbr3,Hmox1,Cyba,Sox10} {Lgr6,Nkd2,Tgfbr2,Tgfbr3,Hmox1,Cyba,Plp1} {Lgr6,Nkd2,Tgfbr2,Tgfbr3,Hmox1,Cyba,Lpr5} {Lgr6,Nkd2,Tgfbr2,Tgfbr3,Hmox1,Cyba,dkk3} {Lgr6,Nkd2,Tgfbr2,Tgfbr3,Hmox1,Cyba,Nrf2} {Lgr6,Nkd2,Tgfbr2,Tgfbr3,Hmox1,Sox10,Plp1} {Lgr6,Nkd2,Tgfbr2,Tgfbr3,Hmox1,Sox10,Lpr5} {Lgr6,Nkd2,Tgfbr2,Tgfbr3,Hmox1,Sox10,dkk3} {Lgr6,Nkd2,Tgfbr2,Tgfbr3,Hmox1,Sox10,Nrf2} {Lgr6,Nkd2,Tgfbr2,Tgfbr3,Hmox1,Plp1,Lpr5} {Lgr6,Nkd2,Tgfbr2,Tgfbr3,Hmox1,Plp1,dkk3} {Lgr6,Nkd2,Tgfbr2,Tgfbr3,Hmox1,Plp1,Nrf2} {Lgr6,Nkd2,Tgfbr2,Tgfbr3,Hmox1,Lpr5,dkk3} {Lgr6,Nkd2,Tgfbr2,Tgfbr3,Hmox1,Lpr5,Nrf2} {Lgr6,Nkd2,Tgfbr2,Tgfbr3,Hmox1,dkk3,Nrf2} {Lgr6,Nkd2,Tgfbr2,Tgfbr3,Cyba,Sox10,Plp1} {Lgr6,Nkd2,Tgfbr2,Tgfbr3,Cyba,Sox10,Lpr5} {Lgr6,Nkd2,Tgfbr2,Tgfbr3,Cyba,Sox10,dkk3} {Lgr6,Nkd2,Tgfbr2,Tgfbr3,Cyba,Sox10,Nrf2} {Lgr6,Nkd2,Tgfbr2,Tgfbr3,Cyba,Plp1,Lpr5} {Lgr6,Nkd2,Tgfbr2,Tgfbr3,Cyba,Plp1,dkk3} {Lgr6,Nkd2,Tgfbr2,Tgfbr3,Cyba,Plp1,Nrf2} {Lgr6,Nkd2,Tgfbr2,Tgfbr3,Cyba,Lpr5,dkk3} {Lgr6,Nkd2,Tgfbr2,Tgfbr3,Cyba,Lpr5,Nrf2} {Lgr6,Nkd2,Tgfbr2,Tgfbr3,Cyba,dkk3,Nrf2} {Lgr6,Nkd2,Tgfbr2,Tgfbr3,Sox10,Plp1,Lpr5} {Lgr6,Nkd2,Tgfbr2,Tgfbr3,Sox10,Plp1,dkk3} {Lgr6,Nkd2,Tgfbr2,Tgfbr3,Sox10,Plp1,Nrf2} {Lgr6,Nkd2,Tgfbr2,Tgfbr3,Sox10,Lpr5,dkk3} {Lgr6,Nkd2,Tgfbr2,Tgfbr3,Sox10,Lpr5,Nrf2} {Lgr6,Nkd2,Tgfbr2,Tgfbr3,Sox10,dkk3,Nrf2} {Lgr6,Nkd2,Tgfbr2,Tgfbr3,Plp1,Lpr5,dkk3} {Lgr6,Nkd2,Tgfbr2,Tgfbr3,Plp1,Lpr5,Nrf2} {Lgr6,Nkd2,Tgfbr2,Tgfbr3,Plp1,dkk3,Nrf2} {Lgr6,Nkd2,Tgfbr2,Tgfbr3,Lpr5,dkk3,Nrf2} {Lgr6,Nkd2,Tgfbr2,Hmox1,Cyba,Sox10,Plp1} {Lgr6,Nkd2,Tgfbr2,Hmox1,Cyba,Sox10,Lpr5} {Lgr6,Nkd2,Tgfbr2,Hmox1,Cyba,Sox10,dkk3} {Lgr6,Nkd2,Tgfbr2,Hmox1,Cyba,Sox10,Nrf2} {Lgr6,Nkd2,Tgfbr2,Hmox1,Cyba,Plp1,Lpr5} {Lgr6,Nkd2,Tgfbr2,Hmox1,Cyba,Plp1,dkk3} {Lgr6,Nkd2,Tgfbr2,Hmox1,Cyba,Plp1,Nrf2} {Lgr6,Nkd2,Tgfbr2,Hmox1,Cyba,Lpr5,dkk3} {Lgr6,Nkd2,Tgfbr2,Hmox1,Cyba,Lpr5,Nrf2} {Lgr6,Nkd2,Tgfbr2,Hmox1,Cyba,dkk3,Nrf2} {Lgr6,Nkd2,Tgfbr2,Hmox1,Sox10,Plp1,Lpr5} {Lgr6,Nkd2,Tgfbr2,Hmox1,Sox10,Plp1,dkk3} {Lgr6,Nkd2,Tgfbr2,Hmox1,Sox10,Plp1,Nrf2} {Lgr6,Nkd2,Tgfbr2,Hmox1,Sox10,Lpr5,dkk3} {Lgr6,Nkd2,Tgfbr2,Hmox1,Sox10,Lpr5,Nrf2} {Lgr6,Nkd2,Tgfbr2,Hmox1,Sox10,dkk3,Nrf2} {Lgr6,Nkd2,Tgfbr2,Hmox1,Plp1,Lpr5,dkk3} {Lgr6,Nkd2,Tgfbr2,Hmox1,Plp1,Lpr5,Nrf2} {Lgr6,Nkd2,Tgfbr2,Hmox1,Plp1,dkk3,Nrf2} {Lgr6,Nkd2,Tgfbr2,Hmox1,Lpr5,dkk3,Nrf2} {Lgr6,Nkd2,Tgfbr2,Cyba,Sox10,Plp1,Lpr5} {Lgr6,Nkd2,Tgfbr2,Cyba,Sox10,Plp1,dkk3} {Lgr6,Nkd2,Tgfbr2,Cyba,Sox10,Plp1,Nrf2} {Lgr6,Nkd2,Tgfbr2,Cyba,Sox10,Lpr5,dkk3} {Lgr6,Nkd2,Tgfbr2,Cyba,Sox10,Lpr5,Nrf2} {Lgr6,Nkd2,Tgfbr2,Cyba,Sox10,dkk3,Nrf2} {Lgr6,Nkd2,Tgfbr2,Cyba,Plp1,Lpr5,dkk3} {Lgr6,Nkd2,Tgfbr2,Cyba,Plp1,Lpr5,Nrf2} {Lgr6,Nkd2,Tgfbr2,Cyba,Plp1,dkk3,Nrf2} {Lgr6,Nkd2,Tgfbr2,Cyba,Lpr5,dkk3,Nrf2} {Lgr6,Nkd2,Tgfbr2,Sox10,Plp1,Lpr5,dkk3} {Lgr6,Nkd2,Tgfbr2,Sox10,Plp1,Lpr5,Nrf2} {Lgr6,Nkd2,Tgfbr2,Sox10,Plp1,dkk3,Nrf2} {Lgr6,Nkd2,Tgfbr2,Sox10,Lpr5,dkk3,Nrf2} {Lgr6,Nkd2,Tgfbr2,Plp1,Lpr5,dkk3,Nrf2} {Lgr6,Nkd2,Tgfbr3,Hmox1,Cyba,Sox10,Plp1} {Lgr6,Nkd2,Tgfbr3,Hmox1,Cyba,Sox10,Lpr5} {Lgr6,Nkd2,Tgfbr3,Hmox1,Cyba,Sox10,dkk3} {Lgr6,Nkd2,Tgfbr3,Hmox1,Cyba,Sox10,Nrf2} {Lgr6,Nkd2,Tgfbr3,Hmox1,Cyba,Plp1,Lpr5} {Lgr6,Nkd2,Tgfbr3,Hmox1,Cyba,Plp1,dkk3} {Lgr6,Nkd2,Tgfbr3,Hmox1,Cyba,Plp1,Nrf2} {Lgr6,Nkd2,Tgfbr3,Hmox1,Cyba,Lpr5,dkk3} {Lgr6,Nkd2,Tgfbr3,Hmox1,Cyba,Lpr5,Nrf2} {Lgr6,Nkd2,Tgfbr3,Hmox1,Cyba,dkk3,Nrf2} {Lgr6,Nkd2,Tgfbr3,Hmox1,Sox10,Plp1,Lpr5} {Lgr6,Nkd2,Tgfbr3,Hmox1,Sox10,Plp1,dkk3} {Lgr6,Nkd2,Tgfbr3,Hmox1,Sox10,Plp1,Nrf2} {Lgr6,Nkd2,Tgfbr3,Hmox1,Sox10,Lpr5,dkk3} {Lgr6,Nkd2,Tgfbr3,Hmox1,Sox10,Lpr5,Nrf2} {Lgr6,Nkd2,Tgfbr3,Hmox1,Sox10,dkk3,Nrf2} {Lgr6,Nkd2,Tgfbr3,Hmox1,Plp1,Lpr5,dkk3} {Lgr6,Nkd2,Tgfbr3,Hmox1,Plp1,Lpr5,Nrf2} {Lgr6,Nkd2,Tgfbr3,Hmox1,Plp1,dkk3,Nrf2} {Lgr6,Nkd2,Tgfbr3,Hmox1,Lpr5,dkk3,Nrf2} {Lgr6,Nkd2,Tgfbr3,Cyba,Sox10,Plp1,Lpr5} {Lgr6,Nkd2,Tgfbr3,Cyba,Sox10,Plp1,dkk3} {Lgr6,Nkd2,Tgfbr3,Cyba,Sox10,Plp1,Nrf2} {Lgr6,Nkd2,Tgfbr3,Cyba,Sox10,Lpr5,dkk3} {Lgr6,Nkd2,Tgfbr3,Cyba,Sox10,Lpr5,Nrf2} {Lgr6,Nkd2,Tgfbr3,Cyba,Sox10,dkk3,Nrf2} {Lgr6,Nkd2,Tgfbr3,Cyba,Plp1,Lpr5,dkk3} {Lgr6,Nkd2,Tgfbr3,Cyba,Plp1,Lpr5,Nrf2} {Lgr6,Nkd2,Tgfbr3,Cyba,Plp1,dkk3,Nrf2} {Lgr6,Nkd2,Tgfbr3,Cyba,Lpr5,dkk3,Nrf2} {Lgr6,Nkd2,Tgfbr3,Sox10,Plp1,Lpr5,dkk3} {Lgr6,Nkd2,Tgfbr3,Sox10,Plp1,Lpr5,Nrf2} {Lgr6,Nkd2,Tgfbr3,Sox10,Plp1,dkk3,Nrf2} {Lgr6,Nkd2,Tgfbr3,Sox10,Lpr5,dkk3,Nrf2} {Lgr6,Nkd2,Tgfbr3,Plp1,Lpr5,dkk3,Nrf2} {Lgr6,Nkd2,Hmox1,Cyba,Sox10,Plp1,Lpr5} {Lgr6,Nkd2,Hmox1,Cyba,Sox10,Plp1,dkk3} {Lgr6,Nkd2,Hmox1,Cyba,Sox10,Plp1,Nrf2} {Lgr6,Nkd2,Hmox1,Cyba,Sox10,Lpr5,dkk3} {Lgr6,Nkd2,Hmox1,Cyba,Sox10,Lpr5,Nrf2} {Lgr6,Nkd2,Hmox1,Cyba,Sox10,dkk3,Nrf2} {Lgr6,Nkd2,Hmox1,Cyba,Plp1,Lpr5,dkk3} {Lgr6,Nkd2,Hmox1,Cyba,Plp1,Lpr5,Nrf2} {Lgr6,Nkd2,Hmox1,Cyba,Plp1,dkk3,Nrf2} {Lgr6,Nkd2,Hmox1,Cyba,Lpr5,dkk3,Nrf2} {Lgr6,Nkd2,Hmox1,Sox10,Plp1,Lpr5,dkk3} {Lgr6,Nkd2,Hmox1,Sox10,Plp1,Lpr5,Nrf2} {Lgr6,Nkd2,Hmox1,Sox10,Plp1,dkk3,Nrf2} {Lgr6,Nkd2,Hmox1,Sox10,Lpr5,dkk3,Nrf2} {Lgr6,Nkd2,Hmox1,Plp1,Lpr5,dkk3,Nrf2} {Lgr6,Nkd2,Cyba,Sox10,Plp1,Lpr5,dkk3} {Lgr6,Nkd2,Cyba,Sox10,Plp1,Lpr5,Nrf2} {Lgr6,Nkd2,Cyba,Sox10,Plp1,dkk3,Nrf2} {Lgr6,Nkd2,Cyba,Sox10,Lpr5,dkk3,Nrf2} {Lgr6,Nkd2,Cyba,Plp1,Lpr5,dkk3,Nrf2} {Lgr6,Nkd2,Sox10,Plp1,Lpr5,dkk3,Nrf2} {Lgr6,Tgfbr2,Tgfbr3,Hmox1,Cyba,Sox10,Plp1} {Lgr6,Tgfbr2,Tgfbr3,Hmox1,Cyba,Sox10,Lpr5} {Lgr6,Tgfbr2,Tgfbr3,Hmox1,Cyba,Sox10,dkk3} {Lgr6,Tgfbr2,Tgfbr3,Hmox1,Cyba,Sox10,Nrf2} {Lgr6,Tgfbr2,Tgfbr3,Hmox1,Cyba,Plp1,Lpr5} {Lgr6,Tgfbr2,Tgfbr3,Hmox1,Cyba,Plp1,dkk3} {Lgr6,Tgfbr2,Tgfbr3,Hmox1,Cyba,Plp1,Nrf2} {Lgr6,Tgfbr2,Tgfbr3,Hmox1,Cyba,Lpr5,dkk3} {Lgr6,Tgfbr2,Tgfbr3,Hmox1,Cyba,Lpr5,Nrf2} {Lgr6,Tgfbr2,Tgfbr3,Hmox1,Cyba,dkk3,Nrf2} {Lgr6,Tgfbr2,Tgfbr3,Hmox1,Sox10,Plp1,Lpr5} {Lgr6,Tgfbr2,Tgfbr3,Hmox1,Sox10,Plp1,dkk3} {Lgr6,Tgfbr2,Tgfbr3,Hmox1,Sox10,Plp1,Nrf2} {Lgr6,Tgfbr2,Tgfbr3,Hmox1,Sox10,Lpr5,dkk3} {Lgr6,Tgfbr2,Tgfbr3,Hmox1,Sox10,Lpr5,Nrf2} {Lgr6,Tgfbr2,Tgfbr3,Hmox1,Sox10,dkk3,Nrf2} {Lgr6,Tgfbr2,Tgfbr3,Hmox1,Plp1,Lpr5,dkk3} {Lgr6,Tgfbr2,Tgfbr3,Hmox1,Plp1,Lpr5,Nrf2} {Lgr6,Tgfbr2,Tgfbr3,Hmox1,Plp1,dkk3,Nrf2} {Lgr6,Tgfbr2,Tgfbr3,Hmox1,Lpr5,dkk3,Nrf2} {Lgr6,Tgfbr2,Tgfbr3,Cyba,Sox10,Plp1,Lpr5} {Lgr6,Tgfbr2,Tgfbr3,Cyba,Sox10,Plp1,dkk3} {Lgr6,Tgfbr2,Tgfbr3,Cyba,Sox10,Plp1,Nrf2} {Lgr6,Tgfbr2,Tgfbr3,Cyba,Sox10,Lpr5,dkk3} {Lgr6,Tgfbr2,Tgfbr3,Cyba,Sox10,Lpr5,Nrf2} {Lgr6,Tgfbr2,Tgfbr3,Cyba,Sox10,dkk3,Nrf2} {Lgr6,Tgfbr2,Tgfbr3,Cyba,Plp1,Lpr5,dkk3} {Lgr6,Tgfbr2,Tgfbr3,Cyba,Plp1,Lpr5,Nrf2} {Lgr6,Tgfbr2,Tgfbr3,Cyba,Plp1,dkk3,Nrf2} {Lgr6,Tgfbr2,Tgfbr3,Cyba,Lpr5,dkk3,Nrf2} {Lgr6,Tgfbr2,Tgfbr3,Sox10,Plp1,Lpr5,dkk3} {Lgr6,Tgfbr2,Tgfbr3,Sox10,Plp1,Lpr5,Nrf2} {Lgr6,Tgfbr2,Tgfbr3,Sox10,Plp1,dkk3,Nrf2} {Lgr6,Tgfbr2,Tgfbr3,Sox10,Lpr5,dkk3,Nrf2} {Lgr6,Tgfbr2,Tgfbr3,Plp1,Lpr5,dkk3,Nrf2} {Lgr6,Tgfbr2,Hmox1,Cyba,Sox10,Plp1,Lpr5} {Lgr6,Tgfbr2,Hmox1,Cyba,Sox10,Plp1,dkk3} {Lgr6,Tgfbr2,Hmox1,Cyba,Sox10,Plp1,Nrf2} {Lgr6,Tgfbr2,Hmox1,Cyba,Sox10,Lpr5,dkk3} {Lgr6,Tgfbr2,Hmox1,Cyba,Sox10,Lpr5,Nrf2} {Lgr6,Tgfbr2,Hmox1,Cyba,Sox10,dkk3,Nrf2} {Lgr6,Tgfbr2,Hmox1,Cyba,Plp1,Lpr5,dkk3} {Lgr6,Tgfbr2,Hmox1,Cyba,Plp1,Lpr5,Nrf2} {Lgr6,Tgfbr2,Hmox1,Cyba,Plp1,dkk3,Nrf2} {Lgr6,Tgfbr2,Hmox1,Cyba,Lpr5,dkk3,Nrf2} {Lgr6,Tgfbr2,Hmox1,Sox10,Plp1,Lpr5,dkk3} {Lgr6,Tgfbr2,Hmox1,Sox10,Plp1,Lpr5,Nrf2} {Lgr6,Tgfbr2,Hmox1,Sox10,Plp1,dkk3,Nrf2} {Lgr6,Tgfbr2,Hmox1,Sox10,Lpr5,dkk3,Nrf2} {Lgr6,Tgfbr2,Hmox1,Plp1,Lpr5,dkk3,Nrf2} {Lgr6,Tgfbr2,Cyba,Sox10,Plp1,Lpr5,dkk3} {Lgr6,Tgfbr2,Cyba,Sox10,Plp1,Lpr5,Nrf2} {Lgr6,Tgfbr2,Cyba,Sox10,Plp1,dkk3,Nrf2} {Lgr6,Tgfbr2,Cyba,Sox10,Lpr5,dkk3,Nrf2} {Lgr6,Tgfbr2,Cyba,Plp1,Lpr5,dkk3,Nrf2} {Lgr6,Tgfbr2,Sox10,Plp1,Lpr5,dkk3,Nrf2} {Lgr6,Tgfbr3,Hmox1,Cyba,Sox10,Plp1,Lpr5} {Lgr6,Tgfbr3,Hmox1,Cyba,Sox10,Plp1,dkk3} {Lgr6,Tgfbr3,Hmox1,Cyba,Sox10,Plp1,Nrf2} {Lgr6,Tgfbr3,Hmox1,Cyba,Sox10,Lpr5,dkk3} {Lgr6,Tgfbr3,Hmox1,Cyba,Sox10,Lpr5,Nrf2} {Lgr6,Tgfbr3,Hmox1,Cyba,Sox10,dkk3,Nrf2} {Lgr6,Tgfbr3,Hmox1,Cyba,Plp1,Lpr5,dkk3} {Lgr6,Tgfbr3,Hmox1,Cyba,Plp1,Lpr5,Nrf2} {Lgr6,Tgfbr3,Hmox1,Cyba,Plp1,dkk3,Nrf2} {Lgr6,Tgfbr3,Hmox1,Cyba,Lpr5,dkk3,Nrf2} {Lgr6,Tgfbr3,Hmox1,Sox10,Plp1,Lpr5,dkk3} {Lgr6,Tgfbr3,Hmox1,Sox10,Plp1,Lpr5,Nrf2} {Lgr6,Tgfbr3,Hmox1,Sox10,Plp1,dkk3,Nrf2} {Lgr6,Tgfbr3,Hmox1,Sox10,Lpr5,dkk3,Nrf2} {Lgr6,Tgfbr3,Hmox1,Plp1,Lpr5,dkk3,Nrf2} {Lgr6,Tgfbr3,Cyba,Sox10,Plp1,Lpr5,dkk3} {Lgr6,Tgfbr3,Cyba,Sox10,Plp1,Lpr5,Nrf2} {Lgr6,Tgfbr3,Cyba,Sox10,Plp1,dkk3,Nrf2} {Lgr6,Tgfbr3,Cyba,Sox10,Lpr5,dkk3,Nrf2} {Lgr6,Tgfbr3,Cyba,Plp1,Lpr5,dkk3,Nrf2} {Lgr6,Tgfbr3,Sox10,Plp1,Lpr5,dkk3,Nrf2} {Lgr6,Hmox1,Cyba,Sox10,Plp1,Lpr5,dkk3} {Lgr6,Hmox1,Cyba,Sox10,Plp1,Lpr5,Nrf2} {Lgr6,Hmox1,Cyba,Sox10,Plp1,dkk3,Nrf2} {Lgr6,Hmox1,Cyba,Sox10,Lpr5,dkk3,Nrf2} {Lgr6,Hmox1,Cyba,Plp1,Lpr5,dkk3,Nrf2} {Lgr6,Hmox1,Sox10,Plp1,Lpr5,dkk3,Nrf2} {Lgr6,Cyba,Sox10,Plp1,Lpr5,dkk3,Nrf2} {Frb,Nkd2,Tgfbr2,Tgfbr3,Hmox1,Cyba,Sox10} {Frb,Nkd2,Tgfbr2,Tgfbr3,Hmox1,Cyba,Plp1} {Frb,Nkd2,Tgfbr2,Tgfbr3,Hmox1,Cyba,Lpr5} {Frb,Nkd2,Tgfbr2,Tgfbr3,Hmox1,Cyba,dkk3} {Frb,Nkd2,Tgfbr2,Tgfbr3,Hmox1,Cyba,Nrf2} {Frb,Nkd2,Tgfbr2,Tgfbr3,Hmox1,Sox10,Plp1} {Frb,Nkd2,Tgfbr2,Tgfbr3,Hmox1,Sox10,Lpr5} {Frb,Nkd2,Tgfbr2,Tgfbr3,Hmox1,Sox10,dkk3} {Frb,Nkd2,Tgfbr2,Tgfbr3,Hmox1,Sox10,Nrf2} {Frb,Nkd2,Tgfbr2,Tgfbr3,Hmox1,Plp1,Lpr5} {Frb,Nkd2,Tgfbr2,Tgfbr3,Hmox1,Plp1,dkk3} {Frb,Nkd2,Tgfbr2,Tgfbr3,Hmox1,Plp1,Nrf2} {Frb,Nkd2,Tgfbr2,Tgfbr3,Hmox1,Lpr5,dkk3} {Frb,Nkd2,Tgfbr2,Tgfbr3,Hmox1,Lpr5,Nrf2} {Frb,Nkd2,Tgfbr2,Tgfbr3,Hmox1,dkk3,Nrf2} {Frb,Nkd2,Tgfbr2,Tgfbr3,Cyba,Sox10,Plp1} {Frb,Nkd2,Tgfbr2,Tgfbr3,Cyba,Sox10,Lpr5} {Frb,Nkd2,Tgfbr2,Tgfbr3,Cyba,Sox10,dkk3} {Frb,Nkd2,Tgfbr2,Tgfbr3,Cyba,Sox10,Nrf2} {Frb,Nkd2,Tgfbr2,Tgfbr3,Cyba,Plp1,Lpr5} {Frb,Nkd2,Tgfbr2,Tgfbr3,Cyba,Plp1,dkk3} {Frb,Nkd2,Tgfbr2,Tgfbr3,Cyba,Plp1,Nrf2} {Frb,Nkd2,Tgfbr2,Tgfbr3,Cyba,Lpr5,dkk3} {Frb,Nkd2,Tgfbr2,Tgfbr3,Cyba,Lpr5,Nrf2} {Frb,Nkd2,Tgfbr2,Tgfbr3,Cyba,dkk3,Nrf2} {Frb,Nkd2,Tgfbr2,Tgfbr3,Sox10,Plp1,Lpr5} {Frb,Nkd2,Tgfbr2,Tgfbr3,Sox10,Plp1,dkk3} {Frb,Nkd2,Tgfbr2,Tgfbr3,Sox10,Plp1,Nrf2} {Frb,Nkd2,Tgfbr2,Tgfbr3,Sox10,Lpr5,dkk3} {Frb,Nkd2,Tgfbr2,Tgfbr3,Sox10,Lpr5,Nrf2} {Frb,Nkd2,Tgfbr2,Tgfbr3,Sox10,dkk3,Nrf2} {Frb,Nkd2,Tgfbr2,Tgfbr3,Plp1,Lpr5,dkk3} {Frb,Nkd2,Tgfbr2,Tgfbr3,Plp1,Lpr5,Nrf2} {Frb,Nkd2,Tgfbr2,Tgfbr3,Plp1,dkk3,Nrf2} {Frb,Nkd2,Tgfbr2,Tgfbr3,Lpr5,dkk3,Nrf2} {Frb,Nkd2,Tgfbr2,Hmox1,Cyba,Sox10,Plp1} {Frb,Nkd2,Tgfbr2,Hmox1,Cyba,Sox10,Lpr5} {Frb,Nkd2,Tgfbr2,Hmox1,Cyba,Sox10,dkk3} {Frb,Nkd2,Tgfbr2,Hmox1,Cyba,Sox10,Nrf2} {Frb,Nkd2,Tgfbr2,Hmox1,Cyba,Plp1,Lpr5} {Frb,Nkd2,Tgfbr2,Hmox1,Cyba,Plp1,dkk3} {Frb,Nkd2,Tgfbr2,Hmox1,Cyba,Plp1,Nrf2} {Frb,Nkd2,Tgfbr2,Hmox1,Cyba,Lpr5,dkk3} {Frb,Nkd2,Tgfbr2,Hmox1,Cyba,Lpr5,Nrf2} {Frb,Nkd2,Tgfbr2,Hmox1,Cyba,dkk3,Nrf2} {Frb,Nkd2,Tgfbr2,Hmox1,Sox10,Plp1,Lpr5} {Frb,Nkd2,Tgfbr2,Hmox1,Sox10,Plp1,dkk3} {Frb,Nkd2,Tgfbr2,Hmox1,Sox10,Plp1,Nrf2} {Frb,Nkd2,Tgfbr2,Hmox1,Sox10,Lpr5,dkk3} {Frb,Nkd2,Tgfbr2,Hmox1,Sox10,Lpr5,Nrf2} {Frb,Nkd2,Tgfbr2,Hmox1,Sox10,dkk3,Nrf2} {Frb,Nkd2,Tgfbr2,Hmox1,Plp1,Lpr5,dkk3} {Frb,Nkd2,Tgfbr2,Hmox1,Plp1,Lpr5,Nrf2} {Frb,Nkd2,Tgfbr2,Hmox1,Plp1,dkk3,Nrf2} {Frb,Nkd2,Tgfbr2,Hmox1,Lpr5,dkk3,Nrf2} {Frb,Nkd2,Tgfbr2,Cyba,Sox10,Plp1,Lpr5} {Frb,Nkd2,Tgfbr2,Cyba,Sox10,Plp1,dkk3} {Frb,Nkd2,Tgfbr2,Cyba,Sox10,Plp1,Nrf2} {Frb,Nkd2,Tgfbr2,Cyba,Sox10,Lpr5,dkk3} {Frb,Nkd2,Tgfbr2,Cyba,Sox10,Lpr5,Nrf2} {Frb,Nkd2,Tgfbr2,Cyba,Sox10,dkk3,Nrf2} {Frb,Nkd2,Tgfbr2,Cyba,Plp1,Lpr5,dkk3} {Frb,Nkd2,Tgfbr2,Cyba,Plp1,Lpr5,Nrf2} {Frb,Nkd2,Tgfbr2,Cyba,Plp1,dkk3,Nrf2} {Frb,Nkd2,Tgfbr2,Cyba,Lpr5,dkk3,Nrf2} {Frb,Nkd2,Tgfbr2,Sox10,Plp1,Lpr5,dkk3} {Frb,Nkd2,Tgfbr2,Sox10,Plp1,Lpr5,Nrf2} {Frb,Nkd2,Tgfbr2,Sox10,Plp1,dkk3,Nrf2} {Frb,Nkd2,Tgfbr2,Sox10,Lpr5,dkk3,Nrf2} {Frb,Nkd2,Tgfbr2,Plp1,Lpr5,dkk3,Nrf2} {Frb,Nkd2,Tgfbr3,Hmox1,Cyba,Sox10,Plp1} {Frb,Nkd2,Tgfbr3,Hmox1,Cyba,Sox10,Lpr5} {Frb,Nkd2,Tgfbr3,Hmox1,Cyba,Sox10,dkk3} {Frb,Nkd2,Tgfbr3,Hmox1,Cyba,Sox10,Nrf2} {Frb,Nkd2,Tgfbr3,Hmox1,Cyba,Plp1,Lpr5} {Frb,Nkd2,Tgfbr3,Hmox1,Cyba,Plp1,dkk3} {Frb,Nkd2,Tgfbr3,Hmox1,Cyba,Plp1,Nrf2} {Frb,Nkd2,Tgfbr3,Hmox1,Cyba,Lpr5,dkk3} {Frb,Nkd2,Tgfbr3,Hmox1,Cyba,Lpr5,Nrf2} {Frb,Nkd2,Tgfbr3,Hmox1,Cyba,dkk3,Nrf2} {Frb,Nkd2,Tgfbr3,Hmox1,Sox10,Plp1,Lpr5} {Frb,Nkd2,Tgfbr3,Hmox1,Sox10,Plp1,dkk3} {Frb,Nkd2,Tgfbr3,Hmox1,Sox10,Plp1,Nrf2} {Frb,Nkd2,Tgfbr3,Hmox1,Sox10,Lpr5,dkk3} {Frb,Nkd2,Tgfbr3,Hmox1,Sox10,Lpr5,Nrf2} {Frb,Nkd2,Tgfbr3,Hmox1,Sox10,dkk3,Nrf2} {Frb,Nkd2,Tgfbr3,Hmox1,Plp1,Lpr5,dkk3} {Frb,Nkd2,Tgfbr3,Hmox1,Plp1,Lpr5,Nrf2} {Frb,Nkd2,Tgfbr3,Hmox1,Plp1,dkk3,Nrf2} {Frb,Nkd2,Tgfbr3,Hmox1,Lpr5,dkk3,Nrf2} {Frb,Nkd2,Tgfbr3,Cyba,Sox10,Plp1,Lpr5} {Frb,Nkd2,Tgfbr3,Cyba,Sox10,Plp1,dkk3} {Frb,Nkd2,Tgfbr3,Cyba,Sox10,Plp1,Nrf2} {Frb,Nkd2,Tgfbr3,Cyba,Sox10,Lpr5,dkk3} {Frb,Nkd2,Tgfbr3,Cyba,Sox10,Lpr5,Nrf2} {Frb,Nkd2,Tgfbr3,Cyba,Sox10,dkk3,Nrf2} {Frb,Nkd2,Tgfbr3,Cyba,Plp1,Lpr5,dkk3} {Frb,Nkd2,Tgfbr3,Cyba,Plp1,Lpr5,Nrf2} {Frb,Nkd2,Tgfbr3,Cyba,Plp1,dkk3,Nrf2} {Frb,Nkd2,Tgfbr3,Cyba,Lpr5,dkk3,Nrf2} {Frb,Nkd2,Tgfbr3,Sox10,Plp1,Lpr5,dkk3} {Frb,Nkd2,Tgfbr3,Sox10,Plp1,Lpr5,Nrf2} {Frb,Nkd2,Tgfbr3,Sox10,Plp1,dkk3,Nrf2} {Frb,Nkd2,Tgfbr3,Sox10,Lpr5,dkk3,Nrf2} {Frb,Nkd2,Tgfbr3,Plp1,Lpr5,dkk3,Nrf2} {Frb,Nkd2,Hmox1,Cyba,Sox10,Plp1,Lpr5} {Frb,Nkd2,Hmox1,Cyba,Sox10,Plp1,dkk3} {Frb,Nkd2,Hmox1,Cyba,Sox10,Plp1,Nrf2} {Frb,Nkd2,Hmox1,Cyba,Sox10,Lpr5,dkk3} {Frb,Nkd2,Hmox1,Cyba,Sox10,Lpr5,Nrf2} {Frb,Nkd2,Hmox1,Cyba,Sox10,dkk3,Nrf2} {Frb,Nkd2,Hmox1,Cyba,Plp1,Lpr5,dkk3} {Frb,Nkd2,Hmox1,Cyba,Plp1,Lpr5,Nrf2} {Frb,Nkd2,Hmox1,Cyba,Plp1,dkk3,Nrf2} {Frb,Nkd2,Hmox1,Cyba,Lpr5,dkk3,Nrf2} {Frb,Nkd2,Hmox1,Sox10,Plp1,Lpr5,dkk3} {Frb,Nkd2,Hmox1,Sox10,Plp1,Lpr5,Nrf2} {Frb,Nkd2,Hmox1,Sox10,Plp1,dkk3,Nrf2} {Frb,Nkd2,Hmox1,Sox10,Lpr5,dkk3,Nrf2} {Frb,Nkd2,Hmox1,Plp1,Lpr5,dkk3,Nrf2} {Frb,Nkd2,Cyba,Sox10,Plp1,Lpr5,dkk3} {Frb,Nkd2,Cyba,Sox10,Plp1,Lpr5,Nrf2} {Frb,Nkd2,Cyba,Sox10,Plp1,dkk3,Nrf2} {Frb,Nkd2,Cyba,Sox10,Lpr5,dkk3,Nrf2} {Frb,Nkd2,Cyba,Plp1,Lpr5,dkk3,Nrf2} {Frb,Nkd2,Sox10,Plp1,Lpr5,dkk3,Nrf2} {Frb,Tgfbr2,Tgfbr3,Hmox1,Cyba,Sox10,Plp1} {Frb,Tgfbr2,Tgfbr3,Hmox1,Cyba,Sox10,Lpr5} {Frb,Tgfbr2,Tgfbr3,Hmox1,Cyba,Sox10,dkk3} {Frb,Tgfbr2,Tgfbr3,Hmox1,Cyba,Sox10,Nrf2} {Frb,Tgfbr2,Tgfbr3,Hmox1,Cyba,Plp1,Lpr5} {Frb,Tgfbr2,Tgfbr3,Hmox1,Cyba,Plp1,dkk3} {Frb,Tgfbr2,Tgfbr3,Hmox1,Cyba,Plp1,Nrf2} {Frb,Tgfbr2,Tgfbr3,Hmox1,Cyba,Lpr5,dkk3} {Frb,Tgfbr2,Tgfbr3,Hmox1,Cyba,Lpr5,Nrf2} {Frb,Tgfbr2,Tgfbr3,Hmox1,Cyba,dkk3,Nrf2} {Frb,Tgfbr2,Tgfbr3,Hmox1,Sox10,Plp1,Lpr5} {Frb,Tgfbr2,Tgfbr3,Hmox1,Sox10,Plp1,dkk3} {Frb,Tgfbr2,Tgfbr3,Hmox1,Sox10,Plp1,Nrf2} {Frb,Tgfbr2,Tgfbr3,Hmox1,Sox10,Lpr5,dkk3} {Frb,Tgfbr2,Tgfbr3,Hmox1,Sox10,Lpr5,Nrf2} {Frb,Tgfbr2,Tgfbr3,Hmox1,Sox10,dkk3,Nrf2} {Frb,Tgfbr2,Tgfbr3,Hmox1,Plp1,Lpr5,dkk3} {Frb,Tgfbr2,Tgfbr3,Hmox1,Plp1,Lpr5,Nrf2} {Frb,Tgfbr2,Tgfbr3,Hmox1,Plp1,dkk3,Nrf2} {Frb,Tgfbr2,Tgfbr3,Hmox1,Lpr5,dkk3,Nrf2} {Frb,Tgfbr2,Tgfbr3,Cyba,Sox10,Plp1,Lpr5} {Frb,Tgfbr2,Tgfbr3,Cyba,Sox10,Plp1,dkk3} {Frb,Tgfbr2,Tgfbr3,Cyba,Sox10,Plp1,Nrf2} {Frb,Tgfbr2,Tgfbr3,Cyba,Sox10,Lpr5,dkk3} {Frb,Tgfbr2,Tgfbr3,Cyba,Sox10,Lpr5,Nrf2} {Frb,Tgfbr2,Tgfbr3,Cyba,Sox10,dkk3,Nrf2} {Frb,Tgfbr2,Tgfbr3,Cyba,Plp1,Lpr5,dkk3} {Frb,Tgfbr2,Tgfbr3,Cyba,Plp1,Lpr5,Nrf2} {Frb,Tgfbr2,Tgfbr3,Cyba,Plp1,dkk3,Nrf2} {Frb,Tgfbr2,Tgfbr3,Cyba,Lpr5,dkk3,Nrf2} {Frb,Tgfbr2,Tgfbr3,Sox10,Plp1,Lpr5,dkk3} {Frb,Tgfbr2,Tgfbr3,Sox10,Plp1,Lpr5,Nrf2} {Frb,Tgfbr2,Tgfbr3,Sox10,Plp1,dkk3,Nrf2} {Frb,Tgfbr2,Tgfbr3,Sox10,Lpr5,dkk3,Nrf2} {Frb,Tgfbr2,Tgfbr3,Plp1,Lpr5,dkk3,Nrf2} {Frb,Tgfbr2,Hmox1,Cyba,Sox10,Plp1,Lpr5} {Frb,Tgfbr2,Hmox1,Cyba,Sox10,Plp1,dkk3} {Frb,Tgfbr2,Hmox1,Cyba,Sox10,Plp1,Nrf2} {Frb,Tgfbr2,Hmox1,Cyba,Sox10,Lpr5,dkk3} {Frb,Tgfbr2,Hmox1,Cyba,Sox10,Lpr5,Nrf2} {Frb,Tgfbr2,Hmox1,Cyba,Sox10,dkk3,Nrf2} {Frb,Tgfbr2,Hmox1,Cyba,Plp1,Lpr5,dkk3} {Frb,Tgfbr2,Hmox1,Cyba,Plp1,Lpr5,Nrf2} {Frb,Tgfbr2,Hmox1,Cyba,Plp1,dkk3,Nrf2} {Frb,Tgfbr2,Hmox1,Cyba,Lpr5,dkk3,Nrf2} {Frb,Tgfbr2,Hmox1,Sox10,Plp1,Lpr5,dkk3} {Frb,Tgfbr2,Hmox1,Sox10,Plp1,Lpr5,Nrf2} {Frb,Tgfbr2,Hmox1,Sox10,Plp1,dkk3,Nrf2} {Frb,Tgfbr2,Hmox1,Sox10,Lpr5,dkk3,Nrf2} {Frb,Tgfbr2,Hmox1,Plp1,Lpr5,dkk3,Nrf2} {Frb,Tgfbr2,Cyba,Sox10,Plp1,Lpr5,dkk3} {Frb,Tgfbr2,Cyba,Sox10,Plp1,Lpr5,Nrf2} {Frb,Tgfbr2,Cyba,Sox10,Plp1,dkk3,Nrf2} {Frb,Tgfbr2,Cyba,Sox10,Lpr5,dkk3,Nrf2} {Frb,Tgfbr2,Cyba,Plp1,Lpr5,dkk3,Nrf2} {Frb,Tgfbr2,Sox10,Plp1,Lpr5,dkk3,Nrf2} {Frb,Tgfbr3,Hmox1,Cyba,Sox10,Plp1,Lpr5} {Frb,Tgfbr3,Hmox1,Cyba,Sox10,Plp1,dkk3} {Frb,Tgfbr3,Hmox1,Cyba,Sox10,Plp1,Nrf2} {Frb,Tgfbr3,Hmox1,Cyba,Sox10,Lpr5,dkk3} {Frb,Tgfbr3,Hmox1,Cyba,Sox10,Lpr5,Nrf2} {Frb,Tgfbr3,Hmox1,Cyba,Sox10,dkk3,Nrf2} {Frb,Tgfbr3,Hmox1,Cyba,Plp1,Lpr5,dkk3} {Frb,Tgfbr3,Hmox1,Cyba,Plp1,Lpr5,Nrf2} {Frb,Tgfbr3,Hmox1,Cyba,Plp1,dkk3,Nrf2} {Frb,Tgfbr3,Hmox1,Cyba,Lpr5,dkk3,Nrf2} {Frb,Tgfbr3,Hmox1,Sox10,Plp1,Lpr5,dkk3} {Frb,Tgfbr3,Hmox1,Sox10,Plp1,Lpr5,Nrf2} {Frb,Tgfbr3,Hmox1,Sox10,Plp1,dkk3,Nrf2} {Frb,Tgfbr3,Hmox1,Sox10,Lpr5,dkk3,Nrf2} {Frb,Tgfbr3,Hmox1,Plp1,Lpr5,dkk3,Nrf2} {Frb,Tgfbr3,Cyba,Sox10,Plp1,Lpr5,dkk3} {Frb,Tgfbr3,Cyba,Sox10,Plp1,Lpr5,Nrf2} {Frb,Tgfbr3,Cyba,Sox10,Plp1,dkk3,Nrf2} {Frb,Tgfbr3,Cyba,Sox10,Lpr5,dkk3,Nrf2} {Frb,Tgfbr3,Cyba,Plp1,Lpr5,dkk3,Nrf2} {Frb,Tgfbr3,Sox10,Plp1,Lpr5,dkk3,Nrf2} {Frb,Hmox1,Cyba,Sox10,Plp1,Lpr5,dkk3} {Frb,Hmox1,Cyba,Sox10,Plp1,Lpr5,Nrf2} {Frb,Hmox1,Cyba,Sox10,Plp1,dkk3,Nrf2} {Frb,Hmox1,Cyba,Sox10,Lpr5,dkk3,Nrf2} {Frb,Hmox1,Cyba,Plp1,Lpr5,dkk3,Nrf2} {Frb,Hmox1,Sox10,Plp1,Lpr5,dkk3,Nrf2} {Frb,Cyba,Sox10,Plp1,Lpr5,dkk3,Nrf2} {Nkd2,Tgfbr2,Tgfbr3,Hmox1,Cyba,Sox10,Plp1} {Nkd2,Tgfbr2,Tgfbr3,Hmox1,Cyba,Sox10,Lpr5} {Nkd2,Tgfbr2,Tgfbr3,Hmox1,Cyba,Sox10,dkk3} {Nkd2,Tgfbr2,Tgfbr3,Hmox1,Cyba,Sox10,Nrf2} {Nkd2,Tgfbr2,Tgfbr3,Hmox1,Cyba,Plp1,Lpr5} {Nkd2,Tgfbr2,Tgfbr3,Hmox1,Cyba,Plp1,dkk3} {Nkd2,Tgfbr2,Tgfbr3,Hmox1,Cyba,Plp1,Nrf2} {Nkd2,Tgfbr2,Tgfbr3,Hmox1,Cyba,Lpr5,dkk3} {Nkd2,Tgfbr2,Tgfbr3,Hmox1,Cyba,Lpr5,Nrf2} {Nkd2,Tgfbr2,Tgfbr3,Hmox1,Cyba,dkk3,Nrf2} {Nkd2,Tgfbr2,Tgfbr3,Hmox1,Sox10,Plp1,Lpr5} {Nkd2,Tgfbr2,Tgfbr3,Hmox1,Sox10,Plp1,dkk3} {Nkd2,Tgfbr2,Tgfbr3,Hmox1,Sox10,Plp1,Nrf2} {Nkd2,Tgfbr2,Tgfbr3,Hmox1,Sox10,Lpr5,dkk3} {Nkd2,Tgfbr2,Tgfbr3,Hmox1,Sox10,Lpr5,Nrf2} {Nkd2,Tgfbr2,Tgfbr3,Hmox1,Sox10,dkk3,Nrf2} {Nkd2,Tgfbr2,Tgfbr3,Hmox1,Plp1,Lpr5,dkk3} {Nkd2,Tgfbr2,Tgfbr3,Hmox1,Plp1,Lpr5,Nrf2} {Nkd2,Tgfbr2,Tgfbr3,Hmox1,Plp1,dkk3,Nrf2} {Nkd2,Tgfbr2,Tgfbr3,Hmox1,Lpr5,dkk3,Nrf2} {Nkd2,Tgfbr2,Tgfbr3,Cyba,Sox10,Plp1,Lpr5} {Nkd2,Tgfbr2,Tgfbr3,Cyba,Sox10,Plp1,dkk3} {Nkd2,Tgfbr2,Tgfbr3,Cyba,Sox10,Plp1,Nrf2} {Nkd2,Tgfbr2,Tgfbr3,Cyba,Sox10,Lpr5,dkk3} {Nkd2,Tgfbr2,Tgfbr3,Cyba,Sox10,Lpr5,Nrf2} {Nkd2,Tgfbr2,Tgfbr3,Cyba,Sox10,dkk3,Nrf2} {Nkd2,Tgfbr2,Tgfbr3,Cyba,Plp1,Lpr5,dkk3} {Nkd2,Tgfbr2,Tgfbr3,Cyba,Plp1,Lpr5,Nrf2} {Nkd2,Tgfbr2,Tgfbr3,Cyba,Plp1,dkk3,Nrf2} {Nkd2,Tgfbr2,Tgfbr3,Cyba,Lpr5,dkk3,Nrf2} {Nkd2,Tgfbr2,Tgfbr3,Sox10,Plp1,Lpr5,dkk3} {Nkd2,Tgfbr2,Tgfbr3,Sox10,Plp1,Lpr5,Nrf2} {Nkd2,Tgfbr2,Tgfbr3,Sox10,Plp1,dkk3,Nrf2} {Nkd2,Tgfbr2,Tgfbr3,Sox10,Lpr5,dkk3,Nrf2} {Nkd2,Tgfbr2,Tgfbr3,Plp1,Lpr5,dkk3,Nrf2} {Nkd2,Tgfbr2,Hmox1,Cyba,Sox10,Plp1,Lpr5} {Nkd2,Tgfbr2,Hmox1,Cyba,Sox10,Plp1,dkk3} {Nkd2,Tgfbr2,Hmox1,Cyba,Sox10,Plp1,Nrf2} {Nkd2,Tgfbr2,Hmox1,Cyba,Sox10,Lpr5,dkk3} {Nkd2,Tgfbr2,Hmox1,Cyba,Sox10,Lpr5,Nrf2} {Nkd2,Tgfbr2,Hmox1,Cyba,Sox10,dkk3,Nrf2} {Nkd2,Tgfbr2,Hmox1,Cyba,Plp1,Lpr5,dkk3} {Nkd2,Tgfbr2,Hmox1,Cyba,Plp1,Lpr5,Nrf2} {Nkd2,Tgfbr2,Hmox1,Cyba,Plp1,dkk3,Nrf2} {Nkd2,Tgfbr2,Hmox1,Cyba,Lpr5,dkk3,Nrf2} {Nkd2,Tgfbr2,Hmox1,Sox10,Plp1,Lpr5,dkk3} {Nkd2,Tgfbr2,Hmox1,Sox10,Plp1,Lpr5,Nrf2} {Nkd2,Tgfbr2,Hmox1,Sox10,Plp1,dkk3,Nrf2} {Nkd2,Tgfbr2,Hmox1,Sox10,Lpr5,dkk3,Nrf2} {Nkd2,Tgfbr2,Hmox1,Plp1,Lpr5,dkk3,Nrf2} {Nkd2,Tgfbr2,Cyba,Sox10,Plp1,Lpr5,dkk3} {Nkd2,Tgfbr2,Cyba,Sox10,Plp1,Lpr5,Nrf2} {Nkd2,Tgfbr2,Cyba,Sox10,Plp1,dkk3,Nrf2} {Nkd2,Tgfbr2,Cyba,Sox10,Lpr5,dkk3,Nrf2} {Nkd2,Tgfbr2,Cyba,Plp1,Lpr5,dkk3,Nrf2} {Nkd2,Tgfbr2,Sox10,Plp1,Lpr5,dkk3,Nrf2} {Nkd2,Tgfbr3,Hmox1,Cyba,Sox10,Plp1,Lpr5} {Nkd2,Tgfbr3,Hmox1,Cyba,Sox10,Plp1,dkk3} {Nkd2,Tgfbr3,Hmox1,Cyba,Sox10,Plp1,Nrf2} {Nkd2,Tgfbr3,Hmox1,Cyba,Sox10,Lpr5,dkk3} {Nkd2,Tgfbr3,Hmox1,Cyba,Sox10,Lpr5,Nrf2} {Nkd2,Tgfbr3,Hmox1,Cyba,Sox10,dkk3,Nrf2} {Nkd2,Tgfbr3,Hmox1,Cyba,Plp1,Lpr5,dkk3} {Nkd2,Tgfbr3,Hmox1,Cyba,Plp1,Lpr5,Nrf2} {Nkd2,Tgfbr3,Hmox1,Cyba,Plp1,dkk3,Nrf2} {Nkd2,Tgfbr3,Hmox1,Cyba,Lpr5,dkk3,Nrf2} {Nkd2,Tgfbr3,Hmox1,Sox10,Plp1,Lpr5,dkk3} {Nkd2,Tgfbr3,Hmox1,Sox10,Plp1,Lpr5,Nrf2} {Nkd2,Tgfbr3,Hmox1,Sox10,Plp1,dkk3,Nrf2} {Nkd2,Tgfbr3,Hmox1,Sox10,Lpr5,dkk3,Nrf2} {Nkd2,Tgfbr3,Hmox1,Plp1,Lpr5,dkk3,Nrf2} {Nkd2,Tgfbr3,Cyba,Sox10,Plp1,Lpr5,dkk3} {Nkd2,Tgfbr3,Cyba,Sox10,Plp1,Lpr5,Nrf2} {Nkd2,Tgfbr3,Cyba,Sox10,Plp1,dkk3,Nrf2} {Nkd2,Tgfbr3,Cyba,Sox10,Lpr5,dkk3,Nrf2} {Nkd2,Tgfbr3,Cyba,Plp1,Lpr5,dkk3,Nrf2} {Nkd2,Tgfbr3,Sox10,Plp1,Lpr5,dkk3,Nrf2} {Nkd2,Hmox1,Cyba,Sox10,Plp1,Lpr5,dkk3} {Nkd2,Hmox1,Cyba,Sox10,Plp1,Lpr5,Nrf2} {Nkd2,Hmox1,Cyba,Sox10,Plp1,dkk3,Nrf2} {Nkd2,Hmox1,Cyba,Sox10,Lpr5,dkk3,Nrf2} {Nkd2,Hmox1,Cyba,Plp1,Lpr5,dkk3,Nrf2} {Nkd2,Hmox1,Sox10,Plp1,Lpr5,dkk3,Nrf2} {Nkd2,Cyba,Sox10,Plp1,Lpr5,dkk3,Nrf2} {Tgfbr2,Tgfbr3,Hmox1,Cyba,Sox10,Plp1,Lpr5} {Tgfbr2,Tgfbr3,Hmox1,Cyba,Sox10,Plp1,dkk3} {Tgfbr2,Tgfbr3,Hmox1,Cyba,Sox10,Plp1,Nrf2} {Tgfbr2,Tgfbr3,Hmox1,Cyba,Sox10,Lpr5,dkk3} {Tgfbr2,Tgfbr3,Hmox1,Cyba,Sox10,Lpr5,Nrf2} {Tgfbr2,Tgfbr3,Hmox1,Cyba,Sox10,dkk3,Nrf2} {Tgfbr2,Tgfbr3,Hmox1,Cyba,Plp1,Lpr5,dkk3} {Tgfbr2,Tgfbr3,Hmox1,Cyba,Plp1,Lpr5,Nrf2} {Tgfbr2,Tgfbr3,Hmox1,Cyba,Plp1,dkk3,Nrf2} {Tgfbr2,Tgfbr3,Hmox1,Cyba,Lpr5,dkk3,Nrf2} {Tgfbr2,Tgfbr3,Hmox1,Sox10,Plp1,Lpr5,dkk3} {Tgfbr2,Tgfbr3,Hmox1,Sox10,Plp1,Lpr5,Nrf2} {Tgfbr2,Tgfbr3,Hmox1,Sox10,Plp1,dkk3,Nrf2} {Tgfbr2,Tgfbr3,Hmox1,Sox10,Lpr5,dkk3,Nrf2} {Tgfbr2,Tgfbr3,Hmox1,Plp1,Lpr5,dkk3,Nrf2} {Tgfbr2,Tgfbr3,Cyba,Sox10,Plp1,Lpr5,dkk3} {Tgfbr2,Tgfbr3,Cyba,Sox10,Plp1,Lpr5,Nrf2} {Tgfbr2,Tgfbr3,Cyba,Sox10,Plp1,dkk3,Nrf2} {Tgfbr2,Tgfbr3,Cyba,Sox10,Lpr5,dkk3,Nrf2} {Tgfbr2,Tgfbr3,Cyba,Plp1,Lpr5,dkk3,Nrf2} {Tgfbr2,Tgfbr3,Sox10,Plp1,Lpr5,dkk3,Nrf2} {Tgfbr2,Hmox1,Cyba,Sox10,Plp1,Lpr5,dkk3} {Tgfbr2,Hmox1,Cyba,Sox10,Plp1,Lpr5,Nrf2} {Tgfbr2,Hmox1,Cyba,Sox10,Plp1,dkk3,Nrf2} {Tgfbr2,Hmox1,Cyba,Sox10,Lpr5,dkk3,Nrf2} {Tgfbr2,Hmox1,Cyba,Plp1,Lpr5,dkk3,Nrf2} {Tgfbr2,Hmox1,Sox10,Plp1,Lpr5,dkk3,Nrf2} {Tgfbr2,Cyba,Sox10,Plp1,Lpr5,dkk3,Nrf2} {Tgfbr3,Hmox1,Cyba,Sox10,Plp1,Lpr5,dkk3} {Tgfbr3,Hmox1,Cyba,Sox10,Plp1,Lpr5,Nrf2} {Tgfbr3,Hmox1,Cyba,Sox10,Plp1,dkk3,Nrf2} {Tgfbr3,Hmox1,Cyba,Sox10,Lpr5,dkk3,Nrf2} {Tgfbr3,Hmox1,Cyba,Plp1,Lpr5,dkk3,Nrf2} {Tgfbr3,Hmox1,Sox10,Plp1,Lpr5,dkk3,Nrf2} {Tgfbr3,Cyba,Sox10,Plp1,Lpr5,dkk3,Nrf2} {Hmox1,Cyba,Sox10,Plp1,Lpr5,dkk3,Nrf2} {Lgr6,Frb,Nkd2,Tgfbr2,Tgfbr3,Hmox1,Cyba,Sox10} {Lgr6,Frb,Nkd2,Tgfbr2,Tgfbr3,Hmox1,Cyba,Plp1} {Lgr6,Frb,Nkd2,Tgfbr2,Tgfbr3,Hmox1,Cyba,Lpr5} {Lgr6,Frb,Nkd2,Tgfbr2,Tgfbr3,Hmox1,Cyba,dkk3} {Lgr6,Frb,Nkd2,Tgfbr2,Tgfbr3,Hmox1,Cyba,Nrf2} {Lgr6,Frb,Nkd2,Tgfbr2,Tgfbr3,Hmox1,Sox10,Plp1} {Lgr6,Frb,Nkd2,Tgfbr2,Tgfbr3,Hmox1,Sox10,Lpr5} {Lgr6,Frb,Nkd2,Tgfbr2,Tgfbr3,Hmox1,Sox10,dkk3} {Lgr6,Frb,Nkd2,Tgfbr2,Tgfbr3,Hmox1,Sox10,Nrf2} {Lgr6,Frb,Nkd2,Tgfbr2,Tgfbr3,Hmox1,Plp1,Lpr5} {Lgr6,Frb,Nkd2,Tgfbr2,Tgfbr3,Hmox1,Plp1,dkk3} {Lgr6,Frb,Nkd2,Tgfbr2,Tgfbr3,Hmox1,Plp1,Nrf2} {Lgr6,Frb,Nkd2,Tgfbr2,Tgfbr3,Hmox1,Lpr5,dkk3} {Lgr6,Frb,Nkd2,Tgfbr2,Tgfbr3,Hmox1,Lpr5,Nrf2} {Lgr6,Frb,Nkd2,Tgfbr2,Tgfbr3,Hmox1,dkk3,Nrf2} {Lgr6,Frb,Nkd2,Tgfbr2,Tgfbr3,Cyba,Sox10,Plp1} {Lgr6,Frb,Nkd2,Tgfbr2,Tgfbr3,Cyba,Sox10,Lpr5} {Lgr6,Frb,Nkd2,Tgfbr2,Tgfbr3,Cyba,Sox10,dkk3} {Lgr6,Frb,Nkd2,Tgfbr2,Tgfbr3,Cyba,Sox10,Nrf2} {Lgr6,Frb,Nkd2,Tgfbr2,Tgfbr3,Cyba,Plp1,Lpr5} {Lgr6,Frb,Nkd2,Tgfbr2,Tgfbr3,Cyba,Plp1,dkk3} {Lgr6,Frb,Nkd2,Tgfbr2,Tgfbr3,Cyba,Plp1,Nrf2} {Lgr6,Frb,Nkd2,Tgfbr2,Tgfbr3,Cyba,Lpr5,dkk3} {Lgr6,Frb,Nkd2,Tgfbr2,Tgfbr3,Cyba,Lpr5,Nrf2} {Lgr6,Frb,Nkd2,Tgfbr2,Tgfbr3,Cyba,dkk3,Nrf2} {Lgr6,Frb,Nkd2,Tgfbr2,Tgfbr3,Sox10,Plp1,Lpr5} {Lgr6,Frb,Nkd2,Tgfbr2,Tgfbr3,Sox10,Plp1,dkk3} {Lgr6,Frb,Nkd2,Tgfbr2,Tgfbr3,Sox10,Plp1,Nrf2} {Lgr6,Frb,Nkd2,Tgfbr2,Tgfbr3,Sox10,Lpr5,dkk3} {Lgr6,Frb,Nkd2,Tgfbr2,Tgfbr3,Sox10,Lpr5,Nrf2} {Lgr6,Frb,Nkd2,Tgfbr2,Tgfbr3,Sox10,dkk3,Nrf2} {Lgr6,Frb,Nkd2,Tgfbr2,Tgfbr3,Plp1,Lpr5,dkk3} {Lgr6,Frb,Nkd2,Tgfbr2,Tgfbr3,Plp1,Lpr5,Nrf2} {Lgr6,Frb,Nkd2,Tgfbr2,Tgfbr3,Plp1,dkk3,Nrf2} {Lgr6,Frb,Nkd2,Tgfbr2,Tgfbr3,Lpr5,dkk3,Nrf2} {Lgr6,Frb,Nkd2,Tgfbr2,Hmox1,Cyba,Sox10,Plp1} {Lgr6,Frb,Nkd2,Tgfbr2,Hmox1,Cyba,Sox10,Lpr5} {Lgr6,Frb,Nkd2,Tgfbr2,Hmox1,Cyba,Sox10,dkk3} {Lgr6,Frb,Nkd2,Tgfbr2,Hmox1,Cyba,Sox10,Nrf2} {Lgr6,Frb,Nkd2,Tgfbr2,Hmox1,Cyba,Plp1,Lpr5} {Lgr6,Frb,Nkd2,Tgfbr2,Hmox1,Cyba,Plp1,dkk3} {Lgr6,Frb,Nkd2,Tgfbr2,Hmox1,Cyba,Plp1,Nrf2} {Lgr6,Frb,Nkd2,Tgfbr2,Hmox1,Cyba,Lpr5,dkk3} {Lgr6,Frb,Nkd2,Tgfbr2,Hmox1,Cyba,Lpr5,Nrf2} {Lgr6,Frb,Nkd2,Tgfbr2,Hmox1,Cyba,dkk3,Nrf2} {Lgr6,Frb,Nkd2,Tgfbr2,Hmox1,Sox10,Plp1,Lpr5} {Lgr6,Frb,Nkd2,Tgfbr2,Hmox1,Sox10,Plp1,dkk3} {Lgr6,Frb,Nkd2,Tgfbr2,Hmox1,Sox10,Plp1,Nrf2} {Lgr6,Frb,Nkd2,Tgfbr2,Hmox1,Sox10,Lpr5,dkk3} {Lgr6,Frb,Nkd2,Tgfbr2,Hmox1,Sox10,Lpr5,Nrf2} {Lgr6,Frb,Nkd2,Tgfbr2,Hmox1,Sox10,dkk3,Nrf2} {Lgr6,Frb,Nkd2,Tgfbr2,Hmox1,Plp1,Lpr5,dkk3} {Lgr6,Frb,Nkd2,Tgfbr2,Hmox1,Plp1,Lpr5,Nrf2} {Lgr6,Frb,Nkd2,Tgfbr2,Hmox1,Plp1,dkk3,Nrf2} {Lgr6,Frb,Nkd2,Tgfbr2,Hmox1,Lpr5,dkk3,Nrf2} {Lgr6,Frb,Nkd2,Tgfbr2,Cyba,Sox10,Plp1,Lpr5} {Lgr6,Frb,Nkd2,Tgfbr2,Cyba,Sox10,Plp1,dkk3} {Lgr6,Frb,Nkd2,Tgfbr2,Cyba,Sox10,Plp1,Nrf2} {Lgr6,Frb,Nkd2,Tgfbr2,Cyba,Sox10,Lpr5,dkk3} {Lgr6,Frb,Nkd2,Tgfbr2,Cyba,Sox10,Lpr5,Nrf2} {Lgr6,Frb,Nkd2,Tgfbr2,Cyba,Sox10,dkk3,Nrf2} {Lgr6,Frb,Nkd2,Tgfbr2,Cyba,Plp1,Lpr5,dkk3} {Lgr6,Frb,Nkd2,Tgfbr2,Cyba,Plp1,Lpr5,Nrf2} {Lgr6,Frb,Nkd2,Tgfbr2,Cyba,Plp1,dkk3,Nrf2} {Lgr6,Frb,Nkd2,Tgfbr2,Cyba,Lpr5,dkk3,Nrf2} {Lgr6,Frb,Nkd2,Tgfbr2,Sox10,Plp1,Lpr5,dkk3} {Lgr6,Frb,Nkd2,Tgfbr2,Sox10,Plp1,Lpr5,Nrf2} {Lgr6,Frb,Nkd2,Tgfbr2,Sox10,Plp1,dkk3,Nrf2} {Lgr6,Frb,Nkd2,Tgfbr2,Sox10,Lpr5,dkk3,Nrf2} {Lgr6,Frb,Nkd2,Tgfbr2,Plp1,Lpr5,dkk3,Nrf2} {Lgr6,Frb,Nkd2,Tgfbr3,Hmox1,Cyba,Sox10,Plp1} {Lgr6,Frb,Nkd2,Tgfbr3,Hmox1,Cyba,Sox10,Lpr5} {Lgr6,Frb,Nkd2,Tgfbr3,Hmox1,Cyba,Sox10,dkk3} {Lgr6,Frb,Nkd2,Tgfbr3,Hmox1,Cyba,Sox10,Nrf2} {Lgr6,Frb,Nkd2,Tgfbr3,Hmox1,Cyba,Plp1,Lpr5} {Lgr6,Frb,Nkd2,Tgfbr3,Hmox1,Cyba,Plp1,dkk3} {Lgr6,Frb,Nkd2,Tgfbr3,Hmox1,Cyba,Plp1,Nrf2} {Lgr6,Frb,Nkd2,Tgfbr3,Hmox1,Cyba,Lpr5,dkk3} {Lgr6,Frb,Nkd2,Tgfbr3,Hmox1,Cyba,Lpr5,Nrf2} {Lgr6,Frb,Nkd2,Tgfbr3,Hmox1,Cyba,dkk3,Nrf2} {Lgr6,Frb,Nkd2,Tgfbr3,Hmox1,Sox10,Plp1,Lpr5} {Lgr6,Frb,Nkd2,Tgfbr3,Hmox1,Sox10,Plp1,dkk3} {Lgr6,Frb,Nkd2,Tgfbr3,Hmox1,Sox10,Plp1,Nrf2} {Lgr6,Frb,Nkd2,Tgfbr3,Hmox1,Sox10,Lpr5,dkk3} {Lgr6,Frb,Nkd2,Tgfbr3,Hmox1,Sox10,Lpr5,Nrf2} {Lgr6,Frb,Nkd2,Tgfbr3,Hmox1,Sox10,dkk3,Nrf2} {Lgr6,Frb,Nkd2,Tgfbr3,Hmox1,Plp1,Lpr5,dkk3} {Lgr6,Frb,Nkd2,Tgfbr3,Hmox1,Plp1,Lpr5,Nrf2} {Lgr6,Frb,Nkd2,Tgfbr3,Hmox1,Plp1,dkk3,Nrf2} {Lgr6,Frb,Nkd2,Tgfbr3,Hmox1,Lpr5,dkk3,Nrf2} {Lgr6,Frb,Nkd2,Tgfbr3,Cyba,Sox10,Plp1,Lpr5} {Lgr6,Frb,Nkd2,Tgfbr3,Cyba,Sox10,Plp1,dkk3} {Lgr6,Frb,Nkd2,Tgfbr3,Cyba,Sox10,Plp1,Nrf2} {Lgr6,Frb,Nkd2,Tgfbr3,Cyba,Sox10,Lpr5,dkk3} {Lgr6,Frb,Nkd2,Tgfbr3,Cyba,Sox10,Lpr5,Nrf2} {Lgr6,Frb,Nkd2,Tgfbr3,Cyba,Sox10,dkk3,Nrf2} {Lgr6,Frb,Nkd2,Tgfbr3,Cyba,Plp1,Lpr5,dkk3} {Lgr6,Frb,Nkd2,Tgfbr3,Cyba,Plp1,Lpr5,Nrf2} {Lgr6,Frb,Nkd2,Tgfbr3,Cyba,Plp1,dkk3,Nrf2} {Lgr6,Frb,Nkd2,Tgfbr3,Cyba,Lpr5,dkk3,Nrf2} {Lgr6,Frb,Nkd2,Tgfbr3,Sox10,Plp1,Lpr5,dkk3} {Lgr6,Frb,Nkd2,Tgfbr3,Sox10,Plp1,Lpr5,Nrf2} {Lgr6,Frb,Nkd2,Tgfbr3,Sox10,Plp1,dkk3,Nrf2} {Lgr6,Frb,Nkd2,Tgfbr3,Sox10,Lpr5,dkk3,Nrf2} {Lgr6,Frb,Nkd2,Tgfbr3,Plp1,Lpr5,dkk3,Nrf2} {Lgr6,Frb,Nkd2,Hmox1,Cyba,Sox10,Plp1,Lpr5} {Lgr6,Frb,Nkd2,Hmox1,Cyba,Sox10,Plp1,dkk3} {Lgr6,Frb,Nkd2,Hmox1,Cyba,Sox10,Plp1,Nrf2} {Lgr6,Frb,Nkd2,Hmox1,Cyba,Sox10,Lpr5,dkk3} {Lgr6,Frb,Nkd2,Hmox1,Cyba,Sox10,Lpr5,Nrf2} {Lgr6,Frb,Nkd2,Hmox1,Cyba,Sox10,dkk3,Nrf2} {Lgr6,Frb,Nkd2,Hmox1,Cyba,Plp1,Lpr5,dkk3} {Lgr6,Frb,Nkd2,Hmox1,Cyba,Plp1,Lpr5,Nrf2} {Lgr6,Frb,Nkd2,Hmox1,Cyba,Plp1,dkk3,Nrf2} {Lgr6,Frb,Nkd2,Hmox1,Cyba,Lpr5,dkk3,Nrf2} {Lgr6,Frb,Nkd2,Hmox1,Sox10,Plp1,Lpr5,dkk3} {Lgr6,Frb,Nkd2,Hmox1,Sox10,Plp1,Lpr5,Nrf2} {Lgr6,Frb,Nkd2,Hmox1,Sox10,Plp1,dkk3,Nrf2} {Lgr6,Frb,Nkd2,Hmox1,Sox10,Lpr5,dkk3,Nrf2} {Lgr6,Frb,Nkd2,Hmox1,Plp1,Lpr5,dkk3,Nrf2} {Lgr6,Frb,Nkd2,Cyba,Sox10,Plp1,Lpr5,dkk3} {Lgr6,Frb,Nkd2,Cyba,Sox10,Plp1,Lpr5,Nrf2} {Lgr6,Frb,Nkd2,Cyba,Sox10,Plp1,dkk3,Nrf2} {Lgr6,Frb,Nkd2,Cyba,Sox10,Lpr5,dkk3,Nrf2} {Lgr6,Frb,Nkd2,Cyba,Plp1,Lpr5,dkk3,Nrf2} {Lgr6,Frb,Nkd2,Sox10,Plp1,Lpr5,dkk3,Nrf2} {Lgr6,Frb,Tgfbr2,Tgfbr3,Hmox1,Cyba,Sox10,Plp1} {Lgr6,Frb,Tgfbr2,Tgfbr3,Hmox1,Cyba,Sox10,Lpr5} {Lgr6,Frb,Tgfbr2,Tgfbr3,Hmox1,Cyba,Sox10,dkk3} {Lgr6,Frb,Tgfbr2,Tgfbr3,Hmox1,Cyba,Sox10,Nrf2} {Lgr6,Frb,Tgfbr2,Tgfbr3,Hmox1,Cyba,Plp1,Lpr5} {Lgr6,Frb,Tgfbr2,Tgfbr3,Hmox1,Cyba,Plp1,dkk3} {Lgr6,Frb,Tgfbr2,Tgfbr3,Hmox1,Cyba,Plp1,Nrf2} {Lgr6,Frb,Tgfbr2,Tgfbr3,Hmox1,Cyba,Lpr5,dkk3} {Lgr6,Frb,Tgfbr2,Tgfbr3,Hmox1,Cyba,Lpr5,Nrf2} {Lgr6,Frb,Tgfbr2,Tgfbr3,Hmox1,Cyba,dkk3,Nrf2} {Lgr6,Frb,Tgfbr2,Tgfbr3,Hmox1,Sox10,Plp1,Lpr5} {Lgr6,Frb,Tgfbr2,Tgfbr3,Hmox1,Sox10,Plp1,dkk3} {Lgr6,Frb,Tgfbr2,Tgfbr3,Hmox1,Sox10,Plp1,Nrf2} {Lgr6,Frb,Tgfbr2,Tgfbr3,Hmox1,Sox10,Lpr5,dkk3} {Lgr6,Frb,Tgfbr2,Tgfbr3,Hmox1,Sox10,Lpr5,Nrf2} {Lgr6,Frb,Tgfbr2,Tgfbr3,Hmox1,Sox10,dkk3,Nrf2} {Lgr6,Frb,Tgfbr2,Tgfbr3,Hmox1,Plp1,Lpr5,dkk3} {Lgr6,Frb,Tgfbr2,Tgfbr3,Hmox1,Plp1,Lpr5,Nrf2} {Lgr6,Frb,Tgfbr2,Tgfbr3,Hmox1,Plp1,dkk3,Nrf2} {Lgr6,Frb,Tgfbr2,Tgfbr3,Hmox1,Lpr5,dkk3,Nrf2} {Lgr6,Frb,Tgfbr2,Tgfbr3,Cyba,Sox10,Plp1,Lpr5} {Lgr6,Frb,Tgfbr2,Tgfbr3,Cyba,Sox10,Plp1,dkk3} {Lgr6,Frb,Tgfbr2,Tgfbr3,Cyba,Sox10,Plp1,Nrf2} {Lgr6,Frb,Tgfbr2,Tgfbr3,Cyba,Sox10,Lpr5,dkk3} {Lgr6,Frb,Tgfbr2,Tgfbr3,Cyba,Sox10,Lpr5,Nrf2} {Lgr6,Frb,Tgfbr2,Tgfbr3,Cyba,Sox10,dkk3,Nrf2} {Lgr6,Frb,Tgfbr2,Tgfbr3,Cyba,Plp1,Lpr5,dkk3} {Lgr6,Frb,Tgfbr2,Tgfbr3,Cyba,Plp1,Lpr5,Nrf2} {Lgr6,Frb,Tgfbr2,Tgfbr3,Cyba,Plp1,dkk3,Nrf2} {Lgr6,Frb,Tgfbr2,Tgfbr3,Cyba,Lpr5,dkk3,Nrf2} {Lgr6,Frb,Tgfbr2,Tgfbr3,Sox10,Plp1,Lpr5,dkk3} {Lgr6,Frb,Tgfbr2,Tgfbr3,Sox10,Plp1,Lpr5,Nrf2} {Lgr6,Frb,Tgfbr2,Tgfbr3,Sox10,Plp1,dkk3,Nrf2} {Lgr6,Frb,Tgfbr2,Tgfbr3,Sox10,Lpr5,dkk3,Nrf2} {Lgr6,Frb,Tgfbr2,Tgfbr3,Plp1,Lpr5,dkk3,Nrf2} {Lgr6,Frb,Tgfbr2,Hmox1,Cyba,Sox10,Plp1,Lpr5} {Lgr6,Frb,Tgfbr2,Hmox1,Cyba,Sox10,Plp1,dkk3} {Lgr6,Frb,Tgfbr2,Hmox1,Cyba,Sox10,Plp1,Nrf2} {Lgr6,Frb,Tgfbr2,Hmox1,Cyba,Sox10,Lpr5,dkk3} {Lgr6,Frb,Tgfbr2,Hmox1,Cyba,Sox10,Lpr5,Nrf2} {Lgr6,Frb,Tgfbr2,Hmox1,Cyba,Sox10,dkk3,Nrf2} {Lgr6,Frb,Tgfbr2,Hmox1,Cyba,Plp1,Lpr5,dkk3} {Lgr6,Frb,Tgfbr2,Hmox1,Cyba,Plp1,Lpr5,Nrf2} {Lgr6,Frb,Tgfbr2,Hmox1,Cyba,Plp1,dkk3,Nrf2} {Lgr6,Frb,Tgfbr2,Hmox1,Cyba,Lpr5,dkk3,Nrf2} {Lgr6,Frb,Tgfbr2,Hmox1,Sox10,Plp1,Lpr5,dkk3} {Lgr6,Frb,Tgfbr2,Hmox1,Sox10,Plp1,Lpr5,Nrf2} {Lgr6,Frb,Tgfbr2,Hmox1,Sox10,Plp1,dkk3,Nrf2} {Lgr6,Frb,Tgfbr2,Hmox1,Sox10,Lpr5,dkk3,Nrf2} {Lgr6,Frb,Tgfbr2,Hmox1,Plp1,Lpr5,dkk3,Nrf2} {Lgr6,Frb,Tgfbr2,Cyba,Sox10,Plp1,Lpr5,dkk3} {Lgr6,Frb,Tgfbr2,Cyba,Sox10,Plp1,Lpr5,Nrf2} {Lgr6,Frb,Tgfbr2,Cyba,Sox10,Plp1,dkk3,Nrf2} {Lgr6,Frb,Tgfbr2,Cyba,Sox10,Lpr5,dkk3,Nrf2} {Lgr6,Frb,Tgfbr2,Cyba,Plp1,Lpr5,dkk3,Nrf2} {Lgr6,Frb,Tgfbr2,Sox10,Plp1,Lpr5,dkk3,Nrf2} {Lgr6,Frb,Tgfbr3,Hmox1,Cyba,Sox10,Plp1,Lpr5} {Lgr6,Frb,Tgfbr3,Hmox1,Cyba,Sox10,Plp1,dkk3} {Lgr6,Frb,Tgfbr3,Hmox1,Cyba,Sox10,Plp1,Nrf2} {Lgr6,Frb,Tgfbr3,Hmox1,Cyba,Sox10,Lpr5,dkk3} {Lgr6,Frb,Tgfbr3,Hmox1,Cyba,Sox10,Lpr5,Nrf2} {Lgr6,Frb,Tgfbr3,Hmox1,Cyba,Sox10,dkk3,Nrf2} {Lgr6,Frb,Tgfbr3,Hmox1,Cyba,Plp1,Lpr5,dkk3} {Lgr6,Frb,Tgfbr3,Hmox1,Cyba,Plp1,Lpr5,Nrf2} {Lgr6,Frb,Tgfbr3,Hmox1,Cyba,Plp1,dkk3,Nrf2} {Lgr6,Frb,Tgfbr3,Hmox1,Cyba,Lpr5,dkk3,Nrf2} {Lgr6,Frb,Tgfbr3,Hmox1,Sox10,Plp1,Lpr5,dkk3} {Lgr6,Frb,Tgfbr3,Hmox1,Sox10,Plp1,Lpr5,Nrf2} {Lgr6,Frb,Tgfbr3,Hmox1,Sox10,Plp1,dkk3,Nrf2} {Lgr6,Frb,Tgfbr3,Hmox1,Sox10,Lpr5,dkk3,Nrf2} {Lgr6,Frb,Tgfbr3,Hmox1,Plp1,Lpr5,dkk3,Nrf2} {Lgr6,Frb,Tgfbr3,Cyba,Sox10,Plp1,Lpr5,dkk3} {Lgr6,Frb,Tgfbr3,Cyba,Sox10,Plp1,Lpr5,Nrf2} {Lgr6,Frb,Tgfbr3,Cyba,Sox10,Plp1,dkk3,Nrf2} {Lgr6,Frb,Tgfbr3,Cyba,Sox10,Lpr5,dkk3,Nrf2} {Lgr6,Frb,Tgfbr3,Cyba,Plp1,Lpr5,dkk3,Nrf2} {Lgr6,Frb,Tgfbr3,Sox10,Plp1,Lpr5,dkk3,Nrf2} {Lgr6,Frb,Hmox1,Cyba,Sox10,Plp1,Lpr5,dkk3} {Lgr6,Frb,Hmox1,Cyba,Sox10,Plp1,Lpr5,Nrf2} {Lgr6,Frb,Hmox1,Cyba,Sox10,Plp1,dkk3,Nrf2} {Lgr6,Frb,Hmox1,Cyba,Sox10,Lpr5,dkk3,Nrf2} {Lgr6,Frb,Hmox1,Cyba,Plp1,Lpr5,dkk3,Nrf2} {Lgr6,Frb,Hmox1,Sox10,Plp1,Lpr5,dkk3,Nrf2} {Lgr6,Frb,Cyba,Sox10,Plp1,Lpr5,dkk3,Nrf2} {Lgr6,Nkd2,Tgfbr2,Tgfbr3,Hmox1,Cyba,Sox10,Plp1} {Lgr6,Nkd2,Tgfbr2,Tgfbr3,Hmox1,Cyba,Sox10,Lpr5} {Lgr6,Nkd2,Tgfbr2,Tgfbr3,Hmox1,Cyba,Sox10,dkk3} {Lgr6,Nkd2,Tgfbr2,Tgfbr3,Hmox1,Cyba,Sox10,Nrf2} {Lgr6,Nkd2,Tgfbr2,Tgfbr3,Hmox1,Cyba,Plp1,Lpr5} {Lgr6,Nkd2,Tgfbr2,Tgfbr3,Hmox1,Cyba,Plp1,dkk3} {Lgr6,Nkd2,Tgfbr2,Tgfbr3,Hmox1,Cyba,Plp1,Nrf2} {Lgr6,Nkd2,Tgfbr2,Tgfbr3,Hmox1,Cyba,Lpr5,dkk3} {Lgr6,Nkd2,Tgfbr2,Tgfbr3,Hmox1,Cyba,Lpr5,Nrf2} {Lgr6,Nkd2,Tgfbr2,Tgfbr3,Hmox1,Cyba,dkk3,Nrf2} {Lgr6,Nkd2,Tgfbr2,Tgfbr3,Hmox1,Sox10,Plp1,Lpr5} {Lgr6,Nkd2,Tgfbr2,Tgfbr3,Hmox1,Sox10,Plp1,dkk3} {Lgr6,Nkd2,Tgfbr2,Tgfbr3,Hmox1,Sox10,Plp1,Nrf2} {Lgr6,Nkd2,Tgfbr2,Tgfbr3,Hmox1,Sox10,Lpr5,dkk3} {Lgr6,Nkd2,Tgfbr2,Tgfbr3,Hmox1,Sox10,Lpr5,Nrf2} {Lgr6,Nkd2,Tgfbr2,Tgfbr3,Hmox1,Sox10,dkk3,Nrf2} {Lgr6,Nkd2,Tgfbr2,Tgfbr3,Hmox1,Plp1,Lpr5,dkk3} {Lgr6,Nkd2,Tgfbr2,Tgfbr3,Hmox1,Plp1,Lpr5,Nrf2} {Lgr6,Nkd2,Tgfbr2,Tgfbr3,Hmox1,Plp1,dkk3,Nrf2} {Lgr6,Nkd2,Tgfbr2,Tgfbr3,Hmox1,Lpr5,dkk3,Nrf2} {Lgr6,Nkd2,Tgfbr2,Tgfbr3,Cyba,Sox10,Plp1,Lpr5} {Lgr6,Nkd2,Tgfbr2,Tgfbr3,Cyba,Sox10,Plp1,dkk3} {Lgr6,Nkd2,Tgfbr2,Tgfbr3,Cyba,Sox10,Plp1,Nrf2} {Lgr6,Nkd2,Tgfbr2,Tgfbr3,Cyba,Sox10,Lpr5,dkk3} {Lgr6,Nkd2,Tgfbr2,Tgfbr3,Cyba,Sox10,Lpr5,Nrf2} {Lgr6,Nkd2,Tgfbr2,Tgfbr3,Cyba,Sox10,dkk3,Nrf2} {Lgr6,Nkd2,Tgfbr2,Tgfbr3,Cyba,Plp1,Lpr5,dkk3} {Lgr6,Nkd2,Tgfbr2,Tgfbr3,Cyba,Plp1,Lpr5,Nrf2} {Lgr6,Nkd2,Tgfbr2,Tgfbr3,Cyba,Plp1,dkk3,Nrf2} {Lgr6,Nkd2,Tgfbr2,Tgfbr3,Cyba,Lpr5,dkk3,Nrf2} {Lgr6,Nkd2,Tgfbr2,Tgfbr3,Sox10,Plp1,Lpr5,dkk3} {Lgr6,Nkd2,Tgfbr2,Tgfbr3,Sox10,Plp1,Lpr5,Nrf2} {Lgr6,Nkd2,Tgfbr2,Tgfbr3,Sox10,Plp1,dkk3,Nrf2} {Lgr6,Nkd2,Tgfbr2,Tgfbr3,Sox10,Lpr5,dkk3,Nrf2} {Lgr6,Nkd2,Tgfbr2,Tgfbr3,Plp1,Lpr5,dkk3,Nrf2} {Lgr6,Nkd2,Tgfbr2,Hmox1,Cyba,Sox10,Plp1,Lpr5} {Lgr6,Nkd2,Tgfbr2,Hmox1,Cyba,Sox10,Plp1,dkk3} {Lgr6,Nkd2,Tgfbr2,Hmox1,Cyba,Sox10,Plp1,Nrf2} {Lgr6,Nkd2,Tgfbr2,Hmox1,Cyba,Sox10,Lpr5,dkk3} {Lgr6,Nkd2,Tgfbr2,Hmox1,Cyba,Sox10,Lpr5,Nrf2} {Lgr6,Nkd2,Tgfbr2,Hmox1,Cyba,Sox10,dkk3,Nrf2} {Lgr6,Nkd2,Tgfbr2,Hmox1,Cyba,Plp1,Lpr5,dkk3} {Lgr6,Nkd2,Tgfbr2,Hmox1,Cyba,Plp1,Lpr5,Nrf2} {Lgr6,Nkd2,Tgfbr2,Hmox1,Cyba,Plp1,dkk3,Nrf2} {Lgr6,Nkd2,Tgfbr2,Hmox1,Cyba,Lpr5,dkk3,Nrf2} {Lgr6,Nkd2,Tgfbr2,Hmox1,Sox10,Plp1,Lpr5,dkk3} {Lgr6,Nkd2,Tgfbr2,Hmox1,Sox10,Plp1,Lpr5,Nrf2} {Lgr6,Nkd2,Tgfbr2,Hmox1,Sox10,Plp1,dkk3,Nrf2} {Lgr6,Nkd2,Tgfbr2,Hmox1,Sox10,Lpr5,dkk3,Nrf2} {Lgr6,Nkd2,Tgfbr2,Hmox1,Plp1,Lpr5,dkk3,Nrf2} {Lgr6,Nkd2,Tgfbr2,Cyba,Sox10,Plp1,Lpr5,dkk3} {Lgr6,Nkd2,Tgfbr2,Cyba,Sox10,Plp1,Lpr5,Nrf2} {Lgr6,Nkd2,Tgfbr2,Cyba,Sox10,Plp1,dkk3,Nrf2} {Lgr6,Nkd2,Tgfbr2,Cyba,Sox10,Lpr5,dkk3,Nrf2} {Lgr6,Nkd2,Tgfbr2,Cyba,Plp1,Lpr5,dkk3,Nrf2} {Lgr6,Nkd2,Tgfbr2,Sox10,Plp1,Lpr5,dkk3,Nrf2} {Lgr6,Nkd2,Tgfbr3,Hmox1,Cyba,Sox10,Plp1,Lpr5} {Lgr6,Nkd2,Tgfbr3,Hmox1,Cyba,Sox10,Plp1,dkk3} {Lgr6,Nkd2,Tgfbr3,Hmox1,Cyba,Sox10,Plp1,Nrf2} {Lgr6,Nkd2,Tgfbr3,Hmox1,Cyba,Sox10,Lpr5,dkk3} {Lgr6,Nkd2,Tgfbr3,Hmox1,Cyba,Sox10,Lpr5,Nrf2} {Lgr6,Nkd2,Tgfbr3,Hmox1,Cyba,Sox10,dkk3,Nrf2} {Lgr6,Nkd2,Tgfbr3,Hmox1,Cyba,Plp1,Lpr5,dkk3} {Lgr6,Nkd2,Tgfbr3,Hmox1,Cyba,Plp1,Lpr5,Nrf2} {Lgr6,Nkd2,Tgfbr3,Hmox1,Cyba,Plp1,dkk3,Nrf2} {Lgr6,Nkd2,Tgfbr3,Hmox1,Cyba,Lpr5,dkk3,Nrf2} {Lgr6,Nkd2,Tgfbr3,Hmox1,Sox10,Plp1,Lpr5,dkk3} {Lgr6,Nkd2,Tgfbr3,Hmox1,Sox10,Plp1,Lpr5,Nrf2} {Lgr6,Nkd2,Tgfbr3,Hmox1,Sox10,Plp1,dkk3,Nrf2} {Lgr6,Nkd2,Tgfbr3,Hmox1,Sox10,Lpr5,dkk3,Nrf2} {Lgr6,Nkd2,Tgfbr3,Hmox1,Plp1,Lpr5,dkk3,Nrf2} {Lgr6,Nkd2,Tgfbr3,Cyba,Sox10,Plp1,Lpr5,dkk3} {Lgr6,Nkd2,Tgfbr3,Cyba,Sox10,Plp1,Lpr5,Nrf2} {Lgr6,Nkd2,Tgfbr3,Cyba,Sox10,Plp1,dkk3,Nrf2} {Lgr6,Nkd2,Tgfbr3,Cyba,Sox10,Lpr5,dkk3,Nrf2} {Lgr6,Nkd2,Tgfbr3,Cyba,Plp1,Lpr5,dkk3,Nrf2} {Lgr6,Nkd2,Tgfbr3,Sox10,Plp1,Lpr5,dkk3,Nrf2} {Lgr6,Nkd2,Hmox1,Cyba,Sox10,Plp1,Lpr5,dkk3} {Lgr6,Nkd2,Hmox1,Cyba,Sox10,Plp1,Lpr5,Nrf2} {Lgr6,Nkd2,Hmox1,Cyba,Sox10,Plp1,dkk3,Nrf2} {Lgr6,Nkd2,Hmox1,Cyba,Sox10,Lpr5,dkk3,Nrf2} {Lgr6,Nkd2,Hmox1,Cyba,Plp1,Lpr5,dkk3,Nrf2} {Lgr6,Nkd2,Hmox1,Sox10,Plp1,Lpr5,dkk3,Nrf2} {Lgr6,Nkd2,Cyba,Sox10,Plp1,Lpr5,dkk3,Nrf2} {Lgr6,Tgfbr2,Tgfbr3,Hmox1,Cyba,Sox10,Plp1,Lpr5} {Lgr6,Tgfbr2,Tgfbr3,Hmox1,Cyba,Sox10,Plp1,dkk3} {Lgr6,Tgfbr2,Tgfbr3,Hmox1,Cyba,Sox10,Plp1,Nrf2} {Lgr6,Tgfbr2,Tgfbr3,Hmox1,Cyba,Sox10,Lpr5,dkk3} {Lgr6,Tgfbr2,Tgfbr3,Hmox1,Cyba,Sox10,Lpr5,Nrf2} {Lgr6,Tgfbr2,Tgfbr3,Hmox1,Cyba,Sox10,dkk3,Nrf2} {Lgr6,Tgfbr2,Tgfbr3,Hmox1,Cyba,Plp1,Lpr5,dkk3} {Lgr6,Tgfbr2,Tgfbr3,Hmox1,Cyba,Plp1,Lpr5,Nrf2} {Lgr6,Tgfbr2,Tgfbr3,Hmox1,Cyba,Plp1,dkk3,Nrf2} {Lgr6,Tgfbr2,Tgfbr3,Hmox1,Cyba,Lpr5,dkk3,Nrf2} {Lgr6,Tgfbr2,Tgfbr3,Hmox1,Sox10,Plp1,Lpr5,dkk3} {Lgr6,Tgfbr2,Tgfbr3,Hmox1,Sox10,Plp1,Lpr5,Nrf2} {Lgr6,Tgfbr2,Tgfbr3,Hmox1,Sox10,Plp1,dkk3,Nrf2} {Lgr6,Tgfbr2,Tgfbr3,Hmox1,Sox10,Lpr5,dkk3,Nrf2} {Lgr6,Tgfbr2,Tgfbr3,Hmox1,Plp1,Lpr5,dkk3,Nrf2} {Lgr6,Tgfbr2,Tgfbr3,Cyba,Sox10,Plp1,Lpr5,dkk3} {Lgr6,Tgfbr2,Tgfbr3,Cyba,Sox10,Plp1,Lpr5,Nrf2} {Lgr6,Tgfbr2,Tgfbr3,Cyba,Sox10,Plp1,dkk3,Nrf2} {Lgr6,Tgfbr2,Tgfbr3,Cyba,Sox10,Lpr5,dkk3,Nrf2} {Lgr6,Tgfbr2,Tgfbr3,Cyba,Plp1,Lpr5,dkk3,Nrf2} {Lgr6,Tgfbr2,Tgfbr3,Sox10,Plp1,Lpr5,dkk3,Nrf2} {Lgr6,Tgfbr2,Hmox1,Cyba,Sox10,Plp1,Lpr5,dkk3} {Lgr6,Tgfbr2,Hmox1,Cyba,Sox10,Plp1,Lpr5,Nrf2} {Lgr6,Tgfbr2,Hmox1,Cyba,Sox10,Plp1,dkk3,Nrf2} {Lgr6,Tgfbr2,Hmox1,Cyba,Sox10,Lpr5,dkk3,Nrf2} {Lgr6,Tgfbr2,Hmox1,Cyba,Plp1,Lpr5,dkk3,Nrf2} {Lgr6,Tgfbr2,Hmox1,Sox10,Plp1,Lpr5,dkk3,Nrf2} {Lgr6,Tgfbr2,Cyba,Sox10,Plp1,Lpr5,dkk3,Nrf2} {Lgr6,Tgfbr3,Hmox1,Cyba,Sox10,Plp1,Lpr5,dkk3} {Lgr6,Tgfbr3,Hmox1,Cyba,Sox10,Plp1,Lpr5,Nrf2} {Lgr6,Tgfbr3,Hmox1,Cyba,Sox10,Plp1,dkk3,Nrf2} {Lgr6,Tgfbr3,Hmox1,Cyba,Sox10,Lpr5,dkk3,Nrf2} {Lgr6,Tgfbr3,Hmox1,Cyba,Plp1,Lpr5,dkk3,Nrf2} {Lgr6,Tgfbr3,Hmox1,Sox10,Plp1,Lpr5,dkk3,Nrf2} {Lgr6,Tgfbr3,Cyba,Sox10,Plp1,Lpr5,dkk3,Nrf2} {Lgr6,Hmox1,Cyba,Sox10,Plp1,Lpr5,dkk3,Nrf2} {Frb,Nkd2,Tgfbr2,Tgfbr3,Hmox1,Cyba,Sox10,Plp1} {Frb,Nkd2,Tgfbr2,Tgfbr3,Hmox1,Cyba,Sox10,Lpr5} {Frb,Nkd2,Tgfbr2,Tgfbr3,Hmox1,Cyba,Sox10,dkk3} {Frb,Nkd2,Tgfbr2,Tgfbr3,Hmox1,Cyba,Sox10,Nrf2} {Frb,Nkd2,Tgfbr2,Tgfbr3,Hmox1,Cyba,Plp1,Lpr5} {Frb,Nkd2,Tgfbr2,Tgfbr3,Hmox1,Cyba,Plp1,dkk3} {Frb,Nkd2,Tgfbr2,Tgfbr3,Hmox1,Cyba,Plp1,Nrf2} {Frb,Nkd2,Tgfbr2,Tgfbr3,Hmox1,Cyba,Lpr5,dkk3} {Frb,Nkd2,Tgfbr2,Tgfbr3,Hmox1,Cyba,Lpr5,Nrf2} {Frb,Nkd2,Tgfbr2,Tgfbr3,Hmox1,Cyba,dkk3,Nrf2} {Frb,Nkd2,Tgfbr2,Tgfbr3,Hmox1,Sox10,Plp1,Lpr5} {Frb,Nkd2,Tgfbr2,Tgfbr3,Hmox1,Sox10,Plp1,dkk3} {Frb,Nkd2,Tgfbr2,Tgfbr3,Hmox1,Sox10,Plp1,Nrf2} {Frb,Nkd2,Tgfbr2,Tgfbr3,Hmox1,Sox10,Lpr5,dkk3} {Frb,Nkd2,Tgfbr2,Tgfbr3,Hmox1,Sox10,Lpr5,Nrf2} {Frb,Nkd2,Tgfbr2,Tgfbr3,Hmox1,Sox10,dkk3,Nrf2} {Frb,Nkd2,Tgfbr2,Tgfbr3,Hmox1,Plp1,Lpr5,dkk3} {Frb,Nkd2,Tgfbr2,Tgfbr3,Hmox1,Plp1,Lpr5,Nrf2} {Frb,Nkd2,Tgfbr2,Tgfbr3,Hmox1,Plp1,dkk3,Nrf2} {Frb,Nkd2,Tgfbr2,Tgfbr3,Hmox1,Lpr5,dkk3,Nrf2} {Frb,Nkd2,Tgfbr2,Tgfbr3,Cyba,Sox10,Plp1,Lpr5} {Frb,Nkd2,Tgfbr2,Tgfbr3,Cyba,Sox10,Plp1,dkk3} {Frb,Nkd2,Tgfbr2,Tgfbr3,Cyba,Sox10,Plp1,Nrf2} {Frb,Nkd2,Tgfbr2,Tgfbr3,Cyba,Sox10,Lpr5,dkk3} {Frb,Nkd2,Tgfbr2,Tgfbr3,Cyba,Sox10,Lpr5,Nrf2} {Frb,Nkd2,Tgfbr2,Tgfbr3,Cyba,Sox10,dkk3,Nrf2} {Frb,Nkd2,Tgfbr2,Tgfbr3,Cyba,Plp1,Lpr5,dkk3} {Frb,Nkd2,Tgfbr2,Tgfbr3,Cyba,Plp1,Lpr5,Nrf2} {Frb,Nkd2,Tgfbr2,Tgfbr3,Cyba,Plp1,dkk3,Nrf2} {Frb,Nkd2,Tgfbr2,Tgfbr3,Cyba,Lpr5,dkk3,Nrf2} {Frb,Nkd2,Tgfbr2,Tgfbr3,Sox10,Plp1,Lpr5,dkk3} {Frb,Nkd2,Tgfbr2,Tgfbr3,Sox10,Plp1,Lpr5,Nrf2} {Frb,Nkd2,Tgfbr2,Tgfbr3,Sox10,Plp1,dkk3,Nrf2} {Frb,Nkd2,Tgfbr2,Tgfbr3,Sox10,Lpr5,dkk3,Nrf2} {Frb,Nkd2,Tgfbr2,Tgfbr3,Plp1,Lpr5,dkk3,Nrf2} {Frb,Nkd2,Tgfbr2,Hmox1,Cyba,Sox10,Plp1,Lpr5} {Frb,Nkd2,Tgfbr2,Hmox1,Cyba,Sox10,Plp1,dkk3} {Frb,Nkd2,Tgfbr2,Hmox1,Cyba,Sox10,Plp1,Nrf2} {Frb,Nkd2,Tgfbr2,Hmox1,Cyba,Sox10,Lpr5,dkk3} {Frb,Nkd2,Tgfbr2,Hmox1,Cyba,Sox10,Lpr5,Nrf2} {Frb,Nkd2,Tgfbr2,Hmox1,Cyba,Sox10,dkk3,Nrf2} {Frb,Nkd2,Tgfbr2,Hmox1,Cyba,Plp1,Lpr5,dkk3} {Frb,Nkd2,Tgfbr2,Hmox1,Cyba,Plp1,Lpr5,Nrf2} {Frb,Nkd2,Tgfbr2,Hmox1,Cyba,Plp1,dkk3,Nrf2} {Frb,Nkd2,Tgfbr2,Hmox1,Cyba,Lpr5,dkk3,Nrf2} {Frb,Nkd2,Tgfbr2,Hmox1,Sox10,Plp1,Lpr5,dkk3} {Frb,Nkd2,Tgfbr2,Hmox1,Sox10,Plp1,Lpr5,Nrf2} {Frb,Nkd2,Tgfbr2,Hmox1,Sox10,Plp1,dkk3,Nrf2} {Frb,Nkd2,Tgfbr2,Hmox1,Sox10,Lpr5,dkk3,Nrf2} {Frb,Nkd2,Tgfbr2,Hmox1,Plp1,Lpr5,dkk3,Nrf2} {Frb,Nkd2,Tgfbr2,Cyba,Sox10,Plp1,Lpr5,dkk3} {Frb,Nkd2,Tgfbr2,Cyba,Sox10,Plp1,Lpr5,Nrf2} {Frb,Nkd2,Tgfbr2,Cyba,Sox10,Plp1,dkk3,Nrf2} {Frb,Nkd2,Tgfbr2,Cyba,Sox10,Lpr5,dkk3,Nrf2} {Frb,Nkd2,Tgfbr2,Cyba,Plp1,Lpr5,dkk3,Nrf2} {Frb,Nkd2,Tgfbr2,Sox10,Plp1,Lpr5,dkk3,Nrf2} {Frb,Nkd2,Tgfbr3,Hmox1,Cyba,Sox10,Plp1,Lpr5} {Frb,Nkd2,Tgfbr3,Hmox1,Cyba,Sox10,Plp1,dkk3} {Frb,Nkd2,Tgfbr3,Hmox1,Cyba,Sox10,Plp1,Nrf2} {Frb,Nkd2,Tgfbr3,Hmox1,Cyba,Sox10,Lpr5,dkk3} {Frb,Nkd2,Tgfbr3,Hmox1,Cyba,Sox10,Lpr5,Nrf2} {Frb,Nkd2,Tgfbr3,Hmox1,Cyba,Sox10,dkk3,Nrf2} {Frb,Nkd2,Tgfbr3,Hmox1,Cyba,Plp1,Lpr5,dkk3} {Frb,Nkd2,Tgfbr3,Hmox1,Cyba,Plp1,Lpr5,Nrf2} {Frb,Nkd2,Tgfbr3,Hmox1,Cyba,Plp1,dkk3,Nrf2} {Frb,Nkd2,Tgfbr3,Hmox1,Cyba,Lpr5,dkk3,Nrf2} {Frb,Nkd2,Tgfbr3,Hmox1,Sox10,Plp1,Lpr5,dkk3} {Frb,Nkd2,Tgfbr3,Hmox1,Sox10,Plp1,Lpr5,Nrf2} {Frb,Nkd2,Tgfbr3,Hmox1,Sox10,Plp1,dkk3,Nrf2} {Frb,Nkd2,Tgfbr3,Hmox1,Sox10,Lpr5,dkk3,Nrf2} {Frb,Nkd2,Tgfbr3,Hmox1,Plp1,Lpr5,dkk3,Nrf2} {Frb,Nkd2,Tgfbr3,Cyba,Sox10,Plp1,Lpr5,dkk3} {Frb,Nkd2,Tgfbr3,Cyba,Sox10,Plp1,Lpr5,Nrf2} {Frb,Nkd2,Tgfbr3,Cyba,Sox10,Plp1,dkk3,Nrf2} {Frb,Nkd2,Tgfbr3,Cyba,Sox10,Lpr5,dkk3,Nrf2} {Frb,Nkd2,Tgfbr3,Cyba,Plp1,Lpr5,dkk3,Nrf2} {Frb,Nkd2,Tgfbr3,Sox10,Plp1,Lpr5,dkk3,Nrf2} {Frb,Nkd2,Hmox1,Cyba,Sox10,Plp1,Lpr5,dkk3} {Frb,Nkd2,Hmox1,Cyba,Sox10,Plp1,Lpr5,Nrf2} {Frb,Nkd2,Hmox1,Cyba,Sox10,Plp1,dkk3,Nrf2} {Frb,Nkd2,Hmox1,Cyba,Sox10,Lpr5,dkk3,Nrf2} {Frb,Nkd2,Hmox1,Cyba,Plp1,Lpr5,dkk3,Nrf2} {Frb,Nkd2,Hmox1,Sox10,Plp1,Lpr5,dkk3,Nrf2} {Frb,Nkd2,Cyba,Sox10,Plp1,Lpr5,dkk3,Nrf2} {Frb,Tgfbr2,Tgfbr3,Hmox1,Cyba,Sox10,Plp1,Lpr5} {Frb,Tgfbr2,Tgfbr3,Hmox1,Cyba,Sox10,Plp1,dkk3} {Frb,Tgfbr2,Tgfbr3,Hmox1,Cyba,Sox10,Plp1,Nrf2} {Frb,Tgfbr2,Tgfbr3,Hmox1,Cyba,Sox10,Lpr5,dkk3} {Frb,Tgfbr2,Tgfbr3,Hmox1,Cyba,Sox10,Lpr5,Nrf2} {Frb,Tgfbr2,Tgfbr3,Hmox1,Cyba,Sox10,dkk3,Nrf2} {Frb,Tgfbr2,Tgfbr3,Hmox1,Cyba,Plp1,Lpr5,dkk3} {Frb,Tgfbr2,Tgfbr3,Hmox1,Cyba,Plp1,Lpr5,Nrf2} {Frb,Tgfbr2,Tgfbr3,Hmox1,Cyba,Plp1,dkk3,Nrf2} {Frb,Tgfbr2,Tgfbr3,Hmox1,Cyba,Lpr5,dkk3,Nrf2} {Frb,Tgfbr2,Tgfbr3,Hmox1,Sox10,Plp1,Lpr5,dkk3} {Frb,Tgfbr2,Tgfbr3,Hmox1,Sox10,Plp1,Lpr5,Nrf2} {Frb,Tgfbr2,Tgfbr3,Hmox1,Sox10,Plp1,dkk3,Nrf2} {Frb,Tgfbr2,Tgfbr3,Hmox1,Sox10,Lpr5,dkk3,Nrf2} {Frb,Tgfbr2,Tgfbr3,Hmox1,Plp1,Lpr5,dkk3,Nrf2} {Frb,Tgfbr2,Tgfbr3,Cyba,Sox10,Plp1,Lpr5,dkk3} {Frb,Tgfbr2,Tgfbr3,Cyba,Sox10,Plp1,Lpr5,Nrf2} {Frb,Tgfbr2,Tgfbr3,Cyba,Sox10,Plp1,dkk3,Nrf2} {Frb,Tgfbr2,Tgfbr3,Cyba,Sox10,Lpr5,dkk3,Nrf2} {Frb,Tgfbr2,Tgfbr3,Cyba,Plp1,Lpr5,dkk3,Nrf2} {Frb,Tgfbr2,Tgfbr3,Sox10,Plp1,Lpr5,dkk3,Nrf2} {Frb,Tgfbr2,Hmox1,Cyba,Sox10,Plp1,Lpr5,dkk3} {Frb,Tgfbr2,Hmox1,Cyba,Sox10,Plp1,Lpr5,Nrf2} {Frb,Tgfbr2,Hmox1,Cyba,Sox10,Plp1,dkk3,Nrf2} {Frb,Tgfbr2,Hmox1,Cyba,Sox10,Lpr5,dkk3,Nrf2} {Frb,Tgfbr2,Hmox1,Cyba,Plp1,Lpr5,dkk3,Nrf2} {Frb,Tgfbr2,Hmox1,Sox10,Plp1,Lpr5,dkk3,Nrf2} {Frb,Tgfbr2,Cyba,Sox10,Plp1,Lpr5,dkk3,Nrf2} {Frb,Tgfbr3,Hmox1,Cyba,Sox10,Plp1,Lpr5,dkk3} {Frb,Tgfbr3,Hmox1,Cyba,Sox10,Plp1,Lpr5,Nrf2} {Frb,Tgfbr3,Hmox1,Cyba,Sox10,Plp1,dkk3,Nrf2} {Frb,Tgfbr3,Hmox1,Cyba,Sox10,Lpr5,dkk3,Nrf2} {Frb,Tgfbr3,Hmox1,Cyba,Plp1,Lpr5,dkk3,Nrf2} {Frb,Tgfbr3,Hmox1,Sox10,Plp1,Lpr5,dkk3,Nrf2} {Frb,Tgfbr3,Cyba,Sox10,Plp1,Lpr5,dkk3,Nrf2} {Frb,Hmox1,Cyba,Sox10,Plp1,Lpr5,dkk3,Nrf2} {Nkd2,Tgfbr2,Tgfbr3,Hmox1,Cyba,Sox10,Plp1,Lpr5} {Nkd2,Tgfbr2,Tgfbr3,Hmox1,Cyba,Sox10,Plp1,dkk3} {Nkd2,Tgfbr2,Tgfbr3,Hmox1,Cyba,Sox10,Plp1,Nrf2} {Nkd2,Tgfbr2,Tgfbr3,Hmox1,Cyba,Sox10,Lpr5,dkk3} {Nkd2,Tgfbr2,Tgfbr3,Hmox1,Cyba,Sox10,Lpr5,Nrf2} {Nkd2,Tgfbr2,Tgfbr3,Hmox1,Cyba,Sox10,dkk3,Nrf2} {Nkd2,Tgfbr2,Tgfbr3,Hmox1,Cyba,Plp1,Lpr5,dkk3} {Nkd2,Tgfbr2,Tgfbr3,Hmox1,Cyba,Plp1,Lpr5,Nrf2} {Nkd2,Tgfbr2,Tgfbr3,Hmox1,Cyba,Plp1,dkk3,Nrf2} {Nkd2,Tgfbr2,Tgfbr3,Hmox1,Cyba,Lpr5,dkk3,Nrf2} {Nkd2,Tgfbr2,Tgfbr3,Hmox1,Sox10,Plp1,Lpr5,dkk3} {Nkd2,Tgfbr2,Tgfbr3,Hmox1,Sox10,Plp1,Lpr5,Nrf2} {Nkd2,Tgfbr2,Tgfbr3,Hmox1,Sox10,Plp1,dkk3,Nrf2} {Nkd2,Tgfbr2,Tgfbr3,Hmox1,Sox10,Lpr5,dkk3,Nrf2} {Nkd2,Tgfbr2,Tgfbr3,Hmox1,Plp1,Lpr5,dkk3,Nrf2} {Nkd2,Tgfbr2,Tgfbr3,Cyba,Sox10,Plp1,Lpr5,dkk3} {Nkd2,Tgfbr2,Tgfbr3,Cyba,Sox10,Plp1,Lpr5,Nrf2} {Nkd2,Tgfbr2,Tgfbr3,Cyba,Sox10,Plp1,dkk3,Nrf2} {Nkd2,Tgfbr2,Tgfbr3,Cyba,Sox10,Lpr5,dkk3,Nrf2} {Nkd2,Tgfbr2,Tgfbr3,Cyba,Plp1,Lpr5,dkk3,Nrf2} {Nkd2,Tgfbr2,Tgfbr3,Sox10,Plp1,Lpr5,dkk3,Nrf2} {Nkd2,Tgfbr2,Hmox1,Cyba,Sox10,Plp1,Lpr5,dkk3} {Nkd2,Tgfbr2,Hmox1,Cyba,Sox10,Plp1,Lpr5,Nrf2} {Nkd2,Tgfbr2,Hmox1,Cyba,Sox10,Plp1,dkk3,Nrf2} {Nkd2,Tgfbr2,Hmox1,Cyba,Sox10,Lpr5,dkk3,Nrf2} {Nkd2,Tgfbr2,Hmox1,Cyba,Plp1,Lpr5,dkk3,Nrf2} {Nkd2,Tgfbr2,Hmox1,Sox10,Plp1,Lpr5,dkk3,Nrf2} {Nkd2,Tgfbr2,Cyba,Sox10,Plp1,Lpr5,dkk3,Nrf2} {Nkd2,Tgfbr3,Hmox1,Cyba,Sox10,Plp1,Lpr5,dkk3} {Nkd2,Tgfbr3,Hmox1,Cyba,Sox10,Plp1,Lpr5,Nrf2} {Nkd2,Tgfbr3,Hmox1,Cyba,Sox10,Plp1,dkk3,Nrf2} {Nkd2,Tgfbr3,Hmox1,Cyba,Sox10,Lpr5,dkk3,Nrf2} {Nkd2,Tgfbr3,Hmox1,Cyba,Plp1,Lpr5,dkk3,Nrf2} {Nkd2,Tgfbr3,Hmox1,Sox10,Plp1,Lpr5,dkk3,Nrf2} {Nkd2,Tgfbr3,Cyba,Sox10,Plp1,Lpr5,dkk3,Nrf2} {Nkd2,Hmox1,Cyba,Sox10,Plp1,Lpr5,dkk3,Nrf2} {Tgfbr2,Tgfbr3,Hmox1,Cyba,Sox10,Plp1,Lpr5,dkk3} {Tgfbr2,Tgfbr3,Hmox1,Cyba,Sox10,Plp1,Lpr5,Nrf2} {Tgfbr2,Tgfbr3,Hmox1,Cyba,Sox10,Plp1,dkk3,Nrf2} {Tgfbr2,Tgfbr3,Hmox1,Cyba,Sox10,Lpr5,dkk3,Nrf2} {Tgfbr2,Tgfbr3,Hmox1,Cyba,Plp1,Lpr5,dkk3,Nrf2} {Tgfbr2,Tgfbr3,Hmox1,Sox10,Plp1,Lpr5,dkk3,Nrf2} {Tgfbr2,Tgfbr3,Cyba,Sox10,Plp1,Lpr5,dkk3,Nrf2} {Tgfbr2,Hmox1,Cyba,Sox10,Plp1,Lpr5,dkk3,Nrf2} {Tgfbr3,Hmox1,Cyba,Sox10,Plp1,Lpr5,dkk3,Nrf2} {Lgr6,Frb,Nkd2,Tgfbr2,Tgfbr3,Hmox1,Cyba,Sox10,Plp1} {Lgr6,Frb,Nkd2,Tgfbr2,Tgfbr3,Hmox1,Cyba,Sox10,Lpr5} {Lgr6,Frb,Nkd2,Tgfbr2,Tgfbr3,Hmox1,Cyba,Sox10,dkk3} {Lgr6,Frb,Nkd2,Tgfbr2,Tgfbr3,Hmox1,Cyba,Sox10,Nrf2} {Lgr6,Frb,Nkd2,Tgfbr2,Tgfbr3,Hmox1,Cyba,Plp1,Lpr5} {Lgr6,Frb,Nkd2,Tgfbr2,Tgfbr3,Hmox1,Cyba,Plp1,dkk3} {Lgr6,Frb,Nkd2,Tgfbr2,Tgfbr3,Hmox1,Cyba,Plp1,Nrf2} {Lgr6,Frb,Nkd2,Tgfbr2,Tgfbr3,Hmox1,Cyba,Lpr5,dkk3} {Lgr6,Frb,Nkd2,Tgfbr2,Tgfbr3,Hmox1,Cyba,Lpr5,Nrf2} {Lgr6,Frb,Nkd2,Tgfbr2,Tgfbr3,Hmox1,Cyba,dkk3,Nrf2} {Lgr6,Frb,Nkd2,Tgfbr2,Tgfbr3,Hmox1,Sox10,Plp1,Lpr5} {Lgr6,Frb,Nkd2,Tgfbr2,Tgfbr3,Hmox1,Sox10,Plp1,dkk3} {Lgr6,Frb,Nkd2,Tgfbr2,Tgfbr3,Hmox1,Sox10,Plp1,Nrf2} {Lgr6,Frb,Nkd2,Tgfbr2,Tgfbr3,Hmox1,Sox10,Lpr5,dkk3} {Lgr6,Frb,Nkd2,Tgfbr2,Tgfbr3,Hmox1,Sox10,Lpr5,Nrf2} {Lgr6,Frb,Nkd2,Tgfbr2,Tgfbr3,Hmox1,Sox10,dkk3,Nrf2} {Lgr6,Frb,Nkd2,Tgfbr2,Tgfbr3,Hmox1,Plp1,Lpr5,dkk3} {Lgr6,Frb,Nkd2,Tgfbr2,Tgfbr3,Hmox1,Plp1,Lpr5,Nrf2} {Lgr6,Frb,Nkd2,Tgfbr2,Tgfbr3,Hmox1,Plp1,dkk3,Nrf2} {Lgr6,Frb,Nkd2,Tgfbr2,Tgfbr3,Hmox1,Lpr5,dkk3,Nrf2} {Lgr6,Frb,Nkd2,Tgfbr2,Tgfbr3,Cyba,Sox10,Plp1,Lpr5} {Lgr6,Frb,Nkd2,Tgfbr2,Tgfbr3,Cyba,Sox10,Plp1,dkk3} {Lgr6,Frb,Nkd2,Tgfbr2,Tgfbr3,Cyba,Sox10,Plp1,Nrf2} {Lgr6,Frb,Nkd2,Tgfbr2,Tgfbr3,Cyba,Sox10,Lpr5,dkk3} {Lgr6,Frb,Nkd2,Tgfbr2,Tgfbr3,Cyba,Sox10,Lpr5,Nrf2} {Lgr6,Frb,Nkd2,Tgfbr2,Tgfbr3,Cyba,Sox10,dkk3,Nrf2} {Lgr6,Frb,Nkd2,Tgfbr2,Tgfbr3,Cyba,Plp1,Lpr5,dkk3} {Lgr6,Frb,Nkd2,Tgfbr2,Tgfbr3,Cyba,Plp1,Lpr5,Nrf2} {Lgr6,Frb,Nkd2,Tgfbr2,Tgfbr3,Cyba,Plp1,dkk3,Nrf2} {Lgr6,Frb,Nkd2,Tgfbr2,Tgfbr3,Cyba,Lpr5,dkk3,Nrf2} {Lgr6,Frb,Nkd2,Tgfbr2,Tgfbr3,Sox10,Plp1,Lpr5,dkk3} {Lgr6,Frb,Nkd2,Tgfbr2,Tgfbr3,Sox10,Plp1,Lpr5,Nrf2} {Lgr6,Frb,Nkd2,Tgfbr2,Tgfbr3,Sox10,Plp1,dkk3,Nrf2} {Lgr6,Frb,Nkd2,Tgfbr2,Tgfbr3,Sox10,Lpr5,dkk3,Nrf2} {Lgr6,Frb,Nkd2,Tgfbr2,Tgfbr3,Plp1,Lpr5,dkk3,Nrf2} {Lgr6,Frb,Nkd2,Tgfbr2,Hmox1,Cyba,Sox10,Plp1,Lpr5} {Lgr6,Frb,Nkd2,Tgfbr2,Hmox1,Cyba,Sox10,Plp1,dkk3} {Lgr6,Frb,Nkd2,Tgfbr2,Hmox1,Cyba,Sox10,Plp1,Nrf2} {Lgr6,Frb,Nkd2,Tgfbr2,Hmox1,Cyba,Sox10,Lpr5,dkk3} {Lgr6,Frb,Nkd2,Tgfbr2,Hmox1,Cyba,Sox10,Lpr5,Nrf2} {Lgr6,Frb,Nkd2,Tgfbr2,Hmox1,Cyba,Sox10,dkk3,Nrf2} {Lgr6,Frb,Nkd2,Tgfbr2,Hmox1,Cyba,Plp1,Lpr5,dkk3} {Lgr6,Frb,Nkd2,Tgfbr2,Hmox1,Cyba,Plp1,Lpr5,Nrf2} {Lgr6,Frb,Nkd2,Tgfbr2,Hmox1,Cyba,Plp1,dkk3,Nrf2} {Lgr6,Frb,Nkd2,Tgfbr2,Hmox1,Cyba,Lpr5,dkk3,Nrf2} {Lgr6,Frb,Nkd2,Tgfbr2,Hmox1,Sox10,Plp1,Lpr5,dkk3} {Lgr6,Frb,Nkd2,Tgfbr2,Hmox1,Sox10,Plp1,Lpr5,Nrf2} {Lgr6,Frb,Nkd2,Tgfbr2,Hmox1,Sox10,Plp1,dkk3,Nrf2} {Lgr6,Frb,Nkd2,Tgfbr2,Hmox1,Sox10,Lpr5,dkk3,Nrf2} {Lgr6,Frb,Nkd2,Tgfbr2,Hmox1,Plp1,Lpr5,dkk3,Nrf2} {Lgr6,Frb,Nkd2,Tgfbr2,Cyba,Sox10,Plp1,Lpr5,dkk3} {Lgr6,Frb,Nkd2,Tgfbr2,Cyba,Sox10,Plp1,Lpr5,Nrf2} {Lgr6,Frb,Nkd2,Tgfbr2,Cyba,Sox10,Plp1,dkk3,Nrf2} {Lgr6,Frb,Nkd2,Tgfbr2,Cyba,Sox10,Lpr5,dkk3,Nrf2} {Lgr6,Frb,Nkd2,Tgfbr2,Cyba,Plp1,Lpr5,dkk3,Nrf2} {Lgr6,Frb,Nkd2,Tgfbr2,Sox10,Plp1,Lpr5,dkk3,Nrf2} {Lgr6,Frb,Nkd2,Tgfbr3,Hmox1,Cyba,Sox10,Plp1,Lpr5} {Lgr6,Frb,Nkd2,Tgfbr3,Hmox1,Cyba,Sox10,Plp1,dkk3} {Lgr6,Frb,Nkd2,Tgfbr3,Hmox1,Cyba,Sox10,Plp1,Nrf2} {Lgr6,Frb,Nkd2,Tgfbr3,Hmox1,Cyba,Sox10,Lpr5,dkk3} {Lgr6,Frb,Nkd2,Tgfbr3,Hmox1,Cyba,Sox10,Lpr5,Nrf2} {Lgr6,Frb,Nkd2,Tgfbr3,Hmox1,Cyba,Sox10,dkk3,Nrf2} {Lgr6,Frb,Nkd2,Tgfbr3,Hmox1,Cyba,Plp1,Lpr5,dkk3} {Lgr6,Frb,Nkd2,Tgfbr3,Hmox1,Cyba,Plp1,Lpr5,Nrf2} {Lgr6,Frb,Nkd2,Tgfbr3,Hmox1,Cyba,Plp1,dkk3,Nrf2} {Lgr6,Frb,Nkd2,Tgfbr3,Hmox1,Cyba,Lpr5,dkk3,Nrf2} {Lgr6,Frb,Nkd2,Tgfbr3,Hmox1,Sox10,Plp1,Lpr5,dkk3} {Lgr6,Frb,Nkd2,Tgfbr3,Hmox1,Sox10,Plp1,Lpr5,Nrf2} {Lgr6,Frb,Nkd2,Tgfbr3,Hmox1,Sox10,Plp1,dkk3,Nrf2} {Lgr6,Frb,Nkd2,Tgfbr3,Hmox1,Sox10,Lpr5,dkk3,Nrf2} {Lgr6,Frb,Nkd2,Tgfbr3,Hmox1,Plp1,Lpr5,dkk3,Nrf2} {Lgr6,Frb,Nkd2,Tgfbr3,Cyba,Sox10,Plp1,Lpr5,dkk3} {Lgr6,Frb,Nkd2,Tgfbr3,Cyba,Sox10,Plp1,Lpr5,Nrf2} {Lgr6,Frb,Nkd2,Tgfbr3,Cyba,Sox10,Plp1,dkk3,Nrf2} {Lgr6,Frb,Nkd2,Tgfbr3,Cyba,Sox10,Lpr5,dkk3,Nrf2} {Lgr6,Frb,Nkd2,Tgfbr3,Cyba,Plp1,Lpr5,dkk3,Nrf2} {Lgr6,Frb,Nkd2,Tgfbr3,Sox10,Plp1,Lpr5,dkk3,Nrf2} {Lgr6,Frb,Nkd2,Hmox1,Cyba,Sox10,Plp1,Lpr5,dkk3} {Lgr6,Frb,Nkd2,Hmox1,Cyba,Sox10,Plp1,Lpr5,Nrf2} {Lgr6,Frb,Nkd2,Hmox1,Cyba,Sox10,Plp1,dkk3,Nrf2} {Lgr6,Frb,Nkd2,Hmox1,Cyba,Sox10,Lpr5,dkk3,Nrf2} {Lgr6,Frb,Nkd2,Hmox1,Cyba,Plp1,Lpr5,dkk3,Nrf2} {Lgr6,Frb,Nkd2,Hmox1,Sox10,Plp1,Lpr5,dkk3,Nrf2} {Lgr6,Frb,Nkd2,Cyba,Sox10,Plp1,Lpr5,dkk3,Nrf2} {Lgr6,Frb,Tgfbr2,Tgfbr3,Hmox1,Cyba,Sox10,Plp1,Lpr5} {Lgr6,Frb,Tgfbr2,Tgfbr3,Hmox1,Cyba,Sox10,Plp1,dkk3} {Lgr6,Frb,Tgfbr2,Tgfbr3,Hmox1,Cyba,Sox10,Plp1,Nrf2} {Lgr6,Frb,Tgfbr2,Tgfbr3,Hmox1,Cyba,Sox10,Lpr5,dkk3} {Lgr6,Frb,Tgfbr2,Tgfbr3,Hmox1,Cyba,Sox10,Lpr5,Nrf2} {Lgr6,Frb,Tgfbr2,Tgfbr3,Hmox1,Cyba,Sox10,dkk3,Nrf2} {Lgr6,Frb,Tgfbr2,Tgfbr3,Hmox1,Cyba,Plp1,Lpr5,dkk3} {Lgr6,Frb,Tgfbr2,Tgfbr3,Hmox1,Cyba,Plp1,Lpr5,Nrf2} {Lgr6,Frb,Tgfbr2,Tgfbr3,Hmox1,Cyba,Plp1,dkk3,Nrf2} {Lgr6,Frb,Tgfbr2,Tgfbr3,Hmox1,Cyba,Lpr5,dkk3,Nrf2} {Lgr6,Frb,Tgfbr2,Tgfbr3,Hmox1,Sox10,Plp1,Lpr5,dkk3} {Lgr6,Frb,Tgfbr2,Tgfbr3,Hmox1,Sox10,Plp1,Lpr5,Nrf2} {Lgr6,Frb,Tgfbr2,Tgfbr3,Hmox1,Sox10,Plp1,dkk3,Nrf2} {Lgr6,Frb,Tgfbr2,Tgfbr3,Hmox1,Sox10,Lpr5,dkk3,Nrf2} {Lgr6,Frb,Tgfbr2,Tgfbr3,Hmox1,Plp1,Lpr5,dkk3,Nrf2} {Lgr6,Frb,Tgfbr2,Tgfbr3,Cyba,Sox10,Plp1,Lpr5,dkk3} {Lgr6,Frb,Tgfbr2,Tgfbr3,Cyba,Sox10,Plp1,Lpr5,Nrf2} {Lgr6,Frb,Tgfbr2,Tgfbr3,Cyba,Sox10,Plp1,dkk3,Nrf2} {Lgr6,Frb,Tgfbr2,Tgfbr3,Cyba,Sox10,Lpr5,dkk3,Nrf2} {Lgr6,Frb,Tgfbr2,Tgfbr3,Cyba,Plp1,Lpr5,dkk3,Nrf2} {Lgr6,Frb,Tgfbr2,Tgfbr3,Sox10,Plp1,Lpr5,dkk3,Nrf2} {Lgr6,Frb,Tgfbr2,Hmox1,Cyba,Sox10,Plp1,Lpr5,dkk3} {Lgr6,Frb,Tgfbr2,Hmox1,Cyba,Sox10,Plp1,Lpr5,Nrf2} {Lgr6,Frb,Tgfbr2,Hmox1,Cyba,Sox10,Plp1,dkk3,Nrf2} {Lgr6,Frb,Tgfbr2,Hmox1,Cyba,Sox10,Lpr5,dkk3,Nrf2} {Lgr6,Frb,Tgfbr2,Hmox1,Cyba,Plp1,Lpr5,dkk3,Nrf2} {Lgr6,Frb,Tgfbr2,Hmox1,Sox10,Plp1,Lpr5,dkk3,Nrf2} {Lgr6,Frb,Tgfbr2,Cyba,Sox10,Plp1,Lpr5,dkk3,Nrf2} {Lgr6,Frb,Tgfbr3,Hmox1,Cyba,Sox10,Plp1,Lpr5,dkk3} {Lgr6,Frb,Tgfbr3,Hmox1,Cyba,Sox10,Plp1,Lpr5,Nrf2} {Lgr6,Frb,Tgfbr3,Hmox1,Cyba,Sox10,Plp1,dkk3,Nrf2} {Lgr6,Frb,Tgfbr3,Hmox1,Cyba,Sox10,Lpr5,dkk3,Nrf2} {Lgr6,Frb,Tgfbr3,Hmox1,Cyba,Plp1,Lpr5,dkk3,Nrf2} {Lgr6,Frb,Tgfbr3,Hmox1,Sox10,Plp1,Lpr5,dkk3,Nrf2} {Lgr6,Frb,Tgfbr3,Cyba,Sox10,Plp1,Lpr5,dkk3,Nrf2} {Lgr6,Frb,Hmox1,Cyba,Sox10,Plp1,Lpr5,dkk3,Nrf2} {Lgr6,Nkd2,Tgfbr2,Tgfbr3,Hmox1,Cyba,Sox10,Plp1,Lpr5} {Lgr6,Nkd2,Tgfbr2,Tgfbr3,Hmox1,Cyba,Sox10,Plp1,dkk3} {Lgr6,Nkd2,Tgfbr2,Tgfbr3,Hmox1,Cyba,Sox10,Plp1,Nrf2} {Lgr6,Nkd2,Tgfbr2,Tgfbr3,Hmox1,Cyba,Sox10,Lpr5,dkk3} {Lgr6,Nkd2,Tgfbr2,Tgfbr3,Hmox1,Cyba,Sox10,Lpr5,Nrf2} {Lgr6,Nkd2,Tgfbr2,Tgfbr3,Hmox1,Cyba,Sox10,dkk3,Nrf2} {Lgr6,Nkd2,Tgfbr2,Tgfbr3,Hmox1,Cyba,Plp1,Lpr5,dkk3} {Lgr6,Nkd2,Tgfbr2,Tgfbr3,Hmox1,Cyba,Plp1,Lpr5,Nrf2} {Lgr6,Nkd2,Tgfbr2,Tgfbr3,Hmox1,Cyba,Plp1,dkk3,Nrf2} {Lgr6,Nkd2,Tgfbr2,Tgfbr3,Hmox1,Cyba,Lpr5,dkk3,Nrf2} {Lgr6,Nkd2,Tgfbr2,Tgfbr3,Hmox1,Sox10,Plp1,Lpr5,dkk3} {Lgr6,Nkd2,Tgfbr2,Tgfbr3,Hmox1,Sox10,Plp1,Lpr5,Nrf2} {Lgr6,Nkd2,Tgfbr2,Tgfbr3,Hmox1,Sox10,Plp1,dkk3,Nrf2} {Lgr6,Nkd2,Tgfbr2,Tgfbr3,Hmox1,Sox10,Lpr5,dkk3,Nrf2} {Lgr6,Nkd2,Tgfbr2,Tgfbr3,Hmox1,Plp1,Lpr5,dkk3,Nrf2} {Lgr6,Nkd2,Tgfbr2,Tgfbr3,Cyba,Sox10,Plp1,Lpr5,dkk3} {Lgr6,Nkd2,Tgfbr2,Tgfbr3,Cyba,Sox10,Plp1,Lpr5,Nrf2} {Lgr6,Nkd2,Tgfbr2,Tgfbr3,Cyba,Sox10,Plp1,dkk3,Nrf2} {Lgr6,Nkd2,Tgfbr2,Tgfbr3,Cyba,Sox10,Lpr5,dkk3,Nrf2} {Lgr6,Nkd2,Tgfbr2,Tgfbr3,Cyba,Plp1,Lpr5,dkk3,Nrf2} {Lgr6,Nkd2,Tgfbr2,Tgfbr3,Sox10,Plp1,Lpr5,dkk3,Nrf2} {Lgr6,Nkd2,Tgfbr2,Hmox1,Cyba,Sox10,Plp1,Lpr5,dkk3} {Lgr6,Nkd2,Tgfbr2,Hmox1,Cyba,Sox10,Plp1,Lpr5,Nrf2} {Lgr6,Nkd2,Tgfbr2,Hmox1,Cyba,Sox10,Plp1,dkk3,Nrf2} {Lgr6,Nkd2,Tgfbr2,Hmox1,Cyba,Sox10,Lpr5,dkk3,Nrf2} {Lgr6,Nkd2,Tgfbr2,Hmox1,Cyba,Plp1,Lpr5,dkk3,Nrf2} {Lgr6,Nkd2,Tgfbr2,Hmox1,Sox10,Plp1,Lpr5,dkk3,Nrf2} {Lgr6,Nkd2,Tgfbr2,Cyba,Sox10,Plp1,Lpr5,dkk3,Nrf2} {Lgr6,Nkd2,Tgfbr3,Hmox1,Cyba,Sox10,Plp1,Lpr5,dkk3} {Lgr6,Nkd2,Tgfbr3,Hmox1,Cyba,Sox10,Plp1,Lpr5,Nrf2} {Lgr6,Nkd2,Tgfbr3,Hmox1,Cyba,Sox10,Plp1,dkk3,Nrf2} {Lgr6,Nkd2,Tgfbr3,Hmox1,Cyba,Sox10,Lpr5,dkk3,Nrf2} {Lgr6,Nkd2,Tgfbr3,Hmox1,Cyba,Plp1,Lpr5,dkk3,Nrf2} {Lgr6,Nkd2,Tgfbr3,Hmox1,Sox10,Plp1,Lpr5,dkk3,Nrf2} {Lgr6,Nkd2,Tgfbr3,Cyba,Sox10,Plp1,Lpr5,dkk3,Nrf2} {Lgr6,Nkd2,Hmox1,Cyba,Sox10,Plp1,Lpr5,dkk3,Nrf2} {Lgr6,Tgfbr2,Tgfbr3,Hmox1,Cyba,Sox10,Plp1,Lpr5,dkk3} {Lgr6,Tgfbr2,Tgfbr3,Hmox1,Cyba,Sox10,Plp1,Lpr5,Nrf2} {Lgr6,Tgfbr2,Tgfbr3,Hmox1,Cyba,Sox10,Plp1,dkk3,Nrf2} {Lgr6,Tgfbr2,Tgfbr3,Hmox1,Cyba,Sox10,Lpr5,dkk3,Nrf2} {Lgr6,Tgfbr2,Tgfbr3,Hmox1,Cyba,Plp1,Lpr5,dkk3,Nrf2} {Lgr6,Tgfbr2,Tgfbr3,Hmox1,Sox10,Plp1,Lpr5,dkk3,Nrf2} {Lgr6,Tgfbr2,Tgfbr3,Cyba,Sox10,Plp1,Lpr5,dkk3,Nrf2} {Lgr6,Tgfbr2,Hmox1,Cyba,Sox10,Plp1,Lpr5,dkk3,Nrf2} {Lgr6,Tgfbr3,Hmox1,Cyba,Sox10,Plp1,Lpr5,dkk3,Nrf2} {Frb,Nkd2,Tgfbr2,Tgfbr3,Hmox1,Cyba,Sox10,Plp1,Lpr5} {Frb,Nkd2,Tgfbr2,Tgfbr3,Hmox1,Cyba,Sox10,Plp1,dkk3} {Frb,Nkd2,Tgfbr2,Tgfbr3,Hmox1,Cyba,Sox10,Plp1,Nrf2} {Frb,Nkd2,Tgfbr2,Tgfbr3,Hmox1,Cyba,Sox10,Lpr5,dkk3} {Frb,Nkd2,Tgfbr2,Tgfbr3,Hmox1,Cyba,Sox10,Lpr5,Nrf2} {Frb,Nkd2,Tgfbr2,Tgfbr3,Hmox1,Cyba,Sox10,dkk3,Nrf2} {Frb,Nkd2,Tgfbr2,Tgfbr3,Hmox1,Cyba,Plp1,Lpr5,dkk3} {Frb,Nkd2,Tgfbr2,Tgfbr3,Hmox1,Cyba,Plp1,Lpr5,Nrf2} {Frb,Nkd2,Tgfbr2,Tgfbr3,Hmox1,Cyba,Plp1,dkk3,Nrf2} {Frb,Nkd2,Tgfbr2,Tgfbr3,Hmox1,Cyba,Lpr5,dkk3,Nrf2} {Frb,Nkd2,Tgfbr2,Tgfbr3,Hmox1,Sox10,Plp1,Lpr5,dkk3} {Frb,Nkd2,Tgfbr2,Tgfbr3,Hmox1,Sox10,Plp1,Lpr5,Nrf2} {Frb,Nkd2,Tgfbr2,Tgfbr3,Hmox1,Sox10,Plp1,dkk3,Nrf2} {Frb,Nkd2,Tgfbr2,Tgfbr3,Hmox1,Sox10,Lpr5,dkk3,Nrf2} {Frb,Nkd2,Tgfbr2,Tgfbr3,Hmox1,Plp1,Lpr5,dkk3,Nrf2} {Frb,Nkd2,Tgfbr2,Tgfbr3,Cyba,Sox10,Plp1,Lpr5,dkk3} {Frb,Nkd2,Tgfbr2,Tgfbr3,Cyba,Sox10,Plp1,Lpr5,Nrf2} {Frb,Nkd2,Tgfbr2,Tgfbr3,Cyba,Sox10,Plp1,dkk3,Nrf2} {Frb,Nkd2,Tgfbr2,Tgfbr3,Cyba,Sox10,Lpr5,dkk3,Nrf2} {Frb,Nkd2,Tgfbr2,Tgfbr3,Cyba,Plp1,Lpr5,dkk3,Nrf2} {Frb,Nkd2,Tgfbr2,Tgfbr3,Sox10,Plp1,Lpr5,dkk3,Nrf2} {Frb,Nkd2,Tgfbr2,Hmox1,Cyba,Sox10,Plp1,Lpr5,dkk3} {Frb,Nkd2,Tgfbr2,Hmox1,Cyba,Sox10,Plp1,Lpr5,Nrf2} {Frb,Nkd2,Tgfbr2,Hmox1,Cyba,Sox10,Plp1,dkk3,Nrf2} {Frb,Nkd2,Tgfbr2,Hmox1,Cyba,Sox10,Lpr5,dkk3,Nrf2} {Frb,Nkd2,Tgfbr2,Hmox1,Cyba,Plp1,Lpr5,dkk3,Nrf2} {Frb,Nkd2,Tgfbr2,Hmox1,Sox10,Plp1,Lpr5,dkk3,Nrf2} {Frb,Nkd2,Tgfbr2,Cyba,Sox10,Plp1,Lpr5,dkk3,Nrf2} {Frb,Nkd2,Tgfbr3,Hmox1,Cyba,Sox10,Plp1,Lpr5,dkk3} {Frb,Nkd2,Tgfbr3,Hmox1,Cyba,Sox10,Plp1,Lpr5,Nrf2} {Frb,Nkd2,Tgfbr3,Hmox1,Cyba,Sox10,Plp1,dkk3,Nrf2} {Frb,Nkd2,Tgfbr3,Hmox1,Cyba,Sox10,Lpr5,dkk3,Nrf2} {Frb,Nkd2,Tgfbr3,Hmox1,Cyba,Plp1,Lpr5,dkk3,Nrf2} {Frb,Nkd2,Tgfbr3,Hmox1,Sox10,Plp1,Lpr5,dkk3,Nrf2} {Frb,Nkd2,Tgfbr3,Cyba,Sox10,Plp1,Lpr5,dkk3,Nrf2} {Frb,Nkd2,Hmox1,Cyba,Sox10,Plp1,Lpr5,dkk3,Nrf2} {Frb,Tgfbr2,Tgfbr3,Hmox1,Cyba,Sox10,Plp1,Lpr5,dkk3} {Frb,Tgfbr2,Tgfbr3,Hmox1,Cyba,Sox10,Plp1,Lpr5,Nrf2} {Frb,Tgfbr2,Tgfbr3,Hmox1,Cyba,Sox10,Plp1,dkk3,Nrf2} {Frb,Tgfbr2,Tgfbr3,Hmox1,Cyba,Sox10,Lpr5,dkk3,Nrf2} {Frb,Tgfbr2,Tgfbr3,Hmox1,Cyba,Plp1,Lpr5,dkk3,Nrf2} {Frb,Tgfbr2,Tgfbr3,Hmox1,Sox10,Plp1,Lpr5,dkk3,Nrf2} {Frb,Tgfbr2,Tgfbr3,Cyba,Sox10,Plp1,Lpr5,dkk3,Nrf2} {Frb,Tgfbr2,Hmox1,Cyba,Sox10,Plp1,Lpr5,dkk3,Nrf2} {Frb,Tgfbr3,Hmox1,Cyba,Sox10,Plp1,Lpr5,dkk3,Nrf2} {Nkd2,Tgfbr2,Tgfbr3,Hmox1,Cyba,Sox10,Plp1,Lpr5,dkk3} {Nkd2,Tgfbr2,Tgfbr3,Hmox1,Cyba,Sox10,Plp1,Lpr5,Nrf2} {Nkd2,Tgfbr2,Tgfbr3,Hmox1,Cyba,Sox10,Plp1,dkk3,Nrf2} {Nkd2,Tgfbr2,Tgfbr3,Hmox1,Cyba,Sox10,Lpr5,dkk3,Nrf2} {Nkd2,Tgfbr2,Tgfbr3,Hmox1,Cyba,Plp1,Lpr5,dkk3,Nrf2} {Nkd2,Tgfbr2,Tgfbr3,Hmox1,Sox10,Plp1,Lpr5,dkk3,Nrf2} {Nkd2,Tgfbr2,Tgfbr3,Cyba,Sox10,Plp1,Lpr5,dkk3,Nrf2} {Nkd2,Tgfbr2,Hmox1,Cyba,Sox10,Plp1,Lpr5,dkk3,Nrf2} {Nkd2,Tgfbr3,Hmox1,Cyba,Sox10,Plp1,Lpr5,dkk3,Nrf2} {Tgfbr2,Tgfbr3,Hmox1,Cyba,Sox10,Plp1,Lpr5,dkk3,Nrf2} {Lgr6,Frb,Nkd2,Tgfbr2,Tgfbr3,Hmox1,Cyba,Sox10,Plp1,Lpr5} {Lgr6,Frb,Nkd2,Tgfbr2,Tgfbr3,Hmox1,Cyba,Sox10,Plp1,dkk3} {Lgr6,Frb,Nkd2,Tgfbr2,Tgfbr3,Hmox1,Cyba,Sox10,Plp1,Nrf2} {Lgr6,Frb,Nkd2,Tgfbr2,Tgfbr3,Hmox1,Cyba,Sox10,Lpr5,dkk3} {Lgr6,Frb,Nkd2,Tgfbr2,Tgfbr3,Hmox1,Cyba,Sox10,Lpr5,Nrf2} {Lgr6,Frb,Nkd2,Tgfbr2,Tgfbr3,Hmox1,Cyba,Sox10,dkk3,Nrf2} {Lgr6,Frb,Nkd2,Tgfbr2,Tgfbr3,Hmox1,Cyba,Plp1,Lpr5,dkk3} {Lgr6,Frb,Nkd2,Tgfbr2,Tgfbr3,Hmox1,Cyba,Plp1,Lpr5,Nrf2} {Lgr6,Frb,Nkd2,Tgfbr2,Tgfbr3,Hmox1,Cyba,Plp1,dkk3,Nrf2} {Lgr6,Frb,Nkd2,Tgfbr2,Tgfbr3,Hmox1,Cyba,Lpr5,dkk3,Nrf2} {Lgr6,Frb,Nkd2,Tgfbr2,Tgfbr3,Hmox1,Sox10,Plp1,Lpr5,dkk3} {Lgr6,Frb,Nkd2,Tgfbr2,Tgfbr3,Hmox1,Sox10,Plp1,Lpr5,Nrf2} {Lgr6,Frb,Nkd2,Tgfbr2,Tgfbr3,Hmox1,Sox10,Plp1,dkk3,Nrf2} {Lgr6,Frb,Nkd2,Tgfbr2,Tgfbr3,Hmox1,Sox10,Lpr5,dkk3,Nrf2} {Lgr6,Frb,Nkd2,Tgfbr2,Tgfbr3,Hmox1,Plp1,Lpr5,dkk3,Nrf2} {Lgr6,Frb,Nkd2,Tgfbr2,Tgfbr3,Cyba,Sox10,Plp1,Lpr5,dkk3} {Lgr6,Frb,Nkd2,Tgfbr2,Tgfbr3,Cyba,Sox10,Plp1,Lpr5,Nrf2} {Lgr6,Frb,Nkd2,Tgfbr2,Tgfbr3,Cyba,Sox10,Plp1,dkk3,Nrf2} {Lgr6,Frb,Nkd2,Tgfbr2,Tgfbr3,Cyba,Sox10,Lpr5,dkk3,Nrf2} {Lgr6,Frb,Nkd2,Tgfbr2,Tgfbr3,Cyba,Plp1,Lpr5,dkk3,Nrf2} {Lgr6,Frb,Nkd2,Tgfbr2,Tgfbr3,Sox10,Plp1,Lpr5,dkk3,Nrf2} {Lgr6,Frb,Nkd2,Tgfbr2,Hmox1,Cyba,Sox10,Plp1,Lpr5,dkk3} {Lgr6,Frb,Nkd2,Tgfbr2,Hmox1,Cyba,Sox10,Plp1,Lpr5,Nrf2} {Lgr6,Frb,Nkd2,Tgfbr2,Hmox1,Cyba,Sox10,Plp1,dkk3,Nrf2} {Lgr6,Frb,Nkd2,Tgfbr2,Hmox1,Cyba,Sox10,Lpr5,dkk3,Nrf2} {Lgr6,Frb,Nkd2,Tgfbr2,Hmox1,Cyba,Plp1,Lpr5,dkk3,Nrf2} {Lgr6,Frb,Nkd2,Tgfbr2,Hmox1,Sox10,Plp1,Lpr5,dkk3,Nrf2} {Lgr6,Frb,Nkd2,Tgfbr2,Cyba,Sox10,Plp1,Lpr5,dkk3,Nrf2} {Lgr6,Frb,Nkd2,Tgfbr3,Hmox1,Cyba,Sox10,Plp1,Lpr5,dkk3} {Lgr6,Frb,Nkd2,Tgfbr3,Hmox1,Cyba,Sox10,Plp1,Lpr5,Nrf2} {Lgr6,Frb,Nkd2,Tgfbr3,Hmox1,Cyba,Sox10,Plp1,dkk3,Nrf2} {Lgr6,Frb,Nkd2,Tgfbr3,Hmox1,Cyba,Sox10,Lpr5,dkk3,Nrf2} {Lgr6,Frb,Nkd2,Tgfbr3,Hmox1,Cyba,Plp1,Lpr5,dkk3,Nrf2} {Lgr6,Frb,Nkd2,Tgfbr3,Hmox1,Sox10,Plp1,Lpr5,dkk3,Nrf2} {Lgr6,Frb,Nkd2,Tgfbr3,Cyba,Sox10,Plp1,Lpr5,dkk3,Nrf2} {Lgr6,Frb,Nkd2,Hmox1,Cyba,Sox10,Plp1,Lpr5,dkk3,Nrf2} {Lgr6,Frb,Tgfbr2,Tgfbr3,Hmox1,Cyba,Sox10,Plp1,Lpr5,dkk3} {Lgr6,Frb,Tgfbr2,Tgfbr3,Hmox1,Cyba,Sox10,Plp1,Lpr5,Nrf2} {Lgr6,Frb,Tgfbr2,Tgfbr3,Hmox1,Cyba,Sox10,Plp1,dkk3,Nrf2} {Lgr6,Frb,Tgfbr2,Tgfbr3,Hmox1,Cyba,Sox10,Lpr5,dkk3,Nrf2} {Lgr6,Frb,Tgfbr2,Tgfbr3,Hmox1,Cyba,Plp1,Lpr5,dkk3,Nrf2} {Lgr6,Frb,Tgfbr2,Tgfbr3,Hmox1,Sox10,Plp1,Lpr5,dkk3,Nrf2} {Lgr6,Frb,Tgfbr2,Tgfbr3,Cyba,Sox10,Plp1,Lpr5,dkk3,Nrf2} {Lgr6,Frb,Tgfbr2,Hmox1,Cyba,Sox10,Plp1,Lpr5,dkk3,Nrf2} {Lgr6,Frb,Tgfbr3,Hmox1,Cyba,Sox10,Plp1,Lpr5,dkk3,Nrf2} {Lgr6,Nkd2,Tgfbr2,Tgfbr3,Hmox1,Cyba,Sox10,Plp1,Lpr5,dkk3} {Lgr6,Nkd2,Tgfbr2,Tgfbr3,Hmox1,Cyba,Sox10,Plp1,Lpr5,Nrf2} {Lgr6,Nkd2,Tgfbr2,Tgfbr3,Hmox1,Cyba,Sox10,Plp1,dkk3,Nrf2} {Lgr6,Nkd2,Tgfbr2,Tgfbr3,Hmox1,Cyba,Sox10,Lpr5,dkk3,Nrf2} {Lgr6,Nkd2,Tgfbr2,Tgfbr3,Hmox1,Cyba,Plp1,Lpr5,dkk3,Nrf2} {Lgr6,Nkd2,Tgfbr2,Tgfbr3,Hmox1,Sox10,Plp1,Lpr5,dkk3,Nrf2} {Lgr6,Nkd2,Tgfbr2,Tgfbr3,Cyba,Sox10,Plp1,Lpr5,dkk3,Nrf2} {Lgr6,Nkd2,Tgfbr2,Hmox1,Cyba,Sox10,Plp1,Lpr5,dkk3,Nrf2} {Lgr6,Nkd2,Tgfbr3,Hmox1,Cyba,Sox10,Plp1,Lpr5,dkk3,Nrf2} {Lgr6,Tgfbr2,Tgfbr3,Hmox1,Cyba,Sox10,Plp1,Lpr5,dkk3,Nrf2} {Frb,Nkd2,Tgfbr2,Tgfbr3,Hmox1,Cyba,Sox10,Plp1,Lpr5,dkk3} {Frb,Nkd2,Tgfbr2,Tgfbr3,Hmox1,Cyba,Sox10,Plp1,Lpr5,Nrf2} {Frb,Nkd2,Tgfbr2,Tgfbr3,Hmox1,Cyba,Sox10,Plp1,dkk3,Nrf2} {Frb,Nkd2,Tgfbr2,Tgfbr3,Hmox1,Cyba,Sox10,Lpr5,dkk3,Nrf2} {Frb,Nkd2,Tgfbr2,Tgfbr3,Hmox1,Cyba,Plp1,Lpr5,dkk3,Nrf2} {Frb,Nkd2,Tgfbr2,Tgfbr3,Hmox1,Sox10,Plp1,Lpr5,dkk3,Nrf2} {Frb,Nkd2,Tgfbr2,Tgfbr3,Cyba,Sox10,Plp1,Lpr5,dkk3,Nrf2} {Frb,Nkd2,Tgfbr2,Hmox1,Cyba,Sox10,Plp1,Lpr5,dkk3,Nrf2} {Frb,Nkd2,Tgfbr3,Hmox1,Cyba,Sox10,Plp1,Lpr5,dkk3,Nrf2} {Frb,Tgfbr2,Tgfbr3,Hmox1,Cyba,Sox10,Plp1,Lpr5,dkk3,Nrf2} {Nkd2,Tgfbr2,Tgfbr3,Hmox1,Cyba,Sox10,Plp1,Lpr5,dkk3,Nrf2} {Lgr6,Frb,Nkd2,Tgfbr2,Tgfbr3,Hmox1,Cyba,Sox10,Plp1,Lpr5,dkk3} {Lgr6,Frb,Nkd2,Tgfbr2,Tgfbr3,Hmox1,Cyba,Sox10,Plp1,Lpr5,Nrf2} {Lgr6,Frb,Nkd2,Tgfbr2,Tgfbr3,Hmox1,Cyba,Sox10,Plp1,dkk3,Nrf2} {Lgr6,Frb,Nkd2,Tgfbr2,Tgfbr3,Hmox1,Cyba,Sox10,Lpr5,dkk3,Nrf2} {Lgr6,Frb,Nkd2,Tgfbr2,Tgfbr3,Hmox1,Cyba,Plp1,Lpr5,dkk3,Nrf2} {Lgr6,Frb,Nkd2,Tgfbr2,Tgfbr3,Hmox1,Sox10,Plp1,Lpr5,dkk3,Nrf2} {Lgr6,Frb,Nkd2,Tgfbr2,Tgfbr3,Cyba,Sox10,Plp1,Lpr5,dkk3,Nrf2} {Lgr6,Frb,Nkd2,Tgfbr2,Hmox1,Cyba,Sox10,Plp1,Lpr5,dkk3,Nrf2} {Lgr6,Frb,Nkd2,Tgfbr3,Hmox1,Cyba,Sox10,Plp1,Lpr5,dkk3,Nrf2} {Lgr6,Frb,Tgfbr2,Tgfbr3,Hmox1,Cyba,Sox10,Plp1,Lpr5,dkk3,Nrf2} {Lgr6,Nkd2,Tgfbr2,Tgfbr3,Hmox1,Cyba,Sox10,Plp1,Lpr5,dkk3,Nrf2} {Frb,Nkd2,Tgfbr2,Tgfbr3,Hmox1,Cyba,Sox10,Plp1,Lpr5,dkk3,Nrf2} or {Lgr6,Frb,Nkd2,Tgfbr2,Tgfbr3,Hmox1,Cyba,Sox10,Plp1,Lpr5,dkk3,Nrf2}.

Alternatively or in addition, any of the cells or tissues of the invention (for example: the ANPG cell; stem cell, pluripotent cell or progenitor cell capable of differentiating into an ANPG cell; ANPG cells comprised in a neurosphere and/or comprised in an organoid; auditory neuron; glial cell; sensory epithelial cell; SGN; sensory epithelium of the cochlea; and/or a HC) may underexpress or overexpress one or more genes relating to PNS development, TGFβ, Gliogenesis, Wnt, NADPH oxidase and TYROBP, as set out in Table 2. As such, the underexpressed gene may be one or more genes set out in Table 2. By way of example, the underexpressed gene may be {Sh3tc2}, {Tenm4}, {Gdnf and Spp1}, or {Wnt7a, Lgr5, Hmox1, Cyba and Hlx}.

TABLE 2
Genes that may be underexpressed, grouped by ontogeny

PNS

| Development | Gliogenesis | TYROBP | | TGFβ | Wnt |
|---|---|---|---|---|---|
| Sh3tc2 | Trem2 | Hlx | Cxcl16 | Inhba | Wnt7a |
| Gdnf | Tenm4 | Tmem106a | Itgb2 | Smad7 | Wnt7b |
| Artn | Clcf1 | Hcls1 | Maf | Bambi | Lgr5 |
| Egr3 | Atxn1 | Cd84 | Sh2b3 | Serpine1 | Lgr6 |
| Itgb4 | Mdk | Stat5a | Tnfrsf1b | Tgif1 | Dkk3 |
| Cited2 | Tspo | Nckap1l | Dpyd | Eng | Nkd2 |
| Lgi4 | Bmp2 | Tyrobp | Creb3l2 | Nfkb1 | Frzb |
| Erbb3 | Tnfrsf1b | C1qc | Lhfpl2 | Runx2 | |
| Sox10 | P2rx4 | Plek | Capg | Tgfb1 | |
| NADPH Oxidase | Igf1 | Itgam | C3 | Smad5 | |
| Hmox1 | Tgfb1 | Apbb1ip | Spp1 | Lif | |
| Cyba | Lif | | | Tgfbr3 | |
| Cybb | Cx3cl1 | | | Thbs1 | |
| | Csf1 | | | Tgfbr2 | |
| | Sox10 | | | Zeb2 | |
| | Trf | | | Jak1 | |
| | | | | Spp | |

### Cell and Tissue Culture

The invention relates to a method of obtaining, maintaining and/or expanding an ANPG cell or neurosphere comprising ANPG cells as defined above. The method may comprise differentiating a stem cell and/or progenitor cell as defined above into the ANPG cell and/or neurosphere comprising ANPG cells. Differentiation may take place in any cell culture medium suitable for differentiating the stem and/or progenitor, and may include a differentiation medium known in the art. Alternatively, the method may comprise isolating an ANPG cell from a spiral ganglion. The spiral ganglion may be from any mammal, such as a rodent, bovine, caprine, equine, ovine, porcine, leporine, canine, feline, non-human primate and human spiral ganglion. Preferably, the spiral ganglion may be from a mouse such as an A/J mouse or a human. The obtained ANPG cell or neurosphere comprising ANPG cells may be subjected to maintenance or expansion culture using methods known in the art such as by: (i) fully or partially removing culture medium; (ii) optionally mechanically and/or chemically dissociating the cells from each other (either partially of substantially fully); (iii) optionally transferring the ANPG cell or neurosphere comprising ANPG cells into a new culture vessel; (iv) adding fresh culture medium and allowing for continued culture; and optionally repeating steps (i)-(iv) one or more times. During maintenance and expansion culture, functional and structural characteristics of the ANPG cell or neurosphere comprising ANPG cells do not substantially change. During maintenance culture, the number of NPG cell or neurosphere comprising ANPG cells may remain substantially the same. During expansion culture, the number of ANPG cell or neurosphere comprising ANPG cells may be increased.

The culture medium used for obtaining (including differentiating from a stem and/or progenitor cell), maintaining and/or expanding an ANPG cell or neurosphere comprising ANPG cells may contain at least one growth factor. The growth factor may be a mitogenic growth factor such as VEGF, PDGF and EGF, and/or selected from one or more of FGF, EGF, IGF and Heparan Sulfate. Thus, the growth factor may be: VEGF; PDGF; FGF; EGF; IGF; Heparan Sulfate; VEGF and PDGF; VEGF and FGF; VEGF and EGF; VEGF and IGF; VEGF and Heparan Sulfate; PDGF and FGF; PDGF and EGF; PDGF and IGF; PDGF and Heparan Sulfate; FGF and EGF; FGF and IGF; FGF and Heparan Sulfate; EGF and IGF; EGF and Heparan Sulfate; IGF and Heparan Sulfate; VEGF, PDGF and FGF; VEGF, PDGF and EGF; VEGF, PDGF and IGF; VEGF, PDGF and Heparan Sulfate; VEGF, FGF and EGF; VEGF, FGF and IGF; VEGF, FGF and Heparan Sulfate; VEGF, EGF and IGF; VEGF, EGF and Heparan Sulfate; VEGF, IGF and Heparan Sulfate; PDGF, FGF and EGF; PDGF, FGF and IGF; PDGF, FGF and Heparan Sulfate; PDGF, EGF and IGF; PDGF, EGF and Heparan Sulfate; PDGF, IGF and Heparan Sulfate; FGF, EGF and IGF; FGF, EGF and Heparan Sulfate; FGF, IGF and Heparan Sulfate; EGF, IGF and Heparan Sulfate; VEGF, PDGF, FGF and EGF; VEGF, PDGF, FGF and IGF; VEGF, PDGF, FGF and Heparan Sulfate; VEGF, PDGF, EGF and IGF; VEGF, PDGF, EGF and Heparan Sulfate; VEGF, PDGF, IGF and Heparan Sulfate; VEGF, FGF, EGF and IGF; VEGF, FGF, EGF and Heparan Sulfate; VEGF, FGF, IGF and Heparan Sulfate; VEGF, EGF, IGF and Heparan Sulfate; PDGF, FGF, EGF and IGF; PDGF, FGF, EGF and Heparan Sulfate; PDGF, FGF, IGF and Heparan Sulfate; PDGF, EGF, IGF and Heparan Sulfate; FGF, EGF, IGF and Heparan Sulfate; VEGF, PDGF, FGF, EGF and IGF; VEGF, PDGF, FGF, EGF and Heparan Sulfate; VEGF, PDGF, FGF, IGF and Heparan Sulfate; VEGF, PDGF, EGF, IGF and Heparan Sulfate; VEGF, FGF, EGF, IGF and Heparan Sulfate; PDGF, FGF, EGF, IGF and Heparan Sulfate; or VEGF, PDGF, FGF, EGF, IGF and Heparan Sulfate. The final concentration of each of the individual growth factors in the medium may be about 10 ng/mL, about 25 ng/mL, about 50 ng/mL, about 100 ng/mL or about 250 ng/mL. These values may be combined to obtain a range such as: 10 ng/mL-25 ng/mL, 10 ng/mL-50 ng/mL, 10 ng/mL-100 ng/mL, 10 ng/mL-250 ng/mL, 25 ng/mL-50 ng/mL, 25 ng/mL-100 ng/mL, 25 ng/mL-250 ng/mL, 50 ng/mL-100 ng/mL, 50 ng/mL-250 ng/mL or 100 ng/mL-250 ng/mL. Where multiple growth factors are present, the concentration of each of the growth factors may be the same or different. For example, the concentration of EGF and IGF may both be about 25 ng/mL. Alternatively, the concentration of EGF may be about 25 ng/mL and the concentration of IGF about 50 ng/mL.

The invention also relates to a method of differentiating the ANPG cell or neurosphere comprising ANPG cells as defined above. For example, the ANPG cell or neurosphere comprising ANPG cells may be differentiated into one or more cells selected from: an auditory neuron; glial cell; sensory epithelial cell; SGN; sensory epithelium of the cochlea; and a HC. Differentiation may be achieved via direct conversion of one cell type to another. Alternatively, differentiation may be achieved by conversion via one or more intermediate cell types. At least 1%, at least 10%, at least 25%, at least 50%, at least 75%, at least 80%, at least 90%, at least 95% or at least 99% of the ANPG cell or ANPG cells comprised in a neurosphere may be differentiated into an auditory neuron; a glial cell; a sensory epithelial cell; a SGN; a sensory epithelium of the cochlea or a HC.

The ANPG cell or neurosphere comprising ANPG cells may be differentiated by culturing in a suitable culture medium. The culture medium for differentiating the ANPG cell or neurosphere comprising ANPG cells may be any suitable differentiation culture medium, and may contain at least one growth factor. The at least one growth factor may be the same growth factor or growth factors present in the culture medium described above used for obtaining (including differentiating from a stem and/or progenitor cell), maintaining and/or expanding an ANPG cell or neurosphere comprising ANPG cells. As such, the growth factor in the medium used for differentiating the ANPG cell or neurosphere comprising ANPG cells may be a mitogenic growth factor such as VEGF, PDGF and EGF, and/or selected from one or more of FGF, EGF, IGF and Heparan Sulfate. The concentration of the growth factor in the medium used for differentiating the ANPG cell or neurosphere comprising ANPG cells may be less than the concentration of the same growth factor in the medium described above used for obtaining (including differentiating from a stem and/or progenitor cell), maintaining and/or expanding an ANPG cell or neurosphere comprising ANPG cells. Thus, differentiation of the ANPG cell or neurosphere comprising ANPG cells may be achieved by reducing the concentration of one or more growth factors. The concentration of each of the one or more growth factors may be reduced by at least 1%, at least 5%, at least 10%, at least 20%, at least 30%, at least 40%, at least 50%, at least 60%, at least 70%, at least 80%, at least 90%, at least 95%, at least 99%, or by 100%. For example, the concentration of EGF may be reduced from about 25 ng/mL to about 5 ng/mL (i.e. reduced by 80%), and the concentration of IGF reduced from about 50 ng/mL to about 25 ng/mL (i.e. reduced by 50%).

Additionally or alternatively, the medium for differentiating an ANPG cell or neurosphere comprising ANPG cells may comprise one or more neurotrophinin. The neurotrophinin may be selected from one or more of nerve growth factor (NGF), brain-derived neurotrophic factor (BDNF), neurotrophin-3 (NT-3) and neurotrophin-4 (NT-4). Thus, the medium for differentiating an ANPG cell or neurosphere comprising ANPG cells may comprise: NGF; BDNF; NT-3; NT-4; NGF and BDNF; NGF and NT-3; NGF and NT-4; BDNF and NT-3; BDNF and NT-4; NT-3 and NT-4; NGF, BDNF and NT-3; NGF, BDNF and NT-4; NGF, NT-3 and NT-4; BDNF, NT-3 and NT-4; or NGF, BDNF, NT-3 and NT-4. The final concentration of each of the individual neurotrophinin in the medium may be about 1 μg/mL, about 5 μg/mL, about 10 μg/mL, about 25 μg/mL, about 50 μg/mL or about 100 μg/mL. These values may be combined to obtain a range such as: 1 μg/mL-5 μg/mL, 1 μg/mL-10 μg/mL, 1 μg/mL-25 μg/mL, 1 μg/mL-50 μg/mL, 1 μg/mL-100 μg/mL, 5 μg/mL-10 μg/mL, 5 μg/mL-25 μg/mL, 5 μg/mL-50 μg/mL, 5 μg/mL-100 μg/mL, 10 μg/mL-25 μg/mL, 10 μg/mL-50 μg/mL, 10 μg/mL-100 μg/mL, 25 μg/mL-50 μg/mL, 25 μg/mL-100 μg/mL, or 50 μg/mL-100 μg/mL. Where multiple neurotrophinins are used, the concentration of each of the neurotrophinins may be the same or different. For example, the concentration of NT-3 and BDNF may both be about 10 μg/mL. Alternatively, the concentration of NT-3 may be about 10 μg/mL and the concentration of BDNF about 25 μg/mL.

Culture of any of the cells or tissues herein (for example: the ANPG cell; stem cell, pluripotent cell or progenitor cell capable of differentiating into an ANPG cell; ANPG cells comprised in a neurosphere and/or comprised in an organoid; auditory neuron; glial cell; sensory epithelial cell; SGN; sensory epithelium of the cochlea; and/or a HC) may increase the quantity of said cell or tissue (i.e. expansion culture). The number of cells or quantity of tissue may be expanded (i.e. increased) by at least 2 times, at least 5 times, at least 10 times, at least 1×10² times, at least 1×10³ times, at least 1×10⁴ times, at least 1×10⁵ times, at least 1×10⁶ times, at least 1×10⁷ times, at least 1×10⁸ times, at least 1×10⁹ times, at least 1×10¹⁰ times, at least 1×10¹¹ times, or at least 1×10¹² times. These values may be combined to obtain a range such as: 1×10⁵-1×10⁶, 1×10⁵-1×10⁷, 1×10⁵-1×10⁸, 1×10⁵-1×10⁹, 1×10⁵-1×10¹⁰, 1×10⁵-1×10¹¹, 1×10⁵-1×10¹², 1×10⁶-1×10⁷, 1×10⁶-1×10⁸, 1×10⁶-1×10⁹, 1×10⁶-1×10¹⁰, 1×10⁶-1×10¹¹, 1×10⁶-1×10¹², 1×10⁷-1×10⁸, 1×10⁷-1×10⁹, 1×10⁷-1×10¹⁰, 1×10⁷-1×10¹¹, 1×10⁷-1×10¹², 1×10⁸-1×10⁹, 1×10⁸-1×10¹⁰, 1×10⁸-1×10¹¹, 1×10⁸-1×10¹², 1×10⁹-1×10¹⁰, 1×10⁹-1×10¹¹, 1×10⁹-1×10¹², 1×10¹⁰-1×10¹¹, 1×10¹⁰-1×10¹², or 1×10¹¹-1×10¹².

Culture methods of the invention may or may not require the presence of a feeder cell. A feeder cell may therefore be substantially absent or totally absent. A feeder cell is a cell upon which the isolated cell or tissue may be cultured on to support self-renewal, pluripotency and/or differentiation, typically by secreting growth factors, cytokines and/or extracellular matrix proteins and are themselves unable to divide. A typical example of a feeder cell is a mouse embryonic fibroblast that has been mitotically inactivated.

Two-dimensional (2D) or three-dimensional (3D) cell culture may be used as appropriate. The skilled person would understand the term 3D cell culture, and how it differs from 2D cell culture. For example, if would be understood that 2D cell culture systems involve the growth of cells on substantially flat surfaces typically made of plastic as a monolayer of cells and/or as a cell sheet. These substantially flat surfaces may be coated with, for example, a feeder layer of cells and/or a chemical such as poly-D-lysine. 3D culture is any culture that results in a cluster of cells in three spatial dimensions, as opposed to a monolayer of cells growing substantially in only two spatial dimensions. Thus, in 3D culture systems, cells are not cultured as a monolayer and/or cell sheet. 3D culture may be achieved in a number of non-mutually exclusive ways. Any one or more suitable 3D cell culture system may be used, and the skilled person would be able to readily determine suitable one or more 3D cell culture systems. For example, cells, may be seeded in the non-adherent (i.e. suspension culture) system, which may be a non-adherent plate with or without agitation, or a receptacle such as a flask with or without stirring, and 3D cell structures such as a neurospheres allowed to develop. In such a system, single cells and/or cell aggregates multiply and/or divide in a liquid medium. Alternatively, a 3D cell culture system may comprise culturing substantially single cells and/or clusters of cells in a concentrated medium and/or in medium containing substances with gelling properties (such as dissolved low-melting agarose with cell medium and/or a multiprotein hydrogel such as Matrigel) to obtain 3D cell structures such as a neurosphere and/or an organoid. Alternatively, a 3D cell culture system may comprise culturing cells, on a scaffold to obtain a 3D cell structures such as a neurosphere and/or an organoid. A scaffold may be cell-free and provides a way for cells to grow in three dimensions. These are typically provided through a cell-free matrix. Examples of suitable scaffolds include silk, collagen, laminin and/or alginate.

In any of the cell culture methods described, batch culture and/or continuous culture may be used. Batch culture is a closed system culture in which cells are grown in a fixed amount of culture medium under suitable environmental conditions. The culture medium may be replaced as necessary, for example by substantially isolating the cells in suspension (for example by centrifugation) and re-suspending the substantially isolated cells in culture medium. Examples of batch culture systems include: slowly rotating cultures, shake cultures, spinning cultures and stirred cultures. Generally, it is the substantially continuous shear stress that is beneficial to cell culture in such 3D culture systems. The 3D cell culture of the invention may be stirred at about 10 rpm, about 40 rpm, about 55 rpm, about 75 rpm or about 90 rpm. These values may be combined to obtain ranges such as: 10 rpm-40 rpm, 10 rpm-55 rpm, 10 rpm-75 rpm, 10 rpm-90 rpm, 40 rpm-55 rpm, 40 rpm-75 rpm, 40 rpm-90 rpm, 55 rpm-75 rpm, 55 rpm-90 rpm or 75 rpm-90 rpm. The stirring may be performed in any suitable way known to the skilled person, such as through the use of a magnetic bead or through the use of a spinner flask. Continuous culture can be an open or closed system cultures. In continuous culture systems, fresh medium is continuously added and the build-up of toxins in the medium avoided by continuously removing leftover nutrients and metabolic end products from the medium. Continuous culture may be especially suitable for growing large-scale cultures in bioreactors. In a closed system continuous culture, the addition of fresh medium is balanced by the outflow of old medium. So, cell biomass continues to increase with the growth of the cell. In the open system continuous culture, a balance is maintained by adding the fresh medium while harvesting the same amount of the culture (containing both, cell and the medium). Examples of continuous culture systems include: a chemostat (where fresh medium is added while the same amount of the culture is harvested, maintaining steady cell growth), and a turbidostat (where the biomass of the cells are constantly maintained at the level below the maximum yield with all nutrients being present in excess, unlike one limiting factor as in chemostat culture). In any one of the 3D culture systems, 3D cell structures such as neurospheres comprising ANPG cells and/or organoids containing ANPG cells can be extracted from the culture system and further processed as described.

The ANPG cells obtained by the method of the invention may be comprised in a 3D cluster (i.e. in a neurosphere and/or an organoid). The neurosphere may or may not be dissociated into smaller clusters of cells and/or substantially single cells, for example using chemical and/or mechanical means. Each neurosphere may comprise, on average, at least 10, at least 100, at least 1000, at least 10,000, at least 100,000, or at least 1,000,000 ANPG cells. These values may be combined to obtain a range such as: 10-100, 10-1,000, 10-10,000, 10-100,000, 10-1,000,000, 100-1,000, 100-10,000, 100-100,000, 100-1,000,000, 1,000-10,000, 1,000-100,000, 1,000-1,000,000, 10,000-100,000, 10,000-1,000,000, or 100,000-1,000,000 ANPG cells. In addition or in the alternative, each cluster may comprise on average at least 50%, at least 60%, at least 70%, at least 80%, at least 90%, at least 95% or at least 99% ANPG cells. These values may be combined to obtain a range such as: 50%-60%, 50%-70%, 50%-80%, 50%-90%, 50%-95%, 50%-99%, 60%-70%, 60%-80%, 60%-90%, 60%-95%, 60%-99%, 70%-80%, 70%-90%, 70%-95%, 70%-99%, 80%-90%, 80%-95%, 80%-99%, 90%-95%, 90%-99%, or 95%-99% ANGP cells. The degree of purity of neurosphere may be such that it is not necessary to purify the ANPG cells. For example, the neurospheres may be used directly in further applications such as in screening and/or therapeutic applications set out herein without any further purification. Alternatively, the neurospheres may be partially or fully dissociated and the dissociated cells used directly in further applications such as in screening and/or therapeutic applications set out herein without any further purification, without the need for dissociated ANPG cells to be specifically purified.

The skilled person would readily be able to determine an appropriate amount of time for each culture step. For example, culturing may occur for about 0.5 days, about 1 day, about 2 days, about 3 days, about 4 days, about 5 days, about 6 days, about 7 days, about 8 days, about 9 days, about 10 days, about 12 days, or about 14 days. These values may be combined to obtain a range such as: 0.5-1 days, 0.5-2 days, 0.5-3 days, 0.5-4 days, 0.5-5 days, 0.5-6 days, 0.5-7 days, 0.5-8 days, 0.5-9 days, 0.5-10 days, 0.5-12 days, 0.5-14 days, 1-2 days, 1-3 days, 1-4 days, 1-5 days, 1-6 days, 1-7 days, 1-8 days, 1-9 days, 1-10 days, 1-12 days, 1-14 days, 2-3 days, 2-4 days, 2-5 days, 2-6 days, 2-7 days, 2-8 days, 2-9 days, 2-10 days, 2-12 days, 2-14 days, 3-4 days, 3-5 days, 3-6 days, 3-7 days, 3-8 days, 3-9 days, 3-10 days, 3-12 days, 3-14 days, 4-5 days, 4-6 days, 4-7 days, 4-8 days, 4-9 days, 4-10 days, 4-12 days, 4-14 days, 5-6 days, 5-7 days, 5-8 days, 5-9 days, 5-10 days, 5-12 days, 5-14 days, 6-7 days, 6-8 days, 6-9 days, 6-10 days, 6-12 days, 6-14 days, 7-8 days, 7-9 days, 7-10 days, 7-12 days, 7-14 days, 8-9 days, 8-10 days, 8-12 days, 8-14 days, 9-10 days, 9-12 days, 9-14 days, 10-12 days, 10-14 days, or 12-14 days.

Any of the cells and/or tissues of the invention (and in particular: ANPG cells; ANPG cells comprised in a neurosphere and/or comprised in an organoid; auditory neurons; glial cells; sensory epithelial cells; SGNs; sensory epithelium of the cochlea; and/or a HCs) may be obtained within 1 day, within 2 days, within 3 days, within 4 days, within 5 days, within 6 days, within 1 week, within 2 weeks, within 3 weeks, or within 4 weeks. In general, when longer culture periods are used, more media changes (i.e. more reseeding of cells in fresh media) will be required. The skilled person would readily be able to determine when media change(s) are required. In one embodiment, the invention provides ANPG cells; ANPG cells comprised in a neurosphere; auditory neurons; glial cells; sensory epithelial cells; SGNs; sensory epithelium of the cochlea; and/or HCs obtained or obtainable by the method of the invention set out herein.

To avoid contamination with a chemically-undefined component, any one or more medium to be used in the present invention may be a medium whose components are chemically-defined. In the present invention, any one or more culturing step may be performed under xeno-free conditions (i.e. conditions eliminating components derived from species different from that of the cell to be cultured).

### Agonists and Antagonists

The aforementioned WNT agonist (Wnt signal transduction pathway agonist) is a substance capable of enhancing signal transduction mediated by Wnt. The WNT agonist is not particularly limited as long as it enhances the signal transduction pathway caused by WNT. Examples of the Wnt signal transduction pathway agonist include proteins belonging to the Wnt family (e.g., Wnt1, Wnt3A, Wnt7A, Wnt7b, Wnt2B), a Wnt receptor, a Wnt receptor agonist, an anti-Wnt receptor antibody, a Wnt partial peptide, a β catenin signal transmitter, a GSK3β inhibitor (e.g., 6-Bromoindirubin-3′-oxime (BIO), CHIR99021, Kenpaullone), L-Quebrachitol, Methyl Vanillate, Isoxazole 9 (ISX-9), CGP 57380, CP21R7 (CP21), Wnt agonist 1 (BML-284 HCL, AMBMP HCL), WAY-262611 (BML-WN110), WAY-316606, and SKL2001. In one preferred embodiment, the Wnt agonist is CHIR99021.

The aforementioned TGFβ antagonist (TGFβ family signal transduction pathway inhibitor) refers to a substance that inhibits the TGFβ family signal transduction pathway, i.e., a signal transduction pathway transmitted by the Smad family, and specific examples include a TGFβ signal transduction pathway inhibitor, a Nodal/Activin signal transduction pathway inhibitor and a BMP signal transduction pathway inhibitor. The TGFβ signal transduction pathway inhibitor is not particularly limited as long as it inhibits the signal transduction pathway caused by TGFβ, and may be any of a nucleic acid, a protein, and a low-molecular-weight compound. Examples of the inhibitor can include a substance that act directly on TGFβ (e.g., a protein, antibody, an aptamer), a substance that suppresses expression of a gene encoding TGFβ (e.g., an antisense oligonucleotide such as an siRNA), a substance that inhibits the binding of a TGFβ receptor and TGFβ, and a substance that inhibits a physiological activity caused by signal transduction by TGFβ receptor (e.g., a TGFβ receptor inhibitor or a Smad inhibitor). A protein known as a TGFβ signal transduction pathway inhibitor, Lefty and the like can be mentioned. As a TGFβ signal transduction pathway inhibitor, a compound well known to those of ordinary skill in the art can be used and, such as one or more of Lefty, LDN193189, SB431542 (4[4-(1,3-benzodioxol-5-yl)-5-(2-pyridinyl)-1H-imidazol-2-yl]benzamide), LY-364947 (4-[3-(2-pyridinyl)-1H-pyrazol-4-yl]-quinoline), SB-505124 (2-(5-benzo[1,3]dioxol-5-yl-2-tert-butyl-3H-imidazol-4-yl)-6-methylpyridine), Galunisertib (LY2157299), LY2109761, SB525334, Dorsomorphin, Pirfenidone (5-7701, AMR-69), GW788388, RepSox, Sulfasalazine (NSC 667219, Azulfidine, Salazopyrin, Sulphasalazine), K02288, SD-208, AUDA (compound 43), TP0427736, LDN-214117, SIS3, BIBF-0775, LY 3200882, Vactosertib (TEW-7197), DMH1, LDN-212854, ML347 (LDN-193719), Halofuginone (RU-19110), ITD-1, TA-02, PD 169316, and A-83-01 (3-(6-methyl-2-pyridinyl)-N-phenyl-4-(4-quinolinyl)-1H-pyrazole-1-carbothioamide). In one preferred embodiment, the TGFβ antagonist is one or more Smad inhibitor. In a further preferred embodiment, the TGFβ antagonist is LDN193189 and/or SB431542. The use of both LDN193189 and SB431542 may be referred to as double Smad (DS) inhibition.

The concentration of the WNT agonist and TGFβ antagonist is not particularly limited and would be able to be easily determined by those of ordinary skill in the art. For example, the skilled person would be able to compare a control cell not treated with the WNT agonist and/or TGFβ antagonist with a test cell treated with the WNT agonist and/or TGFβ antagonist. In doing so, and using methods set out herein and known in the art, the skilled person would be able to determine whether or not a particular concentration (and combination) of a WNT agonist and/or TGFβ antagonist results in: enhanced stemness/self-renewal; enhanced differentiation capacity into an auditory neuron and/or glial cell; increased maintenance of self-renewal and/or differentiation capacity after freeze-thawing; overexpression of one or more genes selected from Myc, Sox2, Lgr5, Wnt7a, Wnt7b, Bmi1, Rtkn2 and a gene set out in Table 1; and/or underexpression of one or more genes selected from Lgr6, Frb, Nkd2, Tgfbr2, Tgfbr3, Hmox1, Cyba, Sox10, Plp1, Lpr5, dkk3, Nrf2 and/or a gene set out in Table 2.

Thus, the invention also provides a method for culturing any cell and/or tissue (and in particular: stem cells, pluripotent cells or progenitor cells, ANPG cells, ANPG cells comprised in a neurosphere and/or comprised in an organoid, auditory neurons, glial cells, sensory epithelial cells, SGNs, sensory epithelium of the cochlea, and/or a HCs) with a WNT agonist such as CHIR99021, and a TGFβ antagonist such as LDN193189 and/or SB431542. The cultured cell and/or tissue may be from any origin. Also provided are cells produced and/or producible by such a method. In doing so enhanced stemness/self-renewal; enhanced differentiation capacity into an auditory neuron and/or glial cell; increased maintenance of self-renewal and/or differentiation capacity after freeze-thawing; overexpression of one or more genes selected from Myc, Sox2, Lgr5, Wnt7a, Wnt7b, Bmi1, Rtkn2 and a gene set out in Table 1; and/or underexpression of one or more genes selected from Lgr6, Frb, Nkd2, Tgfbr2, Tgfbr3, Hmox1, Cyba, Sox10, Plp1, Lpr5, dkk3, Nrf2 and/or a gene set out in Table 2 may be achieved.

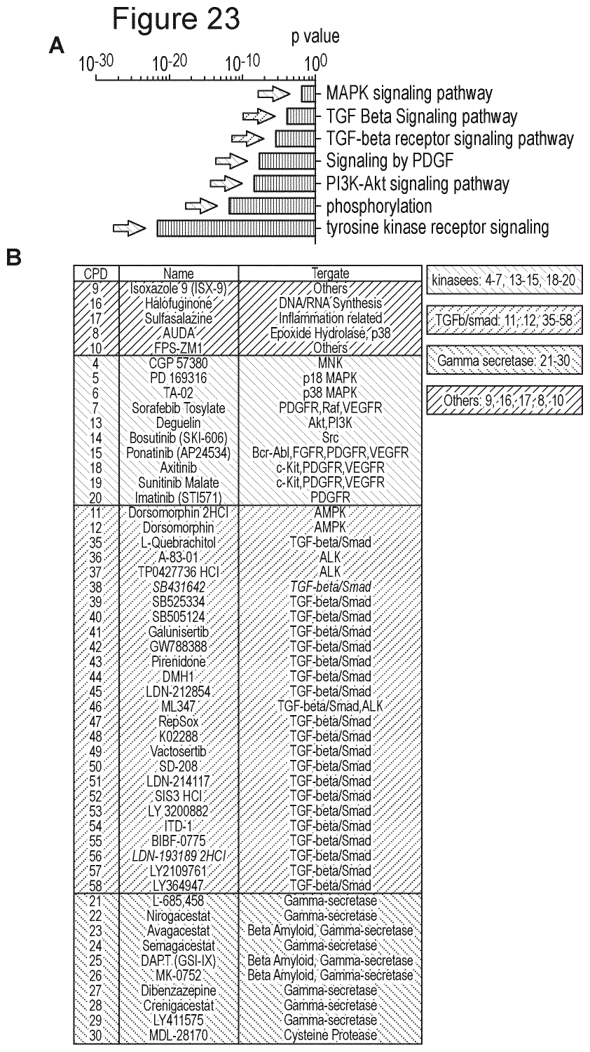

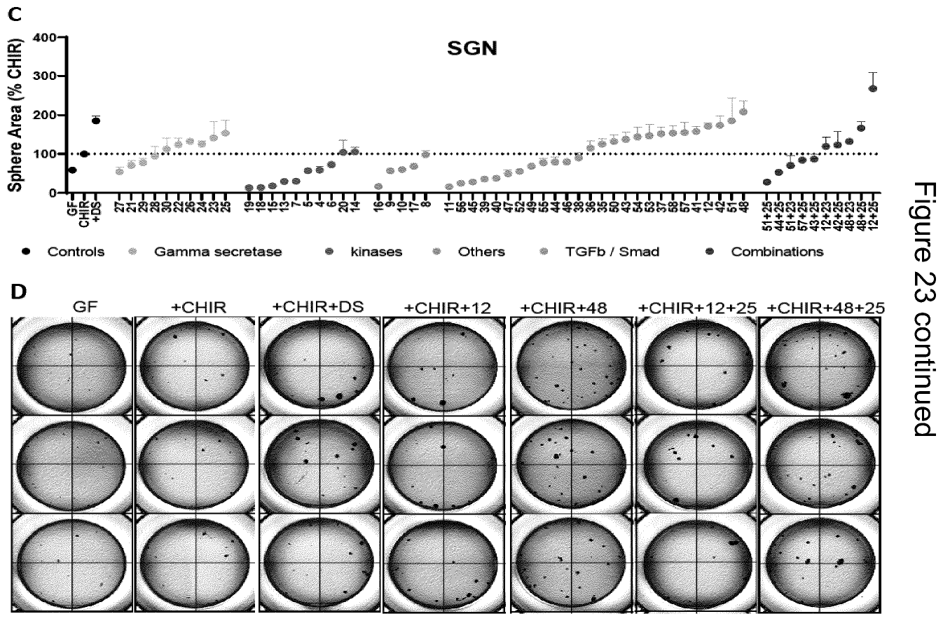

In addition, or in the alternative, any suitable pharmacological modulator may be used to induce and/or enhance the reprogramming and/or regeneration of ANPG cells. The pharmacological modulator may be any one or more modulator selected from the Table in FIG. 23B. The modulator may activate or inhibit one or more of the targets selected from the Table in FIG. 23B. The modulator may be selected from one or more of Isoxazole9 (ISX-9), Halofuginone, Sulfasalazine, AUDA, FPS-ZM1, CGP 57380, PD 169316, TA-02, Sorafenib Tosylate, Deguelin, Bosutinib (SKI-606), Ponatinib (AP24534), Axitinib, SunitinibMalate, Imatinib (STI571), Dorsomorphin 2HCl, Dorsomorphin, L-Quebrachitol, A-83-01, TP0427736 HCl, SB431542, SB525334, SB505124, Galunisertib, GW788388, Pirfenidone, DMH1, LDN-212854, ML347, RepSox, K02288, Vactosertib, SD-208, LDN-214117, SIS3 HCl, LY 3200882, ITD-1, BIBF-0775, LDN-193189 2HCl, LY2109761, LY364947, L-685458, Nirogacestat, Avagacestat, Semagacestat, DAPT (GSI-IX), MK-0752, Dibenzazepine, Crenigacestat, LY411575 and MDL-28170. For example, the modulator may be selected from one or more of dorsomorphin, GW788388, LDN-214117, K0228, K0228 and DAPT, and dorsomorphin and DAPT. For the induction and/or enhanced reprogramming and/or regeneration of human fetal auditory progenitors, the modulator may be selected from one or more of LDN-214117, LY 3200882, dorsomorphin, dorsomorphin and crenigacestat, and dorsomorphin and TA 02. The duration and final concentration of each modulator may be determined by the skilled person using tests and procedures known in the art. For example, the final concentration of each of the individual modulators in the medium may be about 1 μM, about 3 μM, about 5 μM, or about 10 μM. These values may be combined to obtain a range such as: 1 μM-5 μM or 3 μM-10 μM. Where multiple modulators are used, the concentration of each of the modulators may the same or different.

Purifying and Cryopreserving Cells and/or Tissues

Any of the cells and/or tissues discussed herein (for example: the ANPG cell; stem cell, pluripotent cell or progenitor cell capable of differentiating into an ANPG cell; ANPG cells comprised in a neurosphere and/or comprised in an organoid; auditory neuron; glial cell; sensory epithelial cell; SGN; sensory epithelium of the cochlea; and/or a HC), may be selectively purified and/or selectively enriched form a heterologous population. Purification and/or enrichment may be carried out for downstream therapeutic and/or research applications. The cells and/or tissues may be expanded. A population of cells may be purified selectively for cells that exhibit a specific phenotype or characteristic, and from other cells which do not exhibit that phenotype or characteristic, or exhibit it to a lesser degree. For example, a population of cells that expresses a specific marker or markers may be purified from a starting population of cells. Alternatively, or in addition, a population of cells that does not express another marker may be purified. In one embodiment, the cell to be selectively purified and/or selectively enriched may: overexpress of one or more gene selected from Myc, Sox2, Lgr5, Wnt7a, Wnt7b, Bmi1, Rtkn2; and/or underexpress one or more gene selected from Lgr6, Frb, Nkd2, Tgfbr2, Tgfbr3, Hmox1, Cyba, Sox10, Plp1, Lpr5, dkk3 and Nrf2. Alternatively, or in addition, the cell to be selectively purified and/or selectively enriched may: overexpress of one or more gene selected from Table 1; and/or underexpress one or more gene selected from Table 2.

By “enriching” a population of cells for a certain type of cells it is to be understood that the concentration of that type of cells is increased within the population. The concentration of other types of cells may be concomitantly reduced.

Purification or enrichment may result in the population of cells being substantially pure of other types of cell. Purifying or enriching for a population of cells expressing a specific marker may be achieved by using an agent that binds to that marker, preferably substantially specifically to that marker.

An agent that binds to a cellular marker may be an antibody. The term “antibody” refers to complete antibodies or antibody fragments capable of binding to a selected target, and including Fv, ScFv, F(ab′) and F(ab′)2, monoclonal and polyclonal antibodies, engineered antibodies including chimeric, CDR-grafted and humanised antibodies, and artificially selected antibodies produced using phage display or alternative techniques. In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification, or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead, or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing or not expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such biotin columns to separate avidin-labelled agents) and microscopy-based techniques. These techniques may be qualitative in nature, semi-quantitative in nature or quantitative in nature. It may also be possible to perform the separation using a combination of different techniques, such as a magnetic bead-based separation step followed by sorting of the resulting population of cells for one or more additional (positive or negative) markers by flow cytometry. Clinical grade separation may be performed, for example, using the CliniMACS® system (Miltenyi). This is an example of a closed-circuit magnetic bead-based separation technology. It is also envisaged that dye exclusion properties or enzymatic activity may be used to enrich for a desired cell type.

Any of the cells and/or tissues discussed herein (for example: the ANPG cell; stem cell, pluripotent cell or progenitor cell capable of differentiating into an ANPG cell; ANPG cells comprised in a neurosphere and/or comprised in an organoid; auditory neuron; glial cell; sensory epithelial cell; SGN; sensory epithelium of the cochlea; and/or a HC) may be cryopreserved, either directly (e.g. after isolation, after being obtained, after purification and/or after enrichment) or after sub-culture (e.g. after maintenance and/or expansion culture). In particular, auditory neurons may be cryopreserved. Cryopreservation is a process where cells, whole tissues, or any other substances susceptible to damage caused by chemical reactivity or time are preserved by cooling to sub-zero temperatures such as less than 0° C. (i.e. freezing). At such temperatures, any enzymatic or chemical activity which might cause damage to the material in question is effectively stopped. Cryopreservation methods seek to reach low temperatures without causing additional damage caused by the formation of ice during freezing and thus allow storage of the cryopreserved material, including for extended periods of time. A single freeze-thaw cycle involves: (i) freezing the material using standard methodologies known in the art (e.g. freezing in an appropriate buffer to at least −4° C., at least −20° C., at least −70° C. or at least −195° C.); (ii) optionally storing the frozen cell, for example for at least 1 day, at least 1 week, at least 1 month, at least 1 year, at least 5 years, at least 10 years or at least 20 years); and (iii) thawing the frozen material using standard methodologies known in the art, for example thawing the cell to about 25° C., about 32° C. or about 37° C. Techniques for cryopreservation would be well-known to skilled person. When thawed, the viability of the cells and/or tissue may be at least 10%, at least 25%, at least 50%, at least 60%, at least 70%, at least 80%, at least 90%, or at least 95% compared to the viability of the same cells and/or tissue before cryopreservation. Before and/or after a freeze-thaw cycle, cells may be maintained in culture with proliferation media in ultra-low attachment plates. Passaging may be carried out as necessary, for example twice a week, for example using enzymatic digestion with Accutase™ followed by mechanical dissociation as previously described (Oshima et al., 2009).

### Research Applications

Any of the cells and/or tissues set out herein (for example: the ANPG cell; stem cell, pluripotent cell or progenitor cell capable of differentiating into an ANPG cell; neurosphere and/or organoid comprising an ANPG cell; auditory neuron; glial cell; sensory epithelial cell; SGN; sensory epithelium of the cochlea; and/or a HC), may be useful as a research material.

For example, the cells and/or tissues, and in particular the stem cells, may be used to produce a non-human organism. Methods known in the art may be used to generate the non-human organism, which may be used as a model organism in various research applications. A non-human organism may be provided which comprises the cells and/or tissue by stably or transiently introducing cells and/or tissue into the non-human organism using methods known in the art. Alternatively, or additionally, a non-human organism described above may be treated, contacted or otherwise exposed to the cells and/or tissue, for example in a method for assessing the in vivo safety, efficacy and/or toxicity of the cells and/or tissue. The non-human organisms described above may be selected from a mammal, such a mouse, rat or non-human primate. The cells and/or tissue that are introduced into the non-human organism may be from a species that is the same or different to the non-human organism. For example, where the non-human organism is a mouse, the cells and/or tissue that are introduced into the mouse may be from a mouse or from a human. Similarly, the cells and/or tissue that are contacted, exposed or used to treat the non-human organism may be from a species that is the same or different to the non-human organism. For example, where the non-human organism is a mouse, the cells and/or tissue that are contacted, exposed or used to treat the mouse may be from a mouse or from a human.

The cells and/or tissues set out herein (for example: the ANPG cell; stem cell, pluripotent cell or progenitor cell capable of differentiating into an ANPG cell; neurosphere and/or organoid comprising an ANPG cell; auditory neuron; glial cell; sensory epithelial cell; SGN; sensory epithelium of the cochlea; and/or a HC), and/or the non-human organism set out above, may be used in a screening method, for example to assess the safety, efficacy and/or toxicity of a test substance. For example, the cells, tissue and/or non-human organism may be treated with a test substance (the “test”) or a control (the “control”/“control substance”), and a functional and/or structural characteristic in the test and control groups compared using methods known in the art. When cells and/or tissue are use, the characteristic that is compared may be selected from cell morphology, cell viability, cell proliferation, cell differentiation, and/or cell death. Methods known in the art may be used. By way of example, cellular toxicity may be due to any one or more of altered (in particularly reduced in the test group) cell proliferation, altered (in particularly increased in the test group) cell death, altered gene and/or protein expression, altered migration velocity and/or potential, altered (in particular reduced in the test group) cell viability, altered (in particular reduced in the test group) regenerative capacity, and disrupted cell function and/or morphology in particular disrupted neurosphere function and/or morphology. Gene and/or protein expression of one or more auditory cell marker may be assayed. The cells and/or tissue of the invention may also allow cellular toxicity in response to mechanical stimuli to be tested. When a non-human organism is used, the characteristic that is compared may be selected from morphology of the non-human organism or an organ or tissue contained therein, behaviour, growth, locomotion, organ function, blood composition, urine composition, hearing, and/or lifespan.

Thus, the cells, tissues and/or non-human organisms of the invention allow for the development of new therapeutics, in particular those treating hearing loss, such as conductive hearing loss, sensorineural hearing loss and/or mixed hearing loss, for example through the generation and/or regeneration of one or more of sensory epithelia, sensory epithelium of the cochlea, olfactory epithelium and spiral ganglion neurons. The cells, tissues and/or non-human organisms of the invention also allow the skilled person to calculate a maximum tolerated dose of a test compound. Here, “a test compound” is exemplified by one or more substance whose efficacy and/or safety as therapeutic drugs for the above-mentioned disorders is to be determined and substances that are therapeutic drugs for other diseases whose influences (e.g. toxicity) on cells, tissues and whole organisms must be determined. The test compound may be any one or more of low-molecular compounds, high-molecular compounds, proteins, genes (DNA, RNA and the like), cells, viruses, antibodies or fragments thereof, and the like. Such substances can be chosen as appropriate by the skilled person. Thus, the test compound may comprise multiple individual types of molecules.

As set out above, the screening may be carried out in vitro or ex vivo. It is an advantage of the invention that sufficient quantities of high-quality cells and tissues can be generated for such screening purposes, thus reducing the need for the in vivo testing. For example, sufficient cells can be generated to facilitate high throughput and/or high content screening, for example across several 96-well, 384-well and/or 1536-well, or equivalent.

### Therapeutic Applications

Any of the cells and/or tissues set out herein (for example: the ANPG cell; stem cell, pluripotent cell or progenitor cell capable of differentiating into an ANPG cell; neurosphere and/or organoid comprising an ANPG cell; auditory neuron; glial cell; sensory epithelial cell; SGN; sensory epithelium of the cochlea; and/or a HC) may have utility in therapy (i.e. have a therapeutic effect). The term “therapy” is to be construed in its broadest sense to comprise treatment and/or prevention of a disorder. Also provided is a method for treating and/or preventing a disorder in a patient in need thereof. Also provided is the use of the cells and/or tissue described herein, for treating and/or preventing a disorder in a patient in need thereof. Also provided is the use of the cells and/or tissue described herein, for the use in the manufacture of a medicament for the treatment and/or prevention of a disorder in a patient in need thereof. In any of the examples above, the disorder that is treated and/or prevented may be hearing loss, such as conductive hearing loss, sensorineural hearing loss and/or mixed hearing loss. Treatment and/or prevention of said hearing loss, and in particular sensorineural hearing loss, may occur through the generation and/or regeneration of one or more of sensory epithelia, sensory epithelium of the cochlea, olfactory epithelium and spiral ganglion neurons. The therapeutic effect may be achieved with cells and/or tissue that have not been genetically transformed. Alternatively, the therapeutic effect may be achieved with cells and/or tissue that has been genetically transformed, for example by gene therapy. Alternatively, or in addition, the therapeutic effect may be achieved by gene therapy and/or immunotherapy. Gene therapy focuses on the utilization of the therapeutic delivery of nucleic acids into a patient's cells as a drug to treat disease. For example, one or more gene selected from Myc, Sox2, Lgr5, Wnt7a, Wnt7b, Bmi1, Rtkn2 and a gene set out in Table 1 may be overexpressed; and/or one or more gene selected from Lgr6, Frb, Nkd2, Tgfbr2, Tgfbr3, Hmox1, Cyba, Sox10, Plp1, Lpr5, dkk3, Nrf2 may be knocked down. Immunotherapy is the treatment of disease by activating or suppressing the immune system.

In one embodiment, cells and/or tissue of the present invention are administered as part of an autologous transplant procedure. In another embodiment, cells and/or tissue of the present invention are administered as part of an allogeneic cell transplant procedure. By “autologous cell transplant procedure” it is to be understood that the starting cells and/or tissue is obtained from the same subject as that to which the cells and/or tissue is administered. Autologous transplant procedures are advantageous as they avoid problems associated with immunological incompatibility and are available to subjects irrespective of the availability of a genetically matched donor. By “allogeneic cell transplant procedure” it is to be understood that the starting cells and/or tissue are obtained from a different subject as that to which the cells and/or tissue are administered. Preferably, the donor will be genetically matched to the subject to which the cells are administered to minimise the risk of immunological incompatibility.

The invention provides means whereby pathological phenotypes associated with the indications provided herein can be corrected, treated, arrested, palliated and/or prevented. Correction can refer to both partial, total correction and hyper-correction. Correction may be achieved after about 10 days, 20 days, 30 days, 40 days, 50 days, 60 days, 70 days, 80 days, 90 days, 100 days, 125 days, 150 days, 175 days, 200 days, 250 days, 300 days, 1 year, 1.5 years, 2 years, 2.5 year, 3 years, 4 year or 5 years. The effect of correcting, treating, arresting, palliating and/or preventing a phenotype may be transient. Alternatively, the effect of correcting, treating, arresting, palliating and/or preventing a phenotype may be long term, or sustained.

The treatment of mammals, particularly humans, is preferred. However, both human and veterinary treatments may be within the scope of the present invention.

### Pharmaceutical Compositions, Dosages and Administration

Suitable doses of any of the cells and/or tissues set out herein (for example: the ANPG cell; stem cell, pluripotent cell or progenitor cell capable of differentiating into an ANPG cell; neurosphere and/or organoid comprising an ANPG cell; auditory neuron; glial cell; sensory epithelial cell; SGN; sensory epithelium of the cochlea; and/or a HC) may be such as to be therapeutically and/or prophylactically effective. The dose to be administered may depend on the subject and condition to be treated, and may be readily determined by a skilled person. In addition, the dose of the cells and/or tissues of the invention may be determined according to various parameters, especially according to the age, weight and condition of the patient to be treated; the route of administration; and the required regimen. A physician will be able to determine the required route of administration and dosage for any particular patient. The dose may be provided as a single dose, but may be repeated. The treatment is preferably a single permanent treatment, but repeat injections, for example in future years and/or with different therapies may be considered.

The cells and/or tissues can be formulated into pharmaceutical compositions. These compositions may comprise, in addition to the cell and/or tissue, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may be determined by the skilled person according to the route of administration and may include isotonic saline solutions, for example phosphate-buffered saline, and potentially contain human serum albumin.

The pharmaceutical composition is typically in liquid form containing a suspension of the cells and/or tissue. Such “liquid” pharmaceutical compositions generally include buffered liquid carriers. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used.

For injection at the site of affliction and/or at a site remote of affliction, the active ingredient will be in the form of an aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection, Hartmann's solution. Preservatives, stabilisers, buffers, antioxidants and/or other additives may be included, as required.

For delayed release, the pharmaceutical composition comprising the cells and/or tissues may be formulated for slow release, such as in microcapsules formed from biocompatible polymers or in liposomal carrier systems according to methods known in the art.

The pharmaceutical composition may be co-administered with one or more other agent. The one or more other agent may be administered separately to the pharmaceutical composition, at substantially the same time as the composition of the invention, or as a single composition comprising the pharmaceutical composition in combination with the one or more other agent. Thus, combination therapy comprising the pharmaceutical composition and one or more other agent is envisaged. The one or more other agent may be loaded into the cell of the invention. For example the cells and/or tissues may be further modified to comprise the one or more other agent, such as an agent a targeting moiety to allow for cell-preferential targeting.

The pharmaceutical composition may be administered by any suitable means. Administration to a human or animal subject may be selected from parenteral, intramuscular, intracerebral, intravascular (including intravenous), subcutaneous, intranasal, intracardiac, intracerebroventricular, intraperitoneal or transdermal administration. Typically the method of delivery is by injection. Preferably the injection is intramuscular or intravascular (e.g. intravenous). A physician will be able to determine the required route of administration for each particular patient. The cells and/or tissues may be delivered as a pharmaceutical composition. The pharmaceutical composition may be formulated for any suitable means of administration, including parenteral, intramuscular, intracerebral, intravascular (including intravenous), intracardiac, intracerebroventricular, intraperitoneal, subcutaneous, intraaural, intraocular, intranasal or transdermal administration. Pharmaceutical compositions for parenteral administration may include sterile aqueous solutions which may also contain buffers, diluents and other suitable additives.

The pharmaceutical composition may additionally contain other adjunct components conventionally found in pharmaceutical compositions. Thus, for example, the pharmaceutical composition may contain additional compatible pharmaceutically-active materials or may contain additional materials useful in physically formulating various dosage forms of the composition of present invention, such as dyes, flavouring agents, preservatives, antioxidants, opacifiers, thickening agents and stabilizers. However, such materials, when added, should not unduly interfere with the biological activities of the components of the pharmaceutical composition.

A therapeutically effective amount of pharmaceutical composition is administered. The dose may be determined according to various parameters, especially according to the nature and severity of the condition, age, and weight of the patient to be treated; the route of administration; and the required regimen. A physician will be able to determine the required route of administration and dosage for any particular patient.

### Kits

Any of the cells or tissues set out herein (for example: the ANPG cell; stem cell, pluripotent cell or progenitor cell capable of differentiating into an ANPG cell; neurosphere and/or organoid comprising an ANPG cell; auditory neuron; glial cell; sensory epithelial cell; SGN; sensory epithelium of the cochlea; and/or a HC) can be packaged into a kit. Cells or tissues of the kit may be cryopreserved according to standard methods known in the art. The kit may comprise a buffer, carrier, diluent and/or excipient, which may be readily determined by the skilled person. The kit may comprise a pharmaceutically acceptable excipient. The pharmaceutically acceptable excipient may readily determined by the skilled person and may, for example, comprise or consist of saline and 2% human albumin solution. The kit may be contained in a transfer bag.

### EXAMPLES

The invention is described in more detail below with reference to the following non-limiting Examples.

### Example 1—Materials and Methods

### Study Approval

All animal procedures were approved by the local veterinary office and the Commission for Animal experimentation of the Canton of Geneva, Switzerland, authorization number GE/189/17.

### Spiral Ganglion Cells Isolation and In Vitro Culture

Collection of mouse inner ear spiral ganglion cells was done as previously described in (Oshima et al., 2009). Day 5 postnatal A.B6 Tyr⁺-Cyba^{nmf333}/J (Stock No: 002565, Jackson; carrying the wild type allele of Tyr in a A/J strain genetic background (Stock No: 000646)) and C57Bl6/J (Stock No: 000664) pups were used. They were euthanized by decapitation. The head surface was sterilized with 70% Ethanol and was sectioned along the mid-sagittal plane. Brain and brainstem were removed and both half-skulls were immersed in 2.5 ml of ice-cold Hanks' Balanced Salt Solution (HBSS) in a 35 mm Petri dish. The cochleae were removed under a stereo microscope (Nikkon, Japan). The cartilaginous cochlear capsule was opened and the stria vascularis and the organ of Corti (OC) were removed in one piece from base to apex using 5.5 forceps. The modiolus (with the spiral ganglion cells) was then transferred into 1.5 mL Eppendorf tube with ice-cold HBSS. Tissue dissociation was done by enzymatic digestion followed by mechanical trituration. The modiolus was treated with Accutase (StemPro™ Accutase™ Cell Dissociation Reagent) for 15 minutes, the reaction was stopped by adding proliferation media: DMEM:F12 with 15 mM HEPES buffer and 2 mM L-glutamine supplemented with 1×N2 and B27 supplement (Thermo Fisher), 1× Penicillin streptomycin (100 U/ml) (Thermo Fisher), in presence of bFGF (10 ng/ml, ProSpec), IGF1 (50 ng/ml, Cell Guidance Systems), Heparan sulfate (50 ng/ml, Sigma Aldrich) and EGF (20 ng/ml, Cell Guidance Systems) and mechanical trituration was performed with P1000 (10-15 up and downs). Following Accutase removal with a centrifugation step, the cell suspension was filtered with a 70 μm filter to eliminate undissociated tissue or bony parts and select small cluster of spiral ganglion cells. Isolated cells were maintained in culture with proliferation media in ultra-low attachment 6 well plates (Corning, Sigma Aldrich). Passaging of spiral ganglion cells-derived neurospheres was done twice a week using enzymatic digestion with Accutase™ followed by mechanical trituration as previously described (Oshima et al., 2009).

### Cell Counting

Following sphere/tissue dissociation with Accutase, neuroprogenitors were counted manually using a FAST READ 102@(Biosigma) hemocytometer, according to the manufacturer's instructions. Cell number was determined for three independent cultures up to passage 10, allowing the calculation of generation time (G=5, 697 days) using the formula LOG 10(2)/average slope (0.0528±0.00165) and the theoretical global amplification after 40 passages (corresponding to 42.125 generations): 2\hat{ }42.125=4,79513E+12-fold.

### Cell Cycle Analysis by Flow Cytometry (FACS)

The cell cycle of auditory neurospheres was studied by propidium iodide staining of the DNA followed by FACS analysis. Following dissociation, 1×10⁵ to 2×10⁵ cells were centrifuged, fixed with a 70% ice-cold ethanol solution, treated with RNase A and then stained with the DNA binding agent Propidium Iodide (Sigma Aldrich) in PBS buffer overnight at 4° C. The cell sorting analysis was performed on a BD Accuri C6. FLOWJO (v10.6.2) software was used to determine the percentage of cells engaged in the cell cycle (proliferating).

### Measurement of Induced Ca²⁺ Cytosolic Release

A total of 32,000 cells/well were differentiated to auditory neurons on a 96-well plate coated with Matrigel, as previously described (Rousset et al., 2020). Differentiated cells were loaded with FLUO-8 (Interchim), according to the manufacturer's protocol. After an incubation period of typically 45 min at 37° C. with the inducing agent or control, the induced cytosolic calcium release was assessed in a FDSS/pCELL Functional Drug Screening System (Hamamatsu). The neuronal kinetics of calcium release was followed over about 10 min following treatment, with one measure every 0.5 s.

### Immunohistochemistry and Confocal Microscopy

Auditory neuroprogenitors were plated on Matrigel-coated coverslips in 24-well plates. For immunostaining of differentiated cells, isolated neuroprogenitors were cultured 7 days in differentiation media. For immunostaining of auditory progenitors, undissociated neurospheres were left untouched for 4 h in the incubator, in order to attach to the coverslip. Fixation of the samples was performed using a 4% paraformaldehyde solution for 15 min. Specimens were then permeabilized (0.2% Triton-X 100 in PBS 1×) for 30 min at room temperature and incubated overnight at 4° C. with the primary antibody diluted in a blocking buffer containing 2% bovine serum albumin and 0.01% Triton-X 100 in PBS. The following day, the cells were rinsed 10 min three times with PBS and incubated for 2 h at room temperature with the secondary antibodies in blocking buffer. The stained samples were washed three times with PBS and mounted on glass slides with Fluoroshield containing DAPI (Sigma-Aldrich). The labelled specimens were visualized using a Super Resolutive confocal laser-scanning microscope (LSM800 Airyscan). During the image acquisition, five Z-stack planes were imaged using a 20× or 40× objectives. The final Z-stack projection was edited using FIJI (ImageJ-win64, version 1.53a) software.

### RNA Sequencing

Transcriptomic comparison was performed between low stemness ANPGs organoids, obtained from C57Bl/6 and A/J (at passage 2) and high stemness phoenix ANPGs obtained from A/J ANPGs at passage 5. RNA extraction was performed using a Qiagen RNA extraction minikit (Qiagen), according to the manufacturer's protocol. RNA concentration was determined using a Nanodrop spectrometer and 500 ng of RNA were used for cDNA synthesis using the Takara PrimeScript RT reagent kit, following manufacturer's instruction. TruSeq ribodepleted stranded mRNA was applied to both progenitors and differentiated cells, in triplicates, to eliminate ribosomic RNA and sequenced using Illumina TruSeq protocol. The sequencing quality control was done with FastQC v.0.11.5. The sequences were mapped with the STAR v.2.7.0 software to the UCSC Mus musculus mm10 reference with an average mapping rate of 91.78%. The biological quality control and summarization were performed using the PicardTools v.1.141. The table of counts with the number of reads mapping each gene feature of UCSC mm10 was prepared with HTSeq v0.9p1. The differential expression analysis was performed with the statistical analysis R/Bioconductor package edgeR v. 3.26.8. Briefly, the counts were normalized according to the library size and filtered. The genes having a count above 1 count per million reads (cpm) in at least 4 samples were kept for the analysis. The initial number of genes in the set was 24′420 and after the poorly or non-expressed genes were filtered out, 14′407 genes were left. The differentially expressed genes tests were done with a GLM (general linear model) with a negative binomial distribution using the quasi-likelihood test. The p values of differentially expressed gene analysis were corrected for multiple testing error with a 5% FDR (false discovery rate) using the Benjamini-Hochberg (BH) procedure. Main differentially expressed gene ontologies were determined using G:profiler (https://biit.cs.ut.ee/gprofiler/gost) and gene ontologies network built using EnrichmentMap (version 3.3.1) and AutoAnnotate (version 1.3.3) applications (Merico et al., 2010) in the cytoscape software environment (Shannon et al., 2003) as previously described (Reimand et al., 2019).

### Example 2—Neuroprogenitors Exhibit High Intrinsic Self-Renewal

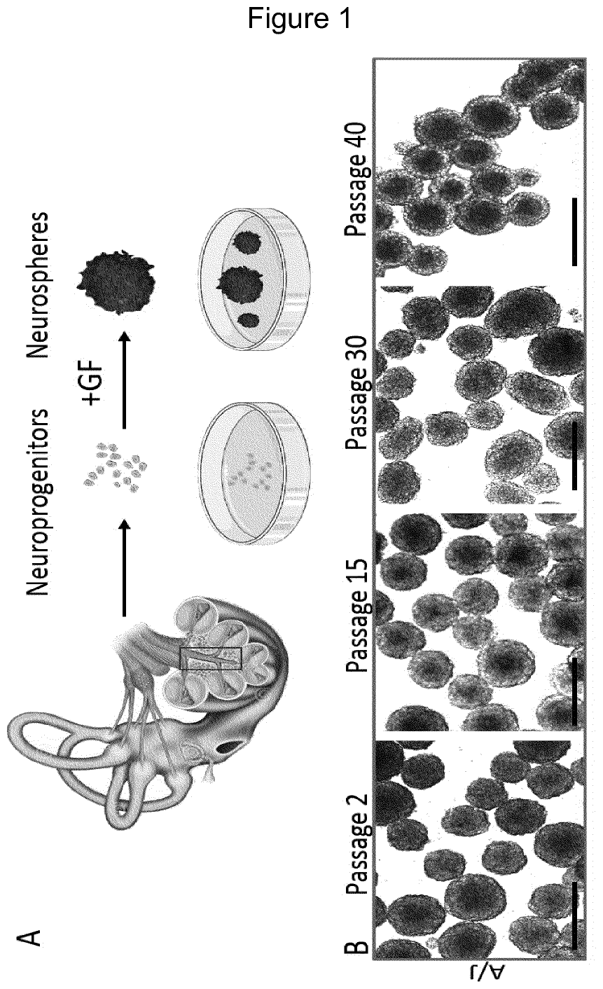

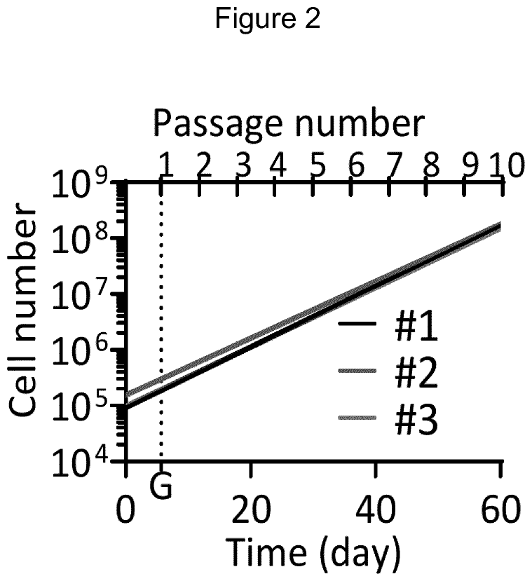

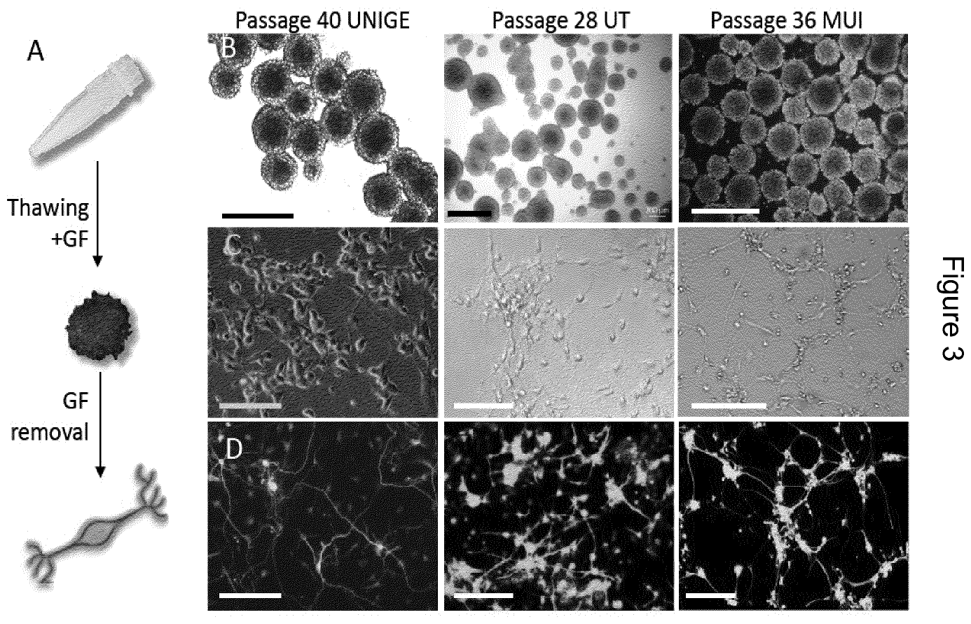

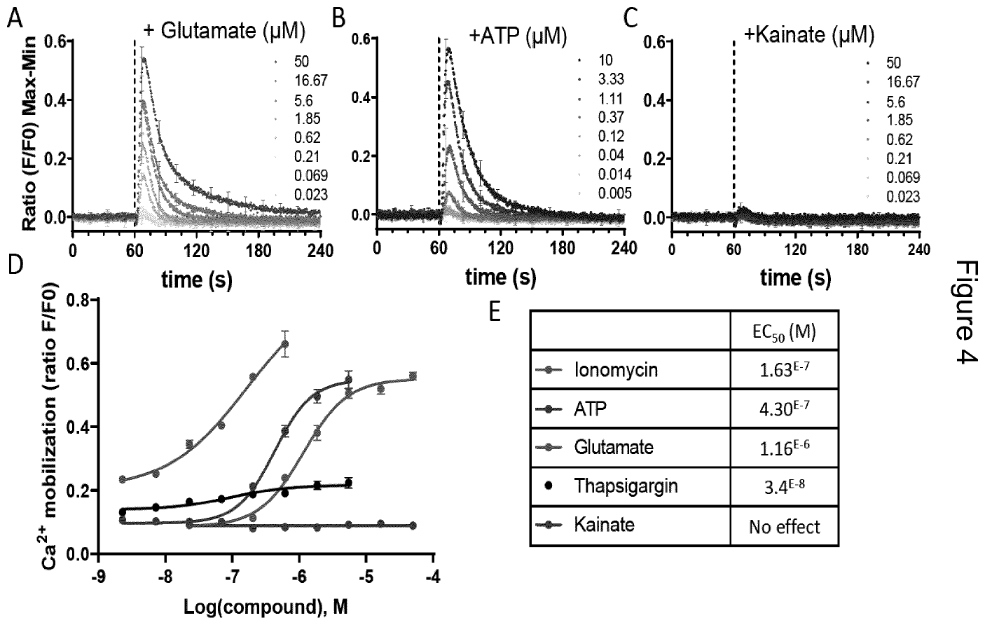

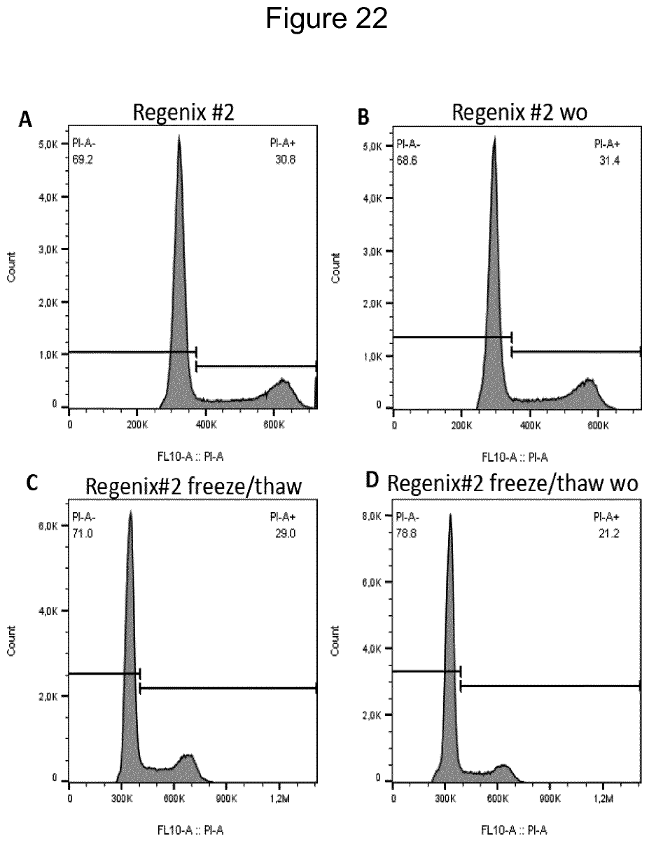

Sphere-derived auditory neurons provide a convenient cell model for morphological and electrophysiological studies, in vitro. However, the propagation potential of auditory neurospheres is generally limited to a few passages before reaching senescence. Cochlear progenitors were isolated from two different genetic backgrounds, namely, from C57BL6 and A/J mice P5 postnatal (FIG. 1A). As described in a previous report (Oshima et al., 2009), neuroprogenitors from either genetic background formed neurospheres in vitro, with no obvious visual differences between strains. However, while C57Bl6 progenitors failed to expand after the third passage, neuroprogenitors from A/J mice (phoenix) exhibited continuous growth up to over 40 passages (FIG. 1B and FIG. 2). Cells were subjected to several freezing and thawing cycles and sent to Universities of Tübingen and Innsbruck (FIG. 3). In three different laboratories, phoenix neuroprogenitors could be easily propagated up to at least passage 40 (FIG. 3B) and, upon growth factor removal, differentiated into auditory neuron-like cells (FIG. 3C), expressing BIII-tubulin (FIG. 3D). Together, the data demonstrate that phoenix auditory neuroprogenitors have virtually unlimited intrinsic self-renewal properties and can easily endure freeze and thaw cycles. Therefore, cells coming from a single primary source can constitute a nearly unlimited source of auditory neurons. Indeed, neither freeze/thaw cycles nor removal of reprogramming factors affect proliferation of stemness-induced C57Bl/6 ANPGs (FIG. 22). In all conditions tested, a significant amount of progenitor cells are proliferating, demonstrating that stemness-induced ANPGs, like phoenix cells, can survive to freeze/thaw cycles and that reprogramming is long lasting, even following reprogramming factors withdrawal.

### Example 3—Excitatory Function of Auditory Neuronal Cells

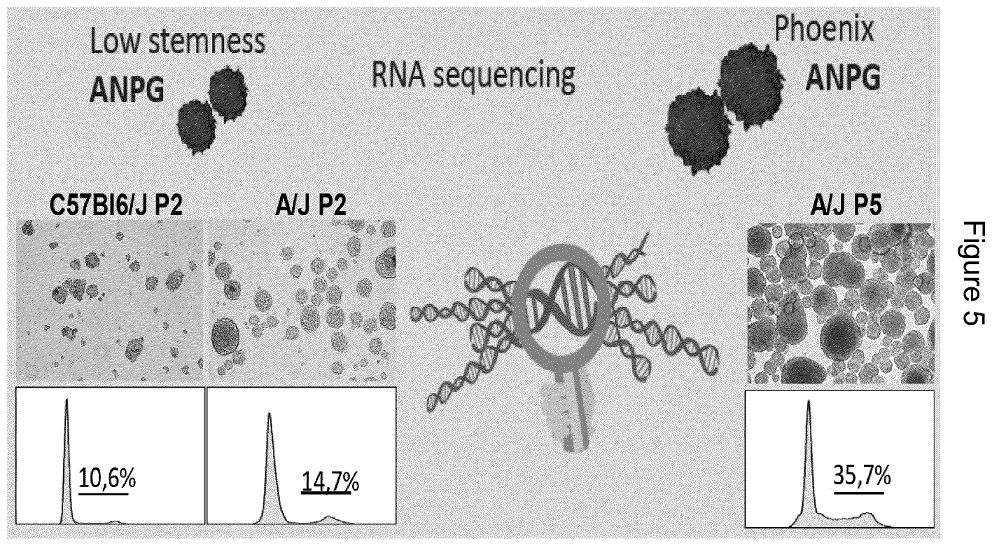

To further explore the excitatory function of differentiated phoenix SGCs, Ca2+ mobilization was studied in response to several physiological stimuli, namely glutamate, ATP, and kainate (FIG. 4). Robust Ca²⁺ transients were induced in D7 differentiated SGCs upon glutamate (FIG. 4A) and ATP (FIG. 4B) stimulation with respective EC50 values of 1.16 and 0.43 μM (FIGS. 4D,E). However, kainate failed to induce Ca2+ excitatory transmission in phoenix SGCs (FIGS. 4C,D). Ionomycin, an ionophore facilitating the transport of divalent cations across cell membranes, and thapsigargin, inhibiting the SERCA pump and therefore the transfer of Ca²⁺ toward endoplasmic reticulum, were used as positive controls (FIG. 4D). The excitatory pattern of phoenix SGCs was also compared with human fetal sphere-derived ones and mouse spiral ganglion explants using live Ca²⁺ imaging. Similarly to phoenix SGCs, human fetal sphere-derived SGCs and mouse spiral ganglion explants exhibited glutamatergic and purinergic evoked Ca²⁺ transients. However, the cultures did not respond to kainate. Together, this demonstrates similar excitatory properties of both human and phoenix auditory SGC. Therefore, at any passage and following differentiation, phoenix auditory SGCs resemble the auditory neuron physiology.

### Example 4—Phenotypic Characterization of Auditory Neuroprogenitors

A transcriptomic comparison of low and high stemness auditory neuroprogenitors (ANPGs) organoids was conducted. ANPG cells were isolated from the mouse pup spiral ganglion and cultured as single cell suspension. Upon growth factor addition (FGF, EGF, IGF, heparan sulfate), auditory neuroprogenitor form organoids in suspension (see bright-field microscopy pictures in FIG. 5). Following DNA staining, at passage 2 (P2), about 10% of the presenescent C57Bl/6 ANPG cells are still engaged in cell cycle. At the same passage, A/J ANPG cells exhibit relatively similar proportion of cycling cells (about 15%); however, whereas C57Bl/6 reach senescence (around passage 3), A/J ANPG cells increase their proliferation rate along passages to about 35%. This is shown in the histograms in FIG. 5 and suggests the existence of a high sternness subpopulation, which is enriched with passages, in at least A/J derived organoids. Transcriptomic comparison was performed between low stemness ANPG organoids, obtained from C57Bl/6 (left, at passage 2 in FIG. 5) and A/J (middle, at passage 2 in FIG. 5) and high stemness phoenix ANPG obtained from A/J ANPG at passage 5 (right in FIG. 5).

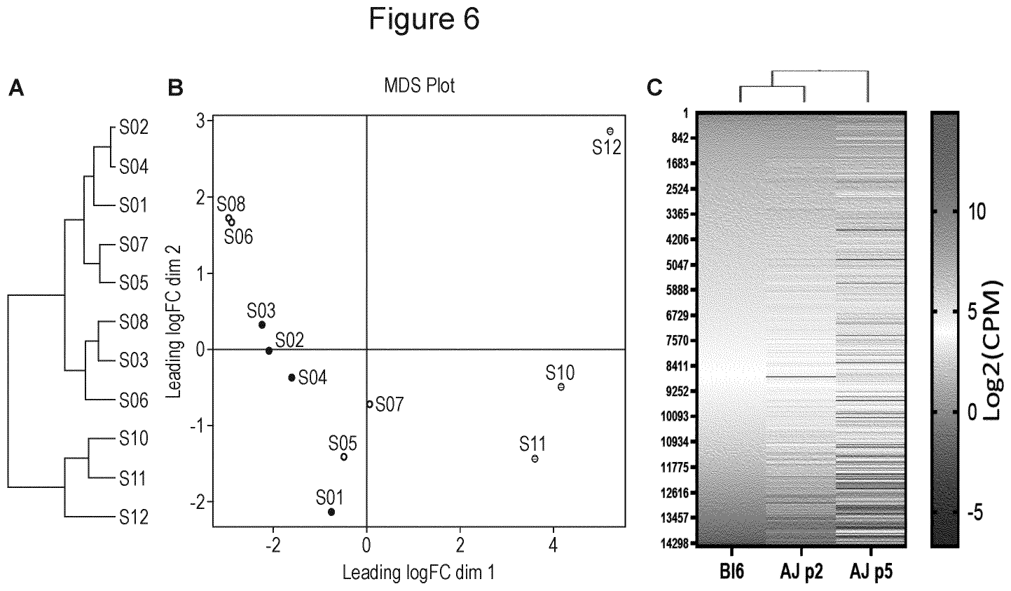

Relative distance between low and high stemness ANPG organoids samples were graphed. FIG. 6A presents a tree recapitulating distance between samples. Low propagating ANPG: S01 to S04 represent C57Bl/6 passage 2 quadruplicate, S05 to S08 represent A/J passage 2 quadruplicate. High propagating ANPGs: S10 to S12 represent A/J passage 5 triplicate. FIG. 6B presents a multidimensional scanning plot based on the fold changes between all the pairs of samples. The heatmap in FIG. 6C shows relative gene expression level in low stemness ANPG cells from C57Bl/6 passage 2 (lane 1) and A/J passage 2 (lane 2) and high stemness A/J passage 5 ANPG (lane 3). The data shows relatively similar transcriptomic signature between C57BL/6 and A/J ANPG at passage 2, whereas the pattern of gene expression in A/J (phoenix) ANPG is more distant.

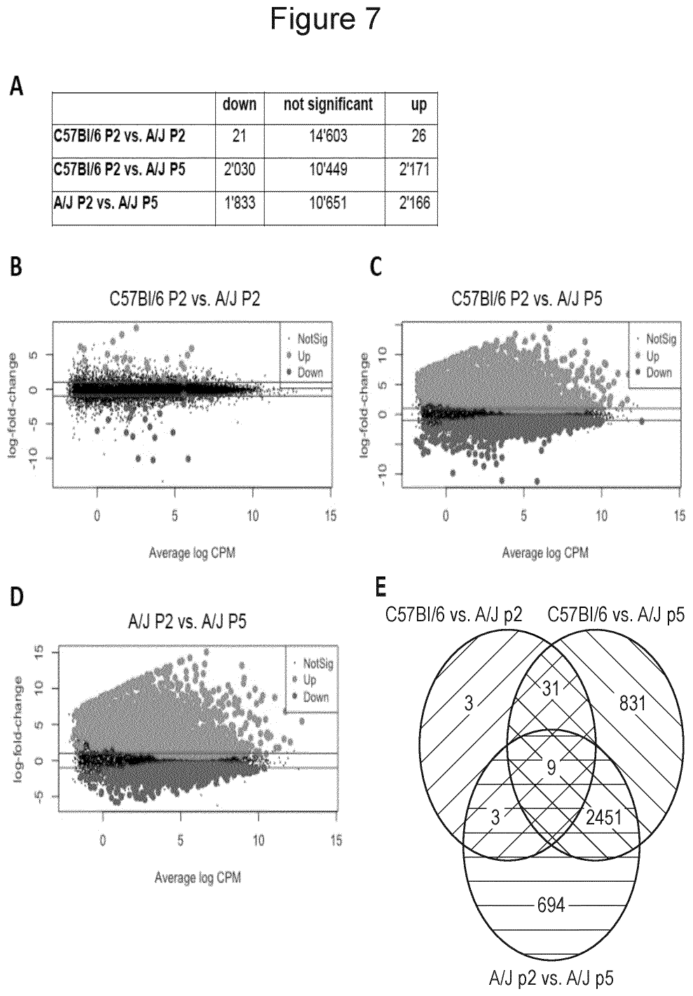

Bulk RNAseq comparison of low (passage 2 C57Bl/6 and A/J) and high (passage 5 A/J) stemness ANPG was carried out. The differentially expressed genes p-values are corrected for multiple testing error with a 5% FDR (false discovery rate) following quasi-likelihood statistical test (FIG. 7A). The correction used is Benjamini-Hochberg (BH). The table gives the differentially expressed genes statistics (FDR5%) and the number of genes with a fold change >2. FIG. 7 B-D presents mean difference plots (MDplots) of expression data showing significantly differentially expressed genes with a FDR of 5%, highlighted in large dots for down and up differentially expressed genes. The horizontal lines between 0 and 5 and 0 and −5 represents the fold change 2 threshold. FIG. 7B C57Bl/6 P2 vs. A/J P2, FIG. 7C C57Bl/6 P2 vs. A/J P5 and FIG. 7D A/J P2 vs. A/J P5. FIG. 7E presents a Venn diagram representation of the differentially expressed genes with an FDR<5% in C57Bl/6 vs. A/J at passage 2, C57Bl/6 vs. A/J at passage 5 and A/J at passage 2 vs. A/J at passage 5. Relatively similar gene expression profile could be observed between passage 2 A/J and C57B/6, however major changes were observed in passage 5 A/J.

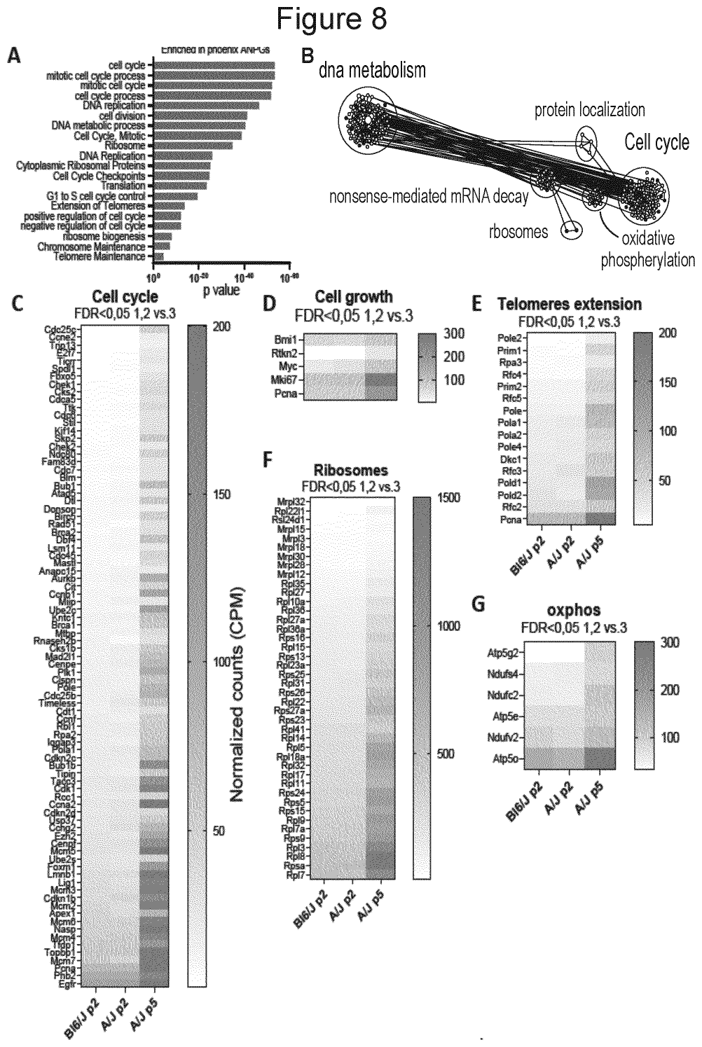

Gene ontologies enriched in high stemness ANPGs organoids from A/J passage 5 were analysed. FIG. 8A shows the main relevant gene ontologies showing a significantly enrichment in phoenix ANPGs vs. C57Bl/6 and A/J ANPGs. FIG. 8B shows a gene ontology (GO) network. FIGS. 8 C-G presents heatmaps showing relative expression level of genes belonging to the cell cycle (FIG. 8C), cell growth (FIG. 8D), telomeres extension (FIG. 8E), ribosomes (FIG. 8F) and oxidative phosphorylation (FIG. 8G), in low sternness ANPG from C57Bl/6 passage 2 (lane 1) and A/J passage 2 (lane 2) and high sternness A/J passage 5 ANPG (lane 3). All genes displayed are significantly differentially expressed (FDR<5%) between low sternness (C57Bl/6 and A/J ANPGs) and phoenix ANPGs.

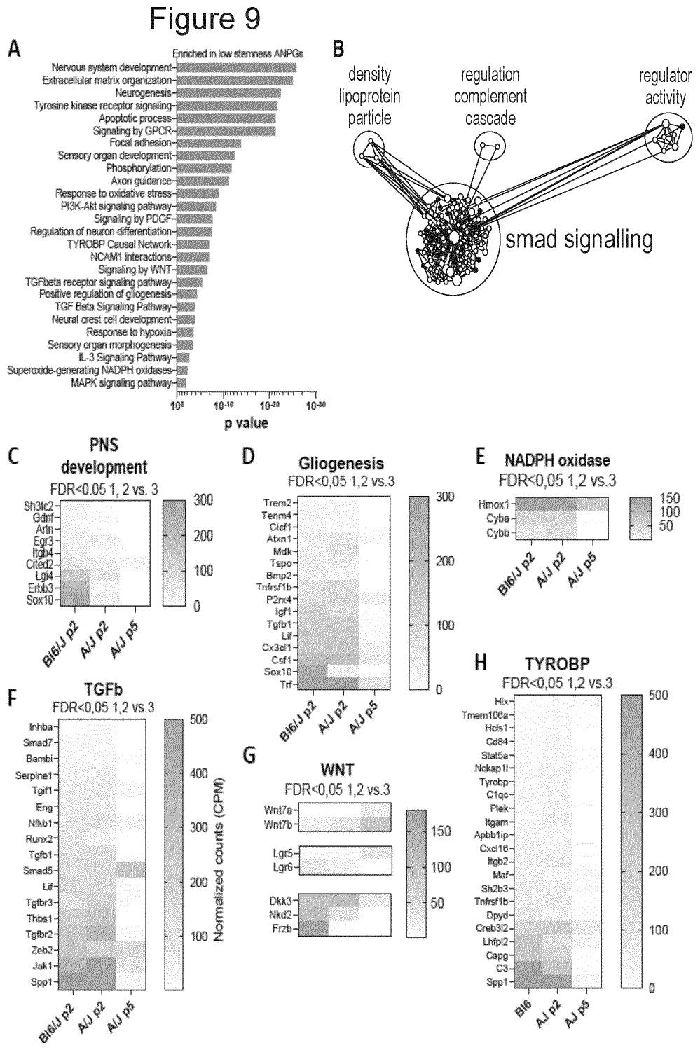

Gene ontologies enriched in low sternness ANPG organoids from C57Bl/6 and A/J passage 2 were also analyses. FIG. 9A shows the main relevant gene ontologies showing a significantly enrichment in C57Bl/6 and A/J ANPGs vs. phoenix ANPGs. FIG. 9B shows a gene ontology network. FIGS. 9 C-H presents heatmaps showing relative expression level of genes related to the peripheral nervous system development (FIG. 9C), gliogenesis (FIG. 9D), NADPH oxidase (FIG. 9E), TGF-b pathway (FIG. 9F), WNT pathway and TYROBP pathway (FIG. 9H) in low sternness ANPG from C57Bl/6 passage 2 (lane 1) and A/J passage 2 (lane 2) and high sternness A/J passage 5 ANPG (lane 3). All genes displayed are significantly differentially expressed (FDR<5%) between low sternness (C57Bl/6 and A/J ANPGs) and phoenix ANPGs.

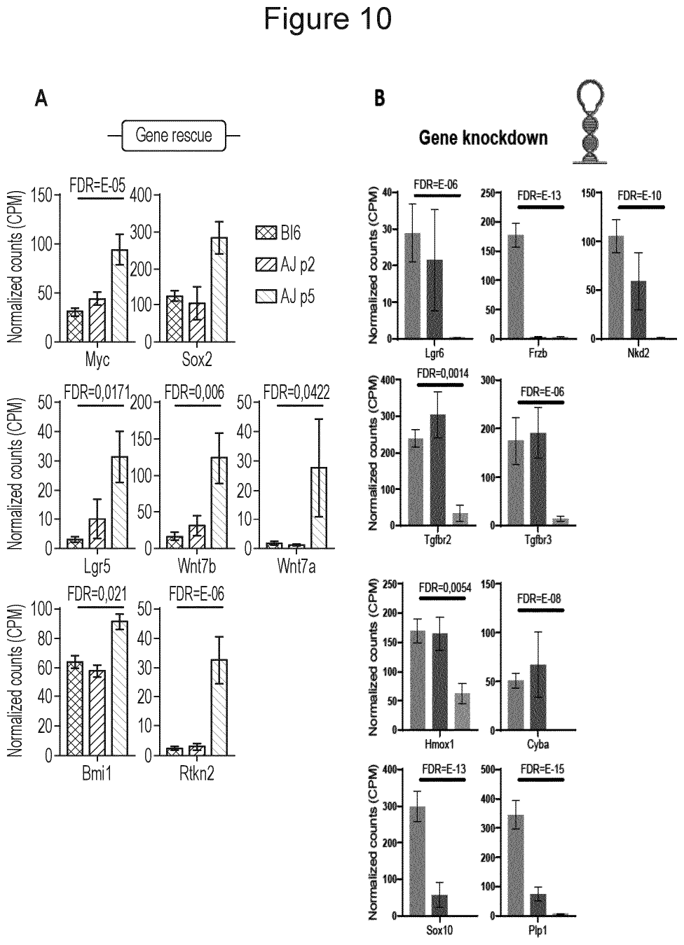

Molecular targets for sternness induction in low propagating ANPGs were analyzed. Bar graphs showing the expression level of a non exhaustive number of candidate genes, differentially expressed in passage 5 A/J vs. passage 2 A/J and C57Bl/6 derived organoids and thus possibly involved in A/J ANPG sternness are presented in FIG. 10. These suggests that inducing expression (rescue) of candidate genes (FIG. 10A) or alternatively knocking down the candidate genes (FIG. 10B) or a combination of the two represents an approach to induce sternness in low propagating ANPGs, as well as other cell types.

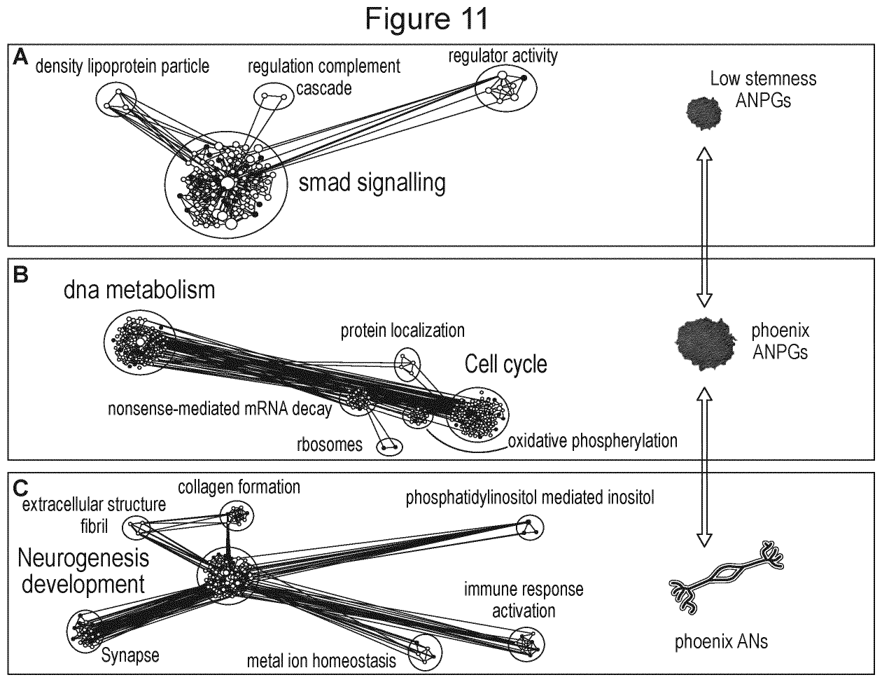

Gene ontology networks of low propagation ANPGs and phoenix ANPGs and derived neurons were also built from differentially regulated gene ontologies between low sternness (C57Bl/6 and A/J p2), high sternness (A/J p5) and derived auditory neurons (FIG. 11).

### Example 5—Auditory Neurons as a Tool for Neurogenesis Screening

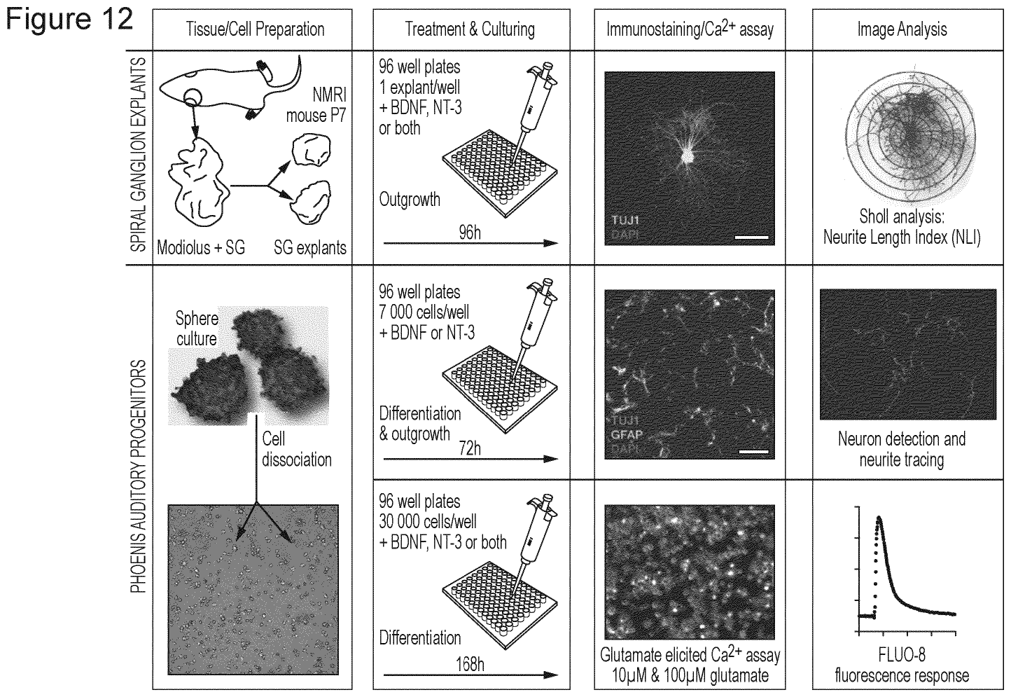

Organotypic spiral ganglion explant culture was carried out prior to in vitro phoenix sphere derived neuron assays, as shown in the workflow in FIG. 12. The upper row shows spiral ganglion explant (SGE) cultures from 7-day old NMRI mouse pups in 96 well plates, cultured in presence of NT-3 and BDNF alone or in combination for 96 h. After fixation, specimens were stained with DAPI and for β-III-tubulin (TuJI antibody) to address the effect of neurotrophins on neurite outgrowth and neuron numbers, quantified with a modified Sholl analysis routine. For each explant, the neurite length index (NLI) was computed, and associated intersection counts served as approximation for neuron numbers. The lower rows show phoenix auditory neuroprogenitor sphere dissociation and induction of differentiation. To assess the outgrowth morphology, cells were seeded at 7,000 cells/well with presence of NT-3 or BDNF for 72 h. Differentiated cells were stained for β-III-tubulin, GFAP and DAPI for morphological analysis. Parameters such as the total neurite outgrowth, the number of neurons, the number of glial cells and the mean neurite length were assessed using a custom-made Imaris procedure. For a functional evaluation by means of a calcium response assay to glutamate, 30,000 cells were seeded per well and differentiated with supplementation of BDNF, NT-3 or combinations thereof. Via a fluorescent calcium binding dye, the cellular response to glutamate was quantified.

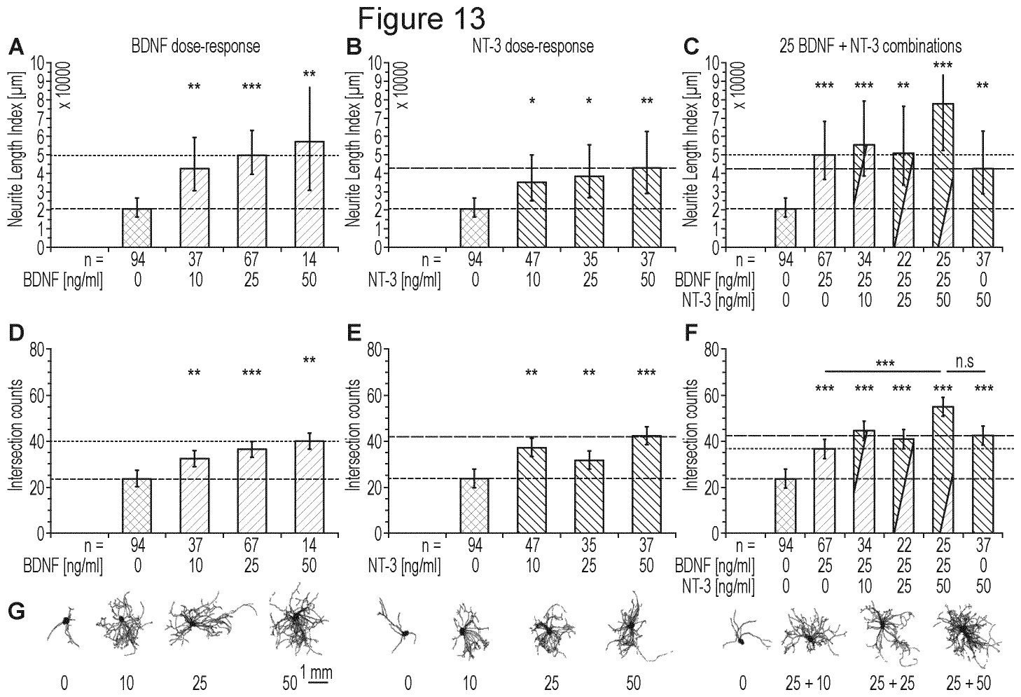

The effect of BDNF and NT-3 alone or in combination on organotypic spiral ganglion explant culture morphology was assessed. Spiral ganglion explants (SGE) of freshly dissected P7 old mouse pups were seeded in poly-D-lysin and laminin coated 96 well plates and incubated without (n=94) or with BDNF, NT-3 or combinations of these for 96 h. Neurite outgrowth (FIG. 13 A-C) and intersections (FIG. 13 D-F) were quantified. Representative explant micrographs are presented in FIG. 13G for the given treatments. Numbers indicate the amount of neurotrophin added in ng/ml. For the data in Figures A-C, with BDNF, NT-3 or combinations thereof, stimulated neurite outgrowth was significantly enhanced compared to spontaneous neurite outgrowth in P7 SGEs. In controls, a substantial outgrowth with a mean NLI of 20828 (±5647/4442; n=94), was registered. The back transformed mean log values rose significantly with rising BDNF (FIG. 13A) or NT-3 (FIG. 13B content. A further increase could be observed when combining 25 ng/mL BDNF and 50 ng/mL NT-3 (FIG. 13C). The bar graphs in Figures D-F show the intersection quantification, representing the approximated number of neurons with outgrowth. Neuron numbers rose significantly in a dose-dependent manner upon addition of BDNF (FIG. 13D), NT-3 (FIG. 13E) or combinations thereof.

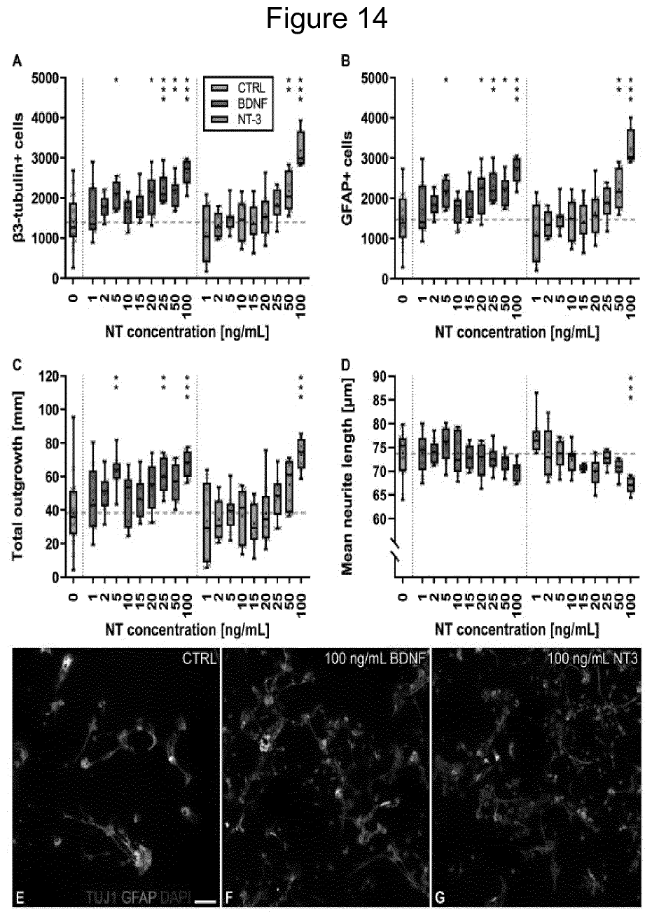

The impact of BDNF and NT-3 supplementation on morphology of phoenix auditory neurons was also assessed. Phoenix neuroprogenitors were differentiated for 72 h in presence of various concentrations of BDNF or NT-3 in 96-well plates. The effect of BDNF and NT-3 supplementation on the number of neurons (exclusively beta-III-tubulin positive cells, (FIG. 14A), the number of glial cells (exclusively GFAP positive cells, FIG. 14B), the total neurite outgrowth (FIG. 14C) and the mean neurite length per neuron exhibiting outgrowth (FIG. 14D), were assessed using a custom-made Imaris procedure. The number of neurons as well as the number of glial cells and the total outgrowth increased in general in a dose depended manner, peaking at 100 ng/mL BDNF or NT-3 respectively (FIGS. 14 A-C). On the contrary, the mean neurite length within a well slightly decreased with higher neurotrophin concentrations (FIG. 14D).

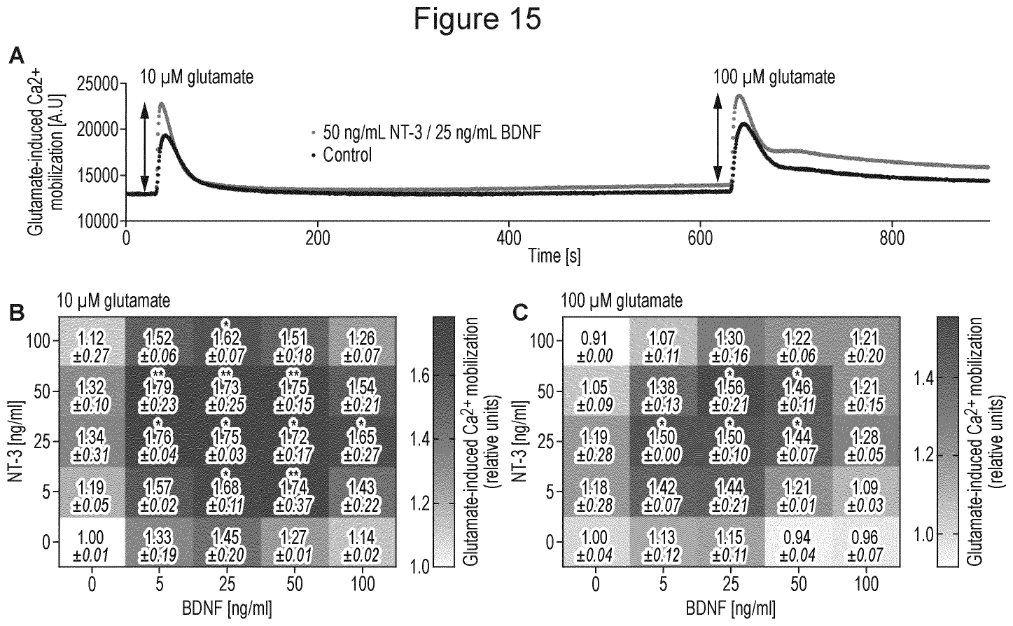

Further, the impact of BDNF and NT-3 alone or in combination on phoenix auditory neurons' glutamatergic function was assessed. Phoenix neuroprogenitors were plated on poly-ornithine and laminin coated 96 well plates and differentiated for 7 days with or without BDNF and NT-3 alone or in combination. To assess effect of neurotrophins, the auditory neuron were stimulated twice with glutamate: first with 10 μM (EC90) and 10 min later with 100 μM, eliciting a maximal glutamatergic response (FIG. 15A). Traces show the Ca2+ mobilization kinetics with 50 ng/mLNT-3+25 ng/mL BDNF (top light curve) or without neurotrophins (bottom dark curve) upon glutamate stimulation as recorded with Ca2+ sensitive ratiometric probe (FLUO-8). Arrows show the max-min amplitude of the response following the first (10 μM) or the second (100 μM) glutamate stimulation. Heatmaps showing the effect of BDNF and NT-3 alone or in combination on Ca2+ release (min-max amplitude) induced by 10 μM glutamate (FIG. 15B) and 100 μM glutamate (FIG. 15C).

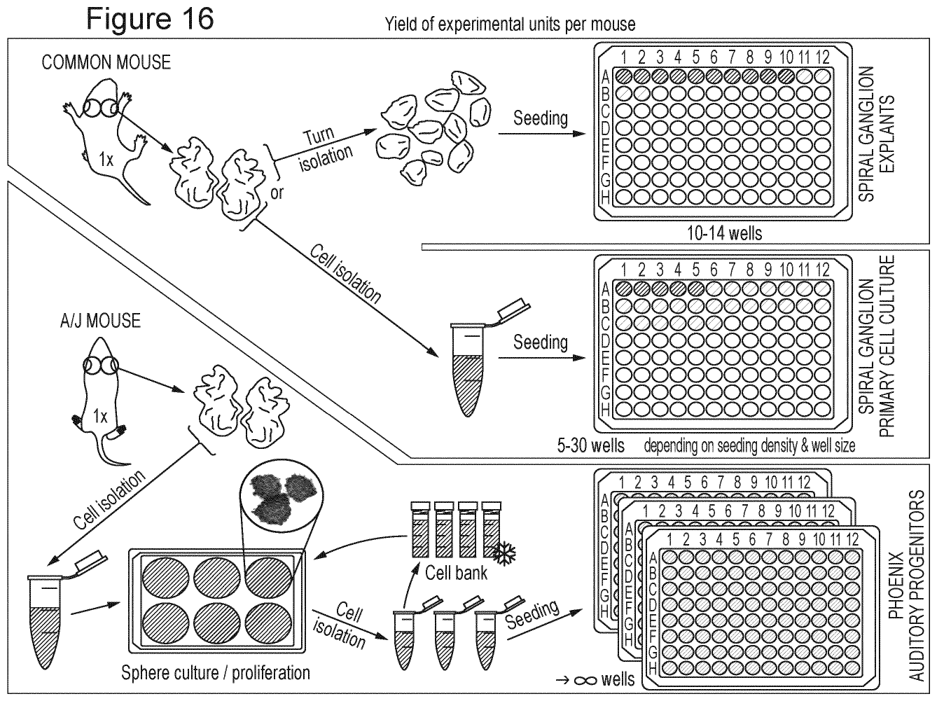

The two most widely used methods for assays on auditory neurons are organotypic spiral ganglion explant preparations and spiral ganglion single cell dissociations. Both methods yield only a limited number of experimental units per sacrificed mouse. Roughly 10-14 SGE can be extracted from two inner ears while single cell preparations can yield, depending on seeding densities and well sizes, approximately 5-30 experimental units. On the contrary, dissociated cell preparations gathered from the A/J mouse can be cultured as spheroids, expanded and passaged, as well as stored and reused in a cell bank without the need of mutagenesis. Therefore, almost indefinite experimental units can be generated (FIG. 16).

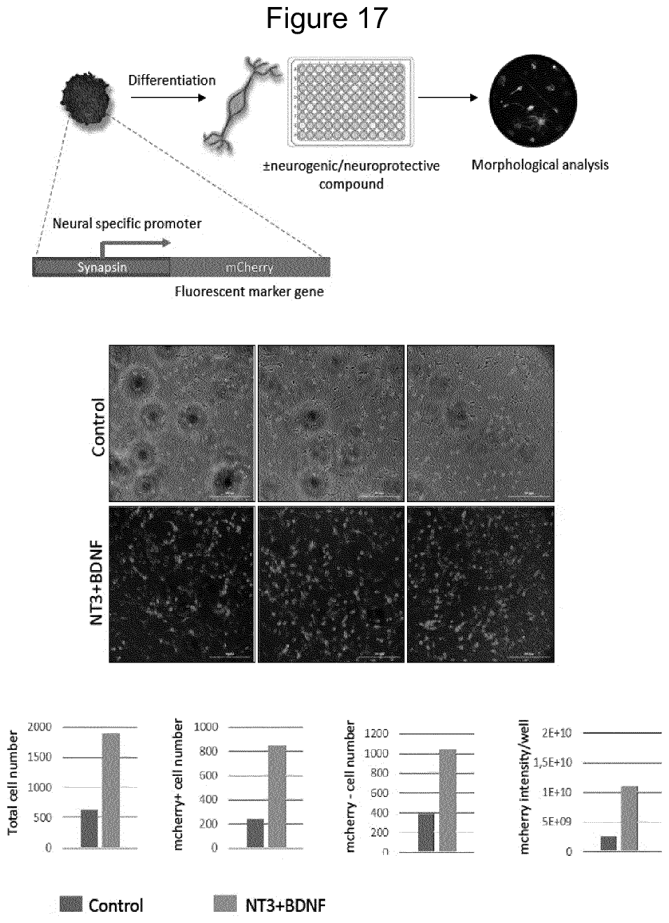

High throughput evaluation of auditory neurogenesis were carried out in an all in one assay (FIG. 17). Stable phoenix cell lines expressing mcherry fluorescent marker gene upon neural specific synapsin promoter can be established through lentiviral transduction. Micrograph images show representative examples of phoenix differentiated for 6 days without (control) or with neurotrophins (BDNF and NT-3). Features including total mcherry positive cell number or mcherry intensity/cell or/well were easily extrapolated using cytation device (biotek). Density: 7500 cells/well.

### Example 6—3D Auditory Neuron Organoids Generation from Phoenix ANPGs Neurospheres

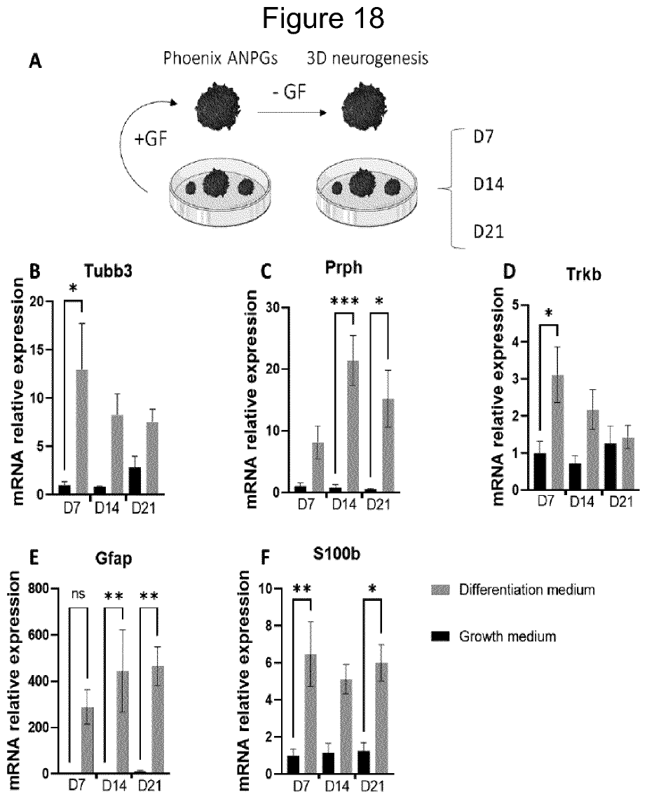

Phoenix ANPGs neurospheres were cultured in classical growth medium (DMEM:F12+N2+B27+GF) and were transferred in differentiation medium (in absence of GF, but with LIF, NT3 and BDNF) for up to 21 days, At day 7, 14 and 21, organoids were harvested for quantitative assessment of neurogenesis gene expression (qPCR) (FIG. 18A). Relative mRNA expression was quantified (FIG. 18B-F). Relative expression of Tubb3, Prph, Trkb, Gfap, and S100b genes were measured at day 7, 14 and 21. Undifferentiated neurospheres in proliferation media (growth) were used as control. Robust induction of neural marker genes (Tubb3, Prph and TrkB) as well as glial markers (Gfap and S100b) was observed after 7 days of differentiation in the 3D setup with gene expression lasting up to day 21. Results represent the average of 4 independently generated phoenix cell lines.

### Example 7—Neurosphere Growth Induction in Low Propagating ANPGs and Induction of Self-Propagation in Low Stemness ANPG

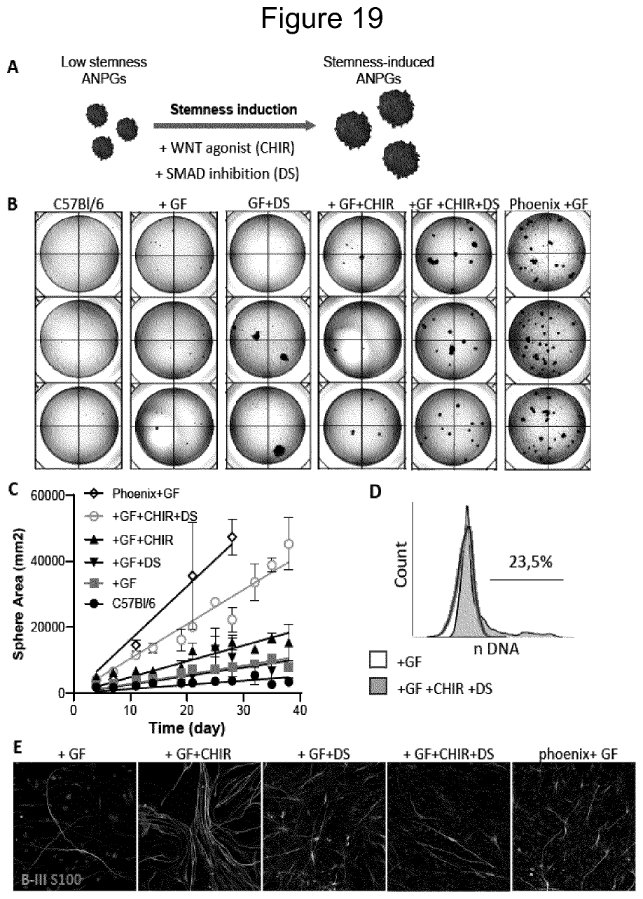

C57Bl/6 ANPGs, exhibiting a limited self-renewal capacity (typically to 2-3 passages), were treated with WNT agonist (CHIR99021; 3 μM) or TGFb antagonist (dual SMAD (DS) inhibitors; LDN193189 0.5 μM, SB431542 10 μM) aiming at replicating phoenix ANPG pattern of gene expression and subsequent stemness phenotype (FIG. 19A). The WNT and SMAD pathways were the two main differentially expressed pathways between low (C57Bl/6) and high stemness (A/J, phoenix) ANPG. ANPG spheres were cultured for 1 month in a 96 well plate. Phoenix cells were used as positive control. C57Bl/6: ANPGs were cultured in DMEM:F12+N2 and B27 supplements without growth factors. +GF: DMEM:F12+N2 and B27+IGF+EFG+HS+FGF. GF+DS:DMEM:F12+N2 and B27+IGF+EFG+HS+FGF+LDN193189 0.5 μM+SB431542 10 μM. GF+CHIR: DMEM:F12+N2 and B27+IGF+EFG+HS+FGF+CHIR99021 3 μM. +GF+CHIR+DS: DMEM:F12+N2 and B27+IGF+EFG+HS+FGF+LDN193189 0.5 μM+SB431542 10 μM+CHIR99021 3 μM. Phoenix+GF: phoenix ANPG were cultured in standard conditions (DMEM:F12+N2 and B27+IGF+EFG+HS+FGF). Picture of representative wells are presented in triplicate in FIG. 19B. Average sphere area at different time points (up to 38 days) was also measured (FIG. 19C). By both enhancing WNT pathway and repressing the dual smads (DS), growth of low stemness C57Bl/6 ANPG was enhanced significantly compared to previous “state of the art” conditions, to a level approaching phoenix ANPGs. Following DNA staining, flow cytometry was performed to determine the percentage of cells engaged in the cell cycle (phase S/G2M; proliferating). As shown in FIG. 19D, whereas C57Bl/6 ANPGs cultured only in presence of growth factors (GF) were virtually senescent, ANPGs treated with WNT agonist (CHIR) and dual smad inhibitors (DS) exhibited significant (>20%) fraction of cycling ANPGs. As shown in FIG. 19E, after reprogramming, stemness induction was induced by CHIR99021; 3 μM(CHIR); dual SMAD inhibitors LDN193189 0.5 M and SB431542 10 μM (DS) and both treatments together (CHIR+DS). In every cases, treated ANPGs were able to differentiate into neurons (expressing BIII-tubulin) and glial cells (expressing S-100).

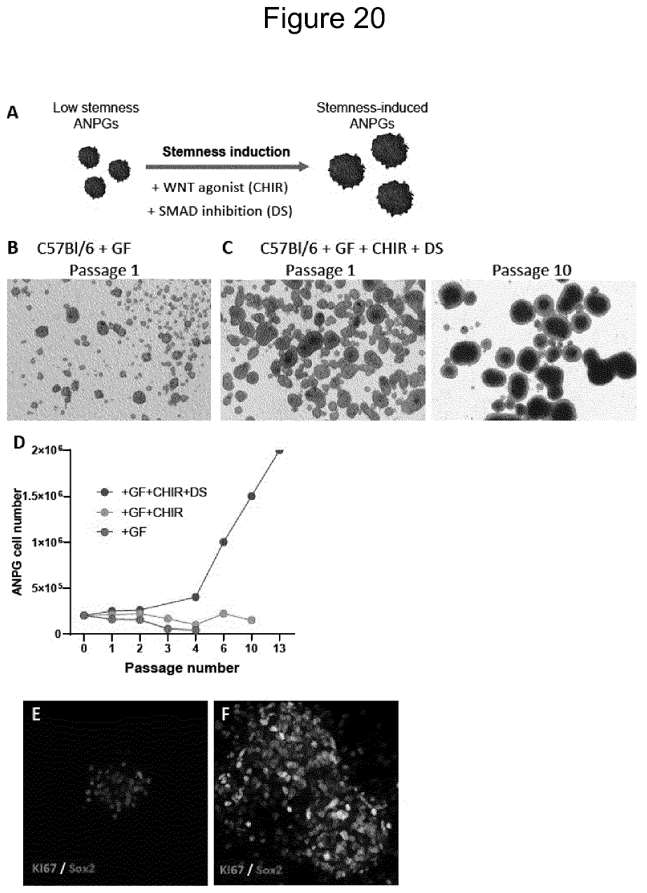

Additionally, C57Bl/6 ANPGs, exhibiting a limited self-renewal capacity (typically to 2-3 passages) were treated with WNT agonist (CHIR99021; 3 μM) or TGFb antagonist (dual SMAD inhibitors; LDN193189 0.5 μM, SB431542 10 μM) with the aim of replicating phoenix ANPG pattern of gene expression and subsequent stemness phenotype (FIG. 20A). The WNT and SMAD pathways were the two main differentially expressed pathways between low (C57Bl/6) and high stemness (A/J, phoenix) ANPG. Representative pictures of ANPG spheres 4 days following the first passage cultured in “standard” (previously described) conditions (GF) or conditions to induce stemness (GF+LDN193189 0.5 μM+SB431542 10 μM+CHIR99021 3 μM); together with representative pictures of stemness induced ANPG spheres, cultured in presence of GF+LDN193189 0.5 μM+SB431542 10 μM+CHIR99021 3 μM, 4 days following the 10th passage, are presented in FIGS. 20B and C. At every passage, following dissociation with Accutase, ANPGs were counted with a fast read counting chamber. A graph showing the number of cells/passage up to passage 13 is presented in FIG. 20D. Whereas C57Bl/6 ANPGs cultured in standard conditions were not able to expand, stemness induced ANPGs could be expanded beyond 10 passages reaching about 2 million cells at passage 13. As shown in FIG. 20E-F, reprogrammed ANPGs exhibit strong KI67 staining demonstrating important proliferation.

### Example 8—Phenotypical Characterization of Sternness Induced Auditory Neurons (ANs)

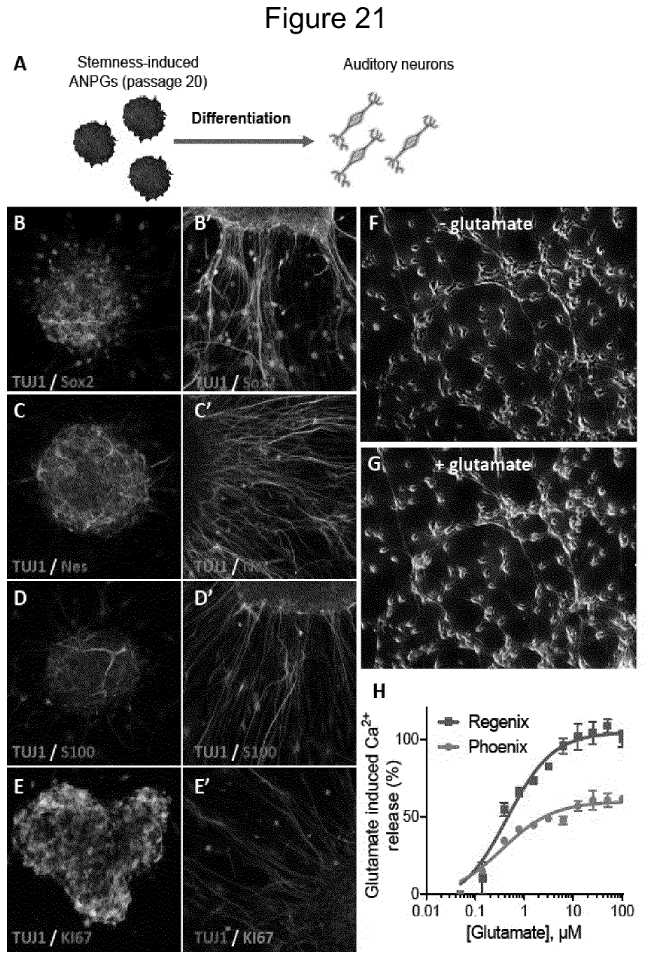

C57Bl/6 ANPGs were treated with WNT agonist (CHIR99021; 3 μM) or TGFb antagonist (dual SMAD inhibitors; LDN193189 0.5 μM, SB431542 10 μM) with the aim of replicating the phoenix ANPG sternness phenotype (FIG. 21A). Immunostainings in differentiated ANs were compared to stemness-induced ANPGs neurospheres at the same passage. Strong induction of neuronal markers (TUJ and S100) upon differentiation was demonstrated (FIG. 21B-E), whereas the expression of stem cells/neuroprogenitors markers Nestin and the proliferation marker K167 strongly decreases. Note that the expression of Sox2 also remains in some differentiating cells, suggesting that a proportion of the stemness-induced ANs are not fully mature. Excitatory potential of stemness-induced ANs was tested using live Ca2+ imaging (FIGS. 21F and G). Stemness-induced ANPGs were differentiated for 7 days in absence of growth factors. The resulting ANs cells were loaded with the Ca2+ sensitive ratiometric probe FLUO-8 and treated with glutamate 100 uM. Upon glutamate addition, siANs exhibited a robust Ca2+ response. Gluatamate-induced Ca2+ response in Phoenix and Regenix (stemness-induced ANPGs), following glutamate increments (0-100 M, ½ serial dilutions) was demonstrated (FIG. 21H). Both phoenix and stemness-induced ANs exhibited robust Ca2+ response with EC50 within the μM range.

### Example 9—Protocol for Sternness Reprogramming in C57Bl/6 ANPGs Based on Pharmacological Inhibition of Pathways Enriched in Low Stemness Progenitors

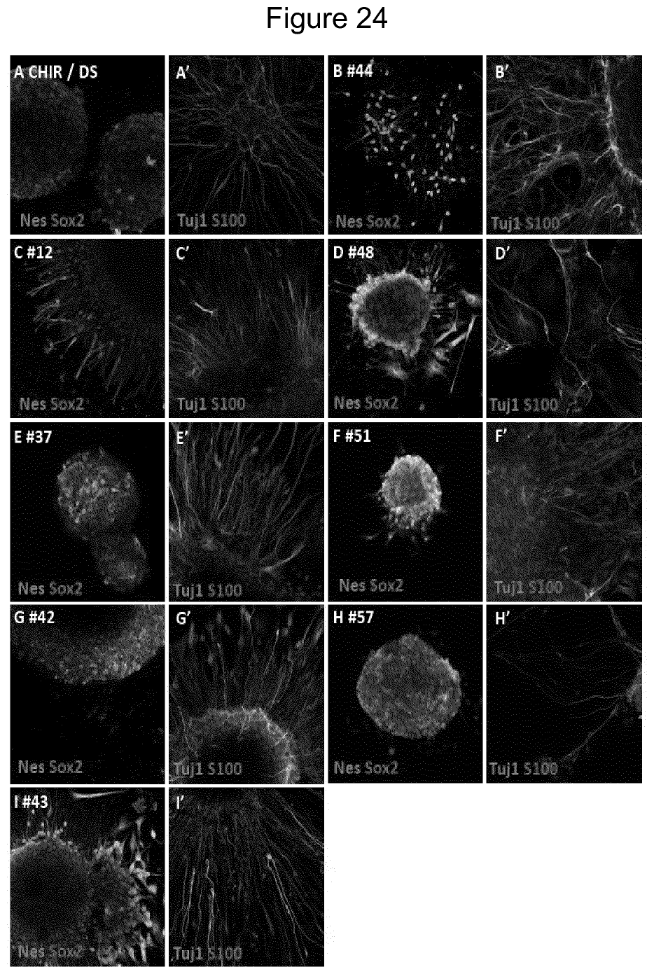

It was demonstrated that inhibition of various signalling pathways could enhance reprogramming of stemness. The pharmacological modulators identified in FIG. 23(B) were selected. C57Bl/6 ANPGs were plated at 10,000 cells/well of a 96 well plate and treated with the WNT agonist (CHIR99021; 3 μM) and various modulator(s). Results are presented in FIGS. 23(C) and (D). It was also demonstrated that ANPG stemness reprogramming did not alter their ability to differentiate into auditory neuron and glial cells. As shown in FIG. 24, ANPGs treated with most of the tested hits exhibit unaltered neural stem cells markers expression (Sox2 and Nestin), whereas they express markers of neurons and glial cells upon differentiation.

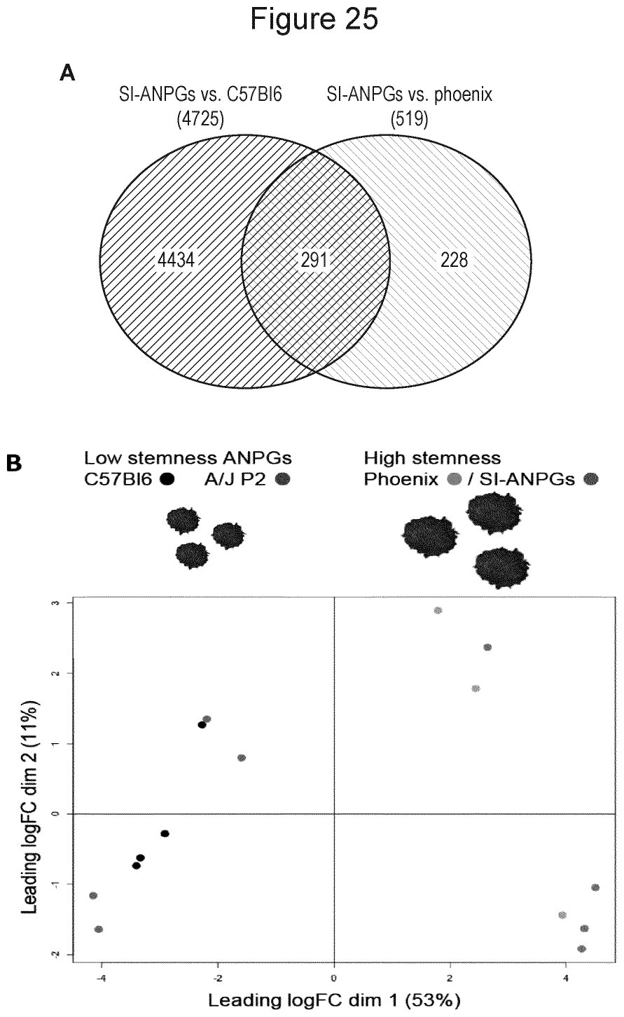

Stemness-Induced (SI) ANPGs also exhibited comparable transcriptomic profile as phoenix cells. The transcriptomic comparison of SI-ANPGs to low stemness ANPGs (C57Bl/6 and A/J passage 2) and phoenix ANPGs was performed with the statistical analysis R/Bioconductor package edgeR 1.34.1. with a multiple testing Benjamini and Hochberg correction FDR 5% and a fold change threshold of 2, exact test. As shown in FIG. 25, more than 4700 genes are differentially expressed between SI-ANPGs and C57Bl/6, only about 500 are different in phoenix and thus, SI-ANPGs, originating from C57Bl/6 background are transcriptionally much closer from phoenix following reprogramming. FIG. 25 goes on to show a Multidimensional Scale Plot (MDS), which gives an indication of the similarity, based on the fold changes between all the pairs of samples. The MDS plot shows segregation between low stemness ANPGs, namely C57Bl/6 and A/J passage2 and high stemness ANPGs, namely phoenix and SI-ANPGs.

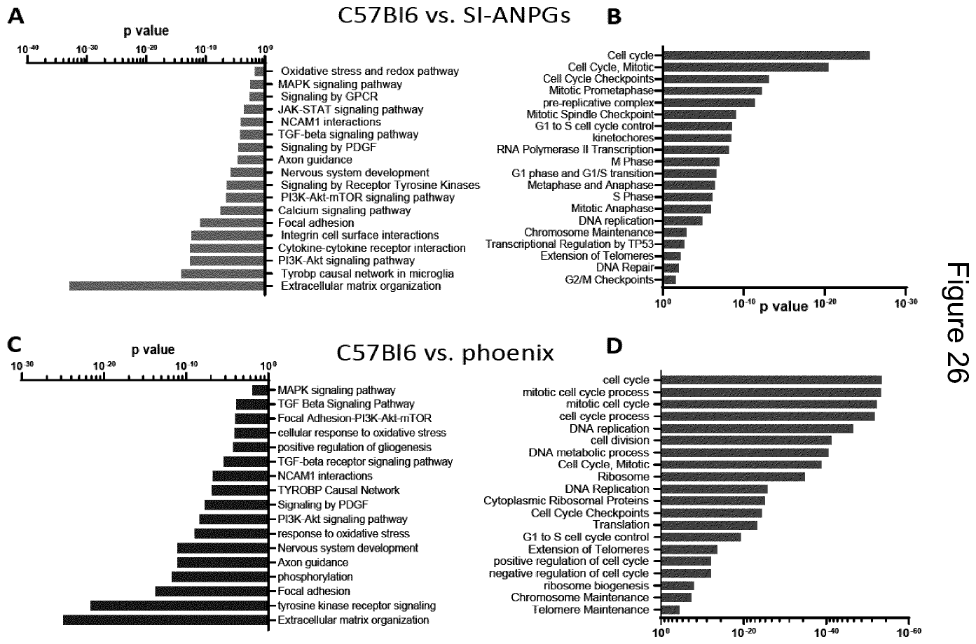

The main relevant gene ontologies enriched in C57Bl/6 (A) vs. SI-ANPGs, and enriched in C57Bl/6 vs. phoenix is presented in FIG. 26. Relatively comparable ontology profiles were observed between phoenix and SI-ANPGs when compared to the presenescent C57Bl/6 ANPGs.

### Example 10—Reprogramming Stemness of Human Fetal Auditory Progenitors and Auditory Hair Cells

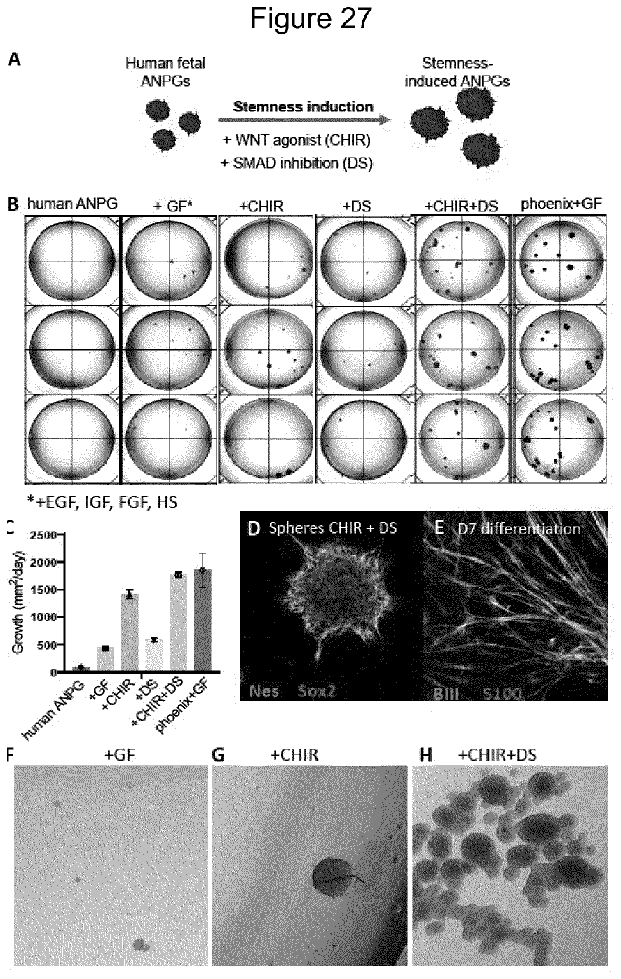

Human fetal ANPGs were isolated 10 weeks post abortion and were plated at 10,000 cells/well of a 96 well plate. They were treated with WNT agonist (CHIR99021; 3 μM) and/or TGFβ Smad antagonist (dual SMAD inhibitors; LDN193189 0.5 M, SB431542 10 μM) aiming at replicating phoenix ANPG pattern of gene expression and subsequent stemness phenotype. Growth of ANPG neurospheres was followed twice a week over 32 days and a macroscopic overview of some conditions of progenitors sphere triplicates is given at day 32. Human ANPGs: ANPGs were cultured in DMEM:F12+N2 and B27 supplements without growth factors. +GF: DMEM:F12+N2 and B27+IGF+EFG+HS+FGF. +DS: DMEM:F12+N2 and B27+IGF+EFG+HS+FGF+LDN193189 0.5 M+SB431542 10 μM. +CHIR: DMEM:F12+N2 and B27+IGF+EFG+HS+FGF+CHIR99021 3 μM. +CHIR+DS: DMEM:F12+N2 and B27+IGF+EFG+HS+FGF+LDN193189 0.5 M+SB431542 10 μM+CHIR99021 3 μM. Phoenix+GF: phoenix ANPG cultured in standard conditions (DMEM:F12+N2 and B27+IGF+EFG+HS+FGF). Data is presented in FIG. 27.

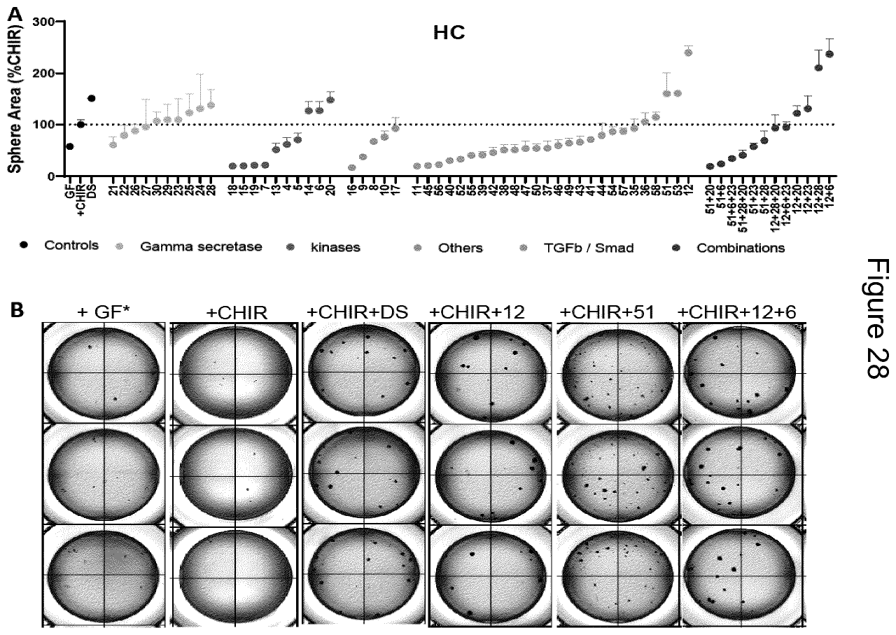

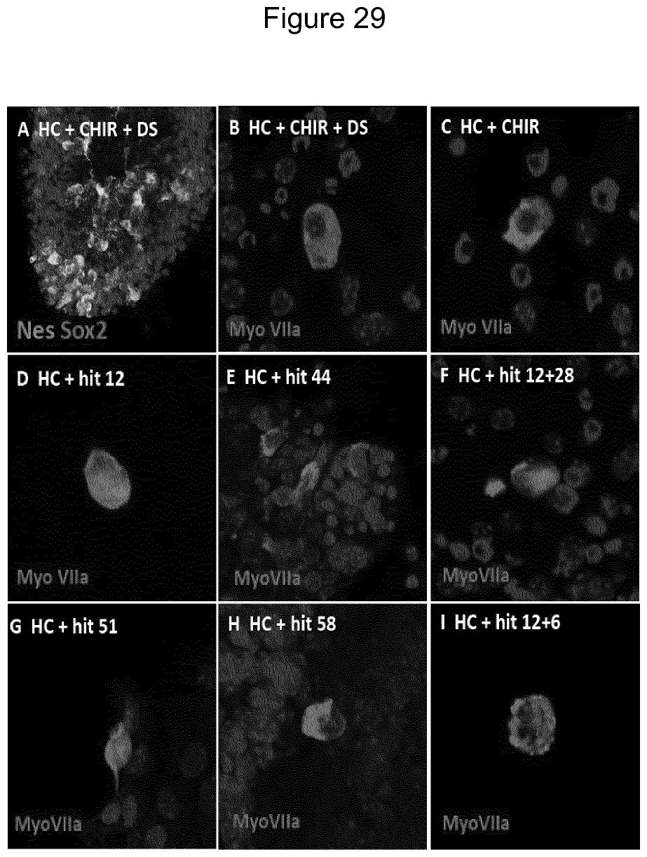

For reprogramming stemness of auditory hair cells, C57Bl/6 otic progenitors were plated at 10,000 cells/well of a 96 well plate and treated with WNT agonist (CHIR99021; 3 μM) and the above mentioned compounds for 30 days. Data is presented in FIG. 28. Further, and as shown in FIG. 29, hair cell progenitors sternness reprogramming did not alter their ability to differentiate into myosin 7a expressing auditory hair cells.

### Example 11—Stemness-Induced Cells can be Used as a Screening Platform to Assess Neurogenesis, Including in the Context of a High-Throughput Screening Platform

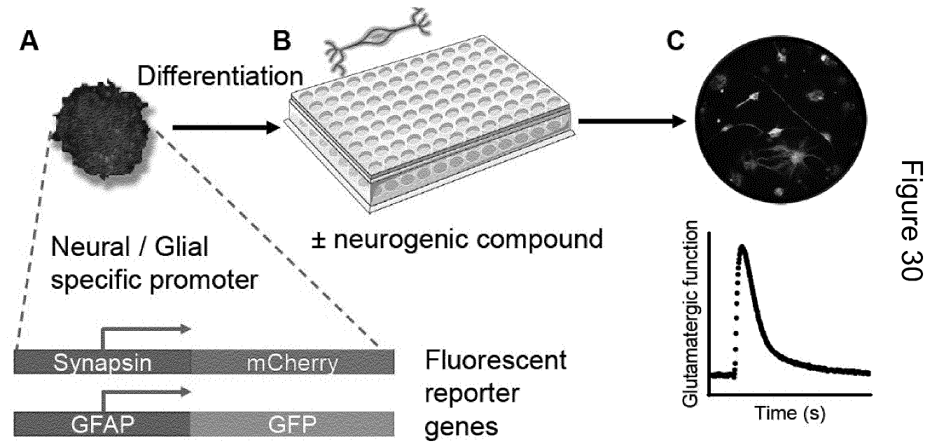

Stemness-Induced ANPGs were transduced with reporter genes and allowing both neurogenesis (mCherry) and gliogenesis (GFP) to be followed in living cells. As set out in FIG. 30, stemness-Induced ANPGs were differentiated in a 96 well plate±neurogenic compounds. Readouts included both fluorescence and functional assays on a single experimental plate.

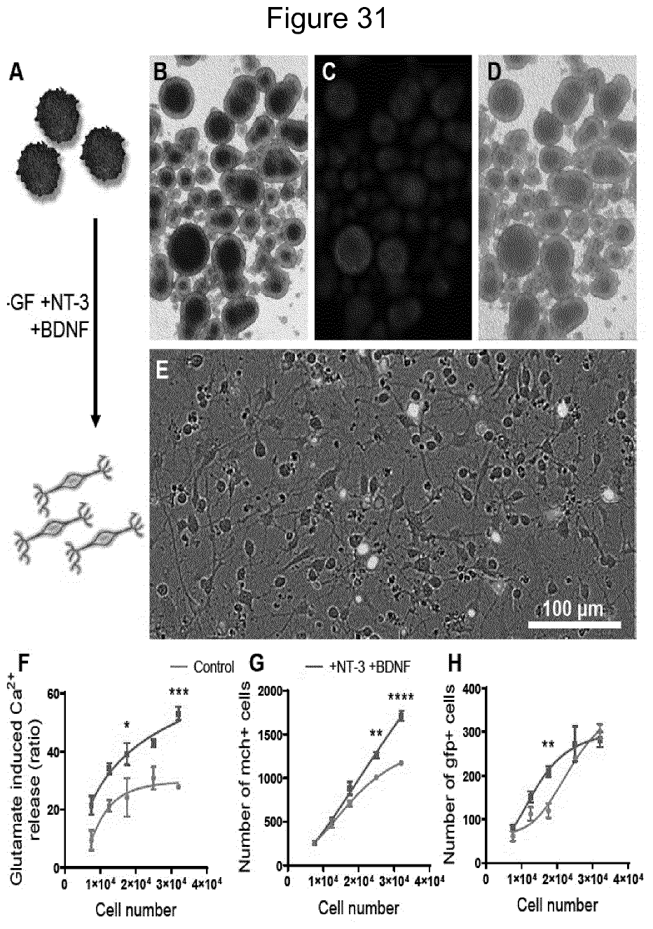

Stemness-induced ANPGs transduced with Synapsin-mCherry and GFAP-GFP reporter genes were differentiated in a 96 well plate±NT-3 (50 ng/mL) and BDNF (10 ng/mL). In undifferentiated ANPGs, virtually no or very few reporter gene expression was visualized. However, upon differentiation, mcherry was expressed in neural cell and visualized in glial cells (FIG. 31A-E). Upon neurotrophin treatment, significant differences were also observed regarding the glutamatergic function and neurogenesis, whereas virtually no impact is observed on the Gliogenesis (FIG. 31F-H).

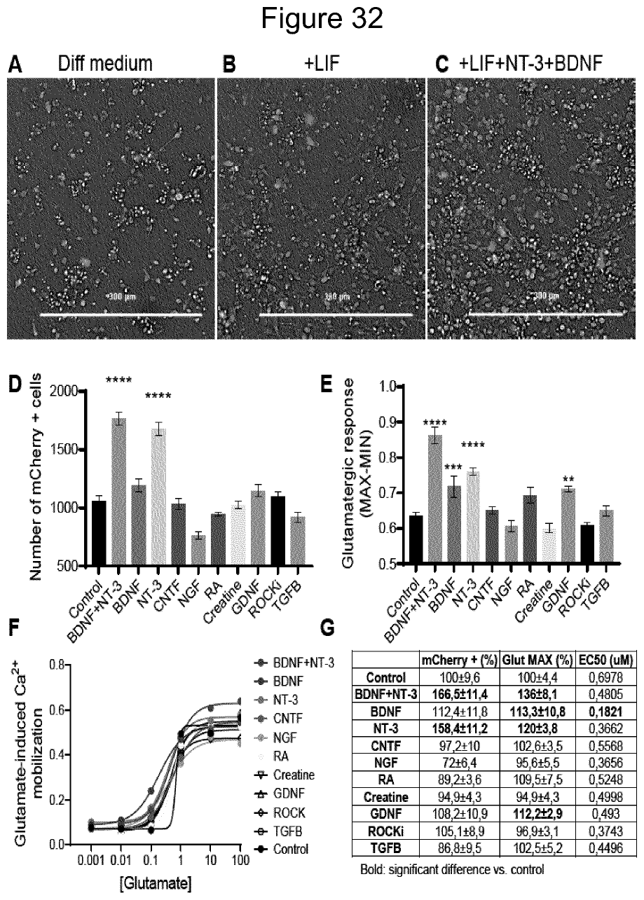

An “all-in-one” phoenix platform was also developed. Phoenix ANPGs were differentiated in a 96 well plate±10 compounds with potent neurogenic activity for 7 days. The neurogenic potential of the compounds was assessed following mCherry fluorescence (expressed upon the SYN promoter). The results in FIG. 32 demonstrate that phoenix platform can be further used for screening, including for high-throughput screening.
Bixenstine, P. J., Maniglia, M. P., Vasanji, A., Alagramam, K. N., and Megerian, C. A. (2008). Spiral ganglion degeneration patterns in endolymphatic hydrops. Laryngoscope 118, 1217-1223. doi: 10.1097/MLG.0b013e31816ba9cd
Chen, W., Jongkamonwiwat, N., Abbas, L., Eshtan, S. J., Johnson, S. L., Kuhn, S., et al. (2012). Restoration of auditory evoked responses by human ES-cell-derived otic progenitors. Nature 490, 278-282. doi: 10.1038/naturel 1415
Corwin, J. T., and Cotanche, D. A. (1988). Regeneration of sensory hair cells after acoustic trauma. Science 240, 1772-1774. doi: 10.1126/science.3381100
Kujawa, S. G., and Liberman, M. C. (2009). Adding insult to injury: cochlear nerve degeneration after “temporary” noise-induced hearing loss. J. Neurosci. 29, 14077-14085. doi: 10.1523/JNEUROSCL2845-09.2009
Li, X., Aleardi, A., Wang, J., Zhou, Y., Andrade, R., and Hu, Z. (2016). Differentiation of spiral ganglion-derived neural stem cells into functional synaptogenetic neurons. Stem Cells Dev. 25, 803-813. doi: 10.1089/scd.2015.0345
Martinez-Monedero, R., Yi, E., Oshima, K., Glowatzki, E., and Edge, A. S. B. (2008). Differentiation of inner ear stem cells to functional sensory neurons. Dev. Neurobiol. 68, 669-684. doi: 10.1002/dneu.20616
Merico, D., Isserlin, R., Stueker, O. et al. (2010). Enrichment map: A network-based method for gene-set enrichment visualization and interpretation. PLoS One 5, e13984. doi:10.1371/journal.pone.0013984
Michelson, R. P., Merzenich, M. M., Pettit, C. R., and Schindler, R. A. (1973). A cochlear prosthesis: further clinical observations; preliminary results of physiological studies. Laryngoscope 83, 1116-1122. doi: 10.1288/00005537-197307000-00015
Moon, B.-S., Ammothumkandy, A., Zhang, N., Peng, L., Ibrayeva, A., Bay, M., et al. (2018). The presence of neural stem cells and changes in stem cell-like activity with age in mouse spiral ganglion cells in vivo and in vitro. Clin. Exp. Otorhinolaryngol. 11, 224-232. doi: 10.21053/ceo.2018.00878
Moser, T., and Starr, A. (2016). Auditory neuropathy-neural and synaptic mechanisms. Nat. Rev. Neurol. 12, 135-149. doi: 10.1038/nrneurol.2016.10
Olusanya, B. O., Davis, A. C., and Hoffman, H. J. (2019). Hearing loss: rising prevalence and impact. Bull. World Health Organ. 97, 646A-646A. doi: 10.2471/BLT.19.224683
Oshima, K., Grimm, C. M., Corrales, C. E., Senn, P., Martinez Monedero, R., Geleoc, G. S., et al. (2007). Differential distribution of stem cells in the auditory and vestibular organs of the inner ear. J. Assoc. Res. Otolaryngol. 8, 18-31. doi: 10.1007/sl0162-006-0058-3
Oshima, K., Senn, P. and Heller, S. (2009). Isolation of sphere-forming stem cells from the mouse inner ear. Methods Mol Biol 493, 141-162. doi:10.1007/978-1-59745-523-7 9
Reimand, J., Isserlin, R., Voisin, V. et al. (2019). Pathway enrichment analysis and visualization of omics data using g:Profiler, gsea, cytoscape and enrichmentmap. Nat Protoc 14, 482-517. doi:10.1038/s41596-018-0103-9
Roccio, M., Pemy, M., Ealy, M., Widmer, H. R., Heller, S., and Senn, P. (2018). Molecular characterization and prospective isolation of human fetal cochlear hair cell progenitors. Nat. Commun. 9:4027. doi: 10.1038/s41467-018-06334-7
Rousset, F., Nacher-Soler, G., Coelho, M., Ilmjarv, S., Kokje, V. B. C., Marteyn, A., et al. (2020). Redox activation of excitatory pathways in auditory neurons as mechanism of age-related hearing loss. Redox Biol. 30:101434. doi: 10.1016/j.redox.2020.101434
Rousset F, B C Kokje V, Sipione R, Schmidbauer D, Nacher-Soler G, Ilmjarv S, Coelho M, Fink S, Voruz F, El Chemaly A, Marteyn A, Lowenheim H, Krause K H, Muller M, Glnckert R, Senn P. Intrinsically Self-renewing Neuroprogenitors From the A/J Mouse Spiral Ganglion as Virtually Unlimited Source of Mature Auditory Neurons. Front Cell Neurosci. 2020 Dec. 9; 14:395. doi: 10.3389/fncel.2020.599152
Ryals, B. M., and Rubel, E. W. (1988). Hair cell regeneration after acoustic trauma in adult Coturnix quail. Science 240, 1774-1776. doi: 10.1126/science.3381101
Senn, P., Oshima, K., Teo, D., Grimm, C., and Heller, S. (2007). Robust postmortem survival of murine vestibular and cochlear stem cells. J. Assoc. Res. Otolaryngol. 8, 194-204. doi: 10.1007/s10162-007-0079-6
Shannon, P., Markiel, A., Ozier, O. et al. (2003). Cytoscape: A software environment for integrated models of biomolecular interaction networks. Genome Res 13, 2498-2504. doi:10.1101/gr.1239303
Vartiainen, E., Karjalainen, S., and Karja, J. (1985). Auditory disorders following head injury in children. Acta Otolaryngol. 99, 529-536. doi: 10.3109/00016488509182257
Warchol, M. E., Lambert, P. R., Goldstein, B. J., Forge, A., and Corwin, J. T. (1993). Regenerative proliferation in inner ear sensory epithelia from adult guinea pigs and humans. Science 259, 1619-1622. doi: 10.1126/science.8456285
Xiong, B., Liu, Z., Liu, Q., Peng, Y., Wu, H., Lin, Y., et al. (2019). Missed hearing loss in tinnitus patients with normal audiograms. Hear. Res. 384:107826. doi: 10.1016/j.heares.2019.107826

1-15. (canceled)

16. An auditory neuroprogenitor (ANPG) cell, wherein the ANPG cell:
(a) is capable of self-renewal for at least 10 passages, at least 20 passages, at least 30 passages or at least 40 passages;
(b) is capable of differentiating into an auditory neuron and/or glial cell; and/or
(c) maintains self-renewal and/or differentiation capacity after freeze-thawing;
further wherein the ANPG cell comprises:
(d) an overexpression of one or more genes selected from Myc, Sox2, Lgr5, Wnt7a, Wnt7b, Bmi1, Rtkn2 and a gene set out in Table 1; and/or
(e) an underexpression of one or more genes selected from Lgr6, Frb, Nkd2, Tgfbr2, Tgfbr3, Hmox1, Cyba, Sox10, Plp1, Lpr5, dkk3, Nrf2 and a gene set out in Table 2.

17. The ANPG cell according to claim 16 wherein the ANPG cell:
(a) is a human or mouse cell; and/or
(b) is isolated from a spiral ganglion; and/or
(c) is isolated from a spiral ganglion, wherein the spiral ganglion is from an A.B6 Tyr⁺-Cyba^{nmf333}/J (A/J) mouse; and/or
(d) is any progenitor cell that is capable of differentiating into one or more cells of the auditory system, such as a cell selected from an auditory progenitor such as a human fetal auditory progenitor, an auditory neuron, a sensory epithelial cell, a spiral ganglion neuron (SGN), a sensory epithelium of the cochlea and/or a hair cell (HC).

18. A stem cell or progenitor cell capable of differentiating into an ANPG cell according to claim 16.

19. The stem cell or progenitor cell according to claim 18, wherein the stem cell or progenitor cell is a human or mouse cell.

20. An ANPG cell deposited under ATCC accession number PTA-127156.

21. The stem cell, progenitor cell or ANPG cell according to claim 18, wherein the stem cell, progenitor cell or ANPG:
(a) is genetically transformed; or
(b) is not genetically transformed.

22. A neurosphere comprising an ANPG cell according to claim 16.

23. The neurosphere comprising an ANPG cell according to claim 22, wherein the ANPG cell or neurosphere exhibits dose-dependent Ca²⁺ mobilisation in response to Glutamate, ATP, Ionomycin and/or Thapsigargin.

24. A method of obtaining, maintaining and/or expanding an ANPG cell or neurosphere according to claim 22, comprising:
(a) differentiating the stem cell or progenitor cell of claim 3 into an ANPG cell or neurosphere in a first culture medium; and/or
(b) isolating an ANPG cell from a spiral ganglion.

25. The method according to claim 24, wherein:
(a) the spiral ganglion is from an A.B6 Tyr⁺-Cyba^{nmf333}/J (A/J) mouse, and culturing the isolated ANPG cell in a first culture medium, and/or
(b) the first culture medium contains:
i. a WNT agonist such as CHIR99021, and a TGFβ antagonist such as LDN193189 and/or SB431542; or
ii. a WNT agonist such as CHIR99021, and at least one modulator selected from Isoxazole9 (ISX-9), Halofuginone, Sulfasalazine, AUDA, FPS-ZM1, CGP 57380, PD 169316, TA-02, Sorafenib Tosylate, Deguelin, Bosutinib (SKI-606), Ponatinib (AP24534), Axitinib, Sunitinib Malate, Imatinib (STI571), Dorsomorphin 2HCl, Dorsomorphin, L-Quebrachitol, A-83-01, TP0427736 HCl, SB431542, SB525334, SB505124, Galunisertib, GW788388, Pirfenidone, DMH1, LDN-212854, ML347, RepSox, K02288, Vactosertib, SD-208, LDN-214117, SIS3 HCl, LY 3200882, ITD-1, BIBF-0775, LDN-193189 2HCl, LY2109761, LY364947, L-685458, Nirogacestat, Avagacestat, Semagacestat, DAPT (GSI-IX), MK-0752, Dibenzazepine, Crenigacestat, LY411575, MDL-28170 and any combination thereof; or
iii. at least one modulator selected from Isoxazole9 (ISX-9), Halofuginone, Sulfasalazine, AUDA, FPS-ZM1, CGP 57380, PD 169316, TA-02, Sorafenib Tosylate, Deguelin, Bosutinib (SKI-606), Ponatinib (AP24534), Axitinib, Sunitinib Malate, Imatinib (STI571), Dorsomorphin 2HCl, Dorsomorphin, L-Quebrachitol, A-83-01, TP0427736 HCl, SB431542, SB525334, SB505124, Galunisertib, GW788388, Pirfenidone, DMH1, LDN-212854, ML347, RepSox, K02288, Vactosertib, SD-208, LDN-214117, SIS3 HCl, LY 3200882, ITD-1, BIBF-0775, LDN-193189 2HCl, LY2109761, LY364947, L-685458, Nirogacestat, Avagacestat, Semagacestat, DAPT (GSI-IX), MK-0752, Dibenzazepine, Crenigacestat, LY411575, MDL-28170 and any combination thereof; and/or
(c) the first culture medium contains at least one growth factor; and/or
(d) the first culture medium contains at least one growth factor wherein:
i. the at least one growth factor is a mitogenic growth factor; and/or
ii. the at least one growth factor is selected from one or more of FGF, EGF, IGF and Heparan Sulfate.

26. The method according to claim 24, further comprising:
(a) freezing the ANPG cell or neurosphere; or
(b) freezing the ANPG cell or neurosphere and thawing the frozen ANPG cell or neurosphere.

27. A method of obtaining an auditory neuron and/or glial cell, comprising differentiating a culture of an ANPG cell or neurosphere according claim 22.

28. The method according to claim 27, wherein the ANPG cell or neurosphere is cultured in a second culture medium, further wherein:
(a) the concentration of a growth factor in the second culture medium is less than the concentration of the same growth factor in the first culture medium according to claim 9; and/or
(b) the second culture medium comprises leukemia inhibitory factor (LIF) and/or one or more neurotrophinin and/or
(c) the second culture medium comprises leukemia inhibitory factor (LIF) and/or one or more neurotrophinin, wherein the neurotrophinin is selected from one or more of nerve growth factor (NGF), brain-derived neurotrophic factor (BDNF), neurotrophin-3 (NT-3) and neurotrophin-4 (NT-4).

29. An ANPG cell or neurosphere, obtainable by the method according to claim 24.

30. An auditory neuron or glial cell obtainable by the method according to claim 27.

31. A method for treating or preventing hearing loss in a subject in need thereof, said method comprising administering an effective amount of:
(a) an ANPG cell according to claim 16 or a neurosphere comprising the ANPG cell;
(b) a stem cell or a progenitor cell, wherein the stem cell or progenitor cell is capable of differentiating into the ANPG cell; or
(c) an auditory neuron or a glial cell differentiated from the ANPG cell.

32. The method according to claim 31, wherein:
(a) the hearing loss is sensorineural hearing loss; or
(b) the hearing loss is sensorineural hearing loss, wherein sensory epithelia, sensory epithelium of the cochlea, olfactory epithelium and/or spiral ganglion neurons are generated and/or regenerated.

33. A non-human organism comprising or treated with:
(a) an ANPG cell according to claim 16 or a neurosphere comprising the ANPG cell;
(b) a stem cell or a progenitor cell, wherein the stem cell or progenitor cell is capable of differentiating into the ANPG cell; or
(c) an auditory neuron or a glial cell differentiated from the ANPG cell.

34. The non-human organism according to claim 33, wherein the non-human organism is a murine organism.
